(19) United States
(12) Patent Application Publication
(10) Pub. No.: US 2003/0109670 A1
(43) Pub. Date: Jun. 12, 2003

(54) Cone snail peptides

(75) Inventors: Baldomero M. Olivera, Salt Lake City, UT (US); J. Michael McIntosh, Salt Lake City, UT (US); Maren Watkins, Salt Lake City, UT (US); James E. Garrett, Salt Lake City, UT (US); Lourdes J. Cruz, Manila (PH); Michelle Grilley, Logan, UT (US); Robert A. Schoenfeld, Sacramento, CA (US); Craig S. Walker, Salt Lake City, UT (US); Reshma P. Shetty, Salt Lake City, UT (US); Robert M. Jones, Park City, UT (US)

Correspondence Address: ROTHWELL, FIGG, ERNST & MANBECK, P.C., 1425 K STREET, N.W., SUITE 800, WASHINGTON, DC, 20005, US

(73) Assignee: University of Utah Research Foundation, Salt Lake City, UT

(21) Appl. No.: 10/072,602

(22) Filed: Feb. 11, 2002

Related U.S. Application Data
(60) Provisional application No. 60/267,408, filed on Feb. 9, 2001.

(51) Int. Cl. A61K 38/00; C07K 5/00; C07K 16/00; C07K 7/00; C07K 17/00; C07H 21/04; C12N 1/20; C12N 15/00; C12N 15/09; C12N 15/63; C12N 15/70; C12N 15/74; C12N 5/00; C12N 5/02

(52) U.S. Cl. 530/324; 514/12; 536/23.5; 435/320.1; 435/252.3; 435/325

(57) ABSTRACT

The present invention is directed to conotoxin peptides, derivatives or pharmaceutically acceptable salts thereof. The present invention is further directed to the use of this peptide, derivatives thereof and pharmaceutically acceptable salts thereof for the treatment of disorders associated with voltage-gated ion channels, voltage-gated ligand channels and/or receptors. The invention is further directed to nucleic acid sequences encoding the conotoxin peptides and encoding propeptides, as well as the propeptides.

### CROSS-REFERENCE TO RELATED APPLICATIONS

The present application is related to and claims priority under 35 USC §119(e) to U.S. provisional patent application Serial No. 60/267,408 filed Feb. 9, 2001, incorporated herein by reference.

This invention was made with Government support under Grant No. PO1 GM48677 awarded by the National Institute of General Medical Sciences, National Institutes of Health, Bethesda, Md. The United States Government has certain rights in the invention.

### BACKGROUND OF THE INVENTION

The present invention is directed to conotoxin peptides, derivatives or pharmaceutically acceptable salts thereof. The present invention is further directed to the use of this peptide, derivatives thereof and pharmaceutically acceptable salts thereof for the treatment of disorders associated with voltage-gated ion channels, ligand-gated ion channels and/or receptors. The invention is further directed to nucleic acid sequences encoding the conotoxin peptides and encoding propeptides, as well as the propeptides.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

Conus is a genus of predatory marine gastropods (snails) which envenomate their prey. Venomous cone snails use a highly developed projectile apparatus to deliver their cocktail of toxic conotoxins into their prey. In fish-eating species such as Conus magus the cone detects the presence of the fish using chemosensors in its siphon and when close enough extends its proboscis and fires a hollow harpoon-like tooth containing venom into the fish. This immobilizes the fish and enables the cone snail to wind it into its mouth via an attached filament. For general information on Conus and their venom see the website address http://grimwade.biochem.unimelb.edu.au/cone/referenc.html. Prey capture is accomplished through a sophisticated arsenal of peptides which target specific ion channel and receptor subtypes. Each Conus species venom appears to contain a unique set of 50-200 peptides. The composition of the venom differs greatly between species and between individual snails within each species, each optimally evolved to paralyse it's prey. The active components of the venom are small peptides toxins, typically 12-30 amino acid residues in length and are typically highly constrained peptides due to their high density of disulphide bonds.

The venoms consist of a large number of different peptide components that when separated exhibit a range of biological activities: when injected into mice they elicit a range of physiological responses from shaking to depression. The paralytic components of the venom that have been the focus of recent investigation are the α-, ω- and μ-conotoxins. All of these conotoxins act by preventing neuronal communication, but each targets a different aspect of the process to achieve this. The α-conotoxins target nicotinic ligand gated channels, the μ-conotoxins target the voltage-gated sodium channels and the ω-conotoxins target the voltage-gated calcium channels (Olivera et al., 1985; Olivera et al., 1990). For example a linkage has been established between α-, αA- & φ-conotoxins and the nicotinic ligand-gated ion channel; ω-conotoxins and the voltage-gated calcium channel; κ-conotoxins and the voltage-gated sodium channel; δ-conotoxins and the voltage-gated sodium channel; κ-conotoxins and the voltage-gated potassium channel; conantokins and the ligand-gated glutamate (NMDA) channel.

However, the structure and function of only a small minority of these peptides have been determined to date. For peptides where function has been determined, three classes of targets have been elucidated: voltage-gated ion channels; ligand-gated ion channels, and G-protein-linked receptors.

Conus peptides which target voltage-gated ion channels include those that delay the inactivation of sodium channels, as well as blockers specific for sodium channels, calcium channels and potassium channels. Peptides that target ligand-gated ion channels include antagonists of NMDA and serotonin receptors, as well as competitive and noncompetitive nicotinic receptor antagonists. Peptides which act on G-protein receptors include neurotensin and vasopressin receptor agonists. The unprecedented pharmaceutical selectivity of conotoxins is at least in part defined by a specific disulfide bond frameworks combined with hypervariable amino acids within disulfide loops (for a review see McIntosh et al., 1998).

There are drugs used in the treatment of pain, which are known in the literature and to the skilled artisan. See, for example, Merck Manual, 16th Ed. (1992). However, there is a demand for more active analgesic agents with diminished side effects and toxicity and which are non-addictive. The ideal analgesic would reduce the awareness of pain, produce analgesia over a wide range of pain types, act satisfactorily whether given orally or parenterally, produce minimal or no side effects, be free from tendency to produce tolerance and drug dependence.

Due to the high potency and exquisite selectivity of the conopeptides, several are in various stages of clinical development for treatment of human disorders. For example, two Conus peptides are being developed for the treatment of pain. The most advanced is ω-conotoxin MVIIA (ziconotide), an N-type calcium channel blocker (see Heading, C., 1999; U.S. Pat. No. 5,859,186). (ω-Conotoxin MVIIA, isolated from Conus magus, is approximately 1000 times more potent than morphine, yet does not produce the tolerance or addictive properties of opiates. ω-Conotoxin MVIIA has completed Phase III (final stages) of human clinical trials and has been approved as a therapeutic agent. ω-Conotoxin MVIIA is introduced into human patients by means of an implantable, programmable pump with a catheter threaded into the intrathecal space. Preclinical testing for use in post-surgical pain is being carried out on another Conus peptide, contulakin-G, isolated from Conus geographus (Craig et al. 1999). Contulakin-G is a 16 amino acid O-linked glycopeptide whose C-terminus resembles neurotensin. It is an agonist of neurotensin receptors, but appears significantly more potent than neurotensin in inhibiting pain in in vivo assays.

In view of a large number of biologically active substances in Conus species it is desirable to further characterize them and to identify peptides capable of treating disorders voltage-gated ion channels, ligand-gated ion channels and/or receptors. Surprisingly, and in accordance with this invention, Applicants have discovered novel conotoxins that can be useful for the treatment of disorders involving voltage-gated ion channels, ligand-gated ion channels and/or receptors and could address a long felt need for a safe and effective treatment.

### SUMMARY OF THE INVENTION

The present invention is directed to conotoxin peptides, derivatives or pharmaceutically acceptable salts thereof. The present invention is further directed to the use of this peptide, derivatives thereof and pharmaceutically acceptable salts thereof for the treatment of disorders associated with voltage-gated ion channels, ligand-gated ion channels and/or receptors. The invention is further directed to nucleic acid sequences encoding the conotoxin peptides and encoding propeptides, as well as the propeptides.

More specifically, the present invention is directed to conotoxin peptides, having the amino acid sequences set forth in Tables 1-14 below.

The present invention is also directed to derivatives or pharmaceutically acceptable salts of the conotoxin peptides or the derivatives. Examples of derivatives include peptides in which the Arg residues may be substituted by Lys, omithine, homoargine, nor-Lys, N-methyl-Lys, N,N-dimethyl-Lys, N,N,N-trimethyl-Lys or any synthetic basic amino acid; the Lys residues may be substituted by Arg, omithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; and the Asn, Ser, Thr or Hyp residues may be glycosylated. The halogen may be iodo, chloro, fluoro or bromo; preferably iodo for halogen substituted-Tyr and bromo for halogen-substituted Trp. The Tyr residues may also be substituted with the 3-hydroxyl or 2-hydroxyl isomers (meta-Tyr or ortho-Tyr, respectively) and corresponding O-sulpho- and O-phospho-derivatives. The acidic amino acid residues may be substituted with any synthetic acidic amino acid, e.g., tetrazolyl derivatives of Gly and Ala. The aliphatic amino acids may be substituted by synthetic derivatives bearing non-natural aliphatic branched or linear side chains CₙH₂ₙ₊₂ up to and including n=8. The Leu residues may be substituted with Leu (D). The Glu residues may be substituted with Gla. The Gla residues may be substituted with Glu. The N-terminal Gln residues may be substituted with pyroGlu. The Met residues may be substituted with norleucine (Nle). The Cys residues may be in D or L configuration and may optionally be substituted with homocysteine (D or L).

Examples of synthetic aromatic amino acid include, but are not limited to, nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acid, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)]-Gly and 2-[3-(2S)pyrrolininyl)]-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also http://www.amino-acids.com), incorporated herein by reference, by and available from RSP Amino Acid Analogues, Inc., Worcester, Mass. The residues containing protecting groups are deprotected using conventional techniques. Examples of synthetic acid amino acids include those derivatives bearing acidic functionality, including carboxyl, phosphate, sulfonate and synthetic tetrazolyl derivatives such as described by Ornstein et al. (1993) and in U.S. Pat. No. 5,331,001, each incorporated herein by reference, and such as shown in the following schemes 1-3.

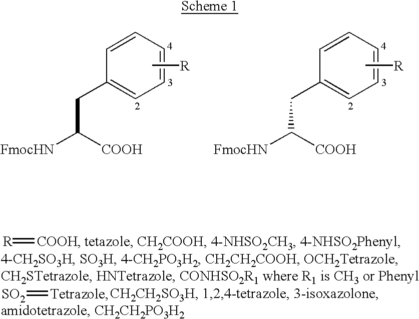

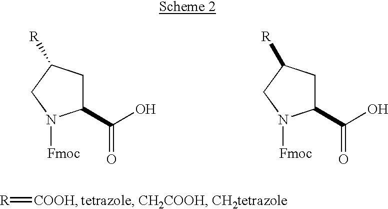

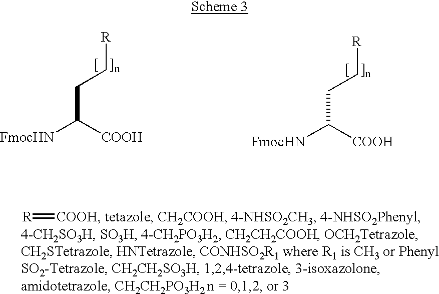

Optionally, in the conotoxin peptides of the present invention, the Asn residues may be modified to contain an N-glycan and the Ser, Thr and Hyp residues may be modified to contain an O-glycan (e.g., g-N, g-S, g-T and g-Hyp). In accordance with the present invention, a glycan shall mean any N-, S- or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The gylcan may also include similar polyhydroxy groups, such as D-penicillamine 2,5 and halogenated derivatives thereof or polypropylene glycol derivatives. The glycosidic linkage is beta and 1-4 or 1-3, preferably 1-3. The linkage between the glycan and the amino acid may be alpha or beta, preferably alpha and is 1-.

Core O-glycans have been described by Van de Steen et al. (1998), incorporated herein by reference. Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have been identified. The type of glycosidic linkage (orientation and connectivities) are defined for each core glycan. Suitable glycans and glycan analogs are described further in U.S. Ser. No. 09/420,797 filed Oct. 19, 1999 and in PCT Application No. PCT/US99/24380 filed Oct. 19, 1999 (PCT Published Application No. WO 00/23092), each incorporated herein by reference. A preferred glycan is Gal(β1 →3GalNAc(β1→).

Optionally, in the conotoxin peptides described above, pairs of Cys residues may be replaced pairwise with isoteric lactam or ester-thioether replacements, such as Ser/(Glu or Asp), Lys/(Glu or Asp), Cys/(Glu or Asp) or Cys/Ala combinations. Sequential coupling by known methods (Barnay et al., 2000; Hruby et al., 1994; Bitan et al., 1997) allows replacement of native Cys bridges with lactam bridges. Thioether analogs may be readily synthesized using halo-Ala residues commercially available from RSP Amino Acid Analogues. In addition, individual Cys residues may be replaced with homoCys, seleno-Cys or penicillamine, so that disulfide bridges may be formed between Cys-homoCys or Cys-penicillamine, or homoCys-penicllamine and the like.

The present invention is further directed to derivatives of the above peptides and peptide derivatives which are acylic permutations in which the cyclic permutants retain the native bridging pattern of native toxin. See, Craik et al. (2001).

The present invention is further directed to a method of treating disorders associated with voltage-gated ion channels, ligand-gated ion channels and/or receptor disorders in a subject comprising administering to the subject an effective amount of the pharmaceutical composition comprising a therapeutically effective amount of a conotoxin peptide described herein or a pharmaceutically acceptable salt or solvate thereof. The present invention is also directed to a pharmaceutical composition comprising a therapeutically effective amount of a conotoxin peptide described herein or a pharmaceutically acceptable salt or solvate thereof and a pharmaceutically acceptable carrier.

More specifically, the present invention is also directed to nucleic acids which encode conotoxin peptides of the present invention or which encodes precursor peptides for these conotoxin peptides, as well as the precursor peptide. The nucleic acid sequences encoding the precursor peptides of other conotoxin peptides of the present invention are set forth in Table 1. Table 1 also sets forth the amino acid sequences of these precursor peptides.

Another embodiment of the invention contemplates a method of identifying compounds that mimic the therapeutic activity of the instant peptide, comprising the steps of: (a) conducting a biological assay on a test compound to determine the therapeutic activity; and (b) comparing the results obtained from the biological assay of the test compound to the results obtained from the biological assay of the peptide. The peptide is labeled with any conventional label, preferably a radioiodine on an available Tyr. Thus, the invention is also directed to radioiodinated conotoxins.

### DETAILED DESCRIPTION OF THE PREFERRED EMBODIMENT

The present invention is directed to conotoxin peptides, derivatives or pharmaceutically acceptable salts thereof. The present invention is further directed to the use of this peptide, derivatives thereof and pharmaceutically acceptable salts thereof for the treatment of disorders associated with voltage-gated ion channels, ligand-gated ion channels and/or receptors. The invention is further directed to nucleic acid sequences encoding the conotoxin peptides and encoding propeptides, as well as the propeptides.

The present invention, in another aspect, relates to a pharmaceutical composition comprising an effective amount of a conotoxin peptides, a mutein thereof, an analog thereof, an active fragment thereof or pharmaceutically acceptable salts or solvates. Such a pharmaceutical composition has the capability of acting at voltage-gated ion channels, ligand-gated ion channels and/or receptors, and are thus useful for treating a disorder or disease of a living animal body, including a human, which disorder or disease is responsive to the partial or complete blockade of such channels or receptors comprising the step of administering to such a living animal body, including a human, in need thereof a therapeutically effective amount of a pharmaceutical composition of the present invention.

Examples of voltage-gated ion channels include the voltage-gated calcium channel, the voltage-gated sodium channel, the voltage-gated potassium channel and the proton-gated ion channel. Examples of ligand-gated channels include the nicotinic ligand-gated ion channel, ligand-gated glutamate (NMDA) channel and the ligand-gated 5HT₃ (serotonin) channel. Examples of receptors include the G-protein receptors. Activity of ψ-conotoxins is described in U.S. Pat. No. 5,969,096 and in Shon et al. (1997). Activity of bromosleeper conotoxins is described in U.S. Pat. No. 5,889,147 and in Craig et al. (1997). Activity of σ-conotoxins is described in U.S. Pat. No. 5,889,147. Activity of contryphan conotoxins is described in U.S. Pat. No. 6,077,934 and in Jimenez et al. (1996). Activity of conopressins is described in Cruz et al. (1987) and in Kruszynski et al. (1990). Activity of γ-conotoxins is described in Fainzilber et al. (1998). Activity of αA-conotoxins (kappaA??) is described in Jacobsen et al. (1997) and in Hopkins et al. (1995). Activity of α-conotoxins is described in U.S. Pat. Nos. 4,447,356 and 5,514,774. Activity of τ-conotoxins is described in U.S. Ser. No. 09/497,491 (PCT/US00/03021, PCT published application WO 00/46371) as an antagonist for acetylcholine receptors and as analgesic agents for the treatment of pain (whether acute or chronic), including migraine, chronic pain, and neuropathic pain, without undesirable side effects. Activity of contulakins is described in U.S. Ser. No. 09/420,797 (PCT/US99/24380, PCT published application WO 00/23092). Each of these references is incorporated herein by reference.

Since σ-conotoxins are antagonists of the 5HT₃ receptor, they are also useful in treating irritable bowel syndrome (IBS) and visceral pain. Visceral pain is a common experience in health and disease. Chronic visceral hyperalgesia in the absence of detectable organic disease has been implicated in many common functional bowel disorders (FDB), such as IBS, non-ulcer dyspepsia (NUD) and non-cardiac chest pain (NCCP).

Pain in IBS cannot be explained by normal perception of abnormal motility. In the majority of patients, sensory perception itself is abnormal. Most visceral afferent information is part of the reflex activity of digestion and does not reach concious perception. Increasing evidence suggests that long term changes in the the thresholds and gain of the visceral afferent pathways are present in patients with FDBs. This has been referred to as visceral hyperalgesia (Mayer et al., 1994).

It has been proposed that FDBs are a result of increased excitability of spinal neurones. According to their model, many inputs can result in transient, short term, or life long sensitization of afferent pathways involved in visceral reflexes and sensations from the gut. The increased sensory input to interneurons and/or dorsal horn neurons in the spinal cord will result in secondary hyperalgesia, in which adjacent, undamaged viscera develop sensitivity to normal innocuous stimuli (allodynia), and central hyperexcitability as a consequence of changes in the circuitary of the dorsal horn. This central sensitization may subsequently extend to supraspinal centers also.

Altered spinal processing of visceral sensory information can explain altered sensory thresholds and altered referral patterns, the perception of visceral sensations without stimulation of visceral mechnoreceptors (sensation of incomplete evacuation), and the symptomatic involvement of multiple sites in the GI tract, including extra intestinal sites. Increased excitability of dorsal horn neurones, resulting in the recruitment of previously sub-threshold inputs, may explain cutaneous allodynia in some patients with IBS, burning sensations referred to different parts of the body, cold hypersensitivity and pain referral to upper and lower extremities.

A number of compounds have been shown to modulate visceral sensitivity in IBS patients. These include octreotide (sst₂; Novartis), the 5-HT₃ antgonists odansetron (Glaxo) and granisetron (SKB) and the peripheral kappa opioid agonist, fedotozine (Jouveinal SA). The 5-HT₃ antagonist alosteron (Glaxo), cuurrently in development for IBS, is active in modifying the perception of colonic distension and gut compliance in IBS patients. New drugs in development for the treatment of IBS that are targeted at pain control as well as dysmotility include 5-HT₃ and 5-HT₄ receptor antagonists. 5-HT₃ receptors are located throughout the central and peripheral nervous system—their role in modulating the activity of visceral afferent and enteric neurones has led to the proposal that 5-HT acts as a sensitizing agent via these receptors on visceral afferent neurones. 5-HT₃ receptor antagonists have been widely reported to attenuate blood pressure responses to intestinal distension. 5-HT₃ antagonists in development for IBS include Alosteron (phase III), which is reported to reduce abdominal pain, slow colonic transit and increase colon compliance in IBS patients. Other compounds with positive effects include the antiemetic Ramosteron (Yamanouchi), Cilansteron (Solvay) and YM-114 (Yamanouchi). An animal model for dysmotility of the GI tract has been described by Maric et al. (1989).

The conotoxin peptides described herein are sufficiently small to be chemically synthesized. General chemical syntheses for preparing the foregoing conotoxin peptides are described hereinafter. Various ones of the conotoxin peptides can also be obtained by isolation and purification from specific Conus species using the technique described in U.S. Pat. Nos. 4,447,356 (Olivera et al., 1984); 5,514,774; 5,719,264; and 5,591,821, as well as in PCT published application WO 98/03189, the disclosures of which are incorporated herein by reference.

Although the conotoxin peptides of the present invention can be obtained by purification from cone snails, because the amounts of conotoxin peptides obtainable from individual snails are very small, the desired substantially pure conotoxin peptides are best practically obtained in commercially valuable amounts by chemical synthesis using solid-phase strategy. For example, the yield from a single cone snail may be about 10 micrograms or less of conotoxin peptides peptide. By “substantially pure” is meant that the peptide is present in the substantial absence of other biological molecules of the same type; it is preferably present in an amount of at least about 85% purity and preferably at least about 95% purity. Chemical synthesis of biologically active conotoxin peptides peptides depends of course upon correct determination of the amino acid sequence.

The conotoxin peptides can also be produced by recombinant DNA techniques well known in the art. Such techniques are described by Sambrook et al. (1989). A gene of interest (i.e., a gene that encodes a suitable conotoxin peptides) can be inserted into a cloning site of a suitable expression vector by using standard techniques. These techniques are well known to those skilled in the art. The expression vector containing the gene of interest may then be used to transfect the desired cell line. Standard transfection techniques such as calcium phosphate co-precipitation, DEAE-dextran transfection or electroporation may be utilized. A wide variety of host/expression vector combinations may be used to express a gene encoding a conotoxin peptide of interest. Such combinations are well known to a skilled artisan. The peptides produced in this manner are isolated, reduced if necessary, and oxidized to form the correct disulfide bonds.

One method of forming disulfide bonds in the conotoxin peptides of the present invention is the air oxidation of the linear peptides for prolonged periods under cold room temperatures or at room temperature. This procedure results in the creation of a substantial amount of the bioactive, disulfide-linked peptides. The oxidized peptides are fractionated using reverse-phase high performance liquid chromatography (HPLC) or the like, to separate peptides having different linked configurations. Thereafter, either by comparing these fractions with the elution of the native material or by using a simple assay, the particular fraction having the correct linkage for maximum biological potency is easily determined. However, because of the dilution resulting from the presence of other fractions of less biopotency, a somewhat higher dosage may be required.

The peptides are synthesized by a suitable method, such as by exclusively solid-phase techniques, by partial solid-phase techniques, by fragment condensation or by classical solution couplings.

In conventional solution phase peptide synthesis, the peptide chain can be prepared by a series of coupling reactions in which constituent amino acids are added to the growing peptide chain in the desired sequence. Use of various coupling reagents, e.g., dicyclohexylcarbodiimide or diisopropylcarbonyldimidazole, various active esters, e.g., esters of N-hydroxyphthalimide or N-hydroxy-succinimide, and the various cleavage reagents, to carry out reaction in solution, with subsequent isolation and purification of intermediates, is well known classical peptide methodology. Classical solution synthesis is described in detail in the treatise, “Methoden der Organischen Chemie (Houben-Weyl): Synthese von Peptiden,” (1974). Techniques of exclusively solid-phase synthesis are set forth in the textbook, “Solid-Phase Peptide Synthesis,” (Stewart and Young, 1969), and are exemplified by the disclosure of U.S. Pat. No. 4,105,603 (Vale et al., 1978). The fragment condensation method of synthesis is exemplified in U.S. Pat. No. 3,972,859 (1976). Other available syntheses are exemplified by U.S. Pat. Nos. 3,842,067 (1974) and 3,862,925 (1975). The synthesis of peptides containing γ-carboxyglutamic acid residues is exemplified by Rivier et al. (1987), Nishiuchi et al. (1993) and Zhou et al. (1996).

Common to such chemical syntheses is the protection of the labile side chain groups of the various amino acid moieties with suitable protecting groups which will prevent a chemical reaction from occurring at that site until the group is ultimately removed. Usually also common is the protection of an α-amino group on an amino acid or a fragment while that entity reacts at the carboxyl group, followed by the selective removal of the α-amino protecting group to allow subsequent reaction to take place at that location. Accordingly, it is common that, as a step in such a synthesis, an intermediate compound is produced which includes each of the amino acid residues located in its desired sequence in the peptide chain with appropriate side-chain protecting groups linked to various ones of the residues having labile side chains.

As far as the selection of a side chain amino protecting group is concerned, generally one is chosen which is not removed during deprotection of the α-amino groups during the synthesis. However, for some amino acids, e.g., His, protection is not generally necessary. In selecting a particular side chain protecting group to be used in the synthesis of the peptides, the following general rules are followed: (a) the protecting group preferably retains its protecting properties and is not split off under coupling conditions, (b) the protecting group should be stable under the reaction conditions selected for removing the α-amino protecting group at each step of the synthesis, and (c) the side chain protecting group must be removable, upon the completion of the synthesis containing the desired amino acid sequence, under reaction conditions that will not undesirably alter the peptide chain.

It should be possible to prepare many, or even all, of these peptides using recombinant DNA technology. However, when peptides are not so prepared, they are preferably prepared using the Merrifield solid-phase synthesis, although other equivalent chemical syntheses known in the art can also be used as previously mentioned. Solid-phase synthesis is commenced from the C-terminus of the peptide by coupling a protected α-amino acid to a suitable resin. Such a starting material can be prepared by attaching an α-amino-protected amino acid by an ester linkage to a chloromethylated resin or a hydroxymethyl resin, or by an amide bond to a benzhydrylamine (BHA) resin or paramethylbenzhydrylamine (MBHA) resin. Preparation of the hydroxymethyl resin is described by Bodansky et al. (1966). Chloromethylated resins are commercially available from Bio Rad Laboratories (Richmond, Calif.) and from Lab. Systems, Inc. The preparation of such a resin is described by Stewart and Young (1969). BHA and MBHA resin supports are commercially available, and are generally used when the desired polypeptide being synthesized has an unsubstituted amide at the C-terminus. Thus, solid resin supports may be any of those known in the art, such as one having the formulae —O—CH₂-resin support, —NH BHA resin support, or —NH—MBHA resin support. When the unsubstituted amide is desired, use of a BHA or MBHA resin is preferred, because cleavage directly gives the amide. In case the N-methyl amide is desired, it can be generated from an N-methyl BHA resin. Should other substituted amides be desired, the teaching of U.S. Pat. No. 4,569,967 (Kornreich et al., 1986) can be used, or should still other groups than the free acid be desired at the C-terminus, it may be preferable to synthesize the peptide using classical methods as set forth in the Houben-Weyl text (1974).

The C-terminal amino acid, protected by Boc or Fmoc and by a side-chain protecting group, if appropriate, can be first coupled to a chloromethylated resin according to the procedure set forth in K. Horiki et al. (1978), using KF in DMF at about 60° C. for 24 hours with stirring, when a peptide having free acid at the C-terminus is to be synthesized. Following the coupling of the BOC-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific α-amino protecting groups may be used as described in Schroder & Lubke (1965).

After removal of the α-amino-protecting group, the remaining α-amino- and side chain-protected amino acids are coupled step-wise in the desired order to obtain the intermediate compound defined hereinbefore, or as an alternative to adding each amino acid separately in the synthesis, some of them may be coupled to one another prior to addition to the solid phase reactor. Selection of an appropriate coupling reagent is within the skill of the art. Particularly suitable as a coupling reagent is N,N′-dicyclohexylcarbodiimide (DCC, DIC, HBTU, HATU, TBTU in the presence of HoBt or HoAt).

The activating reagents used in the solid phase synthesis of the peptides are well known in the peptide art. Examples of suitable activating reagents are carbodiimides, such as N,N′-diisopropylcarbodiimide and N-ethyl-N′-(3-dimethylaminopropyl)carbodiimide. Other activating reagents and their use in peptide coupling are described by Schroder & Lubke (1965) and Kapoor (1970).

Each protected amino acid or amino acid sequence is introduced into the solid-phase reactor in about a twofold or more excess, and the coupling may be carried out in a it medium of dimethylformamide (DMF):CH₂Cl₂ (1:1) or in DMF or CH₂Cl₂ alone. In cases where intermediate coupling occurs, the coupling procedure is repeated before removal of the α-amino protecting group prior to the coupling of the next amino acid. The success of the coupling reaction at each stage of the synthesis, if performed manually, is preferably monitored by the ninhydrin reaction, as described by Kaiser et al. (1970). Coupling reactions can be performed automatically, as on a Beckman 990 automatic synthesizer, using a program such as that reported in Rivier et al. (1978).

After the desired amino acid sequence has been completed, the intermediate peptide can be removed from the resin support by treatment with a reagent, such as liquid hydrogen fluoride or TFA (if using Fmoc chemistry), which not only cleaves the peptide from the resin but also cleaves all remaining side chain protecting groups and also the -amino protecting group at the N-terminus if it was not previously removed to obtain the peptide in the form of the free acid. If Met is present in the sequence, the Boc protecting group is preferably first removed using trifluoroacetic acid (TFA)/ethanedithiol prior to cleaving the peptide from the resin with HF to eliminate potential S-alkylation. When using hydrogen fluoride or TFA for cleaving, one or more scavengers such as anisole, cresol, dimethyl sulfide and methylethyl sulfide are included in the reaction vessel.

Cyclization of the linear peptide is preferably affected, as opposed to cyclizing the peptide while a part of the peptido-resin, to create bonds between Cys residues. To effect such a disulfide cyclizing linkage, fully protected peptide can be cleaved from a hydroxymethylated resin or a chloromethylated resin support by ammonolysis, as is well known the art, to yield the fully protected amide intermediate, which is thereafter suitably cyclized and deprotected. Alternatively, deprotection, as well as cleavage of the peptide from the above resins or a benzhydrylamine (BHA) resin or a methylbenzhydrylamine (MBHA), can take place at 0° C. with hydrofluoric acid (HF) or TFA, followed by oxidation as described above.

The peptides are also synthesized using an automatic synthesizer. Amino acids are sequentially coupled to an MBHA Rink resin (typically 100 mg of resin) beginning at the C-terminus using an Advanced Chemtech 357 Automatic Peptide Synthesizer. Couplings are carried out using 1,3-diisopropylcarbodimide in N-methylpyrrolidinone (NMP) or by 2-(1H-benzotriazole-1-yl)-1,1,3,3-tetramethyluronium hexafluorophosphate (HBTU) and diethylisopropylethylamine (DIEA). The FMOC protecting group is removed by treatment with a 20% solution of piperidine in dimethylformamide(DMF). Resins are subsequently washed with DMF (twice), followed by methanol and NMP.

Muteins, analogs or active fragments, of the foregoing conotoxin peptides are also contemplated here. See, e.g., Hammerland et al. (1992). Derivative muteins, analogs or active fragments of the conotoxin peptides may be synthesized according to known techniques, including conservative amino acid substitutions, such as outlined in U.S. Pat. Nos. 5,545,723 (see particularly col. 2, line 50—col. 3, line 8); 5,534,615 (see particularly col. 19, line 45—col. 22, line 33); and 5,364,769 (see particularly col. 4, line 55—col. 7, line 26), each herein incorporated by reference.

Pharmaceutical compositions containing a compound of the present invention as the active ingredient can be prepared according to conventional pharmaceutical compounding techniques. See, for example, Remington's Pharmaceutical Sciences, 18th Ed. (1990, Mack Publishing Co., Easton, Pa.). Typically, an antagonistic amount of active ingredient will be admixed with a pharmaceutically acceptable carrier. The carrier may take a wide variety of forms depending on the form of preparation desired for administration, e.g., intravenous, oral, parenteral or intrathecally. For examples of delivery methods see U.S. Pat. No. 5,844,077, incorporated herein by reference.

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion.

For example, administration of the active agent according to this invention may be achieved using any suitable delivery means, including:

(a) pump (see, e.g., Luer & Hatton (1993), Zimm et al. (1984) and Ettinger et al. (1978));

(b), microencapsulation (see, e.g., U.S. Pat. Nos. 4,352,883; 4,353,888; and 5,084,350);

(c) continuous release polymer implants (see, e.g., U.S. Pat. No. 4,883,666);

(d) macroencapsulation (see, e.g., U.S. Pat. Nos. 5,284,761, 5,158,881, 4,976,859 and 4,968,733 and published PCT patent applications WO92/19195, WO 95/05452);

(e) naked or unencapsulated cell grafts to the CNS (see, e.g., U.S. Pat. Nos. 5,082,670 and 5,618,531);

(f) injection, either subcutaneously, intravenously, intra-arterially, intramuscularly, or to other suitable site; or

(g) oral administration, in capsule, liquid, tablet, pill, or prolonged release formulation.

In one embodiment of this invention, an active agent is delivered directly into the CNS, preferably to the brain ventricles, brain parenchyma, the intrathecal space or other suitable CNS location, most preferably intrathecally.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

Exemplary methods for administering such muscle relaxant compounds (e.g., so as to achieve sterile or aseptic conditions) will be apparent to the skilled artisan. Certain methods suitable for administering compounds useful according to the present invention are set forth in Goodman and Gilman's The Pharmacological Basis of Therapeutics, 7th Ed. (1985). The administration to the patient can be intermittent; or at a gradual, continuous, constant or controlled rate. Administration can be to a warm-blooded animal (e.g. a mammal, such as a mouse, rat, cat, rabbit, dog, pig, cow or monkey); but advantageously is administered to a human being. Administration occurs after general anesthesia is administered. The frequency of administration normally is determined by an anesthesiologist, and typically varies from patient to patient.

The active agent is preferably administered in an therapeutically effective amount. By a “therapeutically effective amount” or simply “effective amount” of an active compound is meant a sufficient amount of the compound to treat the desired condition at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or spealists, and typically takes account of the disorder to be treated, the condition of the individual patient, the site of delivery, the method of administration and other factors known to practitioners. Examples of techniques and protocols can be found in Remington 's Parmaceutical Sciences.

Dosage may be adjusted appropriately to achieve desired drug levels, locally or systemically. Typically the active agents of the present invention exhibit their effect at a dosage range from about 0.001 mg/kg to about 250 mg/kg, preferably from about 0.01 mg/kg to about 100 mg/kg of the active ingredient, more preferably from a bout 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved. In the event that the response in a subject is insufficient at such doses, even higher doses (or effective higher doses by a different, more localized delivery route) may be employed to the extent that patient tolerance permits. Continuous dosing over, for example 24 hours or multiple doses per day are contemplated to achieve appropriate systemic levels of compounds.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use humans or animals.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, analgesic agents, cytokines and therapeutic agents in all of the major areas of clinical medicine. When used with other pharmaceutically active compounds, the conopeptides of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

The present invention also relates to rational drug design for the indentification of additional drugs which can be used for the pursposes described herein. The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. Several approaches for use in rational drug design include analysis of three-dimensional structure, alanine scans, molecular modeling and use of anti-id antibodies. These techniques are well known to those skilled in the art. Such techniques may include providing atomic coordinates defining a three-dimensional structure of a protein complex formed by said first polypeptide and said second polypeptide, and designing or selecting compounds capable of interfering with the interaction between a first polypeptide and a second polypeptide based on said atomic coordinates.

Following identification of a substance which modulates or affects polypeptide activity, the substance may be further investigated. Furthermore, it may be manufactured and/or used in preparation, i.e., manufacture or formulation, or a composition such as a medicament, pharmaceutical composition or drug. These may be administered to individuals.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing is generally used to avoid randomly screening large numbers of molecules for a target property.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

The present invention further relates to the use of a labeled (e.g., radiolabel, fluorophore, chromophore or the like) of the conotoxins described herein as a molecular tool both in vitro and in vivo, for discovery of small molecules that exert their action at or partially at the same functional site as the native toxin and capable of elucidation similar functional responses as the native toxin. In one embodiment, the displacement of a labeled conotoxin from its receptor or other complex by a candidate drug agent is used to identify suitable candidate drugs. In a second embodiment, a biological assay on a test compound to determine the therapeutic activity is conducted and compared to the results obtained from the biological assay of a conotoxin. In a third embodiment, the binding affinity of a small molecule to the receptor of a conotoxin is measured and compared to the binding affinity of a conotoxin to its receptor.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982; Sambrook et al., 1989; Ausubel et al., 1992; Glover, 1985; Anand, 1992; Guthrie and Fink, 1991; Harlow and Lane, 1988; Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986).

### EXAMPLES

The present invention is described by reference to the following Examples, which are offered by way of illustration and are not intended to limit the invention in any manner. Standard techniques well known in the art or the techniques specifically described below were utilized.

### Example 1

### Isolation of Conotoxin Peptides

Crude venom was extracted from venom ducts (Cruz et al., 1976), and the components were purified as previously described (Cartier et al., 1996). The crude extract from venom ducts was purified by reverse phase liquid chromatography (RPLC) using a Vydac Cl₁₈ semi-preparative column (10×250 mm). Further purification of bioactive peaks was done on a Vydac C₁₈ analytical column (4.6×220 mm). The effluents were monitored at 220 nm. Peaks were collected, and aliquots were assayed for activity. Throughout purification, HPLC fractions were assayed by means of intracerebral ventricular (i.c.v.) injection into mice (Clark et al., 1981).

The amino acid sequence of the purified peptides were determined by standard methods. The purified peptides were reduced and alkylated prior to sequencing by automated Edman degradation on an Applied Biosystems 477A Protein Sequencer with a 120A Analyzer (DNA/Peptide Facility, University of Utah) (Martinez et al., 1995; Shon et al., 1994).

In accordance with this method, the conotoxin peptides described as “isolated” in Table 1 were obtained. These conotoxin peptides, as well as the other conotoxin peptides and the conotoxin peptide precursors set forth in Table 1 are synthesized as described in U.S. Pat. No. 5,670,622.

### EXAMPLE 2

### Isolation of DNA Encoding Conopeptides

DNA coding for conotoxin peptides was isolated and cloned in accordance with conventional techniques using general procedures well known in the art, such as described in Olivera et al. (1996), including using primers based on the DNA sequence of known conotoxin peptides. For example, primers based on the DNA sequence for the Contulakin-G propeptide were used to identify contulakin homologs. The propeptides of these contulakin homologs are homologous on the basis of primer amplification, even though the sequence of the mature toxins are not homologous with the Contulakin-G mature toxin. Alternatively, cDNA libraries was prepared from Conus venom duct using conventional techniques. DNA from single clones was amplified by conventional techniques using primers which correspond approximately to the M13 universal priming site and the M13 reverse universal priming site. Clones having a size of approximately 300-500 nucleotides were sequenced and screened for similarity in sequence to known conotoxins. The DNA sequences and encoded propeptide sequences are set forth in Table 1. DNA sequences coding for the mature toxin can also be prepared on the basis of the DNA sequences set forth in Table 1. An alignment of the conopeptides of the present invention is set forth in Tables 2-14.

TABLE 1

Name: Af6.1
Species: ammiralis
Cloned: Yes
DNA Sequence:

(SEQ ID NO:1)

ATCATGGAGAAACTGATAATTCTGCTTCTTGTTGCTGCTGTACTGATGTC
GACCCAGGCCCTGGTTGAACGTGCTGGAGAAAACCGCTCAAAGGAGAACA
TCAATTTTTTATTAAAAAGAAAGAGAGCTGCTGACAGGGGGATGTGGGGC
GATTGCAAAGATGGGTTAACGACATGTTTTGCGCCCTCAGAGTGTTGTTC
TGAGGATTGTGAAGGGAGCTGCACGATGTGGTGATGACCTCTGACCACAA
GCCATCTGACATCACCACTCTCCTCTTCAGAGGCTTCAAG
Translation:

(SEQ ID NO:2)

MERLIILLLVAAVLMSTQALVERAGENRSKENINFLLKRKRAADRGMWGD
CKDGLTTCFAPSECCSEDCEGSCTMW
Toxin Sequence:

(SEQ ID NO:3)

Gly-Met-Xaa4-Gly-Asp-Cys-Lys-Asp-Gly-Leu-Thr-Thr-
Cys-Phe-Ala-Xaa3-Ser-Xaa1-Cys-Cys-Ser-Xaa1-Asp-
Cys-Xaa1-Gly-Ser-Cys-Thr-Met-Xaa4-\hat{ }
Name: Af6.2
Species: ammiralis
Cloned: Yes
DNA Sequence:

(SEQ ID NO:4)

ATCATGGAGAAACTGACAATTCTGCTTCTTGTTGCTGCTGTACTGATGTC
GACCCAGGCCCTGCCTCAAGGTGGTGGAGAAAAACGCCCAAGGGAGAATA
TCAGATTTTTATCAAAAAGAAAGACAAATGCTGAGCGTTGGAGGGAGGGC
AGTTGCACCTCTTGGTTAGCGACGTGTACGCAAGACCAGCAATGCTGTAC
TGATGTTTGTTACAAAAGGGACTACTGCGCCTTGTGGGATGACCGCTGAC
CACAAGCCATCTGACATCACCACTCTCCTGTTCAGAGTCTTCAAG
Translation:

(SEQ ID NO:5)

MEKLTILLLVAAVLMSTQALPQGGGEKRPRLNIRFLSKRKTNAERWREGS
CTSWLATCTQDQQCCTDVCYKRDYCALWDDR
Toxin Sequence:

(SEQ ID NO:6)

Xaa4-Arg-Xaa1-Gly-Ser-Cys-Thr-Ser-Xaa4-Leu-Ala-
Thr-Cys-Thr-Gln-Asp-Gln-Gln-Cys-Cys-Thr-Asp-Val-
Cys-Xaa5-Lys-Arg-Xaa5-Cys-Ala-Leu-Xaa4-Asp-Asp-
Arg-\hat{ }
Name: Af6.3
Species: ammiralis
Cloned: Yes
DNA Sequence:

(SEQ ID NO:7)

ATCATGCAGAAACTGATAATTCTGCTTCTTGTTGCTGCTGTGCTGATGTC
GACCCAGGCCCTGTTTCAAGAAAAACGCACAATGAAGAAGATCGATTTTT
TATCAAAGGGAAAGGCAGATGCTGAGAAGCAGAGGAAGCGCAATTGCTCG
GATGATTGGCAGTATTGTGAAAGTCCCAGTGACTGCTGTAGTTGGGATTG
TGATGTGGTCTGCTCGGGATGAACTCTGACCACAAGTCATCCGACATCAC
CACTCTCCTGTTCAGAGGCTTCAAG
Translation:

(SEQ ID NO:8)

MQKLIILLLVAAVLMSTQALFQEKRTMKKIDFLSKGKADAFKQRKRNCSD
DWQYCESPSDCCSWDCDVVCSG
Toxin Sequence:

(SEQ ID NO:9)

Asn-Cys-Ser-Asp-Asp-Xaa4-Gln-Xaa5-Cys-Xaa1-Ser-
Xaa3-Ser-Asp-Cys-Cys-Ser-Xaa4-Asp-Cys-Asp-Val-
Val-Cys-Ser-#
Name: Af6.4
Species: ammiralis
Cloned: Yes
DNA Sequence:

(SEQ ID NO:10)

ATCATGCAGAAACTGATAATCCTGCTTCTTGTTGCTGCTCTACTGTTGTC
GATCCAGGCGGTAAATCAAGAAAAACACCAACGGGCAAAGATCAACTTGC
TTTCAAAGAGAAAGCCACCTGCTGAGCGTTGGTGGCGGTGGGGAGGATGC
ATGGCTTGGTTTGGGAAATGTTCGAAGGACTCGGAATGTTGTTCTAATAG
TTGTGACATAACGCGCTGCGAGTTAATGCGATTCCCACCAGACTGGTGAC
ATCGACACTCTCCTGTTCAGAGTCTTCAAG
Translation:

(SEQ ID NO:11)

MQRLIILLLVAALLLSIQAVNQEKIIQRAKTNLLSKRKPPAERWWRWGGC
MAWFGKCSKDSECCSNSCDITRCELMRFPPDW
Toxin Sequence:

(SEQ ID NO:12)

Xaa4-Xaa4-Arg-Xaa4-Gly-Gly-Cys-Met-Ala-Xaa4-Phe-
Gly-Lys-Cys-Ser-Lys-Asp-Ser-Xaa1-Cys-Cys-Ser-Asn-
Ser-Cys-Asp-Ile-Thr-Arg-Cys-Xaa1-Leu-Met-Arg-Phe-
Xaa3-Xaa3-Asp-Xaa4-\hat{ }
Name: Af6.5
Species: ammiralis
Cloned: Yes
DNA Sequence:

(SEQ ID NO:13)

ATCATGGAGAAACTGACAATCCTGCTTCTTGTTGCTGCTGTACTGACGTC
GACCCAGGCCCTGATTCAAGGTGGTGGAGACGAACGCCAAAAGGCAAAGA
TCAACTTTCTTTCAAGGTCGGACCGCGATTGCAGGGGTTACGATGCGCCG
TGTAGCTCTGGCGCGCCATGTTGTGATTGGTGGACATGTTCAGCACGAAC
CGGGCGCTGTTTTTAGGCTGACCACAAGCCATCCGACATCACCACTCTCC
TCTTCAGAGGCTTCAAG
Translation:

(SEQ ID NO:14)

MEKLTILLLVAAVLTSTQALIQGGGDERQKAKINFLSRSDRDCRGYDAPC
SSGAPCCDWWTCSARTGRCF
Toxin Sequence:

(SEQ ID NO:15)

Asp-Cys-Arg-Gly-Xaa5-Asp-Ala-Xaa3-Cys-Ser-Ser-
Gly-Ala-Xaa3-Cys-Cys-Asp-Xaa4-Xaa4-Thr-Cys-Ser-
Ala-Arg-Thr-Gly-Arg-Cys-Phe-\hat{ }
Name: Af6.6
Species: ammiralis
Cloned: Yes
DNA Sequence:

(SEQ ID NO:16)

ATCATGCAGAAACTGACAATTCTGCTTCTTGTTGCTGCTGTGCTGATGTC
GACCCAGGCCGTGCTTCAAGAAAAACGCCCAAAGGAGAAGATCAAGTTTT
TATCAAAGAAAAAGACAGATGCTGAGAAGCAGCAGAAGCGCCTTTGCCCG
GATTACACGGAGCCTTGTTCACATGCCCATGAATGCTGTTCATGGAATTG
TCATAATGGGCACTGCACGGGATGAACTCGGACCACAAGCCATCGACATC
ATCACTCTCCTGTTCAGAGTCTTCAAG
Translation:

(SEQ ID NO:17)

MQKLTILLLVAAVLMSTQAVLQEKRPKEKIKFLSKKKTDAEKQQKRLCPD
YTEPCSHAHECCSWNCHNGHCTG
Toxin Sequence:

(SEQ ID NO:18)

Leu-Cys-Xaa3-Asp-Xaa5-Thr-Xaa1-Xaa3-Cys-Ser-His-
Ala-His-Xaa1-Cys-Cys-Ser-Xaa4-Asn-Cys-His-Asn-Gly-
His-Cys-Thr-#
Name: Af6.7
Species: ammiralis
Cloned: Yes
DNA Sequence:

(SEQ ID NO:19)

ATCATGCAGAAACTGATAATTCTGCTCCTTGTTGCTGCTGTACTGATGTC
GACCCAGGCCATGTTTCAAGGTGATGGAGAAAAATCCCGGAAAGCGGAGA
TCAACTTTTCTAAAACAAGAAATTTGGCGAGAAACAAGCAGAAACGCTGC
AGTAGTTGGGCAAAGTATTGTGAAGTTGACTCGGAATGCTGTTCCGAACA
GTGTGTAAGGTCTTACTGCGCGATGTGGTGATGACCTCTGACCACAAGCC
ATCCGATATCACCACTCTCCTCTTCAGAGACTTCAAG
Translation:

(SEQ ID NO:20)

MQKLIILLLVAAVLMSTQAMFQGDGEKSRKAFThESKTRMARINKQKRCS
SWAKYCEVDSECCSEQCVRSYCAMW
Toxin Sequence:

(SEQ ID NO:21)

Cys-Ser-Ser-Xaa4-Ala-Lys-Xaa5-Cys-Xaa1-Val-Asp-
Ser-Xaa1-Cys-Cys-Ser-Xaa1-Gin-Cys-Val-Arg-Ser-
Xaa5-Cys-Ala-Met-Xaa5-\hat{ }
Name: Af9.1
Species: ammiralis
Cloned: Yes
DNA Sequence:

(SEQ ID NO:22)

GTTAAAATGCATCTGTCACTGGCACGCTCAGCTGTTTTGATGTTGCTTCT
GCTGTTTGCCTTGGGCAACTTTGTTGTGGTCCAGTCAGGACAGATAACAA
GAGATGTGGACAATGGACAGCTCACGGACAACCGCCGTAACCTGCAATCG
AAGTGGAAGCCAGTGAGTCTCTTCATGTCACGACGGTCTTGTAACAATTC
TTGCAATGAGCATTCCGATTGCGAATCCCATTGTATTTGCACGTTTAGCG
GATGCAAAATTATTTTGATATAAACGGATTGAGTTTGCTCGTCAACAAGA
TGTCGCACTACAGCTCCTCTCTACAGTGTGTACATCGACCAAACGACGCA
TCTTTTATTTCTTTGTCTGTTGTATTTGTTTTCCTGTGTTCATAACGTAC
AGAGCCCTTTAATTACCTTTACTGCTCTTCACTTAACCTGATAACCGGAA
GGTCCAGTGCT
Translation:

(SEQ ID NO:23)

MHLSLARSAVLMLLLLFALGNFVVVQSGQITRDVDNGQLTDNRRNLQSKW
KPVSLFMSRRSCNNSCNEHSDCESHCICTFSGCKIILI
Toxin Sequence:

(SEQ ID NO:24)

Ser-Cys-Asn-Asn-Ser-Cys-Asn-Xaa1-His-Ser-Asp-Cys-
Xaa1-Ser-His-Cys-Ile-Cys-Thr-Phe-Ser-Gly-Cys-Lys-
Ile-Ile-Leu-Ile-\hat{ }
Name: Af9.2
Species: ammiralis
Cloned: Yes
DNA Sequence:

(SEQ ID NO:25)

GTTAAAATGCATCTGTCACTGGCACGCTTAGCTGTTTTGATGTTGCTTCT
GCTGTTTGCCTTGGGCAACTTTGTTGTGGTCCAGTCAGGACAGATAACAA
GAGATGTGGACAATGGACAGCTCACGGACAACCGCCGTAACCTGCAATCG
AAGTGGAAGCCAGTGAGTCTCTTCATGTCACGACGGTCTTGTAACAATTC
TTGCAATGAGCATTCCGATTGCGAATCCCATTGTATTTGCACGTTTAGAG
GATGCGGAGCTGTTAATGGTTGAGTTTGCTCGTCAACATGATGTCGCACT
ACACACTACAGCTCCTCTCTACAGTGTGTACATCGACCAAACGACGCATC
TTTTATTTCTTTGTCTGTTGTGTTTGTTTTCCTGTGTTCATAACGTACAG
AGCCCTTTAATTACTTTTACTGCTCTTCACTTAACCTGATAACCAGAAGG
TCCAGTGCT
Translation:

(SEQ ID NO:26)

MHLSLARLAVLMLLLLFALGNFVVVQSGQITRDVDNGQLTDNRRINLQSK
WRPVSLFMSRRSCNNSCKEHSDCESHCICTFRGCGAVNG
Toxin Sequence:

(SEQ ID NO:27)

Ser-Cys-Asn-Asn-Ser-Cys-Asn-Xaa1-His-Ser-Asp-Cys-
Xaa1-Ser-His-Cys-Ile-Cys-Thr-Phe-Arg-Gly-Cys-Gly-
Ala-Val-Asn-#
Name: Ar6.1
Species: arenatus
Cloned: Yes
DNA Sequence:

(SEQ ID NO:28)

ACCAAAACCATCATCAAAATGAAACTGACGTGCGTGGTGATCGTCGCTGT
GCTGTTCCTGACGGCCTGTCAACTCACTACAGCTGATGACTCCAGAGGTA
CGCAGAAGCATGGTGCCCTGAGATCGACCACCAAACTCTCCATGTTGACT
CGGGGCTGCACGCCTCCTGGTGGAGTTTGTGGTTATCATGGTCACTGCTG
CGATTTTTGCGATACGTTCGGCAATTTATGTGTGAGTGGCTGACCCGGCA
TCTGACCTTTCCCCCTTCTTTGCTCCACTATCCTTTTTCTGCCTGAGTCC
TCCATACCTGAGAGCTGTCATGAACCACTCAACACCTACTCTTCCGGAGG
TTTCTGAGGAGCTGCATTGAAATAAAAGCCGCATTGC
Translation:

(SEQ ID NO:29)

MKLTCVVIVAVLFLTACQLTTADDSRGTQKHGALRSTTKLSMLTRGCTPP
GGVCGYHGHCCDFCDTFGNLCVSG
Toxin Sequence:

(SEQ ID NO:30)

Gly-Cys-Thr-Xaa3-Xaa3-Gly-Gly-Val-Cys-Gly-Xaa5-
His-Gly-His-Cys-Cys-Asp-Phe-Cys-Asp-Thr-Phe-Gly-
Asn-Leu-Cys-Val-Ser-#
Name: Bromosleeper-Ari
Species: arenatus
Cloned: Yes
DNA Sequence:

(SEQ ID NO:31)

GACAGGATTGAACAAAATTCAGGATGTCAGGATTGGGAATCATGGTGCTA
ACCCTTCTACTTCTTGTGTTCATGGCAACCAGTCATCAGGATGCAGGAGA
GAAGAAGGCGATGCAAAGGGACGCAATCAACGTCAGACGGAGAAGATCAC
TCACTCGGGGAGTAGTAACTGAGGCGTGCGAAGAGTCCTGTGAGGAGGAG
GAAAAGCACTGCTGCCACGTAAATAATGGAGTACCCTCTTGTGCCGTTAT
ATGCTGGGGATAGTTTCTCGCACACTGTCTCATTCATTATTTTATCAGTA
CAAGTGTAAACGAGACATGTCAGAAAGTCGAAGGTTGTGCGTATTTGATA
AGTATTGTTTACTGGGATGAACGGA
Translation:

(SEQ ID NO:32)

MSGLGIMVLTLLLLVFMATSHQDAGEKKAMQRDAINVRRRRSLTRGVVTE
ACEESCEEEEKHCCHVNNGVPSCAVICWG
Toxin Sequence:

(SEQ ID NO:33)

Val-Val-Thr-Xaa1-Ala-Cys-Xaa1-Xaa1-Ser-Cys-Xaa1-
Xaa1-Xaa1-Xaa1-Lys-His-Cys-Cys-His-Val-Asn-Asn-
Gly-Val-Xaa3-Ser-Cys-Ala-Val-Ile-Cys-Xaa4-#
Name: Bromosleeper-Ari A
Species: arenatus
Cloned: Yes
DNA Sequence:

(SEQ ID NO:34)

GACAGGATTGAACAAAATTCAGGATGTCAGGATTGGGAATCATGGTGCTA
ACCCTTCTACTTCTTGTGTTCATGGCAACCAGTCATCAGGATGCAGGAGA
GAAGCAGGCGACGGAAAGGGACGCAATCAACATCAGATGGAGAAGATCAC
GCACTCGGAGAATAGTAACTGAGGCGTGCGAAGAGTCCTGTGAGGACGAG
GAAAAGCACTGCTGCCACGTAAATAATGGAGTACCCTCTTGTGCCGTTAT
ATGCTGGGGATAGTTTCTCGCACACTGTCTCATTCATTATTTTATCAGTA
CAAGTGTAAACGAGACATGTCAGAAAGTCGAAGGTTGTGCGTATTTGATA
AGTATTGTTTACTGGGATGAACGGA
Translation:

(SEQ ID NO:35)

MSGLGIMVLTLLLLVFMATSHQDAGEKQATERDAINIRWRRSRTRRIVTE
ACEESCEDEEKIICCHYNNGVPSCAVICWG
Toxin Sequence:

(SEQ ID NO:36)

Ile-Val-Thr-Xaa1-Ala-Cys-Xaa1-Xaa1-Ser-Cys-Xaa1-
Asp-Xaa1-Xaa1-Lys-His-Cys-Cys-His-Val-Asn-Asn-Gly-
Val-Xaa3-Ser-Cys-Ala-Val-Ile-Cys-Xaa4-#
Name: Bromosleeper-Ar2
Species: arenatus
Cloned: Yes
DNA Sequence:

(SEQ ID NO:37)

GACAGGATTGAACAAAATTCAGGATGTCAGAACTGGGAATCATGGTGCTA
ACGCTTCTACTTCTTGTGTTCCTGGTAACCAGTCATCAGGATGCAGGAGA
GAAGCAGGCGACGGAAAGGGACGCAATCAACATCAGATGGAGAAGATCAC
TCACTCGGAGAATAGTAACTGAGGCGTGCGAAGAGCACTGTGAGGATGAG
GAACAGTTCTGCTGCGGCTTAGAGAATGGACAACCCTTTTGTGCCCCTGT
TTGCTTCGGATAGTTTCTGTACACTGTCTCATTAATTATTTTATCAGTAC
AAGTGTAAACAAAACATGTCAGAAAGTCGAAGGTTGTGCGTATTTGATAA
GTATTGTTTGCTGGGACGAACGGA
Translation:

(SEQ ID NO:38)

MSELGIMVLTLLLLVFLVTSHQDAGEKQATERDAINIRWRRSLTRRIVTE
ACEEHCEDEEQFCCGLENGQPFCAPVCFG
Toxin Sequence:

(SEQ ID NO:39)

Ile-Val-Thr-Xaa1-Ala-Cys-Xaa1-Xaa1-His-Cys-Xaa1-
Asp-Xaa1-Xaa1-Gln-Phe-Cys-Cys-Gly-Leu-Xaa1-Asn-
Gly-Gln-Xaa3-Phe-Cys-Ala-Xaa3-Val-Cys-Phe-#
Name: Bromosleeper-Ar3
Species: arenatus
Cloned: Yes
DNA Sequence:

(SEQ ID NO:40)

GACAGGATTGAACAAAATTCAGGATGTCAGGATTGGGAATCATGGTGCTA
ACCCTTCTACTTCTTGTGTTCATGGCAACCAGTCATCAGGATGCAGGAGA
GAAGAAGGTGATGCAAAGGGACGCAATCAACGTCAGACGGAGAAGATCAC
GCACTCGGAGAGTAGTAACTGGGGCGTGCGAAGAGCACTGTGAGGACGAG
GAAAAGCACTGCTGCGGCTTAGAGAATGGACAACCCTTTTGTGCCCGTCT
ATGCTTAGGATAGTTTTCTGTACACTGTCTTATTCATTATTTTATCAGTA
CAAGTGAAAACAAAGCATGTCAGAAAGTCGAAGGTTGTGCGTATTTGATA
AGTATTGTTTACTGGGATGAACGGA
Translation:

(SEQ ID NO:41)

MSGLGIMVLTLLLLVFMATSHQDAGEKKVMQRDAINVRRRRSRTRRVVTG
ACEEHCEDEEKIHCCGLENGQPFCARLCLG
Toxin Sequence:

(SEQ ID NO:42)

Val-Val-Thr-Gly-Ala-Cys-Xaa1-Xaa1-His-Cys-Xaa1-
Asp-Xaa1-Xaa1-Lys-His-Cys-Cys-Gly-Leu-Xaa1-Asn-
Gly-Gln-Xaa3-Phe-Cys-Ala-Arg-Leu-Cys-Leu-#
Name: C. arenatus contryphan 1
Species: arenatus
Cloned: Yes
DNA Sequence:

(SEQ ID NO:43)

ATGGGGAAACTGACAATACTGGTTCTTGTTGCTGCTGTACTGTTGTCGAC
CCAGGTCATGGTTCAAGGTGACGGAGATCAACCTGCAGCTCGCAATGCAG
TGCCAAAAGACGATAACCCAGATGGAGCGAGTGGAAAGTTCATGAATGTT
CTACGTCGGTCTGGATGTCCGTGGCATCCTTGGTGTGGCTGATCGGAATC
CACGATTGCAATGACAGCC
Translation:

(SEQ ID NO:44)

MGRLTILVLVAAVLLSTQVMVQGDGDQPAARNAVPKDDNPDGASGKFMNV
LRRSGCPWHPWCG
Toxin Sequence:

(SEQ ID NO:45)

Ser-Gly-Cys-Xaa3-Xaa4-His-Xaa3-Xaa4-Cys-#
Name: C. arenatus contryphan 1A
Species: arenatus
Cloned: Yes
DNA Sequence:

(SEQ ID NO:46)

ATGGGGAAACTGACAATACTGGTTCTTGTTGCTGCTGTACTGTTGTCGAC
CCAGGTCATGGTTCAAGGTGACGGAGATCAACCTGCAGCTCGCAATGCAG
TGCCAAAAGACGATAACCCAGATGGAGCGAGTGGAAAGTTCATGAATGTT
CTACGTCGGTCTGGATGTCCGTGGCGCCCTTGGTGTGGCTGATCGGAATC
CACGATTGCAATGACAGCC
Translation:

(SEQ ID NO:47)

MGKLTILVLVAAVLLSTQVMVQGDGDQPAARNAVPKDDNPDGASGKIFMN
VLRRSGCPWRPWCG
Toxin Sequence:

(SEQ ID NO:48)

Ala-Ser-Gly-Cys-Xaa3-Xaa4-Arg-Xaa3-Xaa4-Cys-#
Name: C. arenatus contryphan 2
Species: arenatus
Cloned: Yes
DNA Sequence:

(SEQ ID NO:49)

ATGGGGAAACTGACAATACTGGTTCTTGTTGCTGCTGTACTGTTGTCGAC
CCAGGTCATGGTTCAAGGTGACGGAGATCAACCTGCAGGTCGAGATGCAG
TTCCAAGAGACGATAACCCAGGTGGAACGAGTGGAAAGTTCATGAATGCT
CTACGTCAATATGGATGTCCGGTGGGTCTTTGGTGTGACTGATCAGAATC
CACGATTGCAATGACAGCC
Translation:

(SEQ ID NO:50)

MGKLTILVLVAAVLLSTQVMVQGDGDQPAGRDAVPRDDNPGGTSGKFMNA
LRQYGCPVGLWCD
Toxin Sequence:

(SEQ ID NO:51)

Xaa2-Xaa5-Gly-Cys-Xaa3-Val-Gly-Leu-Xaa4-Cys-Asp-\hat{ }
Name: C. arenatus contryphan 4
Species: arenatus
Cloned: Yes
DNA Sequence:

(SEQ ID NO:52)

ATGGGGAAACTGACAATACTGGTTCTTGTTGCTGCTGTACTGTTGTCGAC
CCAGGTCATGTTTCGAGATCAACCTGCACGTCGTGATGCAGTGCCAAGAG
ACGATAGCCCAGATGGAATGAGTGGAGGGTTCATGAATGTCCCACGTCGG
TCTGGATGTCCGTGGCAACCTTGGTGTGGCTGATCGGAATCCACGATTGC
AATGACAGCC
Translation:

(SEQ ID NO:53)

MGKLTILVLVAAVLLSTQVMFRDQPARRDAVPRDDSPDGMSGGFMNVPRR
SGCPWQPWCG
Toxin Sequence:

(SEQ ID NO:54)

Ser-Gly-Cys-Xaa3-Xaa4-Gln-Xaa3-Xaa4-CyS-#
Name: Contryphan-Ar-1
Species: arenatus
Cloned: Yes
DNA Sequence:

(SEQ ID NO:55)

ATGGGGAAACTGACAATACTGGTTCTTGTTGCTGCTGTACTGTTGTCGAC
CCAGGCCATGGTTCAAGATCAACCTGCAGGTCGAGATGCAGTTCCAAGAG
ACGATAACCCAGGTGGAACGAGTGGAAAGTTCGTGAATGCTCAACGTCAA
TATGGATGTCCGCCGGGTCTTTGGTGTCACTGATCAGAATCCACGATTGC
AATGACAGCC
Translation:

(SEQ ID NO:56)

MGKLTILVLVAAVLLSTQAMVQDQPAGRDAVPRDDNPGGTSGKFVNAQRQ
YGCPPGLWCH
Toxin Sequence:

(SEQ ID NO:57)

Xaa2-Xaa5-Gly-Cys-Xaa3-Xaa3-Gly-Leu-Xaa4-Cys-His-\hat{ }
Name: A10.1
Species: aurisiacus
Cloned: Yes
DNA Sequence:

(SEQ ID NO:58)

ATGTTCACCGTGTTTCTGTTGGTTGTCTTGGCAACCACTGTCGTTTCCAT
CCCTTCAGATCGTGCATCTGATGGCAGGAATGCCGCAGTCAACGAGAGAG
CGCCTTGGCTGGTCCCTTCGACAATCACGACTTGCTGTGGATATAATCCG
GGGACAATGTGCCCTCCTTGCAGGTGCGATAATACCTGTTAACCAAAAAA
AAAAAAAAAAAAA
Translation:

(SEQ ID NO:59)

MFTVFLLVVLATTVVSIPSDRASDGRNAAVNERNPWLVPSTITTCCGYNP
GTMCPPCRCDNTC
Toxin Sequence:

(SEQ ID NO:60)

Ala-Xaa3-Xaa4-Leu-Val-Xaa3-Ser-Thr-Ile-Thr-Thr-
Cys-Cys-Gly-Xaa5-Asn-Xaa3-Gly-Thr-Met-Cys-Xaa3-
Xaa3-Cys-Arg-Cys-Asp-Asn-Thr-Cys-\hat{ }
Name: Bnl.5
Species: bandanus
Cloned: Yes
DNA Sequence:

(SEQ ID NO:61)

ATGCGCTGTCTCCCAGTCTTGATCATTCTTCTGCTGCTGACTGCATCTGC
ACCTGGCGTTGATGTCCTACCGAAGACCGAAGATGATGTGCCCCTGTCAT
CTGTCTACGATAATACAAAGAGTATCCTACGAGGACTTCTGGACAAACGT
GCTTGCTGTGGCTACAAGCTTTGCTCACCATGTTAACCAGCATGAAGGAT
CC
Translation:

(SEQ ID NO:62)

MRCLPVLIILLLLTASAPGVDVLPKTEDDVPLSSVYDNTKSILRGLLDKR
ACCGYKLCSPC
Toxin Sequence:

(SEQ ID NO:63)

Ala-Cys-Cys-Gly-Xaa5-Lys-Leu-Cys-Ser-Xaa3-Cys-\hat{ }
Name: Ca6.3
Species: caracteristicus
Cloned: Yes
DNA Sequence:

(SEQ ID NO:64)

GGATCCATGAAACTGACGTGCGTGGTGATCATCGCCGCGCTGTTCCTGAC
GGCCTGTCAGCTCAATACAGCTGATGACTCCAGAGATAAGCAGGAGTACC
GTGCAGTGAGGTTGAGAGACGGAATGCGGAATTTCAAAGGTTCCAAGCGC
AACTGCGGGGAACAAGGTGAAGGTTGTGCTACTCGCCCATGCTGCTCTGG
TCTGAGTTGCGTTGGCAGCCGTCCAGGAGGCCTGTGCCAGTACGGCTAAT
AGTCTGGCATCTGATATTTCCCCTCTGCACTCTACCTTCTTTTGCCTGAT
GCATGTTTACTTGTGTGTGGTCATGAACCACTCAGTAGCTACACCTCCGA
AGGACGTGC
Translation:

(SEQ ID NO:65)

MKLTCVVHAALFLTACQLNTADDSRDKQEYRAVRLRDGMRNFKGSKRNCG
EQGEGCATRPCCSGLSCVGSRPGGLCQYG
Toxin Sequence:

(SEQ ID NO:66)

Asn-Cys-Gly-Xaa1-Gln-Gly-Xaa1-Gly-Cys-Ala-Thr-Arg-
Xaa3-Cys-Cys-Ser-Gly-Leu-Ser-Cys-Val-Gly-Ser-Arg-
Xaa3-Gly-Gly-Leu-Cys-Gln-Xaa5-#
Name: Ca8.1
Species: caracteristicus
Cloned: Yes
DNA Sequence:

(SEQ ID NO:67)

ATGATGTCGAAAATGGGAGCTATGTTTGTCCTTTTGCTTCTTTTCATCCT
GCCATCCAGCCAGCAGGAAGGAGATGTCCAGGCAAGAAAAACGCACCTGA
AGAGAGGCTTCTACGGTACTCTGGCAATGTCTACCAGAGGATGCTCTGGC
ACTTGCCATCGTCGTGAGGACGGCAAGTGTCGGGGTACTTGCGACTGCTC
CGGATACAGCTATTGTCGCTGCGGTGACGCTCACCATTTTTACCGAGGAT
GCACGTGTTCGTGTCAAGGTTGATTAATTGACTCTTTTAACTCGTTGAAC
GATTGAAAAAAAAAATTTTAGAGCAATATGTTCGAGAAAAACCGAAGAC
Translation:

(SEQ ID NO:68)

MMSRMGAMFVLLLLFILPSSQQEGDVQARIKTHLKRGFYGTLAMSTRGCS
GTCHRREDGKCRGTCDCSGYSYCRCGDAHHFYRGCTCSCQG
Toxin Sequence:

(SEQ ID NO:69)

Gly-Cys-Ser-Gly-Thr-Cys-His-Arg-Arg-Xaa1-Asp-Gly-
Lys-Cys-Arg-Gly-Thr-Cys-Asp-Cys-Ser-Gly-Xaa5-Ser-
Xaa5-Cys-Arg-Cys-Gly-Asp-A1a-His-HiS-Phe-Xaa5-Arg-
Gly-Cys-Thr-Cys-Ser-Cys-Gln-#
Name: Ca8.2
Species: caracteristicus
Cloned: Yes
DNA Sequence:

(SEQ ID NO:70)

ATGATGTCGAAAATGGGAGCTATGTTTGTCCTTTTGCTTCTTTTCATCCT
GCCATCCAGCCAGCAGGAAGGAGATGTCCAGGCAAGAAAAACGCACCGGA
AGAGCGGCTTCTACGGTACTCTGGCAATGTCTGCCAGAGGATGCTCTGGC
ACTTGCCATCGTCGTGAGGACGGCAAGTGTCGGGGTACTTGCGACTGCTC
CGGATACAGCTATTGTCGCTGCGGTGACGCTCACCATTTTTACCGAGGAT
GCACGTGTACATGTTAAGGTTGATTAATTGACTCTTTTAACTCGTTGAAC
CGATTAAAAAAAAATTAGACGAATATGTTCGAGAAAACCGAAGAC
Translation:

(SEQ ID NO:71)

MMSKMGAMFVLLLLFILPSSQQEGDVQARKTHIRKSGFYGTLAMSARGCS
GTCHRREDGKCRGTCDCSGYSYCRCGDAHHFYRGCTCTC
Toxin Sequence:

(SEQ ID NO:72)

Gly-Cys-Ser-Gly-Thr-Cys-His-Arg-Arg-Xaa1-Asp-Gly-
Lys-Cys-Arg-Gly-Thr-Cys-Asp-Cys-Ser-Gly-Xaa5-Ser-
Xaa5-Cys-Arg-Cys-Gly-Asp-Ala-His-His-Phe-Xaa5-Arg-
Gly-Cys-Thr-Cys-Thr-Cys-\hat{ }
Name: Ca8.3
Species: caracteristicus
Cloned: Yes
DNA Sequence:

(SEQ ID NO:73)

ATGATGTCGAAAATGGGAGCTATGTTTGTCCTTTTGCTTCTTTTCATCCT
GCCATCCAGCCAGCAGGAAGGAGATGTCCAGGCAAGAAAAACGCACCGGA
AGAGCGGCTTCTACGGTACTCTGGCAATGTCTACCAGAGGATGCTCTGGC
ACTTGCCGTCGTCATCGGGACGGCAAGTGTCGGGGTACTTGCGACTGCTC
CGGATACAGCTATTGTCGCTGCGGTGACGCTCACCATTTTTACCGAGGAT
GCACGTGTACATGTTAAGGTTGATTAATTCGATCTTTTAACTCGTTGAAC
GATTAAAAAAAAAATTTTAGACGAATATGTTCGAGAAAAACCGAAGAC
Translation:

(SEQ ID NO:74)

MMSKMGAMFVLLLLFILPSSQQEGDVQARKTHRKSGFYGTLAMSTRGCSG
TCRRHRDGKCRGTCDCSGYSYCRCGDAHHFYRGCTCTC
Toxin Sequence:

(SEQ ID NO:75)

Gly-Cys-Ser-Gly-Thr-Cys-Arg-Arg-His-Arg-Asp-Gly-
Lys-Cys-Arg-Gly-Thr-Cys-Asp-Cys-Ser-Gly-Xaa5-Ser-
Xaa5-Cys-Arg-Cys-Gly-Asp-Ala-His-His-Phe-Xaa5-Arg-
Gly-Cys-Thr-Cys-Thr-Cys-\hat{ }
Name: Ca8.4
Species: caracteristicus
Cloned: Yes
DNA Sequence:

(SEQ ID NO:76)

ATGATGTCGAAAATGGGAGCTATGTTTGTCCTTTTGCTTCTTTTCATCCT
GCCATCCAGCCAGCAGGAAGGAGATGTCCAGGCAAGAAAAACGCACCTGA
AGAGAGGCTTCTACGGTACTCTGGCAATGTCTACCAGAGGATGCTCTGGC
ACTTGCCGTCGTCATCGGGACGGCAAGTGTCGGGGTACTTGCGACTGCTC
CGGATACAGCTATTGTCGCTGCGGTGACGCTCACCATTTTTACCGAGGAT
GCACGTGTACATGTTAAGGTTGATTAATTGACTCTTTTAACTCGTTGAAC
GATTAAAAAAAAAAATTTTAGAGCAATATGTTCGAGAAAAACCGAAGAC
Translation:

(SEQ ID NO:77)

MMSKMGAMFVLLLLFILPSSQQEGDVQARKTHLKRGFYGTLAMSTRGCSG
TCRRHRDGKCRGTCDCSGYSYCRCGDAHHFYRGCTCTC
Toxin Sequence:

(SEQ ID NO:78)

Gly-Cys-Ser-Gly-Thr-Cys-Arg-Arg-His-Arg-Asp-Gly-
Lys-Cys-Arg-Gly-Thr-Cys-Asp-Ser-Gly-Xaa5-Ser-Xaa5-
Cys-Arg-Cys-Gly-Asp-Ala-His-His-Phe-Xaa5-Arg-Gly-
Cys-Thr-Cys-Thr-Cys-\hat{ }
Name: Ca8.5
Species: caracteristicus
Cloned: Yes
DNA Sequence:

(SEQ ID NO:79)

ATGATGTCGAAAATGGGAGCTATGTTTGTCCTTTTGTTTCTTTTCACCCT
GCCATCCAGCCAGCAGGAAGGAGATGTCCAGGCAAGAAAAACGCACCTGA
AGAGAGGCTTCTACGGTACTCTGGCAATGTCTTCCAGAGGATGCTCTGGC
ACTTGCCATCGTCGTGAGGACGGCAAGTGTCGGGGTACTTGCGACTGCTC
CGGATACAGCTATTGTCGCTGCGGTGACGCTCACCATTTTTACCGAGGAT
GTACGTGTACATGTTAAGGTTGATTAATTGACTCTTTTAACTCGTTGAAC
GATTAAAAAAAAATTTAGAGCAATATGTTCGAGAAAACCGAAGAC
Translation:

(SEQ ID NO:80)

MMSKMGAMFVLLFLFTLPSSQQEGDVQARKTHLKRGFYGTLAMSSRGCSG
TCHRREDGKCRGTCDCSGYSYCRCGDAHHFYRGCTCTC
Toxin Sequence:

(SEQ ID NO:81)

Gly-Cys-Ser-Gly-Thr-Cys-His-Arg-Arg-Xaa1-Asp-Gly-
Lys-Cys-Arg-Gly-Thr-Cys-Asp-Cys-Ser-Gly-Xaa5-Ser-
Xaa5-Cys-Arg-Cys-Gly-Asp-Ala-His-His-Phe-Xaa5-Arg-
Gly-Cys-Thr-Cys-Thr-Cys-\hat{ }
Name: Ca8.6
Species: caracteristicus
Cloned: Yes
DNA Sequence:

(SEQ ID NO:82)

ATGATGTCGAAAATGGGAGCTATGTTTGTCCTTTTGCTTCTTTTCATCCT
GCCATCCAGCCAGCAGGAAGGAGATGTCCAGGCAAGAAAAACGCACCTGA
AGAGCGGCTTCTACGGTACTCTGGCAATGTCTGCCAGAGGATGCTCTGGC
ACTTGCCATCGTCGTCAAAACGGCGAGTGTCAGGGTACTTGCGACTGCGA
CGGACACGACCATTGTGACTGCGGTGACACTCTCGGTACTTACTCAGGAT
GCGTGTGTATATGTTAAGGTTGATTAATTGACTCTTTTAACTCGTTGAAC
GATTAAAAAAATTTAGAGCAATATGTTCGAGAAAAACCGAAGAC
Translation:

(SEQ ID NO:83)

MMSKMGAMFVLLLLFILPSSQQEGDVQARKTHLKSGFYGTLAMSARGCSG
TCHIRRQNGECQGTCDCDGHDLICDCGDTLGTYSGCVCIC
Toxin Sequence:

(SEQ ID NO:84)

Gly-Cys-Ser-Gly-Thr-Cys-His-Arg-Arg-Gln-Asn-Gly-
Xaa1-Cys-Gln-Gly-Thr-Cys-Asp-Cys-Asp-Gly-His-Asp-
His-Cys-Asp-Cys-Gly-Asp-Thr-Leu-Gly-Thr-Xaa5-Ser-
Gly-Cys-Val-Cys-Ile-Cys-\hat{ }
Name: Ca9.1
Species: caracteristicus
Cloned: Yes
DNA Sequence:

(SEQ ID NO:85)

GTTACAATGCATCTGTCACTGGCACGCTCAGCTGTCTTGATGTTGCTTCT
GCTGTTTGCCTTGGACAACTTCGTTGGGGTCCAGCCAGGACAGATAACAA
GAGATGTGGACAACCGCCGTAACCGGCAATCGCGATGGAAGCCAAGGAGT
CTCTTCAAGTCACTTCATAAACGAGCATCGTGTGGAGGGACTTGCACGGA
AAGTGCCGATTGCCCTTCCACGTGTAGTACTTGCTTACATGCTCAATGCG
AGTCAACATGATGTCGCACTACAGCTCTTCTCTACAGTGTGTACATCGAC
CGTACGACGCATCTTTTATTTCTTTGGCTGTTTCATTCGTTTTCTTGTGT
TCATAACATGCGGAGCCCTTCCGTTACCTCTACTGCTCTACACTTAACCT
GATAACCAGAAAATCCAGTACT
Translation:

(SEQ ID NO:86)

MHLSLARSAVLMLLLLFALDNIWGVQPGQITRDVDNRRNRQSRWKPRSLF
KSLHKRASCGGTCTESADCPSTCSTCLHAQCEST
Toxin Sequence:

(SEQ ID NO:87)

Ala-Ser-Cys-Gly-Gly-Thr-Cys-Thr-Xaa1-Ser-Ala-Asp-
Cys-Xaa3-Ser-Thr-Cys-Ser-Thr-Cys-Leu-His-Ala-Gln-
Cys-Xaa1-Ser-Thr-\hat{ }
Name: Ca9.2
Species: caracteristicus
Cloned: Yes
DNA Sequence:

(SEQ ID NO:88)

GTTACAATGCATCTGTCACTGGCACGCTCAGCTGTTTTGATGTTGCTTCT
GCTGTTTGCCTTGGACAACTTCGTTGGGGTCCAACCAGGACAGATAACTA
GAGATGTGGACAACCGCCGTAACCTGCAATCGCGATGGAAGCCAAGGAGT
CTCTTCAAGTCACTTCATAAACGAGCATCGTGTGGAGGGACTTGCACGGA
AAGTGCCGATTGCCCTTCCACGTGTAGTACTTGCTTACATGCTCAATGCG
AGTGAACATGATGTCGCACTACAGCTCTTCTCTACAGTGTGTACATCGAC
CGACCGTACGACGCATCTTTTATTTCTTTGTCTGTTTCATTCGTTTTCTT
GAGTTCATAACATGCGGAGCCCTTCCGTTACCTCTACTGCTCTACACTTA
AGCTGATAACCAGAAAATCCAGTACT
Translation:

(SEQ ID NO:89)

MHLSLARSAVLMLLLLFALDNFVGVQPGQITRDVDNRRINLQSRWKPRSL
FKSLHKRASCGGTCTESADCPSTCSTCLGHAQCE
Toxin Sequence:

(SEQ ID NO:90)

Ser-Cys-Gly-Gly-Thr-Cys-Thr-Xaa1-Ser-Ala-Asp-Cys-
Xaa3-Ser-Thr-Cys-Ser-Thr-Cys-Leu-His-Ala-Gln-Cys-
Xaa1-\hat{ }
Name: Cr10.2
Species: circumcisus
Cloned: Yes
DNA Sequence:

(SEQ ID NO:91)

tgtgtgtgtgtggttctgggtccaGCATTTGATGGCAGGAATGCCGCAGT
CAACGAGAGAGCGCCTTGGACGGTCGTTTTGTCCACCACGAATTGCTGCG
GTTATAATACGATGGAATTCTGCCCTGCTTGCATGTGCACTTATTCCTGT
CCAAAAAAGAAAAAACCAGGAAAAGGCCGCAGAAACAACTGATGCTCCAG
GACCCTCTGAACCACGACGT
Translation:

(SEQ ID NO:92)

FDGRNAAVNERAPWTVVLSTTNCCGYNTMEFCPACMCTYSCPKKKKPGKG
RRNN
Toxin Sequence:

(SEQ ID NO:93)

Ala-Xaa3-Xaa4-Thr-Val-Val-Leu-Ser-Thr-Thr-Asn-Cys-
Cys-Gly-Xaa5-Asn-Thr-Met-Xaa1-Phe-Cys-Xaa3-Ala-
Cys-Met-Cys-Thr-Xaa5-Ser-Cys-Xaa3-Lys-Lys-Lys-Lys-
Xaa3-Gly-Lys-Gly-Arg-Arg-Asn-Asn-\hat{ }
Name: Cn9.1
Species: consors
Cloned: Yes
DNA Sequence:
Translation:

(SEQ ID NO:94)

GIFVGVQPEQITRDVDKGYSTDDGHDLLSLLKQISLRACTGSCNSDSECY
NFCDCIGTRCEAQK
Toxin Sequence:

(SEQ ID NO:95)

Ala-Cys-Thr-Gly-Ser-Cys-Asn-Ser-Asp-Ser-Xaa1-Cys-
Xaa5-Asn-Phe-Cys-Asp-Cys-Ile-Gly-Thr-Arg-Cys-Xaa1-
Ala-Gln-Lys-\hat{ }
Name: De6.1
Species: delessertii
Isolated: Yes
Toxin Sequence:

(SEQ ID NO:96)

Ala-Cys-Lys-Xaa3-Lys-Asn-Asn-Leu-Cys-Ala-Ile-Thr-
Xaa1-Met-Ala-Xaa1-Cys-Cys-Ser-Gly-Phe-Cys-Leu-Ile-
Xaa5-Arg-Cys-\hat{ }
Name: Bromosleeper-Dil
Species: distans
Cloned: Yes
DNA Sequence:

(SEQ ID NO:97)

GACAGGATTGAACAAAATTCAGGATGTCAGGATTGGGAATCATGGTGCTA
ACCCTTCTACTTCTTGTGCCCATGGCAACCAGTCAACAGGATGGAGGAGA
GAAGCAGGCGATGCAAAGGGACGCAATCAACGTCGCACCAGGAACATCAA
TCACTCGGAGAAATGTAGATCAGGAGTGCATTGACGCCTGTCAGCTGGAG
GACAAGAATTGCTGTGGCAGAACAGATGGAGAACCCAGATGTGCGAAAAT
CTGCCTCGGATAATTTCTGTACGCTGTCTCATTCATTATTTCATCCGTAC
GAGTGTAAACGAGACCTATTAGAAAGTCGAAGGTTGTGCGTAATTTGATA
AGCATTGTTTGCTGGGACGAACGGA
Translation:

(SEQ ID NO:98)

MSGLGIMVLTLLLLVPMATSQQDGGEKQAMQRDAINVAPGTSITRRNVDQ
ECIDACQLEDKNCCGRTDGEPRCAKICLG
Toxin Sequence:

(SEQ ID NO:99)

Asn-Val-Asp-Gln-Xaa1-Cys-Ile-Asp-Ala-Cys-Gln-Leu-
Xaa1-Asp-Lys-Asn-Cys-Cys-Gly-Arg-Thr-Asp-Gly-Xaa1-
Xaa3-Arg-Cys-Ala-Lys-Ile-Cys-Leu-#
Name: Bromosleeper-Di2
Species: distans
Cloned: Yes
DNA Sequence:

(SEQ ID NO:100)

GACAGGATTGAACAAAATTCAGGATGTCAGGATTGGGAATCATGGTGCTA
ACCCTTCTACTTCTTGTGCCCATGGCAACCAGTCAACAGGATGGAGGAGA
GAAGCAGGCGATGCAAAGGGACGCAATCAACGTCGCACCAGGAACATCAA
TCACTCGGACAGAAACAGATCAGGAGTGCATTGACATCTGTAAGCAGGAG
GACAAGAAATGCTGCGGCAGATCAAATGGAGAACCCACATGTGCGAAAAT
CTGCCTCGGATAATTTCTGTACGCTGTCTCGTTCATTATTTCGTCAGTAC
GAGTTTAAACGAGACCTATTAGAAAGTCGAAGGTTCGTGCTTAATTTGAT
AAGCATTGTTTGCTGGGATGAACGGA
Translation:

(SEQ ID NO:101)

MSGLGIMVLTLLLLVPMATSQQDGGEKQAMQRDAINVAPGTSITRTETDQ
ECIDICKQEDKKCCGRSNGEPTCAKICLG
Toxin Sequence:

(SEQ ID NO:102)

Xaa1-Thr-Asp-Gln-Xaa1-Cys-Ile-Asp-Ile-Cys-Lys-Gln-
Xaa1-Asp-Lys-Lys-Cys-Cys-Gly-Arg-Ser-Asn-Gly-Xaa1-
Xaa3-Thr-Cys-Ala-Lys-Ile-Cys-Leu-#
Name: Bromosleeper-Di3
Species: distans
Cloned: Yes
DNA Sequence:

(SEQ ID NO:103)

GACAGGATTGAACAAAATTCAGGATGTCAGGATTGGGAATCATGGTGCTA
ACCCTTCTACTTCTTGTGCCCATGGCAACCAGTCAACAGGATGGAGGAGA
GAAGCAGGCGATGCAAAGGGACGCAATCAACGTCGCACCAGGAACATCAA
TCACTCGGAGAGAAACAGATCAGGAGTGCATTGACACCTGTGAGCAGGAG
GACAAGAAATGCTGCGGCAGAACAAATGGAGAACCCGTATGTGCGAAAAT
CTGCTTCGGATAATTTCTGTACGCTGTCTCATTCATAATTTCATCAGTAC
GAGTTTAAACGAGACCTATTAGAAAGTCGAAGGTTCGTGCTTAATTTGAT
AAGCATTGTTTGCTGGGATGAACGGA
Translation:

(SEQ ID NO:104)

MSGLGIMVLTLLLLVPMATSQQDGGEKQAMQRDAINVAPGTSITRRETDQ
ECIDTCEQEDKKCCGRTNGEPVCAKICFG
Toxin Sequence:

(SEQ ID NO:105)

Xaa1-Thr-Asp-Gln-Xaa1-Cys-Ile-Asp-Thr-Cys-Xaa1-
Gln-Xaa1-Asp-Lys-Lys-Cys-Cys-Gly-Arg-Thr-Asn-Gly-
Xaa1-Xaa3-Val-Cys-Ala-Lys-Ils-Cys-Phe-#
Name: αA-EIVB
Species: ermineus
Isolated: Yes
Cloned: Yes
DNA Sequence:

(SEQ ID NO:106)

ATGTTCACCGTGTTTCTGTTGGTTGTCTTGGCAACCACTGTCGTTTCCTT
CACTTCAGATCGTGCATCGGATGACAGGAATACCAACGACAAAGCATCTC
GCCTGCTCTCTCACGTTGTCAGGGGATGCTGTGGTAAGTATCCCAATGCT
GCCTGTCATCCTTGCGGTTGTACAGTGGGTAGGCCACCGTATTGTGACAG
ACCCAGTGGTGGAGGACGCTGATGCTCCAGGACCCTCTGAACCACGACGT
Translation:

(SEQ ID NO:107)

MFTVFLLVVLATTVVSFTSDRASDDRNTNDKASRLLSHVVRGCCGKYPNA
ACHPCGCTVGRPPYCDRPSGGGR
Toxin Sequence:

(SEQ ID NO:108)

Gly-Cys-Cys-Gly-Lys-Xaa5-Xaa3-Asn-Ala-Ala-Cys-His-
Xaa3-Cys-Gly-Cys-Thr-Val-Gly-Arg-Xaa3-Xaa3-Xaa5-
Cys-Asp-Arg-Xaa3-Ser-Gly-Gly-#
Name: Ge3.1
Species: generalis
Cloned: Yes
DNA Sequence:

(SEQ ID NO:109)

GGATCCATGATGTCTAAACTGGGAGTCTTGTTGACCATCTGTCTGGTTCT
GTTTCCCCTTACTGCTCTTCCACTGGATGGAGAACAACCTGTAGACCGAC
ATGCCGAGCATATGCAGGATGACAATTCAGCTGCACAGAACCCCTGGGTT
ATTGCCATCAGACAGTGTTGCACGTTCTGCAACTTTGGATGCCAGCCTTG
TTGCGTCCCCTGATAACGTGTTGATGACCAACTTTCTCGAG
Translation:

(SEQ ID NO:110)

GSMMSKLGVLLTICLVLFPLTALPLDGEQPVDRHAEKMQDDNSAAQNPWV
IAIRQCCTFCNFGCQPCCVP
Toxin Sequence:

(SEQ ID NO:111)

Xaa2-Cys-Cys-Thr-Phe-Cys-Asn-Phe-Gly-Cys-Gln-Xaa3-
Cys-Cys-Val-Xaa3-\hat{ }
Name: C. geographus GS-A
Species: geographus
Cloned: Yes
DNA Sequence:

(SEQ ID NO:112)

GCAAGATCATCAGCAGAATGAACCTGACGTGCGTGTTGATCATCGCCGTG
CTCTTTCTGACGGCCTGCCAGCTCATTGCAGCTGATGACTCCAGAGATAA
CCAGAAGCACCGTGCAGTGAGGATGAGAGACGCATTGAAGAATTTCAAAG
ATTCCAGGGCGTGCTCCGGTAGAGGTTCTAGATGTCCTCCCCAATGCTGC
ATGGGTTTGACGTGCGGTCGTGAGTATCCACCCAGATGCGGTTGATATAC
GGTGAACAACTGATATTTCCCCTCTGTGCTCTACCCTCTTTTGCCTGATT
CACCCACACCTATGTGTGGTCATGAACCACTCAGTACCTACACCTCTGGT
GGCTTCAGAGGACGTATATTAAAATAAAACCACATTGCAATGAAAAAAAA
AAAA
Translation:

(SEQ ID NO:113)

MNLTCVLIIAVLFLTACQLIAADDSRDNQKHRAYRMRDALKNFKDSRACS
GRGSRCPPQCCMGLTCGREYPPRCG
Toxin Sequence:

(SEQ ID NO:114)

| | |
|---|---|
| Ala-Cys-Ser-Gly-Arg-Gly-Ser-Arg-Cys-Xaa3-Xaa3-Gln- | |
| Cys-Cys-Met-Gly-Leu-Thr-Cys-Gly-Arg-Xaa1-Xaa5- | |
| Xaa3-Xaa3-Arg-Cys-# | |
| Name: | Conopressin-G |
| Species: geographus | |
| Isolated: Yes | |
| Toxin Sequence: | |
| Cys-Phe-Ile-Arg-Asn-Cys-Xaa3-Lys-Gly-#(SEQ ID NO:115) | |
| Name: EST66 | |
| Species: geographus | |
| Cloned: Yes | |
| DNA Sequence: | |

(SEQ ID NO:116)

TGCTGCCCGAGTAGCAAAGAGGATTCCCTGAACTGCATTGAGACCATGGC
GACCACGGCCACGTGCATGAAGTCCAACAAGGGGGAGATCTACTCCTATG
CGTGCGGCTACTGCGGCAAGAAGAAGGAGAGCTGTTTCGGCGACAAAAAG
CCAGTGACTGACTACCAGTGCCAGACGCGGAACATTCCCAACCCCTGCGG
CGGCGCTGCTCTCTGAAGGCACCAACAGCACCAACAGCACGATCTCCTGT
GTTTCGTCACTGCATTTATGACGTCAAAACCACGTCATGCATGATGACGA
CGATCTCGGCTATGGCATGTATTGAAGAATGGAAATAAACCTAGTTTTCA
GCTGAAAAAA
Translation:

(SEQ ID NO:117)

CCPSSKEDSLNCIETMATTATCMKSMQGEIYSYACGYCGKKKQESCFGDK
KPVTDYQCQTRMPNPCGGAAL
Toxin Sequence:

(SEQ ID NO:118)

Cys-Cys-Xaa3-Ser-Ser-Lys-Xaa1-Asp-Ser-Leu-Asn-Cys-
Ile-Xaa1-Thr-Met-Ala-Thr-Thr-Ala-Thr-Cys-Met-Lys-
Ser-Asn-Lys-Gly-Xaa1-Ile-Xaa5-Ser-Xaa5-Ala-Cys-
Gly-Xaa5-Cys-Gly-Lys-Lys-Lys-Xaa1-Ser-Cys-Phe-Gly-
Asp-Lys-Lys-Xaa3-Val-Thr-Asp-Xaa5-Gln-Cys-Gln-Thr-
Arg-Asn-Ile-Xaa3-Asn-Xaa3-Cys-Gly-Gly-Ala-Ala-Leu-
\hat{ }
Name: EST87
Species: geographus
Cloned: Yes
DNA Sequence:

(SEQ ID NO:119)

CGGGCGCTGCATTCCGGACGTGAAACAGCATCGCCAGCAAGTGGGCATAG
TGCAAGACACtCAGAACAAtGACGCACAtAGTCTGANAAAATAACCATGG
GTATGCGGATGANGTTTAGTGTGTTTCNGCAGGTTGTCNTGGGNACCACT
GTCGTTTCCTTCACNTCACGTCGTGGTCCAAAATCTCGTCGCGGGGAACC
TATTCCGACCACTGTAATCAACTACGGGGAGTGCTGTAAGGATCCATCCT
GTTGGGTTAAGGTGAAGGATTTCCAGTGTCCTGGAGCAAGTCCTCCCAAC
TGAACCACGACATGTCGCCCTCTGCCTGACCTGCTTCACGTTCCGTCTCT
TTCTGCCACTAGAACTCAACAACTCGATCCAACAGACTCCTACTTTACCT
CCGTATTCTGAAACTACTTGGATTTGATTGTCTTTAATATCTACTCACAC
TTGCTGTTATTACATCATCCAAAATTTAACAAGAACATGAAAGGTGTCTG
TTCAAACAAAATCAGGCAATGACAANGGGGGAAAGTCTCCANTCTATCTG
AAAACTGTCACCTGTCACTCTCTTAACCAGGTTTANAACTGANTACCACT
ANAGCTGTTGTNCCACATCANGATCAGNCCAATTTGTANNGTTTCCTTTG
CAAAACTTTTGCCTGAAATTCTTGAAAAGAAACGCTCACAATGTTGGGAA
GTGCTTTTNATTANCTGACAAINISTTGNCANCATGTTCCNTTTCANTAA
NTCTNAAATGNAAACCTCTGTT
Translation:

(SEQ ID NO:120)

MGMRMMFSVFLQVVLGTTVVSFTSRRGPKSRRGEPIPTTVINYGECCKDP
SCWVKVKDFQCPGASPPN
Toxin Sequence:

(SEQ ID NO:121)

Gly-Xaa1-Xaa3-Ile-Xaa3-Thr-Thr-Val-Ile-Asn-Xaa5-
Gly-Xaa1-Cys-Cys-Lys-Asp-Xaa3-Ser-Cys-Xaa4-Val-
Lys-Val-Lys-Asp-Phe-Gln-Cys-Xaa3-Gly-Ala-Ser-Xaa3-
Xaa3-Asn-\hat{ }
Name: G12.1
Species: geographus
Cloned: Yes
DNA Sequence:

(SEQ ID NO:122)

AGCCTTGATACAGAGCTGGTATCTGCTGTTAATACTTGAAAGAACAAGTG
CTGTGAGCCTTCATCTCTCTCTGACTTTAGTTTGGGTCCTGGAGAAAACC
TTGACGGGCAGTATGAAAATTTACCTGTGTCTTGCTTTTGTTCTGCTCCT
GGCTTCTACCATAGTTGATTCAGGGCTTCTTGATAAAATTGAGACTATAA
GAAACTGGAAACGCGATGACAGCTATTGTGATGGATGCCTATGCACCATA
TTAAAAAAAGAGACTTGCACATCGACTATGAGCTGCAGGGGAACATGCCG
AAAAGAGTGGCCATGTTGGGAAGAAGACTGCTACTGTACTGAAATCCAAG
GTGGAGCTTGCGTCACACCCTCAGAATGCAAACCTGGAGAGTGTTGAGGA
TTGGAGTGGCCAGTTCCAGCACATACAGCACCATGGTGCCCTGGACAATC
GTCTATTGAATTGAATATGCCTGTGGCAGGAATCTGTCCTACAAAATAAA
AAAATCATAAGTTAAAAAA
Translation:

(SEQ ID NO:123)

MKIYLCLAFVLLLASTIVDSGLLDKIETIRNWKRDDSYCDGCLCTILKKE
TCTSTMSCRGTCRKEWPCWEEDCYCTEIQGGACVTPSECKPGEC
Toxin Sequence:

(SEQ ID NO:124)

Asp-Asp-Ser-Xaa5-Cys-Asp-Gly-Cys-Leu-Cys-Thr-Ile-
Leu-Lys-Lys-Xaa1-Thr-Cys-Thr-Ser-Thr-Met-Ser-Cys-
Arg-Gly-Thr-Cys-Arg-Lys-Xaa1-Xaa4-Xaa3-Cys-Xaa4-
Xaa1-Xaa1-Asp-Cys-Xaa5-Cys-Thr-Xaa1-Ile-Gln-Gly-
Gly-Ala-Cys-Val-Thr-Xaa3-Ser-Xaa1-Cys-Lys-Xaa3-
Gly-Xaa1-Cys-\hat{ }
Name: G12.2
Species: geographus
Cloned: Yes
DNA Sequence:

(SEQ ID NO:125)

AACGTTGACGGGCAGTATGAACATTTACCTGTGTCTTGCTTTTCTTCTGT
TCCTGCCTTCTACCATAGTTGATTCAGGGCTTCTTGATAAAATTGAGACA
ATAAGGAATTGGAGACGTGATGAAAGCAAGTGTGATCGATGCAATTGCGC
CGAATTAAGATCATCCAGATGCACACAAGCTATCTTCTGCCTTACACCGG
AGTTATGCACACCGAGCATCTCATGTCCGACAGGTGAATGCCGCTGTACT
AAGTTCCATCAGTCAAGATGCACTAGATTCGTAGAATGCGTACCTAATAA
GTGTAGAGACGCATAGAGGCCAGTTCCAGCACATACAGCACCATGATGCC
CTGGACAATCGTGTTGTTGGATTGAATATGCCCGTGGCAGGAATCTGTCC
TACAAAAAA
Translation:

(SEQ ID NO:126)

MNIYLCLAFLLFLPSTIVDSGLLDKIETIRINWRRDESKCDRCNCAELRS
SRCTQAIFCLTPELCTPSISCPTGECRCTKFHQSRCTRFVECVPNKCRDA
Toxin Sequence:

(SEQ ID NO:127)

Asp-Xaa1-Ser-Lys-Cys-Asp-Arg-Cys-Asn-Cys-Ala-Xaa1-
Leu-Arg-Ser-Ser-Arg-Cys-Thr-Gln-Ala-Ile-Phe-Cys-
Leu-Thr-Xaa3-Xaa1-Leu-Cys-Thr-Xaa3-Ser-Ile-Ser-
Cys-Xaa3-Thr-Gly-Xaa1-Cys-Arg-Cys-Thr-Lys-Phe-His-
Gln-Ser-Arg-Cys-Thr-Arg-Phe-Val-Xaa1-Cys-Val-Xaa3-
Asn-Lys-Cys-Arg-Asp-Ala-\hat{ }
Name: Scratching, convulsion
Species: geographus
Isolated: Yes
Toxin Sequence:

(SEQ ID NO:128)

Lys-Phe-Leu-Ser-Gly-Gly-Phe-Lys-Xaa1-Ile-Val-Cys-
His-Arg-Xaa5-Cys-Ala-Lys-Gly-Ile-Ala-Lys-Xaa1-Phe-
Cys-Asn-Cys-Xaa3-Asp-#
Name: Contryphan-Im
Species: imperialis
Isolated: Yes
Toxin Sequence:

(SEQ ID NO:129)

Xaa2-Cys-Gly-Gln-Ala-Xaa4-Cys-#
Name: Im9.1
Species: imperialis
Cloned: Yes
DNA Sequence:

(SEQ ID NO:130)

GTTAAAATGCATCTGTCACTGGCAAGCTCAGCTGCTTTGATGTTGCTTCT
GCTTTTTGCCTTGGGCAACTTCGTTGGGGTCCAGCCAGGACAAATAAGAG
ATCTGAACAAAGGACAGCTCAAGGACAACCGCCGTAACCTGCAATCGCAG
AGGAAACAAATGAGTCTCCTCAAGTCACTTCATGATCGAAATGGGTGTAA
CGGCAACACGTGTTCCAATAGCCCCTGCCCTAACAACTGTTATTGCGATA
CTGAGGACGACTGCCACCCTGACAGGCGTGAACATTAGAGATTAGAGAGT
TTCCTTGTCAACATGATGTCGCACCACACCTCTGCTCTGCAGTGTGTACA
TCGACCAGTCGACGCATCTGTTATTTCTTTGTCTGTTGGATTGTACATCG
ACCAGTCCACGCATCTGTTATTTCTTTGTCTGTTTGATTTGTTTTCGTGT
GTTCATAACACACAGAGCCTTTCTATTATCTGTATTGCAATACACTTTGC
CTGATAACCAGAAAGTCCAGTGCT
Translation:

(SEQ ID NO:131)

MHLSLASSAALMLLLLFALGNTVGVQPGQIRDLNKGQLKDNRRNLQSQRK
QMSLLKSLHDRNGCNGNTCSNSPCPNNCYCDTEDDCHPDRREH
Toxin Sequence:

(SEQ ID NO:132)

Asn-Gly-Cys-Asn-Gly-Asn-Thr-Cys-Ser-Asn-Ser-Xaa3-
Cys-Xaa3-Asn-Asn-Cys-Xaa5-Cys-Asp-Thr-Xaa1-Asp-
Asp-Cys-His-Xaa3-Asp-Arg-Arg-Xaa1-His-\hat{ }
Name: La8.1
Species: laterculatus
Cloned: Yes
DNA Sequence:

(SEQ ID NO:133)

ATGATGTCGAAAATGGGAGCTATGTTTGTCCTTTTGCTTCTTTTCACCCT
GGCATCCAGCCAGCAGGAAGGAGATGTCCAGGCAAGGAAAACACACCCGA
AGAGAGAGTTCCATCGTATTCTGCTAAGGCCTGACAGACAGTCCGAAACG
GCTTGTAGGTCGCTCGGAAGCTACCAATGTATGGGTAAATGCCAACTCGG
GGTTCATTCCTGGTGTGAATGCATTTATAACCGAGGTAGTCAGAAGTCTG
GATGCGCGTGTAGGTGTCAAALAGTGATTAATTGACTCATTTAACTCGTT
GAACGATTTAAAAAATCCAGAGCAATATGTTCGAGAAAAACCGAAGACGA
C
Translation:

(SEQ ID NO:134)

MMSKMGAMFVLLLLFTLASSQQEGDVQARKTITPKREFHRILLRPDRQSE
TACRSLGSYQCMGKCQLGVHSWCECIYNRGSQKSGCACRCQK
Toxin Sequence:

(SEQ ID NO:135)

Xaa2-Ser-Xaa1-Thr-Ala-Cys-Arg-Ser-Leu-Gly-Ser-
Xaa5-Gln-Cys-Met-Gly-Lys-Cys-Gln-Leu-Gly-Val-His-
Ser-Xaa4-Cys-Xaa1-Cys-Ile-Xaa5-Asn-Arg-Gly-Ser-
Gln-Lys-Ser-Gly-Cys-Ala-Cys-Arg-Cys-Gln-Lys-\hat{ }
Name: Lv6.2
Species: lividus
Cloned: Yes
DNA Sequence:

(SEQ ID NO:136)

GGATCCATGAAACTGACGTGTGTGGTGATCATCGCCGTGCTGTTCCTGAC
GGCCAGTCAGCTCATTACAGCTGATTACTCCAGAGATAAGCAGGAGTATC
GTGCAGAGAGGCTGAGAGACGCAATGGGGAAATTCAAAGGTTCCAGGTCG
TGCGGACATAGTGGTGCAGGTTGTTATACTCGCCCTTGCTGCCCTGGTCT
GCATTGCTCTGGCGGCCAAGCTGGAGGCCTGTGCGTGTAATAGTAATAAT
CTGGCGTCTGATATTTCCAGTCTGTGCTCTACCCTCTTTTGCCTGAGTCA
TCCATACCTGTGCTCGAG
Translation:

(SEQ ID NO:137)

MKLTCVVHAVLFLTASQLITADYSRDKQEYRAERLRDAMGKFKGSRSCGH
SGAGCYTRPCCPGLHCSGGQAGGLCV
Toxin Sequence:

(SEQ ID NO:138)

Ser-Cys-Gly-His-Ser-Gly-Ala-Gly-Cys-Xaa5-Thr-Arg-
Xaa3-Cys-Cys-Xaa3-Gly-Leu-His-Cys-Ser-Gly-Gly-Gln-
Ala-Gly-Gly-Leu-Cys-Val-\hat{ }
Name: Lv6.3
Species: lividus
Cloned: Yes
DNA Sequence:

(SEQ ID NO:139)

GGATCCATGAAACTGACGTGTGTGGTGATCATATCCGTGCTGTTCCTGAC
GGCCAGTGAGTTCCTTACAGCTGATTACTCCAGAGATAAGCGGCAGTACC
GTGCTGTGAGGTTGAGAGACGCAATGCGGAATTTCAAAGGTACCAGGGAC
TGCGGGGAATCAGGTCAAGGTTGCTATAGTGTACGTCCTTGCTGCCCTGG
TCTGATTTGCAAAGGCACCGGTGGTGGAGGCCTGTGCCGGCCCTCTGGCA
TCTGATATCTCCCCTCTGTGCTCCACCCTCTTTTGCCTGAGTCATCCATA
CCTGTGCTCGAG
Translation:

(SEQ ID NO:140)

MKLTCVVIISVLFLTASEFLTADYSRDKRQYRAVRLRDAMRNFKGTRDCG
ESGQGCYSVRPCCPGLICKGTGGGGLCRPSGI
Toxin Sequence:

(SEQ ID NO:141)

Asp-Cys-Gly-Xaa1-Ser-Gly-Gln-Gly-Cys-Xaa5-Ser-Val-
Arg-Xaa3-Cys-Cys-Xaa3-Gly-Leu-Ile-Cys-Lys-Gly-Thr-
Gly-Gly-Gly-Leu-Cys-Arg-Xaa3-Ser-Gly-Ile-\hat{ }
Name: Convulsant
Species: magus
Isolated: Yes
Toxin Sequence:

(SEQ ID NO:142)

Val-Xaa5-Xaa1-Thr-His-Xaa3-\hat{ }
Name: MAG-1
Species: magus
Isolated: Yes
Toxin Sequence:

(SEQ ID NO:143)

Arg-Xaa3-Lys-Asn-Ser-Xaa4-\hat{ }
Name: MAG-2
Species: magus
Isolated: Yes
Toxin Sequence:

(SEQ ID NO:144)

Ala-Arg-Xaa3-Lys-Asn-Ser-Xaa4-?
Name: MAG-3
Species: magus
Isolated: Yes
Toxin Sequence:

(SEQ ID NO:145)

Arg-Xaa3-Lys-Asn-Ser-Xaa4-\hat{ }
Name: Mi6.2
Species: miles
Cloned: Yes
DNA Sequence:

(SEQ ID NO:146)

GGATCCATGAAACTGACGTGCGTGGTGATCGTCGCCGTGCTGTTCCTGAC
GGCCTGTCAACTCATTACTGCTGCGAATTACGCCAGAGATGAACAGGAGT
ACCCCGCTGTGAGGTCGAGCGACGTGATGCAGGATTCCGAAGACTTGACG
TTGACCAAGAAATGCACGGACGATTCTCAGTTCTGTAACCCTTCGAATCA
TGACTGCTGCAGTGGGAAGTGTATCGACGAAGGAGACAACGGCATATGCG
CTATAGTCCCTGAAAACTCTTAACAATGTATACTGACATTTCCCCCTCTG
TGCTCCGCCGTCCGTGGCCTGACTCGTCCATCCTTGGGCGTGGTCATGAA
CCGCTCGGTT
Translation:

(SEQ ID NO:147)

MKLTCVVIVAVLFLTACQLITAANYARDEQEYPAVRSSDVMQDSEDLTLT
KKCTDDSQFCNPSNHDCCSGKCIDEGDNGICAIVPENS
Toxin Sequence:

(SEQ ID NO:148)

Cys-Thr-Asp-Asp-Ser-Gln-Phe-Cys-Asn-Xaa3-Ser-Asn-
His-Asp-Cys-Ser-Gly-Lys-Cys-Ile-Asp-Xaa1-Gly-Asp-
Asn-Gly-Ile-Cys-Ala-Ile-Val-Xaa3-Xaa1-Asn-Ser-\hat{ }
Name: Mi6.3
Species: miles
Cloned: Yes
DNA Sequence:

(SEQ ID NO:149)

GGATCCATGAAACTGACGTGTGTGGTGATCGTCGCCGTGCTGTTCCTGAC
GGCCTGTCAACTCATTACTGCTGCGAATTACGCCAGAGATGAACAGGAGT
ACCCTGCTGTGAGGTCGAGCGACGTGATGCAGGATTCCGAAGACCTGACG
TTGACCAAGAAATGCACGGAGGATTCTCAGTTCTGTAACCCTTCGAATCA
TGACTGCTGCAGTGGGAAGTGTATCGACGAAGGAGACAACGGCATATGCG
CTATAGTCCCTGAAAACTCTTAACAATGTATACTGACATTTCCCCCTCTG
TGCTCCGCCGTCCGTGGCCTGACTCGTCCATCCTTGGGCGTGGTCATGAA
CCGCTCG
Translation:

(SEQ ID NO:150)

MKLTCVVIVAVLFLTACQLITAANYARDEQEYPAVRSSDVMQDSEDLTLT
KKCTEDSQFCNPSKHDCCSGKCIDEGDNGICAIVPENS
Toxin Sequence:

(SEQ ID NO:151)

Cys-Thr-Xaa1-Asp-Ser-Gln-Phe-Cys-Asn-Xaa3-Ser-Asn-
His-Asp-Cys-Cys-Ser-Gly-Lys-Cys-Ile-Asp-Xaa1-Gly-
Asp-Asn-Gly-Ile-Cys-Ala-Ile-Val-Xaa3-Xaa1-Asn-Ser-
\hat{ }
Name: Mf6.1
Species: miliaris
Cloned: Yes
DNA Sequence:

(SEQ ID NO:152)

GGATCCATGAACTGACGTGTGTGGTGATCATCGCCGTGCTGTTCCTGACG
GCCTGTCAACTCACTACAGCTGTGACTTCCTCCAGAGGTCAACAGAAGCA
TCGTGCTCTGAGGTCAACTGACAAAAACTCCAGGATGACCAAGCGTTGCA
CGCCTCCAGGTGGACTCTGTTACCATGCTTATCCCTGCTGCAGCAAGACT
TGCAATCTCGATACCAGCCAATGTGAGCCTAGGTGGTCATGAACCACTCA
ATACCCTCTCCTCTGGAGGCTTCAGAGGAACTACATTGAAATAAAACCGC
ATTGCAACGAAAAAAAAAAAAAAAAAA
Translation:

(SEQ ID NO:153)

LTCVVIIAVLFLTACQLTTAVTSSRGQQKHRALRSTDKNSRMTKRCTPPG
GLCYHAYPCCSKTCNLDTSQCEPRWS
Toxin Sequence:

(SEQ ID NO:154)

Cys-Thr-Xaa3-Xaa3-Gly-Gly-Leu-Cys-Xaa5-His-Ala-
Xaa5-Xaa3-Cys-Cys-Ser-Lys-Thr-Cys-Asn-Leu-Asp-Thr-
Ser-Gln-Cys-Xaa1-Xaa3-Arg-Xaa4-Ser-\hat{ }
Name: Mn10.3
Species: monachus
Cloned: Yes
DNA Sequence:

(SEQ ID NO:155)

tgtgtgtgtgtggttctgggtccaGCATCTGATGTCAGGAATGCCGCAGT
CCACGAAAGACAGAAGGATCTGGTCGTTACGGCCACCACGACTTGCTGTG
GTTATAATCCGATGACAATGTGCCCTCCTTGCATGTGCACTAATACCTGC
AAAAAAAGTGGCTGATGCTCCAGGACCCTCTGAACCACGACGT
Translation:

(SEQ ID NO:156)

SDVRNAAVHERQKDLVVTATTTCCGYNPMTMCPPCMCTNTCKKSG
Toxin Sequence:

(SEQ ID NO:157)

Xaa2-Lys-Asp-Leu-Val-Val-Thr-Ala-Thr-Thr-Thr-Cys-
Cys-Gly-Xaa5-Asn-Xaa3-Met-Thr-Met-Cys-Xaa3-Xaa3-
Cys-Met-Cys-Thr-Asn-Thr-Cys-Lys-Lys-Ser-#
Name: Mn8.1
Species: monachus
Cloned: Yes
DNA Sequence:

(SEQ ID NO:158)

ATGATGTCGAAAATGGGAGCTATGTTTGTCCTTTTGCTTCTTTTCACCCT
GGCATCCAGCCAGCAGGAAGGAGATGTCCAGGCAAGGAAAACAAGCCTGA
AGAGCGACTTCTATCGTGCTCTGAGAGGGTATGACAGACAGTGCACTCTT
GTCAACAATTGTGACCGGAACGGTGAGCGTGCGTGTAACGGTGATTGCTC
TTGCGAGGGCCAGATTTGTAAATGCGGTTATAGAGTCAGTCCTGGGAAGT
CAGGATGCGCGTGTACTTGTAGAAATGCCAAATGAATCATTTAACTCGTT
GAAAGATTTTTTAAAAATCCAGAGCTATATGTTCGAGAAAAACCGAAGAC
Translation:

(SEQ ID NO:159)

MMSKMGAMFVLLLLFTLASSQQEGDVQARKTSLKSDFYRALRGYDRQCTL
VNNCDRNGERACNGDCSCEGQICKCGYRVSPGKSGCACTCRNAK
Toxin Sequence:

(SEQ ID NO:160)

Xaa2-Cys-Thr-Leu-Val-Asn-Asn-Cys-Asp-Arg-Asn-Gly-
Xaa1-Arg-Ala-Cys-Asn-Gly-Asp-Cys-Ser-Cys-Xaa1-Gly-
Gln-Ile-Cys-Lys-Cys-Gly-Xaa5-Arg-Val-Ser-Xaa3-Gly-
Lys-Ser-Gly-Cys-Ala-Cys-Thr-Cys-Arg-Asn-Ala-Lys-\hat{ }
Name: Pn1.3
Species: pennaceus
Cloned: Yes
DNA Sequence:

(SEQ ID NO:161)

ATGCGCTGTCTCCCAGTCTTCGTCATTCTTCTGCTGCTGACTGCATCTGC
ACCTAGCGTTGATGCCAAAGTTCATCTGAAGACCAAAGGTGATGGGCCCC
TGTCATCTTTCCGAGATAATGCAAAGAGTACCCTACAAAGACTTCAGGAC
AAAAGCACTTGCTGTGGCTTTAAGATGTGTATTCCTTGTCGTTAACCAGC
ATGAAGGATCC
Translation:

(SEQ ID NO:162)

MRCLPVFVILLLLTASAPSVDAKVHLKTKGDGPLSSFRDNAKSTLQRLQD
KSTCCGFKMCIPCR
Toxin Sequence:

(SEQ ID NO:163)

Ser-Thr-Cys-Cys-Gly-Phe-Lys-Met-Cys-Ile-Xaa3-Cys-
Arg-\hat{ }
Name: Pn9.1
Species: pennaceus
Cloned: Yes
DNA Sequence:

(SEQ ID NO:164)

ATGTTGCTTCTGCTGTTTGCCTTGGGCAGCTTCGTTGTGGTCCAGTCAGG
ACAGATAACAAGAGATGTGGACAATGGGCAGCTCGCGGACAACCGCCGTA
CCCTGCGATCGCAGTGGAAGCAAGTGAGTTTCTTCAAGTCACTTGATAAA
CGACTGACTTGTAACGATCCTTGCCAGATGCATTCCGATTGCGGCATATG
TGAATGCGTGGAAAATAAATGCATATTTTTCATGTAAACGGATTGAGTTT
GCTTGTCAACACAATGTCGCACTGCAGCTCTTCTCTACCGGTGGGTACAT
CGACCAAACGACGCATCTTTTATTTCTTTGTCTGTTTCGTTTGTTCTCCT
GTGTTCATAACGTACAGAGCCCTTTAACTACCCTTACTGCTCTTCACTTA
ACCTGATAACCTGAAGGTCCGGTGCAGCTGGCGTAGCCTTCACAGTTTCG
Translation:

(SEQ ID NO:165)

MLLLLFALGSFVVVQSGQITRDVDNGQLADNRRTLRSQWKQVSFFKSLDK
RLTCNDPCQMHSDCGICECVENKCIFFM
Toxin Sequence:

(SEQ ID NO:166)

Leu-Tbr-Cys-Asn-Asp-Xaa3-Cys-Gln-Met-His-Ser-Asp-
Cys-Gly-Ile-Cya-Xaa1-Cys-Val-Xaa1-Asn-Lys-Cys-Ile-
Phe-Phe-Met-\hat{ }
Name: Pu6.1
Species: pulicarius
Cloned: Yes
DNA Sequence:

(SEQ ID NO:167)

ATGAAACTGACGTGTGTGGTGATCGTCGCCGTGCTGTTCCTGACGGCCTG
TCAACTCAGTACAGCTGATGACTCCAGAGATGAGCAGCAGGATCCTTTGG
TGAGGTCGCATCGTGAGGAGCAGAAAGCCGAGGACCCCAAGACGGCCGAG
AGATGTTCAGATTTCGGATCCGACTGTGTTCCTGCTACTCATAACTGCTG
CAGTGGTGAATGTTTTGGCTTCGAGGACTTCGGCTTATGCACGTAAAACT
GGTCTGACGTCTGATATTCCCCCCTCTGTCCTTCATCCTCTTTTGCCTGA
TTCATCCATACCTATATGTGCTCCTGAACCGCTGTGTACCTTTACCCTGG
TGGCTTCAGAGGACGTTATATCAAAATAAAACCGCGTTGCAATGACAAAA
AAAAAAAAAAAAA
Translation:

(SEQ ID NO:168)

MRLTCVVIVAVLFLTACQLSTADDSRDEQQDPLVRSHREEQKAEDPKTAE
RCSDFGSDCVPATHNCCSGECFGFEDFGLCT
Toxin Sequence:

(SEQ ID NO:169)

Cys-Ser-Asp-Phe-Gly-Ser-Asp-Cys-Val-Xaa3-Ala-Thr-
His-Asn-Cys-Cys-Ser-Gly-Xaa1-Cys-Phe-Gly-Phe-Xaa1-
Asp-Phe-Gly-Leu-Cys-Thr-\hat{ }
Name: Bromosleeper-P1
Species: purpurascens
Cloned: Yes
DNA Sequence:

(SEQ ID NO:170)

GACAGGATTGAACAAAATTCAGGATGTCAAGATTTGGAATCATGGTGCTA
ACCTTTCTACTTCTTGTGTCCATGGCAACCAGCCATCGTTATGCAAGAGG
GAAGCAGGCGACGCGAAGGAACGCAATCAACATCAGACGGAGAAGCACAC
CAAAAACTGAGGCGTGCGAAGAGGTCTGTGAGCTGGAAGAAAAGCACTGC
TGCTGCATAAGAAGTGACGGACCCAAATGTTCCCGTAAGTGCCTGTTGTC
AATCTTCTGTTAGTTTCTGTACACTGTCTCATTCATTATCTTATCAGTAC
AAGTGTAAACGAGACATGTCAGAAAGTCGAAGGTTGTGCGTAATTTGATA
AGTATTGTTTGCTGGGATGAACGGA
Translation:

(SEQ ID NO:171)

MSRFGIMVLTFLLLVSMATSHRYARGKQATRRNAINTRRRSTPKTEACEE
VCELEEKIICCCIRSDGPKCSRKCLLSIFC
Toxin Sequence:

(SEQ ID NO:172)

Xaa3-Lys-Thr-Xaa1-Ala-Cys-Xaa1-Xaa1-Val-Cys-Xaa1-
Leu-Xaa1-Xaa1-Lys-His-Cys-Cys-Cys-Ile-Arg-Ser-Asp-
Gly-Xaa3-Lys-Cys-Ser-Arg-Lys-Cys-Leu-Leu-Ser-Ile-
Phe-Cys-\hat{ }
Name: Bromosleeper-P2
Species: purpurascens
Cloned: Yes
DNA Sequence:

(SEQ ID NO:173)

GACAGGATTGAACAAAATTCAGGATGTCAGGATTGGGAATCATGGTGCTA
ACCCTTCTACTTCTTGTGTCCATGGCAACCAACCATCAGGATAGAGGAGA
GAAGCAGGTGACGCAAAGGGACGCAATCAACGTCAGACGGAGAAGATCAA
TCACCCAGCAAGTCGTATCTGAGGAGTGCAAAAAGTACTGTAAGAAACAG
AACAAGAATTGCTGCAGCAGTAAACATGAAGAACCCAGATGTGCCAAGAT
ATGCTTCGGATAGTTTCTGTACACGGTCTCATTCATTATTTTATCAGTAC
AAGTTAAACGAGACCTATCAGAAGTCGAAGGTTGTGCATAATTTGATAAA
CATTGTTTGCTGGGATGAACGGA
Translation:

(SEQ ID NO:174)

MSGLGTMVLTLLLLVSMATNIIQDRGEKQVTQRDAINVRRRRSITQQVVS
EECKIKYCKKQNKNCCSSKHEEPRCAIKICFG
Toxin Sequence:

(SEQ ID NO:175)

Val-Val-Ser-Xaa1-Xaa1-Cys-Lys-Lys-Xaa5-Cys-Lys-
Lys-Gln-Asn-Lys-Asn-Cys-Cys-Ser-Ser-Lys-His-Xaa1-
Xaa1-Xaa3-Arg-Cys-Ala-Lys-Ile-Cys-Phe-#
Name: P29
Species: purpurascens
Isolated: Yes
Toxin Sequence:

(SEQ ID NO:176)

Asp-Cys-Cys-Gly-Val-Lys-Leu-Xaa1-Met-Cys-His-Xaa3-
Cys-Leu-Cys-Asp-Asn-Ser-Cys-Lys-Asn-Xaa5-Gly-Lys-#
Name: P4.1
Species: purpurascens
Cloned: Yes
DNA Sequence:

(SEQ ID NO:177)

ATGTTCACCGTGTTTCTGTTGGTTGTCTTGGCAACCACTGTCGTTTCCTT
CACTTCAGATCGTGCATCGGATGACAGGAATACCAACGACAAAGCATCTC
GCCTGCTCTCTCACGTTGTCAGGGGATGCTGTGGTAGCTATCCCAATGCT
GCCTGTCATCCTTGCGGTTGTAAAGATAGGCCATCGTATTGTGGTCAAGG
ACGCTGATGCTCCAGGACCCTCTGAACCACGACGT
Translation:

(SEQ ID NO:178)

MFTVFLLVVLATTVVSFTSDRASDDRNTNDKASRLLSHVVRGCCGSYPNA
ACHPCGCKDRPSYCGQGR
Toxin Sequence:

(SEQ ID NO:179)

Gly-Cys-Cys-Gly-Ser-Xaa5-Xaa3-Asn-Ala-Ala-Cys-His-
Xaa3-Cys-Gly-Cys-Lys-Asp-Arg-Xaa3-Ser-Xaa5-Cys-
Gly-Gln-#
Name: P4.2
Species: purpurascens
Cloned: Yes
DNA Sequence:

(SEQ ID NO:180)

ATGTTCACCGTGTTTCTGTTGGTTGTCTTGGCAACCACTGTCGTTTCCTT
CACCGTAGATCGTGCAACTGATGGCAGGAGTGCTGCAGCCATAGCGTTTG
CCCTGATCGCTCCGACCGTCCGGGAAGGATGCTGTTCTAATCCTGCCTGT
CATCCTTGCGGTTGTAAAGATAGGCCATCGTATTGTGGTCAAGGACGCTG
ATGCTCCAGGACCCTCTGAACCACGACGT
Translation:

(SEQ ID NO:181)

MFTVFLLVVLATTVVSFTVDRATDGRSAAAIAFALIAPTVREGCCSNPAC
HPCGCKDRPSYCGQGR
Toxin Sequence:

(SEQ ID NO:182)

Xaa1-Gly-Cys-Cys-Ser-Asn-Xaa3-Ala-Cys-His-Xaa3-
Cys-Gly-Cys-Lys-Asp-Arg-Xaa3-Ser-Xaa5-Cys-Gly-Gln-
#
Name: P8.1
Species: purpurascens
Cloned: Yes
DNA Sequence:

(SEQ ID NO:183)

ATGATGTCGAAAATGGGAGCTATGTTTGTCCTTTTGCTTCTTTTCACCCT
GGCATCCAGCCAGCAGGAAGGAGATGTCCAGGCAAGGAAAACACGCCTGA
CGAGGGACTTCTATCGTACTCTGCCAGTGTCTACTAGAGGATGCAGCGGC
TCCCCTTGTTTTAAAAACAAAACGTGTCGGGATGAATGCATATGCGGCGG
CTTATCCAATTGTTGGTGTGGCTACGGCGGTAGTCGAGGATGCAAGTGTA
CATGTAGAGAGTGATTAATCGACTCTTTAACTCGTTGAATTATTTAAAAA
ATCCAGAGCAATATGTTCGAGAAAAACCGAAGAC
Translation:

(SEQ ID NO:184)

MMSKMGAMFVLLLLFTLASSQQEGDVQARKTRLTRDFYRTLPVSTRGCSG
SPCFKNKTCRDECICGGLSNCWCGYGGSRGCKCTCRE
Toxin Sequence:

(SEQ ID NO:185)

Gly-Cys-Ser-Gly-Ser-Xaa3-Cys-Phe-Lys-Asn-Lys-Thr-
Cys-Arg-Asp-Xaa1-Cys-Ile-Cys-Gly-Gly-Leu-Ser-Asn-
Cys-Xaa4-Cys-Gly-Xaa5-Gly-Gly-Ser-Arg-Gly-Cys-Lys-
Cys-Thr-Cys-Arg-Xaa1-\hat{ }
Name: U021 homolog
Species: purpurascens
Cloned: Yes
DNA Sequence:

(SEQ ID NO:186)

CGACCTCAAGAGGGATCGATAGCAGTTCATGATGTCTAAACTGGGAGCCT
TGTTGACCATCTGTCTGCTTCTGTTTCCCATTACTGCTCTTCTGATGGAT
GGAGATCAACCTGCAGACCGACCTGCAGAACGTATGGATTACGACATTTC
ATCTGAGGTGCATCGTTTGCTTGAAAGGAGACACCCGCCCTGTTGCATGT
ACGGCAGATGCCGTCGATATCCCGGATGCTCTAGTGCCTCTTGTTGCCAG
GGAGGATAACGTGTTGATGACCAACTTTGTTACACGGCTACGTCAAGTGT
CTACTGAATAAGTAAAACGATTGCAGT
Translation:

(SEQ ID NO:184)

MMSKLGALLTICLLLFPITALLMDGDQPADRPAERMDYDISSEVHRLLER
RHPPCCMYGRCRRYPGCSSASCCQGG
Toxin Sequence:

(SEQ ID NO:188)

His-Xaa3 -Xaa3-Cys-Cys-Met-Xaa5-Gly-Arg-Cys-Arg-
Arg-Xaa5-Xaa3-Gly-Cys-Ser-Ser-Ala-Ser-Cys-Cys-Gln-
Gly-#
Name: ψ-PIIIF
Species: purpurascens
Isolated: Yes
Toxin Sequence:

(SEQ ID NO:189)

Gly-Xaa3-Xaa3-Cys-Cys-Leu-Xaa5-Gly-Ser-Cys-Arg-
Xaa3-Phe-Xaa3-Gly-Cys-Xaa5-Asn-Ala-Leu-Cys-Cys-
Arg-Lys-#
Name: Qc6.4
Species: quercinus
Cloned: Yes
DNA Sequence:

(SEQ ID NO:190)

GGATCCATGAAACTGACGTGCGTGGTGATCATCGCCGTGCTGTTTCTGAC
AGCCAGTCAGCTCGTTACAGCTGATTACACCAGAGATAAATGGCAATACC
CTGCAGCGAGTTTGAGAGGCGGAATGTGGAATTTGAGAGATACCAGGGCG
TGCTCGCAAGTAGGTGAAGCTTGTTTTCCTCAGAAACCTTGCTGCCCTGG
ATTCCTTTGCAATCACATCGGAGGCATGTGCCACCACTAGTAACAGTCTG
GCATCTGATATTTCCCCTCTGCGCTCCACCCTCTTTTGGCTGATTCATCC
TTACCTGTGTGTGGTCATGAACCACTCAGTAGCTACACCTCTGGTGGCTT
CAGAGGACGTATATCAAAATAAAACCACATTGCAAAAAAAAAAAAAAAAA
Translation:

(SEQ ID NO:191)

MKLTCVVIIAVLFLTASQLVTADYTRDKWQYPAASLRGGMWNLRDTRACS
QVGEACFPQKPCCPGFLCNHIGGMCIIII
Toxin Sequence:

(SEQ ID NO:192)

Ala-Cys-Ser-Gln-Val-Gly-Xaa1-Ala-Cys-Phe-Xaa3-Gln-
Lys-Xaa3-Cys-Cys-Xaa3-Gly-Phe-Leu-Cys-Asn-His-Ile-
Gly-Gly-Met-Cys-His-His-\hat{ }
Name: QcII
Species: quercinus
Isolated: Yes
Toxin Sequence:

(SEQ ID NO:193)

Asp-Cys-Gln-Xaa3-Cys-Gly-His-Asn-val-Cys-Cys-\hat{ }
Name: EST17l
Species: radiatus
Cloned: Yes
DNA Sequence:

(SEQ ID NO:194)

CATGAACTGTCTCGTACTGGCTTTGGTTACCATCGGTCTTCTGGCTGCAA
CAACCGCAGCCCCTCTGGACACCACCACGGTCCTCCTCAGCACAACTACA
CGCGATGTCAAGGGCTGTGTGTACGAGGGCATAGAGTACAGTGTCGGAGA
GACCTACCAGGCAGACTGCAACACGTGTCGCTGTGATGGCTTTGACCTGG
CTACATGCACCGTCGCGGGCTGCACAGGCTTTGGACCCGAGTGATTGGTA
CTATTCCACACCTAGCAATGTTCACACTGGAACCGGAACTTGATACTACC
TTCTAAATATAATCAATTTGTTTCAAAAGGCCCAAA
Translation:

(SEQ ID NO:195)

MNCLVLALVTIGLLAATTAAPLDTTTVLLSTTTRDVKGCVYEGIEYSVGE
TYQADCNTCRCDGFDLATCTVAGCTGFGPE
Toxin Sequence:

(SEQ ID NO:196)

Gly-Cys-Val-Xaa5-Xaa1-Gly-Ile-Xaa1-Xaa5-Ser-Val-
Gly-Xaa1-Thr-Xaa5-Gln-Ala-Asp-Cys-Asn-Thr-Cys-Arg-
Cys-Asp-Gly-Phe-Asp-Leu-Ala-Thr-Cys-Thr-Val-Ala-
Gly-Cys-Thr-Gly-Phe-Gly-Xaa3-Xaa1-\hat{ }
Name: EST202
Species: radiatus
Cloned: Yes
DNA Sequence:

(SEQ ID NO:197)

GTGAGAGTCCAACAGCCCAAACCTTTCAACTCACTATGTGGCAGTTGCAG
TTTTCAACGTCTGGACAGGATTCAACAAAATTCAGGATGTCAGGATTGGG
AATCATGGTGCTAACCCTTCTACTTCTTGTGTCCATGGCAACCAGTCGTC
AGGATAGAGGAGTGGGACAGCTGATGCCACGCGTCTCGTTCAAAGCCTGC
AAATCAAATTATGATTGCCCCCAGCGTTTCAAATGCTGCAGTTACACCTG
GAATGGATCCAGTGGATACTGTAAACGTGTTTGCTATCTTTATCGTTAGT
GTAATACACAAAGTGACTCTGTTCATTCCTCTCCATCATCTCTTTAGAAA
CAACACGGTGTCGAGATCGTTTCTTTGTGATGAAGAGTAGTATCACGGGC
AGAGTTCACTAGAGATCTCAAATGAAAAACAAGATTATTTAGTAAGTTGG
GGAAAATCTGGATCTCGAAAAGATTCCTTGAAAACTCCGTATTTAACACG
CTTGAGAGATGATAATAAAGAATTCTGAAAGACAAA
Translation:

(SEQ ID NO:198)

MSGLGIMVLTLLLLVSMATSRQDRGVGQLMPRVSFKACKSNYDCPQRFKC
CSYTWNGSSGYCKRVCYLYR
Toxin Sequence:

(SEQ ID NO:199)

Ala-Cys-Lys-Ser-Asn-Xaa5-Asp-Cys-Xaa3-Gln-Arg-Phe-
Lys-Cys-Cys-Ser-Xaa5-Thr-Xaa4-Asn-Gly-Ser-Ser-Gly-
Xaa5-Cys-Lys-Arg-Val-Cys-Xaa5-Leu-Arg-\hat{ }
Name: R8.1
Species: radiatus
Cloned: Yes
DNA Sequence:

(SEQ ID NO:200)

ATGATGTCGAAAATGGGAGCTATGTTTGTCCTTTTGCTTCTTTTCACCCT
GGCATCCAGCCAGCAGGAAGGAGATGTCCAGGCAAGGAAAACACACCCGA
AGAGAGAGTTCCAACGTATTCTGCTAAGGTCTGGCAGAAAGTGCAATTTC
GACAAATGTAAAGGTACCGGAGTCTACAATTGTGGGGAATCCTGCTCATG
CGAAGGTTTGCACAGTTGTCGCTGCACTTATAACATCGGTTCTATGAAGT
CTGGATGCGCGTGTATTTGTACATACTATTAATGATTAATTGACTCGTTT
AACTCGTTGAACGATTTAAAAAATCCAGAGCAATATGTTCGAGAAAAACC
GAAGAC
Translation:

(SEQ ID NO:201)

MIMSKMGAMFVLLLLFTLASSQQEGDVQARKTHPKREFQRILLRSGRKCN
EDKCKGTGVYNCGESCSCEGLHSCRCTYNIGSMKSGCACICTYY
Toxin Sequence:

(SEQ ID NO:202)

Lys-Cys-Asn-Phe-Asp-Lys-Cys-Lys-Gly-Thr-Gly-Val-
Xaa5-Asn-Cys-Gly-Xaa1-Ser-Cys-Ser-Cys-Xaa1-Gly-
Leu-His-Ser-Cys-Arg-Cys-Thr-Xaa5-Asn-Ile-Gly-Ser-
Met-Lys-Ser-Gly-Cys-Ala-Cys-Ile-Cys-Thr-Xaa5-Xaa5-
\hat{ }
Name: R8.2
Species: radiatus
Cloned: Yes
DNA Sequence:

(SEQ ID NO:203)

ATGATGTCGAAAATGGGAGCTATGTTTGTCCTTTTGCTTCTTTTCACCCT
GGCATCCAGGCAGCAGGAAGGAGATGTCCAGGCAAGGAAAACACGCCTGA
CGAGCGACTTCTATAGTGTTCTGCAAAGGTATGGACTAGGATGCGCTGGC
ACTTGTGGTTCAAGCAGCAATTGTGTTAGAGATTATTGTGACTGCCCAAA
ACCCAATTGTTACTGCACTGGCAAAGGCTTTCGTCAACCAGGATGCGGGT
GTTCATGTTTGGGGTGATTAATTGGCTCTTTTAACTCGTTGAACGATTTA
AAAAATCCAGAGCAATATGTTCGAGAAAAACCGAAGAC
Translation:

(SEQ ID NO:204)

MMSKMGAMFVLLLLFTLASRQQEGDVQARKTRLTSDFYSVLQRYGLGCAG
TCGSSSNCVRDYCDCPKIPNCYCTGKGFRQPGCGCSCLG
Toxin Sequence:

(SEQ ID NO:205)

Xaa5-Gly-Leu-Gly-Cys-Ala-Gly-Thr-Cys-Gly-Ser-Ser-
Ser-Asn-Cys-Val-Arg-Asp-Xaa5-Cys-Asp-Cys-Xaa3-Lys-
Xaa3 -Asn-Cys-Xaa5 -Cys-Thr-Gly-Lys-Gly-Phe-Arg-
Gln-Xaa3-Gly-Cys-Gly-Cys-Ser-Cys-Leu-#
Name: Bromosleeper-Sn
Species: sponsalis
Cloned: Yes
DNA Sequence:

(SEQ ID NO:206)

GACAGGATTGAACAAAATTCAGGATGTCAGGATTGGGAATCATGGTGCTG
ACCCTTTTGCTTCTTGTGTCCATGGCAACCAGCCATAAGGATGGAGGAGA
GAAGCAGGCGATGCAAAGGGACGCAATCAACGTCAGACTGAGAAGATCAC
TCACTCGGAGAGCAGTAACTGAGGCGTGCACGGAGGACTGTAAGACTCAG
GACAAGAAGTGCTGCGGCGAAATGAATGGACAACACACATGTGCCAAGAT
ATGCCTCGGATAGTCTCTGTACGCTGTCTCATTCATTATCTCATCAGTAC
AAGTGTAAACGAGACAGGTCAGAAAGTCGAAGGTTGTTCGAAATTTGATA
AGCATTGTTTACTGGGACGAACGGA
Translation:

(SEQ ID NO:207)

MSGLGIMVLTLLLLVSMATSHKDGGEKQAMQRDAINVRLRRSLTRRAVTE
ACTEDCKTQDKKCCGEMNGQHTCAKICLG
Toxin Sequence:

(SEQ ID NO:208)

Ala-Val-Thr-Xaa1-Ala-Cys-Thr-Xaa1-Asp-Cys-Lys-Thr-
Gln-Asp-Lys-Lys-Cys-Cys-Gly-Xaa1-Met-Asn-Gly-Gln-
His-Thr-Cys-Ala-Lys-Ile-Cys-Leu-#
Name: Contryphan-Sm-dW4, V7
Species: stercusmuscarum
Isolated: Yes
Toxin Sequence:

(SEQ ID NO:209)

Gly-Cys-Xaa3-Xaa4-Gln-Xaa3-Val-Cys-#
Name: Conopressin-S
Species: striatus
Isolated: Yes
Toxin Sequence:

(SEQ ID NO:210)

Cys-Ile-Ile-Arg-Asn-Cys-Xaa3-Arg-Gly-#
Name: S6.4
Species: striatus
Cloned: Yes
DNA Sequence:

(SEQ ID NO:211)

AGGTCGACTCGCTGCTTGCCTGACGGAACGTCTTGCCTTTTTAGTAGGAT
CAGATGCTGCGGTACTTGCAGTTCAATCTTAAAGTCATGTGTGAGCTGAT
CCAGCGGTTGATCTTCCTCCCTCTGTGCTCCATCCTTTTCTGCCTGAGTT
CTCCTTACCTGAGAGTGGTCATGAACCACTCATCACCTACTCTTCTGGAG
GCTTCAGAGGAGCTACAGTGAAATAAAAGCCGCATTGC
Translation:

(SEQ ID NO:212)

STRCLPDGTSCLFSRIRCCGTCSSILKSCVS
Toxin Sequence:

(SEQ ID NO:213)

Cys-Leu-Xaa3-Asp-Gly-Thr-Ser-Cys-Leu-Phe-Ser-Arg-
Ile-Arg-Cys-Cys-Gly-Thr-Cys-Ser-Ser-Ile-Leu-Lys-
Ser-Cys-Val-Ser-\hat{ }
Name: U010 homolog
Species: striatus
Cloned: Yes
DNA Sequence:

(SEQ ID NO:214)

CGGCTTCTAATACGACTCACTATAGGGCAAGCAGTGGTAACAACGCAGAG
TACGCGGGGGGACGGCAGACCAGCTGGGGACCAGACAGACGTCAAACAGC
ATCGCAGTCAGGTGTGGAGATCCCAAGACACCCAGAAGAAGGAGACAGAA
GAGTTATCGTTCGTAACACAATGGCCATGAACATGTCGATGACACTCTGC
ATGTTTGTAATGGTCGTCGTGGCAGCCACTGTCATTGATTCCACTCAGTT
ACAAGAACCAGATCTCAGTCGCATGCGACGCAGCGGGCCTGCTGACTGTT
GCAGGATGAAAGAGTGTTGCACCGACAGAGTGAACGAGTGTCTACAGCGC
TATTCTGGCCGGGAAGATAAATTCGTTTCGTTTTGTTATCAGGAGGCCAC
AGTCACATGTGGATCTTTTAACGAAATCGTGGGCTGTTGCTATGGATATC
AAATGTGCATGATACGAGTTGTGAAACCGAACAGTCTAAGTGGGGCCCAT
GAGGCGTGCAAAACCGTTTCTTGTGGTAACCCTTGCGCTTGAGGTGTCCT
CGCGCCACGTCACCTGTGTACAGCGCCGTCACCAGAGCCCTGATCTTTAT
GCCCTTATCTGTCTTTTTGCTCTTTCACTCTCTGAAGTCTTGAGGTTTGT
TCCATTCTTGTCAATCATCTCACGCGCATCCAAGTAAATAAAGGTGACGT
GACAAAC
Translation:

(SEQ ID NO:215)

MAMNMSMTLCMFVMVVVAATVIDSTQLQEPDLSRMRRSGPADCCRMKECC
TDRVNECLQRYSGREDKFVSFCYQEATVTCGSFNEIVGCCYGYQMCMRVY
KPNSLSGAHEACKTVSCGNPCA
Toxin Sequence:

(SEQ ID NO:216)

Ser-Gly-Xaa3-Ala-Asp-Cys-Cys-Arg-Met-Lys-Xaa1-Cys-
Cys-Thr-Asp-Arg-Val-Asn-Xaa1-Cys-Leu-Gln-Arg-Xaa5-
Ser-Gly-Arg-Xaa1-Asp-Lys-Phe-Val-Ser-Phe-Cys-Xaa5-
Gln-Xaa1-Ala-Thr-Val-Thr-Cys-Gly-Ser-Phe-Asn-Xaa1-
Ile-Val-Gly-Cys-Cys-Xaa5-Gly-Xaa5-Gln-Met-Cys-Met-
Ile-Arg-Val-Val-Lys-Xaa3-Asn-Ser-Leu-Ser-Gly-Ala-
His-Xaa1-Ala-Cys-Lys-Thr-Val-Ser-Cys-Gly-Asn-Xaa3-
Cys-Ala-\hat{ }
Name: WG002
Species: striatus
Isolated: Yes
Toxin Sequence:

(SEQ ID NO:217)

Xaa4-Ser-Xaa4-Arg-Met-Gly-Asn-Gly-Asp-Arg-Arg-Ser-
Asp-Gln-\hat{ }
Name: Sx8.1
Species: striolatus
Cloned: Yes
DNA Sequence:

(SEQ ID NO:218)

ATGATGTCGAAAATGGGAGCTATGTTTGTCCTTTTGCTTCTTTTGACCCT
GGCATCCAGCCAGCAGGAGGGAGATGTCCAGGCAAGGAAAACAAGCCTGA
AGAGCGACTTCTATCGTGCTCTGAGACCGTATGACAGACAGTGCACTTTT
GTCAACAATTGTCAACAGAACGGTGCGTGTAACGGTGATTGCTCTTGCGG
GGACCAGATTTGTAAATGCGGTTATAGAATCAGTCCTGGGAGGTCAGGAT
GCGCGTGTACTTGTAGAAATGCCAAATGAATCACTTAACTCGTTGAAAGA
TTTTTAAAAATCCAGAGCTATATGTTCGAGAAAAACCGAAGAC
Translation:

(SEQ ID NO:219)

MMSKMGAMFVLLLLLTLASSQQEGDVQARKTSLKSDFYRALRIPYDRQCT
FVNNCQQNGACNGDCSCGDQICKCGYRISPGRSGCACTCRNAK
Toxin Sequence:

(SEQ ID NO:220)

Xaa2-Cys-Thr-Phe-Val-Asn-Asn-Cys-Gln-Gln-Asn-Gly-
Ala-Cys-Asn-Gly-Asp-Cys-Ser-Cys-Gly-Asp-Gln-Ile-
Cys-Lys-Cys-Gly-Xaa5-Arg-Ile-Ser-Xaa3-Gly-Arg-Ser-
Gly-Cys-Ala-Cys-Thr-Cys-Arg-Asn-Ala-Lys-\hat{ }
Name: Ts6.3
Species: tessulatus
Cloned: Yes
DNA Sequence:

(SEQ ID NO:221)

GGATCCATGAAACTGACGTGTGTGGTGATCATCGCCGTGCTGTTCCTGAC
GGCCTGTCAATTCATTATAGCTGATTTCTCCAGAGATAAGCGGGTACATC
GTGCAGAGAGGTTGAGAGACATAATGCAGAATTTCAGAGGTACCAGGTCG
TGCGCGGAATTTGGTGAAGTTTGTAGTTCTACCGCTTGCTGCCCTGATTT
GGATTGCGTTGAGGCCTATTCACCCATCTGTCTCTGGGAATAGTCTGGCA
TCTGATATTTCCCGTCTGTGCTCTACCTACTTCTGCCGGATTCATCCATA
CCTATGTGTGGCCATGAACCACTCAGTACCTACACCTCTGGTGGCTTCCT
AGGGACGTATATCAAAATAAAACCACATTGCAAAAAAAAAAAAAAAAA
Translation:

(SEQ ID NO:222)

MKLTCVVIIAVLFLTACQFIIADFSRDKRVHRAERLRDIMQNFRGTRSCA
EFGEVCSSTACCPDLDCVEAYSPICLWE
Toxin Sequence:

(SEQ ID NO:223)

Ser-Cys-Ala-Xaa1-Phe-Gly-Xaa1-Val-Cys-Ser-Ser-Thr-
Ala-Cys-Cys-Xaa3-Asp-Leu-Asp-Cys-Val-Xaa1-Ala-
Xaa5-Ser-Xaa3-Ile-Cys-Leu-Xaa4-Xaa1-\hat{ }
Name: 4/43 SNX
Species: textile
Isolated: Yes
Cloned: Yes
DNA Sequence:

(SEQ ID NO:224)

CGATTGCAGGGGTTACGATGCGCCGTGTAGCTCTGGCGCGCCATGTTGTG
ATTGGTGGACATGTTCAGCACGAACCAACCGCTGTTTTTAGGCTGACCAC
AAGCCATCCGACATCACCACTCTCCTCTTCAGAGGCTTCAAGGCTTTTTG
TTCTCCTTTTGAAGAATCTTTACGAGTGAACAAACAAGTAGAATAGCACG
TTTTTCCCCCTTTGAAAAATCAATAATGGAGGTTAAACAAAACTGTCTTC
TTCAATAAAGATTTTATCATAAT
Translation:

(SEQ ID NO:225)

IQGGGDERQKAKIMThSRSDRDCRGYDAPCSSGAPCCDWWTCSARTNRCF
Toxin Sequence:

(SEQ ID NO:226)

Asp-Cys-Arg-Gly-Xaa5-Asp-Ala-Xaa3-Cys-Ser-Ser-Gly-
Ala-Xaa3-Cys-CyS-Asp-Xaa4-Xaa4-Thr-Cys-Ser-Ala-
Arg-Thr-Asn-Arg-Cys-Phe-\hat{ }
Name: convulsion
Species: textile
Isolated: Yes
Toxin Sequence:

(SEQ ID NO:227)

Asn-Cys-Xaa3-Xaa5-Cys-Val-Val-Xaa5-Cys-Cys-Xaa3-
Xaa3-Ala-Xaa5-Cys-Xaa1-Ala-Set-Gly-Cys-Arg-Xaa3-
Xaa3-#
Name: Tx1.6
Species: textile
Cloned: Yes
DNA Sequence:

(SEQ ID NO:228)

ATGCACTGTCTCCCAATCTTCGTCATTCTTCTGCTGCTGACTGCATCTGG
ACCTAGCGTTGATGCCCAACTGAAGACCAAAGATGATGTGCCCCTGTCAT
CTTTCCGAGATCATGCAAAGAGTACCCTACGAAGACTTCAGGACAAACAG
ACTTGCTGTGGCTATAGGATGTGTGTTCCTTGTGGTTAACCAGCATGAAG
GATCC
Translation:

(SEQ ID NO:229)

MHCLPIFVILLLLTASGPSVDAQLKTKDDVPLSSFRDHAKSTLRRLQDKQ
TCCGYRMCVPCG
Toxin Sequence:

(SEQ ID NO:230)

Xaa2-Thr-Cys-Cys-Gly-Xaa5-Arg-Met-Cys-Val-Xaa3-
Cys-H
Name: Tx6.14
Species: textile
Cloned: Yes
DNA Sequence:

(SEQ ID NO:231)

GTTATGGAGCGATTGCTATAGTTGGTTAGGATCATGTATTGCGCCCTCGC
AGTGTTGTTCTGAGGTTTGTGATTATTACTGCCGCCTATGGCGATGAACT
CGGACCACAAGCCAT
Translation:

(SEQ ID NO:232)

LWSDCYSWLGSCIAPSQCCSEVCDYYCRLWR
Toxin Sequence:

(SEQ ID NO:233)

Asp-Cys-Xaa5-Ser-Xaa4-Leu-Gly-Ser-Cys-Ile-Ala-
Xaa3-Ser-Gln-Cys-Cys-Ser-Xaa1-Val-Cys-Asp-Xaa5-
Xaa5-Cys-Arg-Leu-Xaa4-Arg-\hat{ }
Name: Tx6.3
Species: textile
Cloned: Yes
DNA Sequence:

(SEQ ID NO:234)

AGCTGACGAATGAAAAATTCCGAGAATGTCAAGCTCAGCAAGAGAAAATG
TGTGGAACAATGGAAATACTGCACCCGAGAGTCCTTATGTTGCGCGGGTT
TGTGTTTGTTTAGTTTCTGCATTCTATAACGCTAATCCAGAGTCGTATAT
TCCGTCTAAGCTCCACCTGGCACTGTCTGGTATGTTCCTGCCAGTGACTG
GTCTCATACCGCTTAGACTCTGGTCCGTCTTCTCTGCAACCACAGGAGAA
CGTGCATTATTACAATAAACGCATACTGC
Translation:

(SEQ ID NO:235)

RMKNSENVKLSKRKCVEQWKYCTRESLCCAGLCLFSFCIL
Toxin Sequence:

(SEQ ID NO:236)

Lys-Cys-Val-Xaa1-Gln-Xaa4-Lys-Xaa5-Cys-Thr-Arg-
Xaa1-Ser-Leu-Cys-Cys-Ala-Gly-Leu-Cys-Leu-Phe-Ser-
Phe-Cys-Ile-Leu-\hat{ }
Name: Tx6.7
Species: textile
Cloned: Yes
DNA Sequence:

(SEQ ID NO:237)

CAGAGCCGCTCTGGTGTGCAGACCTGTCTCCAGCCCTCCGTCTCCCTGAT
CGGTGGTTCTGCCTGCATAGCTGTCTTCTCCACGAAGCTTTCCACAGGTA
TAAATAACGCTTCAGTCTCCCGTCCTGTATTGGGCCGCCGTTACAAGCCA
GACCGATACAGCCAGGTCCAGTCTACTTTGCGAGTGAGTTAAAAGCTCCA
GCATTCTACCAGCATCACCAGAATGAAGGTGAGCAGCGTGCTGATCGTGG
CTACGCTGACACTGACCGCAGGCCAGCTGGTTAGTGCTTCTTCCCATTAC
TCAAAAGATGTCCAGATTCTTCCTTCTGTGAGATCAGCTGACGAAGTGGA
AAATTCCGAGAATGTCAGGCTCAGCAAGAGAAGATGTGTGGAACAATGGG
AAGTCTGCGGCATAATCTTGTTCTCCTCATCATGTTGCGGGCAGTTGTGT
TTGTTTGGTTTCTGCGTTCTATAACGCTAATCCAGAGTCGTATATTCCGT
CTAAGCTCCA
Translation:

(SEQ ID NO:238)

MKVSSVLIVATLTLTAGQLVSASSHYSKDVQILPSVRSADEVENSENVRL
SKRRCVEQWEVCGIILFSSSCCGQLCLFGFCVL
Toxin Sequence:

(SEQ ID NO:239)

Cys-Val-Xaa1-Gln-Xaa4-Xaa1-Val-Cys-Gly-Ile-Ile-
Leu-Phe-Ser-Ser-Ser-Cys-Cys-Gly-Gln-Leu-Cys-Leu-
Phe-Gly-Phe-Cys-Val-Leu-\hat{ }
Name: TxVIIA
Species: textile
Isolated: Yes
Toxin Sequence:

(SEQ ID NO:240)

Cys-Gly-Gly-Xaa5-Ser-Thr-Xaa5-Cys-Xaa1-Val-Asp-
Ser-Xaa1-Cys-Cys-Ser-Asp-Asn-Cys-Val-Arg-Ser-Xaa5-
Cys-Thr-Leu-Phe-#
Name: U030
Species: textile
Isolated: Yes
Toxin Sequence:

(SEQ ID NO:241)

Gly-Cys-Asn-Asn-Ser-Cys-Gln-Xaa1-His-Ser-Asp-Cys-
Xaa1-Ser-His-Cys-Ile-Cys-Thr-Ser-Arg-Gly-Cys-Gly-
Ala-Val-Asn-#
Name: Bromosleeper-T1
Species: tulipa
Cloned: Yes
DNA Sequence:

(SEQ ID NO:242)

CAGGATTGAACAAAATTCAGGATGTCAGGATTGGGAATCATGGTGCTAAC
CCTTCTACTTCTTGTGTCCATGGCAACCAGTCATCGTTATGCAAGAGAAA
AGCAGGCGACGCGAAGGGACGCAGTCAACGTCAGACGGAGAAGCAGACCA
AAAACAAAGGAGTGCGAAAGGTACTGTGAGCTGGAGGAAAAGCACTGCTG
CTGCATAAGAAGTAACGGACCCAAATGTTCCAGAATATGCATATTCAAAT
TTTGGTGTTAGTTTTCTGTACACTGTCCATTCATTATCTTATCAGTACAA
GTGTAAACGAGACATGTCAGAAAGTCGAAGGTTGTGCGTAATTTGATAAG
CATTGTTTACTGGGACGAACGGA
Translation:

(SEQ ID NO:243)

MSGLGIMVLTLLLLVSMATSHRYAREKQATRRDAVNVRRRSRPKTKECER
YCELEEKIHCCCIIRSNGPKCSRICIFKFWC
Toxin Sequence:

(SEQ ID NO:244)

Xaa3-Lys-Thr-Lys-Xaa1-Cys-Xaa1-Arg-Xaa5-Cys-Xaa1-
Leu-Xaa1-Xaa1-Lys-His-Cys-Cys-Cys-Ile-Arg-Ser-Asn-
Gly-Xaa3-Lys-Cys-Ser-Arg-Ile-Cys-Ile-Phe-Lys-Phe-
Xaa4-Cys-\hat{ }
Name: Bromosleeper-T2
Species: tulipa
Cloned: Yes
DNA Sequence:

(SEQ ID NO:245)

CAGGATTGAACAAAATTCAGGATGTCAGGATTGGGAATCATGGTGCTAAC
CCTTCTCCTTCTTGTGCTAATGACAACCAGTCATCAGGATGCAGGAGAGA
AGCAGGCGATGCAAAGGGACGCAAAGAACTTCAGTCGGAGAAGATTAGTC
ATTCGGAGACCAAAAACAAGGGAGTGCGAAATGCAGTGTGAGCAGGAGGA
GAAACACTGCTGCCGCGTAAGAGATGGTACGGGCCAATGTGCCCCTAAGT
GCTTGGGAATTAACTGGTAGTTTCTGTACACTGTCTCATTCATTATCTTA
TCAGTACACGTGTAACGAGACATGTCAGAAAGTCGAAGGTAGTGCGTAAT
TTGATAAGCATTGTTTACTGGGACGAACGGA
Translation:

(SEQ ID NO:246)

MSGLGIMVLTLLLLVLMTTSHQDAGEKQAMQRDAKNFSRRRLVIRRPKTR
ECEMQCEQEEKHCCRVRDGTGQCAPKCLGINW
Toxin Sequence:

(SEQ ID NO:247)

Xaa3-Lys-Thr-Arg-Xaa1-Cys-Xaa1-Met-Gln-Cys-Xaa1-
Gln-Xaa1-Xaa1-Lys-His-Cys-Cys-Arg-Val-Arg-Asp-Gly-
Gln-Cys-Ala-Xaa3-Lys-Cys-Leu-Gly-Ile-Asn-Xaa4-\hat{ }
Name: T8.1
Species: tulipa
Cloned: Yes
DNA Sequence:

(SEQ ID NO:248)

ATGATGTCGAAAATGGGAGCTATGTTTGTCCTTTTGCTTCTTTTCACCCT
GGCATCCAGCCAGCAGGAAGGAGATGTCCAGGCAAGGAAAACACGCCTGA
AGAGCGACTTCTATCGTGCTCTGCCAAGGTTTGGCCCAATATGCACTTGT
TTTAAAAGCCAGAACTGTCGGGGTTCTTGTGAATGCATGTCACCTCCCGG
TTGTTACTGCAGTAACAATGGCATTCGTGAACGAGGATGCTCGTGTACAT
GTCCAGGGACTGGTTGAATGATTTGAAAAATTCAGAGCAATATGTTGCAG
AAAAACCGAAGACCGAGACTTCTCACAATAAATCCATAAAGACATTAAAA
AAAAAAAAAAAAA
Translation:

(SEQ ID NO:249)

MMSKMGAMFVLLLLFTLASSQQEGDVQARKTRLKSDFYRALPRFGPICTC
FKSQNCRGSCECMSPPGCYCSNNGIRERGCSCTCPGTG
Toxin Sequence:

(SEQ ID NO:250)

Phe-Gly-Xaa3-Ile-Cys-Thr-Cys-Phe-Lys-Ser-Gln-Asn-
Cys-Arg-Gly-Ser-Cys-Xaa1-Cys-Met-Ser-Xaa3-Xaa3-
Gly-Cys-Xaa5-Cys-Ser-Asn-Asn-Gly-Ile-Arg-Xaa1-Arg-
Gly-Cys-Ser-Cys-Thr-Cys-Xaa3-Gly-Thr-#
Name: T8.2
Species: tulipa
Cloned: Yes
DNA Sequence:

(SEQ ID NO:251)

ATGATGTCGAAAATGGGAGCTATGTTTGTCCTTTTGCTTCTTTTCACCCT
GGCATCCAGCCAGCAGGAAGGAGATGTCCAGGCAAGGAAAACACGCCTGA
AGAGCGACTTCTATCGTACTCTGGCAATATCTGACAGAGGATGCACTGGC
AACTGTGATTGGACGTGTAGCGGTGATTGCAGCTGCCAGGGCACATCTGA
CTCGTGTCACTGCATTCCACCAAAATCAATAGGCAACAGATGCCGGTGTC
AGTGTAAAAGAAAAATCGAAATTGACTGATTCTTTTAACTCGTTGAACGA
TTTAAAAATCAGACCAATATGTAGGCAGAAAACCGAAGACTCTGAGACTC
TCGTAATAATCGTAAGCAAAAAAAAAAAAAAAA
Translation:

(SEQ ID NO:252)

MIMSKMGAMFVLLLLFTLASSQQEGDVQARKTRLKSDFYRTLAISDRGCT
GNCDWTCSGDCSCQGTSDSCHCIPPKSIGNRCRCQCKRKIEID
Toxin Sequence:

(SEQ ID NO:253)

Gly-Cys-Thr-Gly-Asn-Cys-Asp-Xaa4-Thr-Cys-Ser-Gly-
Asp-Gly-Asp-Cys-Ser-Cys-Gln-Gly-Thr-Ser-Asp-Ser-
Cys-His-Cys-Ile-Xaa3-Xaa3-Lys-Ser-Ile-Gly-Asn-Arg-
Cys-Arg-Cys-Gln-Cys-Lys-Arg-Ile-Xaa1-Asp-\hat{ }
Name: Vr6.1
Species: Virgo
Cloned: Yes
DNA Sequence:

(SEQ ID NO:254)

GGATCCATGAAACTGACGTGTGTGGTGATCATCACTGTGCTGTTCCTGAC
GGCCAGTCAGCTCATTACAGCTGATTACTCCAGAGATCAGCGGCAGTACC
GTGCAGTGAGGTTGGGAGATGAAATGCGGAATTTCAAAGGTGCCAGGGAC
TGCGGGGGACAAGGTGAAGGTTGTTATACTCAACCTTGCTGCCCTGGTCT
GCGGTGCCGTGGCGGCGGTACTGGAGGAGGCGTATGCCAGCTGTAGTAAT
AGTTTGGCATCTGATATTTCCCCTCTGTGCTCCACCCTCTTTTGCCTGAT
TCATCCTTACCTATGTGTGGTCATGAACCACTCAGTAGCTACACCTCTGG
TGGATTCAGAGAACGTATATCAAAATAAAACCACATTGCAATAAAAAAAA
AAAA
Translation:

(SEQ ID NO:255)

MKLTCVVIITVLFLTASQLITADYSRDQRQYRAVRLGDEMRNFKGARDCG
GQGEGCYTQPCCPGLRCRGGGTGGGVCQL
Toxin Sequence:

(SEQ ID NO:256)

Asp-Cys-Gly-Gly-Gln-Gly-Xaa1-Gly-Cys-Xaa5-Thr-Gln-
Xaa3-Cys-Cys-Xaa3-Gly-Leu-Arg-Cys-Arg-Gly-Gly-Gly-
Thr-Gly-Gly-Gly-Val-Cys-Gln-Leu-\hat{ }
Name: R6.9
Species: radiatus
Cloned: Yes
DNA Sequence:

(SEQ ID NO:257)

ATCATGCAGAAACTGACAATCCTGCTTCTTGTTGCTGCTATACTGATGTC
GACCCAGGTCCTGATTCAAGGTGGTGGAGAAAAACGCCAAAAAGTCAACA
TTTTTTCAAAAAGAAAGACAGATGCTGAGACCTGGTGGGAGGGCGAATGC
TCTAATTGGTTAGGAAGTTGTTCGACGCCCTCAAATTGCTGTCTCAAGAG
TTGTAATGGGCACTGCACATTGTGGTGATGAACTCTGACCACAAAGCCAT
CCAACATCACCGCTCTCCTCTTCAGAGTCTTCAAG
Translation:

(SEQ ID NO:258)

MQKLTILLLVAMLMSTQVLIQGGGEKRQKVMFSKRLKTDAETWWEGECSN
WLGSCSTPSNCCLKSCNGHCTLW
Toxin Sequence:

(SEQ ID NO:259)

Xaa4-Xaa4-Xaa1-Gly-Xaa1-Cys-Ser-Asn-Xaa4-Leu-Gly-
Ser-Cys-Ser-Thr-Xaa3-Ser-Asn-Cys-Cys-Leu-Lys-Ser-
Cys-Asn-Gly-His-Cys-Thr-Leu-Xaa4-\hat{ }
Name: R6.10
Species: radiatus
Cloned: Yes
DNA Sequence:

(SEQ ID NO:260)

ATCATGCAGAAACTGATAATCCTGCTTCTTGTTGCTGCTGTACTGATGTC
CACCCAGGCCCTGATTCAAGGTGGTGGAGGAAAACGCCAACAGGCAAAGA
GCAAGTATTTTTCCGAAAGAAAGGCACCTGCTAAGCGTTGGTTTGGACAC
GAAGAATGCACTTATTGGTTGGGGCCTTGTGAGGTGGACGACACGTGTTG
TTCTGCCAGTTGTGAGTCCAAGTTCTGCGGGTTGTGGTGATGGACACTGA
CCACAAGTCATCCTACATCGCCACTCTCCTGTTCAGAGTCTTCAAG
Translation:

(SEQ ID NO:261)

MQKLIILLLVAAVLMSTQALIQGGGGKRQQAKSKYFSERKAPAKRWFGHE
ECTYWLGPCEVDDTCCSASCESKFCGLW
Toxin Sequence:

(SEQ ID NO:262)

Xaa4-Phe-Gly-His-Xaa1-Xaa1-Cys-Thr-Xaa5-Xaa4-Leu-
Gly-Xaa3-Cys-Xaa1-Val-Asp-Asp-Thr-Cys-Cys-Ser-Ala-
Ser-Cys-Xaa1-Ser-Ala-Ser-Cys-Xaa1-Ser-Lys-Phe-Cys-
Gly-Leu-Xaa4-\hat{ }
Name: Wi6.1
Species: wittigi
Cloned: Yes
DNA Sequence:

(SEQ ID NO:263)

GGATCCATGAAACTGACGTGTGTGGTGATCATCGCCTTGCTGTTCCTGAC
GGCCTGTCAGCTCATTACGGCTGATTACTCCAGAGATGAGCAGTCTGGCA
GTACAGTGCGGTTTCTAGACAGACCACGGCGTTTTGGTTCGTTCATACCG
TGCGCCCGTTTAGGTGAACCATGTACCATATGCTGCCGTCCTTTGAGGTG
CCGTGAAAGCGGAACACCCACATGTCAAGTGTGATTGTCTGGCATCTGAT
ATTTCCCCTCTGTGCCCTACCCTCTTTTGCCTGAGTCATCCATACCTGTG
CTCGAG
Translation:

(SEQ ID NO:264)

MKLTCVVIIALLFLTACQLITADYSRDEQSGSTVRFLDRPRRFGSFIPCA
RLGEPCTICCRPLRCRESGTPTCQV
Toxin Sequence:

(SEQ ID NO:265)

Phe-Gly-Ser-Phe-Ile-Xaa3-Cys-Ala-Arg-Leu-Gly-Xaa1-
Xaa3-Cys-Thr-Ile-Cys-Cys-Arg-Xaa3-Leu-Arg-Cys-Arg-
Xaa1-Ser-Gly-Thr-Xaa3-Thr-Cys-Gln-Val-\hat{ }
Name: Rg6.6
Species: regius
Cloned: Yes
DNA Sequence:

(SEQ ID NO:266)

GGATCCATGAAACTGACGTGCGTGGTGATCATGGCCTCGCTGTTCCTGGC
GGCCTGTCAATTCCTTACAGCTGGAGGTGACTCAAGAAGTAAGCAGCGGT
ATCCTGATTGGAGGCTGGGCTACCGAAAGTCCAAGTTGATGGCTAAGAAG
ACGTGCCTGGAACATAACAAACTATGTTGGTATGATAGAGACTGCTGCAC
CATATATTGTAATGAAAACAAATGCGGCGTGAAACCTCAATGAATGTTTC
ACACACACACACACACACACACACACACACACACACACACACACACACAC
ACACACACACATCTGGCGTCTGACCATTCCCCCTCTGTGCTCTATCCTCT
TGTTCCTGAGTCATCCATACCTGTGCTCGAG
Translation:

(SEQ ID NO:267)

MKLTCVVIMASLFLAACQFLTAGGDSRSKQRYPDWRLGYRKSKLMAKKTC
LEHNKLCWYDRDCCTIYCNENIKCGVKPQ
Toxin Sequence:

(SEQ ID NO:268)

Thr-Cys-Leu-Xaa1-His-Asn-Lys-Leu-Cys-Xaa4-Xaa5-
Asp-Arg-Asp-Cys-Cys-Thr-Ile-Xaa5-Cys-Asn-Xaa1-Asn-
Lys-Cys-Gly-Val-Lys-Xaa3-Gln-\hat{ }
Name: R6.9
Species: radiatus
Cloned: Yes
DNA Sequence:

(SEQ ID NO:269)

ATCATGCAGAAACTGACAATCCTGCTTCTTGTTGCTGCTATACTGATGTC
GACCCAGGTCCTGATTCAAGGTGGTGGAGAAAAACGCCAAAAAGTCAACA
TTTTTTCAAAAAGAAAGACAGATGCTGAGACCTGGTGGGAGGGCGAATGC
TCTAATTGGTTAGGAAGTTGTTCGACGCCCTCAAATTGCTGTCTCAAGAG
TTGTAATGGGCACTGCACATTGTGGTGATGAACTCTGACCACAAAGCCAT
CCAACATCACCGCTCTCCTCTTCAGAGTCTTCAAG
Translation:

(SEQ ID NO:270)

MQKLTILLLVAAILMSTQVLIQGGGEKRQKVNWSKRKTDAETWWEGECSN
WLGSCSTPSNCCLKSCNGHCTLW
Toxin Sequence:

(SEQ ID NO:271)

Xaa4-Xaa4-Xaa1-Gly-Xaa1-Cys-Ser-Asn-Xaa4-Leu-Gly-
Ser-Cys-Ser-Thr-Xaa3-Ser-Asn-Cys-Cys-Leu-Lys-Ser-
Cys-Asn-Gly-His-Cys-Thr-Leu-Xaa4-\hat{ }
Name: R6.10
Species: radiatus
Isolated: Yes
Cloned: Yes
DNA Sequence:

(SEQ ID NO:272)

ATCATGCAGAAACTGATAATCCTGCTTCTTGTTGCTGCTGTACTGATGTC
CACCCAGGCCCTGATTCAAGGTGGTGGAGGAAAACGCCAACAGGCAAAGA
GCAAGTATTTTTCCGAAAGAAAGGCACCTGCTAAGCGTTGGTTTGGACAC
GAAGAATGCACTTATTGGTTGGGGCCTTGTGAGGTGGACGACACGTGTTG
TTCTGCCAGTTGTGAGTCCAAGTTCTGCGGGTTGTGGTGATGGACACTGA
CCACAAGTCATCCTACATCGCCACTCTCCTGTTCAGAGTCTTCAAG
Translation:

(SEQ ID NO:273)

MQKLIILLLVAAVLMSTQALIQGGGGKRQQAKSKYFSERKAPAKRWFGHE
ECTYWLGPCEVDDTCCSASCESKFCGLW
Toxin Sequence:

(SEQ ID NO:274)

Xaa4-Phe-Gly-His-Xaa1-Xaa1-Cys-Thr-Xaa5-Xaa4-Leu-
Gly-Xaa3-Cys-Xaa1-Val-Asp-Asp-Thr-Cys-Cys-Ser-Ala-
Ser-Cys-Xaa1-Ser-Ala-Ser-Cys-Xaa1-Ser-Lys-Phe-Cys-
Gly-Leu-Xaa4-\hat{ }
Name: Sf5.1
Species: spurius
Cloned: Yes
DNA Sequence:

(SEQ ID NO:275)

GGAAGCTGACTACAAGCAGAATGCGCTGTCTCCCAGTCTTCGTCATTCTT
CTGCTGCTGATTCCATCTGCACCTAGCACTGATGCCCGACCGAAGACCAA
AGATGATGTGCGCCTGGCATCTTTCCACGGTAAGGCAAAGCGAACCCTAC
AAATACCTAGGGGGAATATCCACTGTTGCACAAAATATCAGCCGTGCTGT
TCTTCACCATCATAAAGGGAAATGACTTTGATGAGACCCCTGCGAACTGT
CCCTGGATGTGAAATTTGGAAACGAGACTGTTCCTTTCGCGCGTGTTCGT
GGAATTTCGAATGGTCGTTAATAACACGCTGCCTCTTGCAAACTACAATC
TCTCTGTCCTTTATCTGTGGACTGGATGTCAACACTG
Translation:

(SEQ ID NO:276)

MRCLPVFVILLLLIPSAPSTDARPKTKDDVRLASFHGKAKRTLQIPRGNL
HCCTKYQPCCSSPS
Toxin Sequence:

(SEQ ID NO:277)

Gly-Asn-Ile-His-Cys-Cys-Thr-Lys-Xaa5-Gln-Xaa3-Gln-
Xaa3-Cys-Cys-Ser-Ser-Xaa3-Ser-\hat{ }
Name: Nb5.1
Species: riobilis
Cloned: Yes
DNA Sequence:

(SEQ ID NO:278)

ATGCGCTGTCTCCCAGTCTTCGTCATTCTTCTGCTGCTGACTGCATCTGC
ACCAAGCGTTGATGCCCGACCGAAGACCAAAGATGATGTGCTCCGGGCAT
CTTTCCGCGATAATGCAAAGAGTACCCTACAAAGACTTTGGAACAAACGC
ATCTGCTGCCCCATAATTCTTTGGTGCTGTGGTTAACCAGCATGAAGTTC
CCAGGA
Translation:

(SEQ ID NO:279)

MRCLPVFVILLLLTASAPSVDARPKTKDDVLRASFRDNAKSTLQRLWNKR
ICCPIILWCCG
Toxin Sequence:

(SEQ ID NO:280)

Ile-Cys-Cys-Xaa3-Ile-Ile-Leu-Xaa4-Cys-Cys-#
Name: Bt5.1
Species: betulinus
Cloned: Yes
DNA Sequence:

(SEQ ID NO:281)

ATGCGCTGTCTCCCAGTCTTCATCATTCTTCTGGTGCTGATTGCATCTGC
ACCTACCGTTGATGCCCGACCAAAGATCGAAGATGATGAGTCCCTGGCAT
CTTTCCATGNTCATNAACCACCATNANNGNTNCANCTTTTGAACAAACGC
AATTGCTGCCCAGACTCTCCTCCGTGCTGTCATTAACCAGCATGAAGGTT
CAGGA
Translation:

(SEQ ID NO:282)

MRCLPVFIILLVLIASAPTVDARPKIEDDESLASFH?H?PP????LLKRN
CCPDSPPCCH
Toxin Sequence:

(SEQ ID NO:283)

Asn-Cys-Cys-Xaa3-Asp-Ser-Xaa3-Xaa3-Cys-Cys-His-\hat{ }
Name: t-PVA
Species: purpurascens
Isolated: Yes
Cloned: Yes
DNA Sequence:

(SEQ ID NO:284)

GGAATTCCAAATGATGTAATTACTGACTACATGGTCATAGTGTATACCCA
TTGAAAAATTTCTATGACATTTCAGTTGTTAGATCATCCAGTTCCACAGA
TGGAAAGACAGAGAGATAGTAGCTTGCAAGTGGCAGCGTGTTGTTAACGA
CCATTCGACATTCCATGAACACGTGTGAAAGGAGCAGTCTGCTTTCCAAA
TCTGACATCCAGGGACAGTTTGCAGGGGTCTCATCCAAAGTCATCTTCCT
TTATCCCAAAGTACAGCACCGCATCTGTTTTGGACAGCAACCGCGTTTCT
TCCAAAATCTTTGTAGGGTTCCTTTTGCATTATCGTGGAGATGCCAGGGG
CATATCATCTTTGGTCTTCGGATGAGCATCAACGCAAGGTGCAGATGGAA
TCAGCAGCAGAAGAATGACGAAGACTGGCAGACAGCGCATTCTGCTTGTA
GTCAGCTTCCGAATTCCAAGCCGAATTCTGCAGATATCCATCACACTGGC
GGCCGCTCGAGCATGCATCTAGAGGGCCCAATTCGCCCTATAGTGAGTCG
TATGACAATTCACTGGC
Translation:

(SEQ ID NO:285)

MRCLPFVILLLLIPSAPCVDAHPKTKDDMPLASFHDNAKGTLQRFWKKGC
CPKQMRCCTLG
Toxin Sequence:

(SEQ ID NO:286)

Gly-Cys-Cys-Xaa3-Lys-Gln-Met-Arg-Cys-Cys-Thr-Leu#
Name: Af5.2
Species: ammiralis
Cloned: Yes
DNA Sequence:

(SEQ ID NO:287)

GGAAGCTGACTACAAGCAGAATGCACTGTCTCCCAGTCGTCGTCATTCTT
CTGCTGCTGACTGCATCTGGTGGACCTAGCGTTGATGCCCGACTGAAGAC
CAAAGATGATGTGCCCCTGTCATCTTTCCGCGATAATACAAGAGTATCCT
ACAAACACTTTGGAAGCGAGGCAACTGCTGTGAATTTTGGGAGTTTTGCT
GTGATTAACCAGCATGAAGG
Translation:

(SEQ ID NO:288)

MHCLPVVVILLLLTASGGPSVDARLKTKDDVPLSSFRDNTKSILQRLWKR
GNCCEFWEFCCD
Toxin Sequence:

(SEQ ID NO:289)

Gly-Asn-Cys-Cys-Xaa1-Phe-Xaa4-Xaa1-Phe-Cys-Cys-
Asp-\hat{ }
Name: Da5.1
Species: dalli
Cloned: Yes
DNA Sequence:

(SEQ ID NO:290)

GGAAGCTGACTACAAGCAGAATGCACTGTCTCCCAGTCTTCGTCATTCTT
CTGCTGCTGACTGCATCTGGACCTAGCGTTGATGCCCAACCGAAGACCGA
AGTTGATGTGCCCCTGTCATCTTTCCGCGATAATGCAAAGCGTGCCCTAC
AAAGACTTCCGCGTTGCTGTGAATATTGGAAGTTGTGCTGTGGTTAACCA
GCATGAAGG
Translation:

(SEQ ID NO:291)

MHCLPVFVILLLLTASGPSVDAQPKTEVDVPLSSFRDNAKRALQRLPRCC
EYWKLCCG
Toxin Sequence:

(SEQ ID NO:292)

Cys-Cys-Xaa1-Xaa5-Xaa4-Lys-Leu-Cys-Cys-#
Name: Om5.1
Species: omaria
Cloned: Yes
DNA Sequence:

(SEQ ID NO:293)

GGAAGCTGACTACAAGCAGAATGCGCTGTCTCCCAGTCTTCGTCATTCTT
CTGCTGCTAACTGCATCTGCACCTAGCGTTGATGCCCGACCGAAGGCCAA
AGATGATGTGCCCCTGGCATCTTTCCGTGATAATGCAAAGAGTACCCTAC
AAAGACTTCAGGACAAAACGCGTTTGCTGTGGCTATAAGTTTTTTTGCTG
TCGTTAACCAGCATGAAGG
Translation:

(SEQ ID NO:294)

MRCLPVFVILLLLTASAPSVDARPKAKDDVPLASFRDNAKSTLQRLQDKR
VCCGYKFFCCR
Toxin Sequence:

(SEQ ID NO:295)

Val-Cys-Cys-Gly-Xaa5-Lys-Phe-Phe-Cys-Cys-Arg-\hat{ }
Name: Au5.1
Species: aulicus
Cloned: Yes
DNA Sequence:

(SEQ ID NO:296)

GGAAGCTGACTACAAGCAGAATGCGCTGTCTCCCAGTCTTCGTCATTCTT
CTGCTGCTGACTGCATCTGCACCTAACGTTGATGCCCAACCGAAGACCAA
AGATGATGTGCCCCTGGCATCTTTGCACGATGATGCAAAGAGTGCACTAC
AACATTGGAACCAACGCTGCTGCCCCATGATCTATTGGTGCTGTAGTTAA
CCAGCATGAAGG
Translation:

(SEQ ID NO:297)

MRCLPVFVILLLLTASAPNVDAQPKTKDDVPLASLHDDAKSALQHWNQRC
CPMIYWCCS
Toxin Sequence:

(SEQ ID NO:298)

Cys-Cys-Xaa3-Met-Ile-Xaa5-Xaa4-Cys-Cys-Ser-\hat{ }
Name: Au5.4
Species: aulicus
Cloned: Yes
DNA Sequence:

(SEQ ID NO:299)

GGAAGCTGACTACAAGCAGAATGCACTGTCTCCCAGTCTTCGTCATTCTT
CTGCTGCTGACTGCATCTGCACCTAACGTTGATGCCCAACCGAAGACCAA
AGATGATGTGCCCCTGGCATCTTTGCACGATGATGCAAACAGTGCACTAC
AACATTGGAACCAACGCTGCTGCCCCGAGATCTATTGGTGCTGTAGTTAA
CCAGCATGAAGG
Translation:

(SEQ ID NO:300)

MHCLPVFVILLLLTASAPNVDAQPKTKDDVPLASLHDDAKSALQHWNQRC
CPEIYWCCS
Toxin Sequence:

(SEQ ID NO:301)

Cys-Cys-Xaa3-Xaa1-Ile-Xaa5-Xaa4-Cys-Cys-Ser-\hat{ }
Name: Af5.1
Species: ammiralis
Cloned: Yes
DNA Sequence:

(SEQ ID NO:302)

GGAAGCTGACTACAAGCAGAATGCGCTGTCTCCCAGTCTTCGTCATTCTT
CTGCTGCTGATTGCATCTGCACCTAGCGTTGATGCCCAACCGAAGACCAA
AGATGATGTGTCCCTGGCATCTTTGCACGATAATATAAAGAGTACTCTAC
AAACACTTTGGAACAAACGCTGCTGCCCCCCTGTGATTTGGTGCTGTGGT
TAACCAGCATAAAGG
Translation:

(SEQ ID NO:303)

MRCLPVFVILLLLIASAPSVDAQPKTKDDVSLASLHDNIKSTLQTLWNKR
CCPPVIWCCG
Toxin Sequence:

(SEQ ID NO:304)

Cys-Cys-Xaa3-Xaa3-Val-Ile-Xaa4-Cys-Cys-#
Name: Au5.3
Species: aulicus
Cloned: Yes
DNA Sequence:

(SEQ ID NO:305)

GGAAGCTGACTACAAGCAGAATGCGCTGTCTCCCAGTCTTCGTCATTCTT
CTGCTGCTGACTGCATCTGGACCTAGCGTTGATGCCCGACCGAAGACCAA
AGATGATGTGCCTCTGTCATCTTTCCGCGATAACGCAAAGAGTATCCTAC
AAAGACGTTGGAACAACTATTGCTGCACGAATGAGCTTTGGTGCTGTGGT
TAACCAGCATGAAGG
Translation:

(SEQ ID NO:306)

MRCLPVFVILLLLTASGPSVDARLPKTKDDVPLSSFRDNAKSILQRRWNN
YCCTNLLWCCG
Toxin Sequence:

(SEQ ID NO:307)

Xaa4-Asn-Asn-Xaa5-Cys-Cys-Thr-Asn-Xaa1-Leu-Xaa4-
Cys-Cys-#
Name: Da5.2
Species: dalli
Cloned: Yes
DNA Sequence:

(SEQ ID NO:308)

GGAAGCTGACTACAAGCAGAATGCACTGTCTCCCAGTCTTCGTCATTCTT
CTGCTGCTGACTGCATCTGGACCTAGCGTTGATGCCCGACCGAAGACCGA
AGATGATGTGCCCCTGTCATCTTTCCGCGATAATACAAAGAGTACCCTAC
AAAGACTTTTGAAGCCAGTCAACTGCTGTCCTATTGATCAATCTTGCTGT
TCTTAACCAGCATGAAGG
Translation:

(SEQ ID NO:309)

MHCLPVFVILLLLTASGPSVDARPKTEDDVPLSSFRDNTKSTLQRLLKPV
NCCPIDQSCCS
Toxin Sequence:

(SEQ ID NO:310)

Xaa3-Val-Asn-Cys-Cys-Xaa3-Ile-Asp-Gln-Ser-Cys-Cys-
Ser-\hat{ }
Name: Cn10.3
Species: consors
Cloned: Yes
DNA Sequence:

(SEQ ID NO:311)

GGATCCATGTTCACCGTGTTTCTGTTGGTTGTCTTGGCAACCACTGTCGT
TTCCATCCCTTCAGATCGTGCATCTGAAGGCAGGAATGCCGTAGTCCACG
AGAGAGCGCCTGAGCTGGTCGTTACGGCCACCACGACTTGCTGTGGTTAT
GATCCGATGACAATATGCCCTCCTTGCATGTGCACTCATTCCTGTCCACC
AAAAAGAAAACCAGGCCGCAGAAACGACTGATGCTCGAG
Translation:

(SEQ ID NO:312)

MFTVFLLVVLATTVVSIPSDRASEGRNAVVHERAPELVVTATTTCCGYDP
MTICPPCMCTHSCPPKRKPGRRND
Toxin Sequence:

(SEQ ID NO:313)

Ala-Xaa3-Xaa1-Leu-Val-Val-Thr-Ala-Thr-Thr-Thr-Cys-
Cys-Gly-Xaa5-Asp-Xaa3-Met-Thr-Ile-Cys-Xaa3-Xaa3-
Cys-Met-Cys-Thr-His-Ser-Cys-Xaa3-Xaa3-Lys-Arg-Lys-
Xaa3-#
Name: A10.2
Species: aurisiacus
Cloned: Yes
DNA Sequence:

(SEQ ID NO:314)

GGATCCATGTTCACCGTGTTTCTGTTGGTTGTCTTGGCAACCACTGTCGT
TTCCATCCCTTCAGATCGTGCATCTGATGGCAGGAATGCCGCAGTCAACG
AGAGACAATCTTGGCTGGTCCCTTCGACAATCACGACTTGCTGTGGATAT
GATCCGGGGACAATGTGCCCTCCTTGCAGGTGCAATAATACCTGTAAACC
AAAAAAACCAAAACCAGGAAAAGGCCGCAGAAACGACTGATGCTCCAGGA
CCCTCTGAACCACGACCTCGAG
Translation:

(SEQ ID NO:315)

MFTVFLLVVLATTVVSIPSDRASDGRNAAVNERQSWLVPSTITTCCGYDP
GTMCPPCRCNNTCKPKXPKPGKGRRND
Toxin Sequence:

(SEQ ID NO:316)

Xaa2-Ser-Xaa4-Leu-Val-Xaa3-Ser-Thr-Thr-Cys-Cys-
Gly-Xaa5-Asp-Xaa3-Gly-Thr-Met-Cys-Xaa3-Xaa3-Cys-
Arg-Cys-Asn-Asn-Thr-Cys-Lys-Xaa3-Lys-Lys-Xaa3-Lys-
Xaa3-Gly-Lys-#
Name: Cn10.4
Species: consors
Cloned: Yes
DNA Sequence:

(SEQ ID NO:317)

GGATCCATGTTCACCGTGTTTCTGTTGGTTGTCTTGGCAACCACTGTCGT
TTCCATCCCTTCAGATCGTGCATCTGATGGCAGGAATGCCGTAGTCCACG
AGAGAGCGCCTGAGCTGGTCGTTACGGCCACCACGACTTGCTGTGGTTAT
GATCCGATGACATGGTGCCCTTCTTGCATGTGCACTTATTCCTGTCCCCA
CCAAAGGAAAAAACCAGGCCGCAGAAACGACTGATGCTCCAGGACCCTCT
GAACCACGACCTCGAG
Translation:

(SEQ ID NO:318)

MFTVFLLVVLATTVVSIPSDRASDGRNAVVHERAPELVVTATTTCCGYDP
MTWCPSCMCTYSCPHQRKKPGRRND
Toxin Sequence:

(SEQ ID NO:319)

| | |
|---|---|
| Ala-Xaa3-Xaa1-Leu-Val-Val-Thr-Ala-Thr-Thr-Thr-Cys- | |
| Cys-Gly-Xaa5-Asp-Xaa3-Met-Thr-Xaa4-Cys-Xaa3-Ser- | |
| Cys-Met-Cys-Thr-Xaa5-Ser-Cys-Xaa3-His-Gln-Arg-Lys- | |
| Lys-Xaa3-# | |
| Name: M10.3 | |
| Species: magus | |
| Cloned: | Yes |
| DNA Sequence: | |

(SEQ ID NO:320)

GGATCCATGTTCACCGTGTTTCTGTTGGTTGTCTTGGCAACCAGTGTCGT
TTCCATCCCTTCAGATCGTGCATCTGATGGCGGGAATGCCGTAGTCCACG
AGAGAGCGCCTGAGCTGGTCGTTACGGCCACCACGACTTGCTGTGGTTAT
GATCCGATGACAATATGCCCTCCCTGCATGTGCACTCATTCCTGTCCACC
AAAAGGAAAACCAGGCCGCAGGAACGACTGATGTCCAGGACCTCTGAACC
ACGACNCGAG
Translation:

(SEQ ID NO:321)

MFTVELLVVLATSVVSIPSDRASDGGNAVVHERAPELVVTATTTCCGYDP
MTICPPCMCTHSCPPKGKIPGRRNP
Toxin Sequence:

(SEQ ID NO:322)

Ala-Xaa3-Xaa1-Leu-Val-Val-Thr-Ala-Thr-Thr-Thr-Cys-
Cys-Gly-Xaa5-Asp-Xaa3-Met-Thr-Ile-Cys-Xaa3-Xaa3-
Cys-Met-Cys-Thr-His-Ser-Cys-Xaa3-Xaa3-Lys-Gly-Lys-
Xaa3-#
Name: A10.3
Species: aurisiacus
Cloned: Yes
DNA Sequence:

(SEQ ID NO:323)

GAATTCGCCCTTGAGGATCCGTGTGGTTCTGGGTCCAGAACCTGATGGCA
GGAATGCCGCAGTCAACGAGAGACAGAAATGGCTGGTCCATTCGAANATC
ACGTATTGCTGTGGTTATAATAAGATGGACATGTGCCCTCCTTGCATGTG
CACTTATTCCTGTCCCCCCCTAAAAAAAAAAAGACCAGGCCGCAGAAACG
ACTGATGCTCCAGGACCCTCTGAACCACGACCTCGAGCGAAGGGCGAATT
C
Translation:

(SEQ ID NO:324)

VVLGPEPDGRNAAVNIERQKWLVHSKITYCCGYNKMDMCPPCMCTYSCPP
LKKKRPGRRNP
Toxin Sequence:

(SEQ ID NO:325)

Xaa2-Lys-Xaa4-Leu-Val-His-Ser-Lys-Ile-Thr-Xaa5-
Cys-Cys-Gly-Xaa5-Asn-Lys-Met-Asp-Met-Cys-Xaa3-
Xaa3-Cys-Met-Cys-Thr-Xaa5-Ser-Cys-Xaa3-Xaa3-Leu-
Lys-Lys-Lys-Arg-Xaa3-#
Name: A10.4
Species: aurisiacus
Cloned: Yes
DNA Sequence:

(SEQ ID NO:326)

GAATTCGCCCTTGAGGATCCGTGTGGTTCTGGGTCCAGCATTTGATGGCA
GGAATGCCGCAGTCAACGAGAGAGCGCCTTGGACGGTCGTTACGGCCACC
ACGAATTGCTGCGGTATTACCGGGCCAGGCTGCCTTCCTTGCCGTTGTAC
TCAAACATGTGGCTGATGCTCCAGGACCCTCTGAACCACGACCTCGAGCG
AAGGGCGAATTC
Translation:

(SEQ ID NO:327)

VVLGPAFDGRNAAVNERAPWTVVTATTNCCGITGPGCLPCRCTQTCG
Toxin Sequence:

(SEQ ID NO:328)

Ala-Xaa3-Xaa4-Thr-Val-Val-Thr-Ala-Thr-Thr-Asn-Cys-
Cys-Gly-Ile-Thr-Gly-Xaa3-Gly-Cys-Leu-Xaa3-Cys-Arg-
Cys-Thr-Gln-Thr-Cys-#
Name: Mr1.3
Species: marmoreus
Cloned: Yes
DNA Sequence:

(SEQ ID NO:329)

GGAAGCTGACTACAAGCAGAATGCGCTGTCTCCCAGTCTTGATCATTCTT
CTGCTGCTGACTGCATCTGCACCTGGCGTTGTTGTCCTACCGAAGACCGA
AGATGATGTGCCCATGTCATCTGTCTACGGTAATGGAAAGAGTATCCTAC
GAGGGATTCTGAGGAACGGTGTTTGCTGTGGCTATAAGTTGTGCCTTCCA
TGTTAACCAGCATGAAGG
Translation:

(SEQ ID NO:330)

MRCLPVLIILLLLTASAPGVVVLPKTEDDVPMSSVYGNGKSILRGILRNG
VCCGYKLCLPC
Toxin Sequence:

(SEQ ID NO:331)

Asn-Gly-Val-Cys-Cys-Gly-Xaa5-Lys-Leu-Cys-Leu-Xaa3-
Cys-\hat{ }
Name: Pn1.5
Species: pennaceus
Cloned: Yes
DNA Sequence:

(SEQ ID NO:332)

GGAATTCGGAAGCTGACTACAAGCAGAATGCGCTGTCTCCCAGTCTTCGT
CATTCTTCTGCTGCTGACTGCATCTGCACCTAGCGTTGATGCCAAAGTTC
ATCTGAAGACCAAAGGTGATGGGCCCCTGTCATCTTTCCGAGATAATGCA
AAGAGTACCCTACAAAGACTTCAGGACAAAAGCACTTGCTGTGGCTTTAA
GATGTGTATCCCTTGTAGTTAACCAGCATGAAGGATCC
Translation:

(SEQ ID NO:333)

MRCLPYFVILLLLTASAPSVDAKVHLKTKGDGPLSSFRDNAKSTLQRLQD
KSTCCGFKMCIPCS
Toxin Sequence:

(SEQ ID NO:334)

Ser-Thr-Cys-Cys-Gly-Phe-Lys-Met-Cys-Ile-Xaa3-Cys-
Ser-\hat{ }
Name: Pn1.6
Species: pennaceus
Cloned: Yes
DNA Sequence:

(SEQ ID NO:335)

GAATTCGGAAGCTGACTACAAGCAGAATGCGTTGTCTCCCAGTCTTCGTC
ATTCTTCTGCTGCTGACTGCATCTGGACCTAGCGTTGATGCCCGACTGAA
GACCAAAGATGATGTGCCCCTGTCATCTTTCCGAGATAATGCAAAGAGTA
CCCTACAAAGACTTCAGGACAAACGCCTTTGCTGTGGCTTTTGGATGTGT
ATTCCTTGTAATTAACCAGCATGAAGGATCC
Translation:

(SEQ ID NO:336)

MRCLPVFVILLLLTASGPSVDARLKTKDDVPLSSFRDNAKSTLQRLQDKR
LCCGFWMCIPCN
Toxin Sequence:

(SEQ ID NO:337)

Leu-Cys-Cys-Gly-Phe-Xaa4-Met-Cys-Ile-Xaa3-Cys-Asn-
\hat{ }
Name: Pn1.7
Species: pennaceus
Cloned: Yes
DNA Sequence:

(SEQ ID NO:338)

GAATTCTCCCTTGGAATTCTGAAGCTGACTACAANCAGAATGCGTTGTCT
CCCACTCTTCGTCATTCTTCTGCTGCTGACTGCATCTGGACCTACTGTTG
ATGCCCGACTGAAGACCAAAGATGATGTGCCCCTGTCATCTTTCCGAGAT
AATGCAAAGAGTACCCTACAAAGACTTCAGGACAAAAGCACTTGCTGTGG
CTTTAAGATGTGTATTCCTTGTGGTTAACCAGCATGAAGGATCC
Translation:

(SEQ ID NO:339)

MRCLPLFVILLLLTASGPTVDARLKTKDDVPLSSFRDNAKSTLQRLQDKS
TCCGFKMCIPCG
Toxin Sequence:

(SEQ ID NO:340)

Ser-Thr-Cys-Cys-Gly-Phe-Lys-Met-Cys-Ile-Xaa3-Cys-#
Name: Ep1.5
Species: episcopatus
Cloned: Yes
DNA Sequence:

(SEQ ID NO:341)

GAATTCGCCCTTGGAATTCGGAAGCTGACTACAAGCAGAATGCGCTGTCT
CCCAGTCTTCGTCATTCTTCTGCTGCTGACTGCATCTGGACCTANTGTTG
ATGCCAAAGTTCATCTGAAGACCAAAGGTGATGGGCCCCTGTCATCTTTC
CGAGATAATGCAAAGAGTACCCTACAAAGACTTCAGGACAAAAGCACTTG
CTGTGGCTATAGGATGTGTGTTCCTTGTGGTTAACCAGCATGAAGGATCC
V
Translation:

(SEQ ID NO:342)

MRCLPVFVILLLLTASGPSVDAKVHLKTKGDGPLSSFRDNAKSTLQRLQD
KSTCCGYRMCVPCG
Toxin Sequence:

(SEQ ID NO:343)

Ser-Thr-Cys-Cys-G1y-Xaa5-Arg-Met-Cys-Val-Xaa3-Cys-
#
Name: Mr1.1
Species: marmoreus
Isolated: Yes
Cloned: Yes
DNA Sequence:

(SEQ ID NO:344)

GGCGAATACACCTGGCAGGTACTCAACGAACTTCAGGACACATTCTTTTC
ACCTGGACACTGGAAACTGACAACAGGCAGAATGCGCTGTCTCCCAGTCT
TGATCATTCTTCTGCTGCTGACTGCATCTGCACCTGGCGTTGTTGTCCTA
CCGAAGACCGAAGATGATGTGCCCATGTCATCTGTCTACGGTAATGGAAA
GAGTATCCTACGAGGAATTCTGAGGAACGGTGTTTGCTGTGGCTATAAGT
TGTGCCATCCATGTTAACCAGCATGAAGGGAAATGACTTTGGATGAGACC
CCTGCGAACTGTCCCTGGATGTGAAATTTGGAAAGCAGACTGTTCCTTTC
GCACGTATTCGTGGAATTTCGAATGGTCGTAAACAACACGCTGCCACTTG
CAGGCTACTATCTCTCTGTCCTTTCATCTGTGGAAATGGATGATCTAACA
ACTGAAATATCAGAAATTTTTCAATGGCTATACACTATGACCATGTAGTC
AGTAATTATATCATTTGGACCTTTTGAAATATTTTTCAATATGTAAAGTT
TTTGCACCCTGGAAAGGTCTTTTGGAGTTAAATATTTTAGTATGTTATGT
TTTGCATACAAGTTATAGAATGCTGTCTTTCTTTTTGTTCCCACATCAAT
GGTGGGGGCAGAAATTATTTGTTTTGGTCAATGTAATTATGACCTGCATT
TAGTGCTATAGTGATTGCATTTTCAGCGTGGAATGTTTAATCTGCAAACA
GAAAGTGGTTGATCGACTAATAAAGATTTGCATGGCACAAAAAAAAAAAA
AAAAAGTACTCTGCGTTGTTACTCGAG
Translation:

(SEQ ID NO:345)

MRCLPVLIILLLLTASAPGVVVLPKTEDDVPMSSVYGNGKSILRGILRNG
VCCGYKLCHPC
Toxin Sequence:

(SEQ ID NO:346)

Asn-Gly-Val-Cys-Cys-Gly-Xaa5-Lys-Leu-Cys-His-Xaa3-
Cys-\hat{ }
Name: Mr1.2
Species: marmoreus
Isolated: Yes
Toxin Sequence:

(SEQ ID NO:347)

Gly-Val-Cys-Cys-Gly-Xaa5-Lys-Leu-Cys-His-Xaa3-Cys-
\hat{ }
Name: Bn1.5
Species: bandanus
Cloned: Yes
DNA Sequence:

(SEQ ID NO:348)

ATGCGCTGTCTCCCAGTCTTGATCATTCTTCTGCTGCTGACTGCATCTGC
ACCTGGCGTTGATGTCCTACCGAAGACCGAAGATGATGTGCCCCTGTCAT
CTGTCTACGATAATACAAAGAGTATCCTACGAGGACTTCTGGACAAACGT
GCTTGCTGTGGCTACAAGCTTTGCTCACCATGTTAACCAGCATGAAGGAT
CC
Translation:

(SEQ ID NO:349)

MRCLPVLIILLLLTASAPGVDVLPKTEDDVPLSSVYDNTKSILRGLLDKR
ACCGYKLCSPC
Toxin Sequence:

(SEQ ID NO:350)

Ala-Cys-Cys-Gly-Xaa5-Lys-Leu-Cys-Ser-Xaa3-Cys-\hat{ }
Name: Au1.4
Species: aulicus
Cloned: Yes
DNA Sequence:

(SEQ ID NO:351)

GGAAGCTGACTACAAGCAGAATGCGCTGTCTCCCAGTCTTCGTCATTCTT
CTGCTGCTGACTGCATCTGGACCTAGCGTTGATGCCCGACTGAAGACCAA
AGATGATGTGCCCCTGTCATCTTTCCGAGATAATGCAAAGAGTACCCTAC
AAAGACATCAGGACAAAAGCGTTTGCTGTGGCTATAAGCTGTGTTTTCCT
TGTGGTTAACCAGCATGAAGG
Translation:

(SEQ ID NO:352)

MRCLPVFVILLLLTASGPSVDARLKTKDDVPLSSFRDNAKSTLQRHQDKS
VCCGYKLCFPCG
Toxin Sequence:

(SEQ ID NO:353)

Ser-Val-Cys-Cys-Gly-Xaa5-Lys-Leu-Cys-Phe-Xaa3-Cys-
#
Name: Tx1.7
Species: textile
Cloned: Yes
DNA Sequence:

(SEQ ID NO:354)

CAGGATCCAATGGGGTTTGTTGTGGCTATAGGATGTGTGTTCCTTGTGGT
TAACCAGCATGAAGGGAAATGACTTTGGATGAGACCCCTGCGAACTGTCC
CTGGATGTGAGATTTGGAAAGCAGACTGTTCATTTTGCACGTGTTCGTGG
AATTTCGAATGGTCGTTAACAACACGCTGCCACTTGCAAGCTACTATCTC
TCTGTCCTTTTATCTGTGGAACTGTATGATCTAACAACTGAAATATCATA
NANATTTTTCAATGGGTATNCACTATGCATATGATCATGTAGGGTTCAAG
GGGTCAAGATNC
Translation:

(SEQ ID NO:355)

GSNGVCCGYRMCVPCG
Toxin Sequence:

(SEQ ID NO:356)

Asn-Gly-Val-Cys-Cys-Gly-Xaa5-Arg-Met-Cys-Val-Xaa3-
Cys-#
Name: Tx1.6
Species: textile
Cloned: Yes
DNA Sequence:

(SEQ ID NO:357)

ATGCACTGTCTCCCAATCTTCGTCATTCTTCTGCTGCTGACTGCATCTGG
ACCTAGCGTTGATGCCCAACTGAAGACCAAAGATGATGTGCCCCTGTCAT
CTTTCCGAGATCATGCAAAGAGTACCCTACGAAGACTTCAGGACAAACAG
ACTTGCTGTGGCTATAGGATGTGTGTTCCTTGTGGTTAACCAGCATGAAG
GATCC
Translation:

(SEQ ID NO:358)

MHCLPIFVILLLLTASGPSVDAQLKTKDDVPLSSFRDHAKSTLRRLQDKQ
TCCGYRMCVPCG
Toxin Sequence:

(SEQ ID NO:359)

Xaa2-Thr-Cys-Cys-Gly-Xaa5-Arg-Met-Cys-Val-Xaa3-
Cys-#
Name: Af1.3
Species: ammiralis
Cloned: Yes
DNA Sequence:

(SEQ ID NO:360)

AGAAGCTGACTACAAGCAGAATGCACTACCTCCCAGTCTTCGTCATTCTT
CTGCTGCTGACTGCATCTGGACCTAGCGTTGATGCCCAACTGAAGACCAA
AGATGATGTGCCCCTGTCATCTTTCCGAGATAATGCAAAGAGTACCCTAC
GAAGACTCCAGTACAAACAGGCTTGCTGTGGCTTTAAGATGTGTGTTCCT
TGTGGTTAACCAGCATGAAGG
Translation:

(SEQ ID NO:361)

MHYLPVFVILLLLTASGPSVDAQLKTKDDVPLSSFRDNAKSTLRRLQYKQ
ACCGFKMCVPCG
Toxin Sequence:

(SEQ ID NO:362)

Xaa2-Ala-Cys-Cys-Gly-Phe-Lys-Met-Cys-Val-Xaa3-Cys#
Name: Pn1.3
Species: pennaceus
Cloned: Yes
DNA Sequence:

(SEQ ID NO:363)

ATGCGCTGTCTCCCAGTCTTCGTCATTCTTCTGCTGCTGACTGCATCTGC
ACCTAGCGTTGATGCCAAAGTTCATCTGAAGACCAAAGGTGATGGGCCCC
TGTCATCTTTCCGAGATAATGCAAAGAGTACCCTACAAAGACTTCAGGAC
AAAAGCACTTGCTGTGGCTTTAAGATGTGTATTCCTTGTCGTTAACCAGC
ATGAAGGATCC
Translation:

(SEQ ID NO:364)

MRCLPVFVILLLLTASALPSVDAKVHLKTKGDGPLSSFRDNAKSTLQRLQ
DKSTCCGFKMCIPCR
Toxin Sequence:

(SEQ ID NO:365)

Ser-Thr-Cys-Cys-Gly-Phe-Lys-Met-Cys-Ile-Xaa3-Cys-
Arg-\hat{ }
Name: Pn1.4
Species: pennaceus
Cloned: Yes
DNA Sequence:

(SEQ ID NO:366)

CAGGATCCAATGGGGTTTGTTGTGGCTTTTGGATGTGTATTCCTTGTAAT
TAACCAGCATGAAGGGAAATGACTTTGGATAAGACCCCTGCGAACTGTCC
TTGGATGTGAGATTTGGAAAGCAGACTGTTCCTTTTGCACGTGTTCGTGG
AATTTCGAATGGTCGTTAACAACACGCTGCCACTTGCAAGCTACTATCTC
TCTGTCCTTTCATCTGTGGAACTGTATGATCTAACAACTGAAATATCATA
GAAATTTTTCAATGGGTATACACTATGCATATGACCATGTANGGGTCAAC
AGNC
Translation:

(SEQ ID NO:367)

GSNGVCCGFWMCIPCN
Toxin Sequence:

(SEQ ID NO:368)

Asn-Gly-Val-Cys-Cys-Gly-Phe-Xaa4-Met-Cys-Ile-Xaa3-
Cys-Asn-\hat{ }
Name: Om1.7
Species: omaria
Cloned: Yes
DNA Sequence:

(SEQ ID NO:369)

GGAAGCTGACTACAAGCAGAATGCGCTGTCTCCCAGTCTTCGTCATTCTT
CTGCTGCTGACTGCATCTGCACCTAGCGTTGATGCCCGACCGAAGGCCAA
AGATGATGTGCCCCTGTCATCTTTCCGTGATAATGCAAAGAGTACCCTAC
AAAGACTTCAGGACAAAGACGTTTGCTGTTACGTTAGAATGTGTCCTTGT
CGTTAACCAGCATGAAGG
Translation:

(SEQ ID NO:370)

MRCLPVFVILLLLTASAPSVDARPKADDVPLSSFRDNAKSTLQRLQDKDV
CCYVRMCPCR
Toxin Sequence:

(SEQ ID NO:371)

Asp-Val-Cys-Cys-Xaa5-Val-Arg-Met-Cys-Xaa3-Cys-Arg-
\hat{ }
Name: Conophysin-R
Species: radiatus
Isolated: Yes
Toxin Sequence:

(SEQ ID NO:372)

His-Xaa3-Thr-Lys-Xaa3-Cys-Met-Xaa5-Cys-Ser-Phe-
Gly-Gln-Cys-Val-Gly-Xaa3-His-Ile-Cys-Cys-Gly-Xaa3-
Thr-Gly-Cys-Xaa1-Met-Gly-Thr-Ala-Xaa1-Ala-Asn-Met-
Cys-Ser-Xaa1-Xaa1-Asp-Xaa1-Asp-Xaa3-Ile-Xaa3-Cys-
Gln-Val-Phe-Gly-Ser-Asp-Cys-Ala-Leu-Asn-Asn-Xaa3-
Asp-Asn-Ile-His-Gly-His-Cys-Val-Ala-Asp-Gly-Ile-
Cys-Cys-Val-Asp-Asp-Thr-Cys-Thr-Thr-His-Leu-Gly-
Cys-Leu-\hat{ }
Name: Ts10.1
Species: tessulatus
Cloned: Yes
DNA Sequence:

(SEQ ID NO:373)

GGATCCATGTTCACCGTGTTTCTGTTGGTTGTCTTGGCAACCACTGTTGT
TTCCTTCAGTGCAGATCGTGCCAACGTCAAAGCGTCTGACCTGATCGCCC
AGGCCACCAGAGACGGCTGTCCACCACATCCCGTTCCTGGCATGCATAAG
TGCATGTGTACTAATACATGTGGTTGAAGACGCTGATGCTCCAGGACCCT
CTGAACCACGACCTCGAG
Translation:

(SEQ ID NO:374)

MFTVFLLVVLATTVVSFSADRANVKASDLIAQATRDGCPPHPVPGMHKCM
CTNTCG
Toxin Sequence:

(SEQ ID NO:375)

Asp-Gly-Cys-Xaa3-Xaa3-His-Xaa3-Val-Xaa3-Gly-Met-
His-Lys-Cys-Met-Cys-Thr-Asn-Thr-Cys-#
Name: G1.4
Species: geographus
Cloned: Yes
DNA Sequence:

(SEQ ID NO:376)

ANNTAGANTNYGTCGTANTANNGGATCNAATANTGNNTCGANATGATNAN
GAGTGATAAATGANNGGTGCACTNNTANTTANGNTNNTANGATNNNNATA
TTATTNNTANNNNNTAANANATATNGGTNNGGANNAAGAAGANTAAAAGT
ANNGNTTNGTGAAANAANGANNNNATGTTNNANNTCATAACNNNAATGTA
AATAATANACGNNCCAGTGTGAAANNTNTCNNNNATAAAAATTCTNTNTN
TNANGTNNNTGTNTGNGTGTGTGTGTGTGTGTGTGTGTGTGNGTGTGTGN
GTGTGTGTGTGTGTGTGTGTGTGTGTGNGTGTGTGTNTGTGNGTGTGTGT
GTGTGTGTGTGTGTGTGTGTGTGTGTNTGTGGTTCTGGGTCCAGCATCTG
ATGNCAGGGATGACACAGCCAAAGACGAAGGGTCTNACATGGACAAATTG
GTCGAGAAAAAAGAATGTTGCCATCCTGCCTGTGGCAAACACTACAGTTG
TGGACGCTGATGCTCCAGGGTNTGAAGGANCAA
Translation:

(SEQ ID NO:377)

SDXRDDTAKDEGSXIVIDKLVEKKECCHPACGKHYSCGR
Toxin Sequence:

(SEQ ID NO:378)

Xaa1-Cys-Cys-His-Xaa3-Ala-Cys-GLy-Lys-His-Xaa5-
Ser-Cys-#
Name: G1.5
Species: geographus
Cloned: Yes
DNA Sequence:

(SEQ ID NO:379)

GGATCCATGTTCACCGTGTTTCTGTTGGTGGTCTTGGCAACCACTGTCGT
TTCCTTCCCTTCAGAACGTGCATCTGATGGCAGGGATGACACAGCCAAAG
ACGAAGGGTCTGACATGGAGAAATTGGTCGAGAAAAAAGAATGTTGCAAT
CCTGCCTGTGGCAGACACTTCAGTTGTGGACGCTGATGCTCCAGGACCCT
CTGAACCACGACTCGAG
Translation:

(SEQ ID NO:380)

MFTVFLLVVLATTVVSFPSERASDGRDDTAKDEGSDMEKLVEKKCCNPAC
GRHFSCGR
Toxin Sequence:

(SEQ ID NO:381)

Xaa1-Cys-Cys-Asn-Xaa3-Ala-Cys-Gly-Arg-His-Phe-Ser-
cys#
Name: S1.8
Species: striatus
Cloned: Yes
DNA Sequence:

(SEQ ID NO:382)

GGATCCATGTTCACCGTGTTTCTGTTGGTTGTCTTGGCAACCACTGTCGT
TTCCTTCACTTCAGATCGTGCATCTGATGGCAGGGATGACGAAGCCAAAG
ACGACGGTCTGACATGCACGAATCGGACCGGAAAGGACGCGCATACTGTT
GCCATCCTGCCTGTGGCCCAAACTATAGTTGTGGCACCTCATGCTCCAGG
ACCCTCTGAACCACGACCTCGAG
Translation:

(SEQ ID NO:383)

MFTVFLLVVLATTVVSFTSDRASDGRDDEAIUZERSDMHESDRKGRAYCC
HPACGPNYSCGTSCSRTL
Toxin Sequence:

(SEQ ID NO:384)

Ala-Xaa5-Cys-Cys-His-Xaa3-Ala-Cys-Gly-Xaa3-Asn-
Xaa5-Ser-Cys-Gly-Thr-Ser-Cys-Ser-Arg-Thr-Leu-\hat{ }
Name: S1.9
Species: striatus
Cloned: Yes
DNA Sequence:

(SEQ ID NO:385)

GGATCCATGTTCACCGTGTTTCTGTTGGTTGTCTTGGCAACCACTGTCGT
TTCCTTCACTTCAGATCGTGCATCTGATGGCAGGGATGACGAAGCCAAAG
ACGAAAGGTCTGACATGCACGAATCGGACCGGAAAGGACGCGCATACTGT
TGCCATCCTGTCTGTGGCAAAAACTTTGATTGTGGACGCTGATGCTCCAG
GACCCTCTGAACCACGACCTCGAG
Translation:

(SEQ ID NO:386)

MFTVFLLVVLATTVVSFTSDRASDGRDDEA1CDERSDMHESDRKGRAYCC
HPVCGKNFDCGR
Toxin Sequence:

(SEQ ID NO:387)

Ala-Xaa5-Cys-Cys-His-Xaa3-Val-Cys-Gly-Lys-Asn-Phe-
Asp-Cys-#
Name: Ra1.1
Species: rattus
Cloned: Yes
DNA Sequence:

(SEQ ID NO:388)

GGATCCATGTTCACCGTGTTTCTGTTGGTTGTCTTGGCAACCACTGTCGT
TTCCTTCCCTTCAGATCGTGCATCTGATGGCAGGGATGACGAAGCCAAGA
CGAAAGGTCTGACATGCACGAATCGGACCGGAATGGACGCGGATGCTGTT
GCAATCCTGCCTGTGGCCCAAACTATGGTTGTGGCACCTCATGCTCCAGG
ACCCTCTGAACCACGACCTCGAG
Translation:

(SEQ ID NO:389)

MFTVFLLVVLATTVVSFPSDRASDGRDDEAKDERSDMHESDRNGRGCCCN
PACGPNYGCGTSCSRTL
Toxin Sequence:

(SEQ ID NO:390)

Gly-Cys-Cys-Cys-Asn-Xaa3-Ala-Cys-Gly-Xaa3-Asn-
Xaa5-Gly-Cys-Gly-Thr-Ser-Cys-Ser-Arg-Thr-Leu-\hat{ }
Name: Ar1.1
Species: arenatus
Cloned: Yes
DNA Sequence:

(SEQ ID NO:391)

GGATCCATGTTCACCGTGTTTCTGTTGGTTGTCTTGGCAACCACTGTGGA
TTCCTTCACTCCAGTTCGTACTTCTGTTGGCAGGAGTGCTGCAGCCAACG
CGTTTGACCGGATCGCTCTGACCGCCAGGCAAGATTATTGCTGTACCATT
CCCAGCTGTTGGGATcGCTATAAAGAGAGATGTAGACACATACGCTGATG
CTCCAGGACCCTCTGAACCACGACCTTGAG
Translation:

(SEQ ID NO:392)

MFTVFLLVVLATTVDSFTPVRTSVGRSAAANAFDRIALTARQDYCCTTPS
CWDRYKERCRHIR
Toxin Sequence:

(SEQ ID NO:393)

Xaa2-Asp-Xaa5-Cys-Cys-Tbr-Ile-Xaa3-Ser-Cys-Xaa4-
Asp-Arg-Xaa5-Lys-Xaa1-Arg-Cys-Arg-His-Ile-Arg-\hat{ }
Name: Er1.1
Species: ebumeus
Cloned: Yes
DNA Sequence:

(SEQ ID NO:394)

GGATCCATGTTCACCGTGTTTCTGTTGGTTGTCTTGGCAACCACTGTGGA
TTCCTTCACTTCAGTTCGTACTTCCGTTGGCAGGAGTGCTGCAGCCAACG
CGTTTGACCGGATCGCTCTGACCGCCAGGCAAGATTATTGCTGTACCATT
CCCAGCTGTTGGGATCGCTATAAAGAGAGATGTAGACACATACGCTGATG
CTCCAGGACCCTCTGAACCACGACCTCGAG
Translation:

(SEQ ID NO:395)

MFTVFLLVVLATTVDSFTSVRTSVGRSAAANAFDRIALTARQDYCCTIPS
CWDRYKERCRHIR
Toxin Sequence:

(SEQ ID NO:396)

Xaa2-Asp-Xaa5-Cys-Cys-Thr-Ile-Xaa3-Ser-Cys-Xaa4-
Asp-Arg-Xaa5-Lys-Xaa1-Arg-Cys-Arg-His-Ile-Arg-\hat{ }
Name: Mil.2
Species: miles
Cloned: Yes
DNA Sequence:

(SEQ ID NO:397)

GGATCCATGTTCACCGTGTTTCTGTTGGTTGTCTTGGCAACTGCTGTTCT
TCCAGTCACTTTAGATCGTGCATCTGATGGAAGGAATGCAGCAGCCAACG
CCAAAACGCCTCGCCTGATCGCGCCATTCATCAGGGATTATTGCTGTCAT
AGAGGTCCCTGTATGGTATGGTGTGGTTGAAGCCGCTGCTGCTCCAGGAC
CCTCTGAACCACGACCTCGAG
Translation:

(SEQ ID NO:398)

MFTVFLLVVLATAVLPVTLDRASDGRNAAANAKTPRLIALPFIRDYCCHR
GPCMVWCG
Toxin Sequence:

(SEQ ID NO:399)

Asp-Xaa5-Cys-Cys-His-Arg-Gly-Xaa3-Cys-Met-Val-
Xaa4-Cys-#
Name: Jp1.1
Species: jaspedius
Cloned: Yes
DNA Sequence:

(SEQ ID NO:400)

GGATCCATGTTCACCGTGTTTCTGTTGGTTGTCTTGGCAACCACTGTCGT
TTCCAACTCTTCAGATCGTGGTCCAGCATCTAATAAAAGGAAGAATGCCG
CAATGCTTGACATGATCGCTCAACACGCCATAAGGGGTTGCTGTTCCGAT
CCTCGCTGTAGATATAGATGTCGTTGAAGACGCTGCTGCTCCAGGACCCT
CTGAACCACGACCTCGAG
Translation:

(SEQ ID NO:401)

METVELLVVLATTVVSNSSDRGPASNKRKNAAMLDMIAQHAIRGCCSDPR
CRYRCR
Toxin Sequence:

(SEQ ID NO:402)

Gly-Cys-Cys-Ser-Asp-Xaa3-Arg-Cys-Arg-Xaa5-Arg-Cys-
Arg-\hat{ }
Name: a-OmIA
Species: omaria
Isolated: Yes
Toxin Sequence:

(SEQ ID NO:403)

Gly-Cys-Cys-Ser-His-Xaa3-Ala-Cys-Asn-Val-Asn-Asn-
Xaa3-His-Ile-Cys-Gly-#
Name: a-OmIA [COOH]
Species: omaria
Cloned: No
Toxin Sequence:

(SEQ ID NO:404)

Gly-Cys-Cys-Ser-His-Xaa3-Ala-Cys-Asn-Val-Asn-Asn-
Xaa3-His-Ile-Cys-Gly-\hat{ }
Name: Qc1.1
Species: quercinus
Cloned: Yes
DNA Sequence:

(SEQ ID NO:405)

GGATCCATGTTCACCGTGTTTCTGTTGGTTGTCTTGGCAACCACTGTCAC
TTCAGATCGTGTATCTAATGGCAGGAAAGCTGCAGCCAAATTCAAAGCGC
CTGCCCTGATGGAGCTGTCCGTCAGGCAAGGATGCTGTTCAGATCCTGCC
TGTGCCGTGAGCAATCCAGACATCTGTGGCGGAGGACGCTGATGCTCCAG
GACCCTCTGAACCACGACCTCGAG
Translation:

(SEQ ID NO:406)

MFTVFLLVVLATTVTSDRVSNGRKAAAIKFKAPALMELSVRQGCCSDPAC
AVSNPDICGGGR
Toxin Sequence:

(SEQ ID NO:407)

Xaa2-Gly-Cys-Cys-Ser-Asp-Xaa3-Ala-Cys-Ala-Val-Ser-
Asn-Xaa3-Asp-Ile-Cys-Gly-Gly-#
Name: Bn1.6
Species: bandanus
Cloned: Yes
DNA Sequence:

(SEQ ID NO:408)

GGATCCATGTTCACCGTGTTTCTGTTGGTTGTCTTGGCAACCACTGTTGT
TTCCTTCACTTCAAATCGTGCATTTCGTCGTAGGAATGCCGTAGCCAAAG
CGTCTGACCTGATCGCTCTGAACGCCAGGAGACCAGAATGCTGTACTCAT
CCTGCCTGTCACGTGAGTCATCCAGAACTCTGTGGTTGAAGACGCTGACG
CTCCAGGACCCTCTGAACCACGACCTCGAG
Translation:

(SEQ ID NO:409)

MFTVFLLVVLATTVVSFTSNRAFRRRNAVAKASDLIALNARRPECCTHPA
CHVSHPELCG
Toxin Sequence:

(SEQ ID NO:410)

Xaa3-Xaa1-Cys-Cys-Thr-His-Xaa3-Ala-Cys-His-Val-
Ser-His-Xaa3-Xaa1-Leu-Cys-#
Name: Mr1.5
Species: marmoreus
Cloned: Yes
DNA Sequence:

(SEQ ID NO:411)

GGATCCATGTTCACCGTGTTTCTGTTGGTTGTCTTGGCAACCACTGTTGT
TTCCTTCACTTCAAATCGTGTTCTGGATCCAGCATTTCGTCGTAGGAATG
CCGCAGCCAAAGCGTCTGACCTGATCGCTCTGAACGCCAGGAGACCAGAA
TGCTGTACTCATCCTGCCTGTCACGTGAGTAATCCAGAACTCTGTGGCTG
AAGACGCTGATGCTCCAGGACCCTCTGAACCACGACCTCGAG
Translation:

(SEQ ID NO:412)

MFTVFLLVVLATTVVSFTSNRVLDPAFRRRNAAAKASDLIAINAIUIIEC
CTHPACHVSNPELCG
Toxin Sequence:

(SEQ ID NO:413)

Xaa3-Xaa1-Cys-Cys-Thr-His-Xaa3-Ala-Cys-His-Val-
Ser-Asn-Xaa3-Xaa1-Leu-Cys-#
Name: Mi1.1
Species: miles
Cloned: Yes
DNA Sequence:

(SEQ ID NO:414)

GGATCCATGTTCACCGTGTTTCTGTTGGTTGTCTTGGCAACCACTGTCGT
TTCCGTCACTTCATATCGTGCATCTCATGGCAGGAAGGACGCAGCCGACC
TGAGCGCTCTGAACGACAACAATAATTGCTGTAACCATCCTGCCTGTGCC
GGGAAAAATTCAGATCTTTGTGGTTGAAGACGCTGCTGCTCCAGGACCCT
CTGAACCACGACCTCGAG
Translation:

(SEQ ID NO:415)

MFTVFLLVVLATTVVSVTSYRASHGRKDAADLSAINDNNNCCHPACAGKN
SDLCG
Toxin Sequence:

(SEQ ID NO:416)

Cys-Cys-Asn-His-Xaa3-Ala-Cys-Ala-Gly-Lys-Asn-Ser-
Asp-Leu-Cys-#
Name: MII[YHT]
Species: magus
Toxin Sequence:

(SEQ ID NO:417)

Gly-Cys-Cys-Xaa5-His-Xaa3-Thr-Cys-His-Leu-Xaa1-
His-Ser-Asn-Leu-Cys-#
Name: Nb1.1
Species: nobilis
Cloned: Yes
DNA Sequence:

(SEQ ID NO:418)

GGATCCATGTTCACCGTGTTTCTGTTGGTTGTCTTGGCAACCACTGTTGT
TTCCTTCACTTCAGATCGTGCATCTGATGGCAGGAATGCCGCAGCCAAAG
CTTCTGACCTGATTGCTTTGACCGTCAGGGGATGCTGTGAGCGACCTCCC
TGTCGCTGGCAAAATCCAGATCTTTGTGGTGGAAGGCGCTGANATTCCAG
GACCCTCTGAACCACGACCTCGAG
Translation:

(SEQ ID NO:419)

MFTVFLLVVLATTVVSFTSDRASDGRNAAAKASDLIALTVRGCCERPPCR
WQNPDLCGGRR
Toxin Sequence:

(SEQ ID NO:420)

Gly-Cys-Cys-Xaa1-Arg-Xaa3-Xaa3-Cys-Arg-Xaa4-Gln-
Asn-Xaa3-Asp-Leu-Cys-Gly-#
Name: Ak1.1
Species: atlanticus
Cloned: Yes
DNA Sequence:

(SEQ ID NO:421)

GGATCCATGTTCACCGTGTTTCTGTTGGTTGTCTTGGCAACCACAGTCGT
TTCCTTCACTTCAGATAGTGCATTTGATAGCAGGAATGTCGCAGCCAACG
ACAAAGTGTCTGACATGATCGCTCTGACCGCCAGGAGAACATGCTGTTCC
CGTCCTACCTGTAGAATGGAATATCCAGAACTTTGTGGTGGAAGACGCTG
ATACTCCAGGACCCTCTGAACCACGACCTCGAG
Translation:

(SEQ ID NO:422)

MFTVFLLVVLATTVVSFTSDSAFDSRNVAANDKVSDMIALTARRTCCSRP
TCRMEYPELCGGRR
Toxin Sequence:

(SEQ ID NO:423)

Thr-Cys-Cys-Ser-Arg-Xaa3-Thr-Cys-Arg-Met-Xaa1-
Xaa5-Xaa3-Xaa1-Leu-Cys-Gly-#
Name: Qc1.2
Species: quercinus
Cloned: Yes
DNA Sequence:

(SEQ ID NO:424)

GGATCCATGTTCACCGTGTTTCTGTTGGTTGTCTTGGCAATCACGGTGGT
TTCCTTCACCTCAGATCATGCATCTGATGGCAGGAATACCGCAGCCAACG
ACAAAGCGTCTAAACTGATGGCTCTTACGAACGAATGCTGTGACAATCCT
CCGTGCAAGTCGAGTAATCCAGATTTGTGTGACTGGAGAAGCTGATGCTC
CAGGACCCTNTGAACCACGACCTCGAG
Translation:

(SEQ ID NO:425)

MFTVFLLVVLAITVVSFTSDHASDGRNTAANDKASKLMALTNECCDNPPC
KSSNPDLCDWRS
Toxin Sequence:

(SEQ ID NO:426)

Asn-Xaa1-Cys-Cys-Asp-Asn-Xaa3-Xaa3-Cys-Lys-Ser-
Ser-Asn-Xaa3-Asp-Leu-Cys-Asp-Xaa4-Arg-Ser-\hat{ }
Name: Lp1.1
Species: leopardus
Cloned: Yes
DNA Sequence:

(SEQ ID NO:427)

GGATCCATGTTCACCGTGTTTCTGTTGGTTGTCTTGGCAACCACGGTCGT
TTCCCTCACTTTAGATCGTGCATCTGGTGGCAGGAGATCTGGAGCCGACA
ACATGATTGCTCTTCTGATCATCAGAAAATGCTGTTCCAATCCCGCCTGT
AACAGGTATAATCCAGCAATTTGTGATTGAAGACGCTAATGCTCCAGGAC
CCTCTGAACCACGACCTCGAG
Translation:

(SEQ ID NO:428)

MFTVFLLVVLATTVVSLTLDRASGGRRSGADNMIALLIIRKCCSNPACNR
YNPAICD
Toxin Sequence:

(SEQ ID NO:429)

Cys-Cys-Ser-Asn-Xaa3-Ala-Cys-Asn-Arg-Xaa5-Asn-
Xaa3-Ala-Ile-Cys-Asp-\hat{ }
Name: Em1.1
Species: emaciatus
Cloned: Yes
DNA Sequence:

(SEQ ID NO:430)

GGATCCATGTTCACCGTGTTTCTGTTGGTTCTCTTGCCAACCACTGTCAC
TTTACATCGTGCATCTAATGGCAGGAATGCCGCAGCCAGCAGGAAAGCGT
CTGCCCTGATCGCTCAGATCGCCGGTAGAGACTGCTGTAACTTTCCTGCT
TGTGCCGCGAGTAATCCAGGCCTTTGTACTTGAAGACGCTGCTGCTCCAG
GACCCTCTGAACCACGACCTCGAG
Translation:

(SEQ ID NO:431)

MFTVFLLVLLATTVTLHRASNGRNAAASRKASALIAQIAGRDCCNEPACA
ASNPGLCT
Toxin Sequence:

(SEQ ID NO:432)

Asp-Cys-Cys-Asn-Phe-Xaa3-Ala-Cys-Ala-Ala-Ser-Asn-
Xaa3-Gly-Leu-Cys-Thr-\hat{ }
Name: C. victor alpha
Species: victor
Cloned: Yes
DNA Sequence:

(SEQ ID NO:433)

GGATCCATGTTCACCGTGTTTCTGTTGGTTGTCTTGGCAACCACCATCGT
TTCCTCCACTTTAGATCGTGCATCTGATGGCATGAATGCTGCAGCGTCTG
ACCTGATCGCTCTGAGCATCAGGAGATGCTGTTCTTCTCCTCCCTGTTTC
GCGAGTAATCCAGCTTGTGGTAGACGACGCTGATGCTCCAGGACCCTCTG
AACCACGACCTCGAG
Translation:

(SEQ ID NO:434)

MFTVFLLVVLATTIVSSTLDRASDGMNAAASDLIALSIRRCCSSPPCFAS
NPACGRRR
Toxin Sequence:

(SEQ ID NO:435)

Cys-Cys-Ser-Ser-Xaa3-Xaa3-Cys-Phe-Ala-Ser-Asn-
Xaa3-Ala-Cys-#
Name: Cj1.1
Species: cinereus gubba
Cloned: Yes
DNA Sequence:

(SEQ ID NO:436)

GGATCCATGTTCACCGTGTTTCTGTTGGTTGTCCTGGCAACCACTATCGT
TTCCTCCACTTCAGGTCATGCATTTGATGGCAGGAATGCTGCAGCCGACT
ACAAAGGGTCTGAATTGCTTGCTATGACCGTCAGGGGAGGATGCTGTTCC
TTTCCTCCCTGTATCGCAAATAATCCTTTTTGTGCTGGAAGACGCTGATG
CTCCAGGACCCTCTGAACCACGACCTCGAG
Translation:

(SEQ ID NO:437)

MFTVFLLVVLATTIVSSTSGHAFDGRNAAADYKGSELLAMTVRGGCCSFP
PCIANNPFCAGRR
Toxin Sequence:

(SEQ ID NO:438)

Gly-Gly-Cys-Cys-Ser-Phe-Xaa3-Xaa3-Cys-Ile-Ala-Asn-
Asn-Xaa3-Phe-Cys-Ala-#
Name: Fd1.1
Species: flavidus
Cloned: Yes
DNA Sequence:

(SEQ ID NO:439)

GGATCCATGTTCACCGTGTTTCTGTTGGTTGTCTTCGCATCCTCTGTCAC
TTTAGATCGTGCATCTCATGGCAGGTATATCCCAGTCGTCGACAGAGCGT
CTGCCCTGATGGCTCAGGCCGACCTTAGAGGTTGCTGTTCCAATCCTCCT
TGTTCCTATCTTAATCCAGCCTGTGGTTAAAGACGCTGCCGCTCCAGGAC
CCTCTGAACCACGACCTCGAG
Translation:

(SEQ ID NO:440)

MFTVFLLVVFASSVTLDRASHGRYIPVVDRASALMAQADLRGCCSNPPCS
YLNPACG
Toxin Sequence:

(SEQ ID NO:441)

Gly-Cys-Cys-Ser-Asn-Xaa3-Xaa3-Cys-Ser-Xaa5-Leu-
Asn-Xaa3-Ala-Cys-#
Name: Em1.2
Species: emaciatus
Cloned: Yes
DNA Sequence:

(SEQ ID NO:442)

GGATCCATGTTCACCGTGTTTCTGTTGGTTGTCTTCGCATCCTCTGTCAC
TTTAGATCGTGCATCTCATGGCAGGTATGCCGCAGTCGTCAACAGAGCGT
CTGCCCTGATGGCTCATGCCGCCCTTCGAGATTGCTGTTCCGATCCTCCT
TGTGCTCATAATAATCCAGACTGTCGTTAAAGACGCTGCTGCTCCAGGAC
CCTCTGAACCACGACCTCGAG
Translation:

(SEQ ID NO:443)

MFTVFLLVVFASSVTLDRASHGRYAAVVNRASALMAHAALRDCCSDPPCA
HNNPDCR
Toxin Sequence:

(SEQ ID NO:444)

Asp-Cys-Cys-Ser-Asp-Xaa3-Xaa3-Cys-Ala-His-Asn-Asn-
Xaa3-Asp-Cys-Arg-\hat{ }
Name: Ge1.1
Species: generalis
Cloned: Yes
DNA Sequence:

(SEQ ID NO:445)

GGATCCATGTTCACCGTGTTTCTGTTGGTTGTCTTGGCAACTACTGTCGT
TTCCTTCACTTCAGATCGTGGGTCTGATGGCAGGAATGCCGCAGCCAAGG
ACAAAGCGTCTGACCTGGTCGCTCTGACCGTCAAGGGATGCTGTTCTAAT
CCTCCCTGTTACGCGAATAATCAAGCCTATTGTAATGGAAGACGCTGATG
CTCCAGGACCCTCTGAACCACGACCTCGAG
Translation:

(SEQ ID NO:446)

MFTVFLLVVLATTVVSFTSDRGSDGRNAAAKDKASDLVALTVKGCCSNPP
CYANNQAYCNGRR
Toxin Sequence:

(SEQ ID NO:447)

Gly-Cys-Cys-Ser-Asn-Xaa3-Xaa3-Cys-Xaa5-Ala-Asn-
Asn-Gln-Ala-Xaa5-Cys-Asn-#
Name: Wi1.1
Species: wittigi
Cloned: Yes
DNA Sequence:

(SEQ ID NO:448)

GGATCCATGTTCACCGTGTTTCTGTTGGTTGTCCTGGCAACCACTGTCGT
TTCCCCCACTAGAGATCGTGCATCTGGTGTCAGGAATGTTGTTGCAACAA
GCTTTCAGACTCTGACCCACGATGAATGCTGTGCACACCCTTCCTGTTGG
AAGGCCGAAGACCTGATTTGTACTAATCAACGTCGCAGGACCCTCTGAAC
CACGACCTCGAG
Translation:

(SEQ ID NO:449)

MFTVFLLVVLATTVVSPTRDRASGVRNVVATSFQTLTHDECCAHLPSCWK
AEDLICTNQRRRTL
Toxin Sequence:

(SEQ ID NO:450)

Asp-Xaa1-Cys-Cys-Ala-His-Xaa3-Ser-Cys-Xaa4-Lys-
Ala-Xaa1-Asp-Leu-Ile-Cys-Thr-Asn-Gln-Arg-Arg-Arg-
Thr-Leu-\hat{ }
Name: Ca1.5
Species: caracteristicus
Cloned: Yes
DNA Sequence:

(SEQ ID NO:451)

GGATCCATGTTCACCGTGTTTCTGTTGGTTGTCTTGGCAACCACTGTCGT
TTCCTTCACTTCAGATCGTGCGTCTGAAGGCAGGAATGCTGCAGCCAAGG
ACAAAGCGTCTGACCTGGTGGCTCTGAGAGTCAGGGGATGCTGTGCCATT
CGTGAATGTCGCTTGCAGAATGCAGCGTATTGTGGTGGAATATCCTGATG
CTCCAGGACCCTCTGAACCACGACCTCGAG
Translation:

(SEQ ID NO:452)

MFTVFLLVVLATTVVSFTSDRASEGRNAAAKDKASDLVALRVRGCCAIRE
RLQNAAYCGGIS
Toxin Sequence:

(SEQ ID NO:453)

Gly-Cys-Cys-Ala-Ile-Arg-Xaa1-Cys-Arg-Leu-Gln-Asn-
Ala-Ala-Xaa5-Cys-Gly-Gly-Ile-Ser-\hat{ }
Name: Bt1.10
Species: betulinus
Cloned: Yes
DNA Sequence:

(SEQ ID NO:454)

AGTAATTNATATANNAGAAAGNAANANAAAANNATANAGAATTTAAGTAA
TNTAAGAANGAGANAGTGAATAGNAGNTAAGTAGANNAAGANAGGTAGAN
AGNANANGNGGANGNTAGNTAATAGATANNNTATNGAGANATTANTAGCN
GTATANANAAGAAAAGAGGGNAANNGAAATGNNGNAANNATAANTANTAN
NGATNGANNNGNAAGTGNNAAGNGTANAAGGAANAACAAANTNGTTGTNT
AATNTGNNTGNGTGTGTNTGTGTGNGTGTGTGTGTGTGNGTGNGTGTGTN
TGTGNGNNTGTGTGNGNGNGNGNGTGTGTGTGTGNGTGTGTGTGTGTGTG
TGTGTGTGTGTGTGTGTGTGNGTGTGTGGTTCTGGATCCAGCATCTGGTG
GCAGGAAGGCTGCAGCCAAAGCGTCTAACCGGATCGCTCTGACCGTCAGG
AGTGCAACATGCTGTTATTATCCTCCCTGTTACGAGGCTTATCCAGGGTT
GTCTGTAACGTGAATCATCCAGACCTTTGTGGCTGAAGACCCTGATGCTC
CAGGGGCAAGTTCAA
Translation:

(SEQ ID NO:455)

SGGRKAAAKASNRIALTVRSATCCYYPPCYEAYPESCL
Toxin Sequence:

(SEQ ID NO:456)

Ser-Ala-Thr-Cys-Cys-Xaa5-Xaa5-Xaa3-Xaa3-Cys-Xaa5-
Xaa1-Ala-Xaa5-Xaa3-Xaa1-Ser-Cys-Leu-\hat{ }

Where:
Xaa1 is Glu or γ-carboxy-Glu
Xaa2 is Gln or pyro-Glu
Xaa3 is Pro or hydroxy-Pro
Xaa4 is Trp (D or L) or bromo-Trp (D or L)
Xaa5 is Tyr, ¹²⁵I-Tyr, mono-iodo-Tyr, di-iodo-Tyr, O-sulpho-Tyr or O-phospho-Tyr
\hat{ } is free carboxyl or amidated C-terminus, preferably free carboxyl
# is free carboxyl or amidated C-terminus, preferably amidated
? is free carboxyl or amidated C-terminus

TABLE 2

| | |
|---|---|
| Alignment of γ-Conopeptides¹ | (SEQ ID NO:) |

| | | |
|---|---|---|
| 4/43 SNX | -------DCRGYDAPCSSGAPCCDWWTCS | (457) |

ARTNRCF\hat{ }

| | | |
|---|---|---|
| Af6.1 | GMW---GDCKDGLTTCFAPSECCSE-DC- | (458) |

E-GS-CTMW\hat{ }

| | | |
|---|---|---|
| At6.2 | ---WREGSCTSWLATCTQDQQCCTD-VDY | (459) |

KRDY-CALWDDR\hat{ }

| | | |
|---|---|---|
| Af6.3 | ----N---CSDDWQYCESPSDCCSW-DC- | (460) |

D-VV-CS#

| | | |
|---|---|---|
| Af6.4 | --WWRWGGCMAWFGKCSKDSECCSN-SC- | (461) |

DITR-CELMRFPPDW\hat{ }

| | | |
|---|---|---|
| At6.5 | -------DCRGYDAPCSSGAPCCDWWTCS | (462) |

ARTGRCF\hat{ }

| | | |
|---|---|---|
| At6.6 | ---L----CPDYTEPCSHAHECCSW-NC- | (463) |

HNGH-CT#

| | | |
|---|---|---|
| g-PnVIIA | --------DCTSWFGRCTVNSXCCSN-SC | (465) |

-DQTY-CXLYAFOS\hat{ }²

| | | |
|---|---|---|
| Gm6.7 | --------ECRAWYAPCSPGAQCCSLLMC | (466) |

SKATSRCILAL\hat{ }²

| | | |
|---|---|---|
| J010 | --------CKTYSKYCXADSXCCTX-QC- | (467) |

VRSY-CTLF#²

| | | |
|---|---|---|
| Mr6.1 | ----N-GQCEDVWMPCTSNWXCCSL-DC- | (468) |

E-MY-CTQI#²

| | | |
|---|---|---|
| Mr6.2 | --------CGGWSTYCEVDEXCCSE-SC- | (469) |

VRSY-CTLF#²

| | | |
|---|---|---|
| Mr6.3 | ----N-GGCKATWMSCSSGWXCCSM-SC- | (470) |

D-MY-C#²

| | | |
|---|---|---|
| R6.10 | --UFGHXXCTYULGPCXVDDTCCSA-SC- | (471) |

XSKF-CGLU\hat{ }

| | | |
|---|---|---|
| R6.9 | --WWE-GECSNWLGSCSTPSNCCLK-SC- | (472) |

N-GH-CTLW\hat{ }

| | | |
|---|---|---|
| Tx6.1 | ---L----CODYTXOCSHAHXCCSW-NC- | (473) |

YNGH-CT#²

| | | |
|---|---|---|
| Tx6.14 | -------DCYSWLGSCIAPSQCCSE-VC- | (474) |

D-YY-CRLWR\hat{ }

| | | |
|---|---|---|
| Tx6.4 | --WL---ECSVWFSHCTKDSXCCSN-SC- | (475) |

DQTY-CTLMPPDW\hat{ }²

| | | |
|---|---|---|
| Tx6.5 | GMW---GECKDGLTTCLAPSXCCSE-DC- | (476) |

E-GS-CTMW\hat{ }²

| | | |
|---|---|---|
| Tx6.6 | D-WWD-DGCSV-WGPCTVNAXCCSG-DC- | (477) |

H-ET-CIFGWEV\hat{ }²

| | | |
|---|---|---|
| Tx6.9 | --WWRWGGCMAWFGLCSRDSXCCSN-SC- | (478) |

DVTR-CELMPFPPDW\hat{ }²

| | | |
|---|---|---|
| TxVIIA | --------CGGYSTYCXVDSXCCSD-NC- | (479) |

VRSY-CTLF#

¹The E may be Glu or Gla, the P may be Pro or hydroxy-Pro, and W may be Trp or bromo-Trp.
²Peptide disclosed in U.S. Ser. No. 09/210,952 (PCT/US98/26792).

TABLE 3

| | |
|---|---|
| Alignment of σ-Conopeptides | (SEQ ID NO:) |

| | | |
|---|---|---|
| Ca8.1 | GCS-GT-CHRREDGKC-RGTCDCSG-YSYCR | (480) |

CG-DAHHFYRGCTCSCQ#

| | | |
|---|---|---|
| Ca8.2 | GCSG-T-CHRREDGKC-RGTCDCSG-YSYCR | (481) |

CG-DAHHFYRGCTCTC\hat{ }

| | | |
|---|---|---|
| Ca8.3 | GCSG-T-CRRHRDGKC-RGTCDCSG-YSYCR | (482) |

CG-DAHHFYRGCTCTC\hat{ }

| | | |
|---|---|---|
| Ca8.4 | GCSG-T-CRRHRDGKC-RGTCDCSG-YSYCR | (483) |

CG-DAHHFYRGCTCTC\hat{ }

| | | |
|---|---|---|
| Ca8.5 | GCSG-T-CHRREDGKC-RGTCDCSG-YSYCR | (484) |

CG-DAHHFYRGCTCTC\hat{ }

| | | |
|---|---|---|
| Ca8.6 | GCSG-T-CHRRQNGEC-QGTCDCDG-HDHCD | (485) |

CG-DTLGTYSGCVCIC\hat{ }

| | | |
|---|---|---|
| La8.1 | QSE--TACRSLGWYQCM-GKCQ-LGVHSWCE | (486) |

CIYNRGSQKSGCACRCQK\hat{ }

| | | |
|---|---|---|
| Mn8.1 | QCTLVNNCDRNGERACN-GDCSCEGQI--CK | (487) |

CGYRVSPGKSGCACTCRNAK\hat{ }

| | | |
|---|---|---|
| P8.1 | GCS-GSPCFKNKT--C-RDECICGG-LSNCW | (488) |

CGY-GGS--RGCKCTCRE\hat{ }

| | | |
|---|---|---|
| R8.1 | KCNF-DKCKGTGVYNCG-ESCSCEGLHS-CR | (489) |

CTYNIGSMKSGCACICTYY\hat{ }

| | | |
|---|---|---|
| R8.2 | YGLGCA-GT-CGSSSN--CVRDYCDC-P-KP | (490) |

NCYCT-GKGFRQPGCGCSCL#

| | | |
|---|---|---|
| Sx8.1 | QCTFVNNCQQNG--CAN-GDCSCGDQI--CK | (491) |

CGYRISPGRSGCACTCRNAK\hat{ }

| | | |
|---|---|---|
| T8.1 | FGPIC---T-CFKSQN--C-RGSCECMS-PP | (492) |

GCYCS-NNGIRERGCSCTCPGT#

| | | |
|---|---|---|
| T8.2 | GCT--GNCDW----TCS-GDCSCQGTSDSCH | (493) |

CIPPKSIGNR-CRCQCKRKIEID\hat{ }

TABLE 4

| | |
|---|---|
| Alignment of τ-Conopeptides | (SEQ ID NO:) |

| | | |
|---|---|---|
| Tx5.2a | ---ECCEDGW-CCTAAPLT#¹ | (494) |
| Tx5.2b | ---GCCEDGW-CCTAAPLT# | (495) |
| Mr5.1 | --NGCC-RAGDCCSRFEIKENDF#¹ | (496) |
| Mr5.3 | --NGCC-RAGDCCS\hat{ }¹ | (497) |
| Mr5.2 | --NACC-IVRQCC\hat{ }¹ | (498) |
| Qc5.1 | ---GCCAR-LTCCV#¹ | (499) |
| Qc5.2 | ---GCCAM-LTCCV#¹ | (500) |
| t-PVA | ---GCCPKQMRCCTL#¹ | (501) |
| Ca5.1 | -----CCPRRLACCII#¹ | (502) |
| Ca5.2 | ----CCPNK-PCCFI#¹ | (503) |
| G5.1 | -ZGWCCKENIACCI\hat{ }¹ | (504) |
| G5.2 | -ZGWCCKENIACCV\hat{ }¹ | (505) |
| Im5.1 | DWNSCCGKNPGCCPW#¹ | (506) |
| Bt5.1 | ---NCCPDSPPCCH\hat{ } | (507) |
| Af5.2 | --GNCCEFWEFCCD\hat{ } | (508) |
| Da5.1 | ----CCEYWKLCC# | (509) |
| Om5.1 | ---VCCGYKFFCCR\hat{ } | (510) |
| t-AuVA | ---FCCPVIRYCCW\hat{ }¹ | (511) |
| t-AuVB | ---FCCPFIRYCCW\hat{ }¹ | (512) |
| Au5.1 | ----CCPMIYWCCS\hat{ } | (513) |
| Au5.4 | ----CCPEIYWCCS\hat{ } | (514) |
| Nb5.1 | ---ICCPIILWCC# | (515) |
| Af5.1 | ----CCPPVIWCC# | (516) |
| Tx5.1 | -----CCQTFYWCCVQ# | (517) |
| Au5.3 | WNNYCCTNELWCC# | (518) |
| Gm5.1 | ---LCCVTEDWCCEWW\hat{ }¹ | (519) |
| Gm5.2 | ---VCCRPVQDCCS#¹ | (520) |
| Da5.2 | -PVNCCPIDQSCCS\hat{ } | (521) |
| Sf5.1 | GNIHCCTKYQPCCSSPS\hat{ } | (522) |

¹Peptide disclosed in U.S. Ser. No. 09/497,491 (PCT/US00/03021)

TABLE 5

| | |
|---|---|
| Alignment | |
| of Mar-Type Conopeptides¹ | (SEQ ID NO:) |

| | | | |
|---|---|---|---|
| Tx1.6 | (Q819) | -ZTCCGYRMCVPC# | (523) |
| Bn1.5 | (Q818) | -A-CCGYKLCSPC\hat{ } | (524) |
| Pn1.3 | (Q820) | -STCCGFKMCIPCR\hat{ } | (525) |
| Pn1.5 | (AA200) | -STCCGFKMCIPCS\hat{ } | (526) |
| Pn1.7 | (AA456) | -STCCGFKMCIPC# | (527) |
| Ep1.5 | (AA457) | -STCCGYRMCVPC# | (528) |
| Mr1.3 | | NGVCCGYKLCLPC\hat{ } | (529) |
| Pn1.6 | (AA390) | --LCCGFWMCIPCN\hat{ } | (530) |
| Mr1.1 | | NGVCCGYKLCHOC\hat{ } | (531) |
| Mr1.2 | | -GVCCGYKLCHOC\hat{ } | (532) |
| Bn1.5 | | --ACCGYKLCSPC\hat{ } | (533) |
| Au1.4 | | -SVCCGYKLCFPC# | (534) |
| Tx1.7 | | NGVCCGYRMCVPC# | (535) |
| Tx1.6 | | -ZTCCGYRMCVPC# | (536) |
| Af1.3 | | -ZACCGFKMCVPC# | (537) |
| Pn1.3 | | -STCCGFKMCIPCR\hat{ } | (538) |
| Pn1.4 | | NGVCCGFWMCIPCN\hat{ } | (539) |
| Om1.7 | | -DVCCYVRMC-PCR\hat{ } | (540) |

¹Some peptides disclosed in U.S. Ser. No. 09/580,201. P may also be O and O may also be P.

TABLE 6

| | |
|---|---|
| Alignment of Contryphans* | (SEQ ID NO:) |

| | | |
|---|---|---|
| Contryphan-Im | Z--C-GQAWC# | (541) |
| Contryphan-Sm-dW4, V7 | GCOWQPVC# | (542) |
| Contryphan-Ar-1 | ZYGCOOGLWCH\hat{ } | (543) |
| C. arenatus contryphan | ASGCPWRPWC# | (544) |
| 1A | | |
| C. arenatus contryphan 2 | ZYGCPVGLWCD\hat{ } | (545) |
| C. arenatus contryphan 4 | SGCPWQPWC# | (546) |
| C. arenatus contryphan 1 | SGCPWHPWC# | (547) |

*P may be Pro or hydroxy-Pro; Z may be Gln or pyro-Glu.

TABLE 7

| | |
|---|---|
| Alignment of αA-Conopeptides* | (SEQ ID NO:) |

| | | |
|---|---|---|
| αA- | GCCGKYONAACHOCGCTVGROOYCDROSGG# | (548) |
| EIVB | | |
| P4.1 | GCCGSYPNAACHPCGCK-DRPSYCGQ# | (549) |
| P4.2 | EGCC---SNPACHPCGCK-DRPSYCGQ# | (550) |

*P may be Pro or hydroxy-Pro

TABLE 8

| | |
|---|---|
| Alignment | |
| of Bromosleeper Conopeptides* | (SEQ ID NO:) |

| | | |
|---|---|---|
| Bromosleeper-Ar1 | VVTEACEESCEEEEKHCCH | (551) |

VNNGVPSCAVICW#

| | | |
|---|---|---|
| Bromosleeper-Ar1A | IVTEACEESCEDEEKHCCH | (552) |

VNNGVPSCAVICW#

| | | |
|---|---|---|
| Bromosleeper-Ar2 | IVTEACEEHCEDEEQFCCG | (553) |

LENGQPFCAPVCF#

| | | |
|---|---|---|
| Bromosleeper-Ar3 | VVTGACEEHCEDEEKHCCG | (554) |

LENGQPFCARLCL#

| | | |
|---|---|---|
| Bromosleeper-Di1 | NVDQECIDACQLEDKNCCG | (555) |

RTDGEPRCAKICL#

| | | |
|---|---|---|
| Bromosleeper-Di2 | ETDQECIDICKQEDKKCCG | (556) |

RSNGEPTCAKICL#

| | | |
|---|---|---|
| Bromosleeper-Di3 | ETDQECIDTCEQEDKKCCG | (557) |

RTNGEPVCAKICF#

| | | |
|---|---|---|
| Bromosleeper-P1 | PKTEACEEVCELEEKHCCC | (558) |

IRSDGPKCSRKCLLSIFC\hat{ }

| | | |
|---|---|---|
| Bromosleeper-P2 | VVSEECKKYCKKQNKNCCS | (559) |

SKHEEPRCAKICF#

| | | |
|---|---|---|
| Bromosleeper-Sn | AVTEACTEDCKTQDKKCCG | (560) |

EMNGQHTCAKICL#

| | | |
|---|---|---|
| Bromosleeper-T1 | PKTKECERYCELEEKHCCC | (561) |

IRSNGPKCSRICIFKFWC\hat{ }

| | | |
|---|---|---|
| Bromosleeper-T2 | PKTRECEMQCEQEEKHCCR | (562) |

VRDGTGQCAPKCLGINW\hat{ }

*The E may be Glu or Gla, the P may be Pro or hydroxy-Pro, and W may be Trp or bromo-Trp.

TABLE 9

| | |
|---|---|
| Alignment | |
| of Conopressins | (SEQ ID NO:) |

| | | |
|---|---|---|
| Conopressin-G | CFIRNCPKG# | (563) |
| Conopressin-S | CIIRNCPRG# | (564) |

TABLE 10

| | |
|---|---|
| Alignment of O-Superfamily | (SEQ ID NO:) |

| | | |
|---|---|---|
| Ar6.1 | -----GCTPPGGVCGYHGH---CCD-F-C--DTFGNLCVS# | (565) |
| C. geogr. GS-A | -----ACSGRGSRCPPQ-----CCMGLTC--GREYPPRC# | (566) |
| Ca6.3 (F166) | -----NCGEQGEGCAT--RP--CCSGLSC-VGSRPGGLCQY# | (567) |
| convulsion | -----NCPY----CVVY-----CCPPAYCEASG-----CRPP# | (568) |
| De6.1 | -----ACKOKNNLCAITXMAX-CCSGF-CLIY-----RC\hat{ } | (569) |
| Lv6.2 (I16) | -----SCGHSGAGCYT--RP--CCPGLHC-SGGQAGGLCV\hat{ } | (570) |
| Lv6.3 (I12) | -----DCGESGQGCYSV-RP--CCPGLICKGTG-GGGLCRPSGI\hat{ } | (571) |
| Mi6.1 (F204) | ------CTPPGGLC-YHAYP--CCSKT-C---NLDTSQCEPRWS\hat{ } | (572) |
| Mi6.2 (F162) | ------CTDDSQFCNPSNHD--CCSG-KCIDEGDNG-ICAIVPENS\hat{ } | (573) |
| Mi6.3 (F161) | ------CTEDSQFCNPSNHD--CCSG-KCIDEGDNG-ICAIVPENS\hat{ } | (574) |
| Pu6.1 (JG14) | ------CSDFGSDCVPATHN--CCSG-ECFGFEDFG-LCT\hat{ } | (575) |
| Qc6.4 (F025) | -----ACSQVGEACFPQ-KP--CCPGFLC--NH-IGGMCHH\hat{ } | (576) |
| S6.4 | ------CLPDGTSCLFSRIR--CCGT--C---SSILKSCVS\hat{ } | (577) |
| Ts6.3 (F081) | -----SCAEFGEVC-SS-TA--CCPDLDCVEAYSP--ICLWE\hat{ } | (578) |
| Tx6.3 | -----KCVEQWKYCTR---ESLCCAGL-CLFS-----FCIL\hat{ } | (579) |
| Tx6.7 | ------CVEQWEVCGIILFSSSCCGQL-CLFG-----FCVL\hat{ } | (580) |
| Vr6.1 (F198) | -----DCGGQGEGCYT--QP--CCPGLRCRGGGTGGGVCQL\hat{ } | (581) |
| Wi6.1 (M406) | FGSFIPCARLGEPC-----T-ICCRPLRCRESG--TPTCQV\hat{ } | (582) |
| Rg6.6 (K861) | -----TCLEHNKLCWYD---RDCCTIY-C---N--ENKCGVKPQ\hat{ } | (583) |
| EST202 | -----ACKSNYDCPQRFKCCSYTWNGSSGYCKRVCYLYR\hat{ } | (584) |

TABLE 11

| | |
|---|---|
| Alignment | |
| of ψ-Conopeptides* | (SEQ ID NO:) |

| | | |
|---|---|---|
| ψ-PIIIF | GOOCCLYGSCROFOGCYNALCCRK# | (585) |
| U021 | HPPCCMYGRCRRYPGCSSASCCQG# | (586) |
| homolog | | |

*P may be Pro or hydroxy-Pro

TABLE 12
Alignment of kappaA-Conopeptides* (SEQ ID NO:)

| | | | |
|---|---|---|---|
| Cn10.3 | (J454) | APELVVTATTTCCGYDPMTICPPCMCTHS | (587) |

CPPKRKP#

| | | | |
|---|---|---|---|
| A10.2 | (H350) | ZSWLVPSTITTCCGYDPGTMCPPCRCNNT | (588) |

CKPKKPKPGK#

| | | | |
|---|---|---|---|
| Cn10.4 | (G851) | APELVVTATTTCCGYDPMTWCPSCMCTYS | (589) |

CPHQRKKP#

| | | | |
|---|---|---|---|
| M10.3 | (X003) | APELVVTATTTCCGYDPMTICPPCMCTHS | (590) |

CPPKGKP#

| | | | |
|---|---|---|---|
| A10.3 | (AA400) | ZKWLVHSKITYCCGYNKMDMCPPCMCTYS | (591) |

CPPLKKKRP#

| | | | |
|---|---|---|---|
| A10.4 | (AA401) | APWTVVTATTNCCGITGPG-CLPCRCTQT | (592) |

C#

TABLE 13
Alignment of α-Conopeptides (SEQ ID NO:)

| | | |
|---|---|---|
| G1.4 | -ECCHPACGKHYSC# | (593) |
| G1.5 | -ECCNPACGRHFSC# | (594) |
| S1.8 | AYCCHPACGPNYSCGTSCSRTL\hat{ } | (595) |
| S1.9 | AYCCHPVCGKNFDC# | (596) |
| Ra1.1 | GCCCNPACGPNYGCGTSCSRTL\hat{ } | (597) |
| Ar1.1 | ZDYCCTIPSCWDRYKERCRHIR\hat{ } | (593) |
| Er1.1 | ZDYCCTIPSCWDRYKERCRHIR\hat{ } | (599) |
| Mi1.2 | -DYCCHRGPCMVW----C# | (600) |
| Jp1.1 | --GCCSDPRC--RYR--CR\hat{ } | (601) |
| a-OmIA | --GCCSHPACNVNNPHICG# | (602) |
| a-OmIA [COOH] | --GCCSHPACNVNNPHICG\hat{ } | (603) |
| Qc1.1 | Z-GCCSDPACAVSNPDICGG# | (604) |
| Bn1.6 | PE-CCTHPACHVSHPELC# | (605) |
| Mr1.5 | PE-CCTHPACHVSNPELC# | (606) |
| Mi1.1 | ---CCNHPACAGKNSDLC# | (607) |
| MII [YHT] | --GCCYHPTCHLEHSNLC# | (608) |
| Nb1.1 | --GCCERPPCRWQNPDLCG# | (609) |
| Ak1.1 | --TCCSRPTCRMEYPELCG# | (610) |
| Qc1.2 | NE-CCDNPPCKSSNPDLCDWRS\hat{ } | (611) |
| Lp1.1 | ---CCSNPACNRYNPAICD\hat{ } | (612) |
| Em1.1 | -D-CCNFPACAASNPGLCT\hat{ } | (613) |
| C. victor alpha | ---CCSSPPCFASNPA-C# | (614) |
| Cj1.1 | -GGCCSFPPCIANNPF-CA# | (615) |
| Fd1.1 | --GCCSNPPCSYLNPA-C# | (616) |
| Em1.2 | -D-CCSDPPCAHNNPD-CR\hat{ } | (617) |
| Ge1.1 | --GCCSNPPCYANNQAYCN# | (618 |
| Wi1.1 | DE-CCAHPSCWKAEDLICTNQRRRTL\hat{ } | (619) |
| Ca1.5 | --GCCAIRECRLQNAAYCGGIS\hat{ } | (620) |
| Bt1.10 | SATCCYYPPCYEAYPESCL\hat{ } | (621) |

TABLE 14
Alignment of Conopeptides* (SEQ ID NO:)

| | | |
|---|---|---|
| Convulsant | VYXTHP\hat{ } | (622) |
| WG002 | WSWRMGNGDRRSDQ\hat{ } | (623) |
| QcII | DCQPCGHNVCC\hat{ } | (624) |
| Scratching, | KFLSGGFKXIVCHRYCAKGIAKEFCNCPD# | (625) |
| Convulsion | | |
| MAG-1 | RPKNSW\hat{ } | (626) |
| MAG-2 | AROKNSW$ | (627) |
| MAG-3 | ROKNSW\hat{ } | (628) |
| EST66 | CCPSSKEDSLNCIETMATTATCMKSNKGEIY | (629) |
| | SYACGYCGKKKESCFGDKKPVTDYQCQTRNI | |
| | PNPCGGAAL\hat{ } | |
| G12.2 | DESKCDRCNCAELRSSRCTQAIFCLTPELCT | (630) |
| | PSISCPTGECRCTKFHQSRCTRFVECVPNKC | |
| | RDA\hat{ } | |
| G12.1 | DDSYCDGCLCTILKKETCTSTMSCRGT- CR | (631) |
| | KEWPCWEEDCYCTEIQGGACVTPSECKPGEC | |
| | \hat{ } | |
| EST171 | GCVYEGIEYSVGETYQADCNTCRCDGFDLAT | (632) |
| | CTVAGCTGFGPE\hat{ } | |
| U010 homolog | SGPADCCRMKECCTDRVNECLQRYSGREDKF | (633) |
| | VSFCYQEATVTCGSFNEIVGCCYGYQMCMIR | |
| | VVKPNSLSGAHEACKTVSCGNPCA\hat{ } | |
| P29 | DCCGVKLEMCHPCLCDNSCKNYGK# | (634) |
| EST87 | GEPIPTTVINYGECCKDPSCWVKVKDFQCPG | (635) |
| | ASPPN\hat{ } | |
| Ge3.1 (F590) | QCCTFCNFGCQPCCVP\hat{ } | (636) |
| Ts10.1 | DGCPPHPVPGMHKCMCTNTC | (637) |
| Conophysin-R | HPTKPCMYCSFCQCVGPHICCGPTGCEMGTA | (638) |
| | EANMCSEEDEDPIPCQVFGSDCALNNPDNIH | |
| | GHCVADGICCVDDTCTTHLGCL\hat{ } | |

*Conopeptides grouped together are homologous.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### List of References

Abiko, H. et al. (1986). Brain Res. 38:328-335.

Aldrete, J. A. et al. (1979). Crit. Care Med. 7:466-470.

Barnay, G. et al. (2000). J. Med. Chem.

Bitan, G. et al. (1997). J. Peptide Res. 49:421-426.

Bodansky et al. (1966). Chem. Ind. 38:1597-98.

Bulbring, W. and Wajda, J. (1945). J. Pharmacol. Exp. Ther. 85:78-84.

Cartier, G. E. et al. (1996). J. Biol Chem. 271:7522-7528.

Chandler, P. et al. (1993). J. Biol Chem. 268:17173-17178.

Chaplan S. R. (1994). J. Neuroscience Methods 53:55-63.

Chaplan S. R. (1997). J. Pharmacol. Exp. Ther. 280:829-838.

Clark, C. et al. (1981). Toxicon 19:691-699.

Codere, T. J. (1993). Eur. J. Neurosci. 5:390-393.

Craig, A. G. et al. (1997). J. Biol. Chem 272:4689-4698.

Craik, D. J. et al. (2001). Toxicon 39:43-60.

Cruz, L. J. at al. (1976). Verliger 18:302-308.

Cruz, L. J. et al. (1987). J. Biol. Chem. 262:15821-15824.

Ettinger, L. J. et al. (1978). Cancer 41:1270-1273.

Fainzilber, M. et al. (1998). Biochemistry 37:1470-1477.

Goodman and Gilman 's The Pharmacological Basis of Therapeutics, Seventh Ed., Gilman, A. G. et al., eds., Macmillan Publishing Co., New York (1985).

Hammerland et al. (1992). Eur. J. Pharmacol. 226:239-244.

Heading, C. (1999). Curr. Opin. CPNS Invest. Drugs 1:153-166.

Hopkins, C. et al. (1995). J. Biol. Chem. 270:22361-22367.

Horiki, K. et al. (1978). Chemistry Letters 165-68.

Hubry, V. et al. (1994). Reactive Polymers 22:231-241.

Hylden, J. L. K. and Wilcox, G. (1980). Eur. J. Pharmacol. 67:313-316.

Jacobsen, R. et al. (1997). J. Biol. Chem. 272:22531-22537.

Jimenez, E. C. et al. (1996). J. Biol. Chem. 271:28002-28005.

Kaiser et al. (1970). Anal. Biochem. 34:595.

Kapoor (1970). J. Pharm. Sci. 59:1-27.

Kornreich, W. D. et al. (1986). U.S. Pat. No. 4,569,967.

Kruszynski, M. et al. (1990). Experientia 46:771-773.

Luer, M. S. & Hatton, J. (1993). Annals Pharmcotherapy 27:912-921.

Liu, H. et al. (1997). Nature 386:721-724.

Malmberg, A. B. and Basbaum, A. I. (1998). Pain 76:215-222.

Maric, M. et al. (1989). Physiol. Pharmacol. 67:1437-1441.

Martinez, J. S. et al. (1995). Biochem. 34:14519-14526.

Mayer, E. A. et al. (1994). Gastroenterology 107:271-293.

McIntosh, J. M. et al. (1998). Methods Enzymol. 294:605-624.

The Merck Manual of Diagnosis and Therapy, 16 Ed., Berkow, R. et al., eds., Merck Research Laboratories, Rahway, N.J., pp. 1436-1445 (1992).

Methoden der Organischen Chemie (Houben-Weyl): Synthese von Peptiden, E. Wunsch (Ed.), Georg Thieme Verlag, Stuttgart, Ger. (1974).

Nehlig, A. et al. (1990). Effects of phenobarbital in the developing rat brain. In Neonatal Seizures, Wasterlain, C. G. and Vertt, P. (eds.), Raven Press, New York, pp. 285-194.

Nishiuchi, Y. et al. (1993). Int. J. Pept. Protein Res. 42:533-538.

Olivera, B. M. et al. (1984). U.S. Pat. No. 4,447,356.

Olivera, B. M. et al. (1985). Science 230:1338-1343.

Olivera, B. M. et al. (1990). Science 249:257-263.

Olivera, B. M. et al. (1996). U.S. Pat. No. 5,514,774.

Ornstein, et al. (1993). Biorganic Medicinal Chemistry Letters 3:43-48.

Remington's Pharmaceutical Sciences, 18th Ed. (1990, Mack Publishing Co., Easton, Pa.).

Rivier, J. R. et al. (1978). Biopolymers 17:1927-38.

Rivier, J. R. et al. (1987). Biochem. 26:8508-8512.

Sambrook, J. et al. (1989). Molecular Cloning: A Laboratory Manual, 2nd Ed., Cold Spring Harbor Laboratory, Cold Spring Harbor, N.Y.

Shon, K.-J. et al. (1994). Biochemistry 33:11420-11425.

Shon, K.-J. et al. (1997). Biochemistry 36:9581-9587.

Stewart and Young, Solid-Phase Peptide Synthesis, Freeman & Co., San Francisco, Calif. (1969).

Vale et al. (1978). U.S. Pat. No. 4,105,603.

Troupin, A. S. et al. (1986). MK-801. In New Anticonvulsant Drugs, Current Problems in Epilepsy 4, Meldrum, B. S. and Porter, R. J. (eds.), John Libbey, London, pp. 191-202.

Van de Steen, P. et al. (1998). Critical Rev. in Biochem. and Mol. Biol. 33:151-208.

White, H. S., et al. (1992). Epilepsy Res. 12:217-226.

White, H. S., et al. (1995). Experimental Selection, Quantification, and Evaluation of Antiepileptic Drugs. In Antiepileptic Drugs, 4th Ed., Levy, R. H., eds., Raven Press, N.Y., pp. 99-110.

Wong, E. H. P. et al. (1986). Proc. Natl. Acad. Sci. USA 83:7104-7108.

Zhou L. M., et al. (1996). J. Neurochem. 66:620-628.

Zimm, S. et al. (1984). Cancer Res. 44:1698-1701.

U.S. Pat. No. 3,842,067.

U.S. Pat. No. 3,862,925.

U.S. Pat. No. 3,972,859.

U.S. Pat. No. 5,514,774.

U.S. Pat. No. 5,550,050.

U.S. Pat. No. 5,670,622.

U.S. Pat. No. 5,719,264.

U.S. Pat. No. 5,844,077.

U.S. Pat. No. 5,889,147.

U.S. Pat. No. 5,969,096.

U.S. Pat. No. 6,077,934.

Published PCT Application WO 92/19195.

Published PCT Application WO 94/25503.

Published PCT Application WO 95/01203.

Published PCT Application WO 95/05452.

Published PCT Application WO 96/02286.

Published PCT Application WO 96/02646.

Published PCT Application WO 96/40871.

Published PCT Application WO 96/40959.

Published PCT Application WO 97/12635.

Published PCT Application WO 98/03189.

Published PCT Application WO 00/23092.

What is claimed is:

1. An isolated peptide selected from the group consisting of:
(a) a peptide set forth in Tables 1-14; and
(b) a derivative of the peptide in (a).

2. The isolated peptide of claim 1, wherein Xaa1 is Glu or γ-carboxy-Glu, Xaa2 is Gln or pyro-Glu, Xaa3 is Pro or hydroxy-Pro, Xaa4 is Trp or bromo-Trp, and Xaa5 is Tyr, ¹²⁵I-Tyr, mono-iodo-Tyr, di-iodo-Tyr, O-sulpho-Tyr or O-phospho-Tyr.

3. The derivative of the peptide of claim 1, in which the Arg residues may be substituted by Lys, omithine, homoargine, nor-Lys, N-methyl-Lys, N,N-dimethyl-Lys, N,N,N-trimethyl-Lys or any synthetic basic amino acid; the Lys residues may be substituted by Arg, omithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; the Asn, Ser, Thr or Hyp residues may be glycosylated;. the Tyr residues may also be substituted with the 3-hydroxyl or 2-hydroxyl isomers (meta-Tyr or ortho-Tyr, respectively) and corresponding O-sulpho- and O-phospho-derivatives; the acidic amino acid residues may be substituted with any synthetic acidic amino acid, e.g., tetrazolyl derivatives of Gly and Ala; the aliphatic amino acids may be substituted by synthetic derivatives bearing non-natural aliphatic branched or linear side chains CₙH₂ₙ₊₂ up to and including n=8; the Leu residues may be substituted with Leu (D); the Glu residues may be substituted with Gla; the Gla residues may be substituted with Glu; the N-terminal Gln residues may be substituted with pyroGlu; the Met residues may be subsituted by Nle; the Cys residues may be in D or L configuration and may optionally be substituted with homocysteine (D or L); and pairs of Cys residues may be replaced pairwise with isoteric lactam or ester-thioether replacements, such as Ser/(Glu or Asp), Lys/(Glu or Asp), Cys/(Glu or Asp) or Cys/Ala combinations.

4. An isolated nucleic acid encoding an conotoxin propeptide having an amino acid sequence set forth in Table 1.

5. The isolated nucleic acid of claim 4, wherein the nucleic acid comprises a nucleotide sequence set forth in Table 1.

6. An isolated conotoxin propeptide having an amino acid sequence set forth in Table 1.

7. A method of alleviating pain in an individual which comprises administering to said individual that is either exhibiting pain or is about to be subjected to a pain-causing event a pain-alleviating amount of an active agent comprising a pain-relieving conotoxin peptide of claim 1 or a pharmaceutically acceptable salt thereof.

8. A method for treating or preventing disorders associated with a disorder selected from the group consisting of voltage-gated ion channel disorders, ligand-gated ion channel disorders and receptor disorders in an individual which comprises administering to an individual in need thereof a therapeutically effective amount of a conotoxin peptide of claim 1 or a pharmaceutically acceptable salt thereof.

9. A method of identifying compounds that mimic the therapeutic activity of a conotoxin, comprising the steps of: (a) conducting a biological assay on a test compound to determine the therapeutic activity; and (b) comparing the results obtained from the biological assay of the test compound to the results obtained from the biological assay of a conotoxin.

10. A substantially pure conotoxin peptide derivative comprising a permutant of the peptide of claim 1.

11. A substantially pure conotoxin peptide derivative comprising a permutant of the peptide of claim 2.

12. Use of a radiolabeled conotoxin peptide of claim 1 for characterization of a new site on the aforementioned receptors or channels and use of these peptide probes for screening and identification of novel small molecules that interact at the aforementioned sites.

13. The use of claim 12, wherein said receptor or channel is a monoamine transporter.

14. The use of claim 13, wherein said peptide is selected from the group of peptides set forth in Table 5.


SEQUENCE LISTING (638 sequences)

SEQ ID NO: 1 - DNA, 290 bp, Conus ammiralis
  CDS (4)..(231)
atcatggagaaactgataattctgcttcttgttgctgctgtactgatgtcgacccaggccctggttgaacgtgctggagaaaaccgctcaaaggagaacatcaattttttattaaaaagaaagagagctgctgacagggggatgtggggcgattgcaaagatgggttaacgacatgttttgcgccctcagagtgttgttctgaggattgtgaagggagctgcacgatgtggtgatgacctctgaccacaagccatctgacatcaccactctcctcttcagaggcttcaag

SEQ ID NO: 2 - protein, 76 aa, Conus ammiralis
MEKLIILLLVAAVLMSTQALVERAGENRSKENINFLLKRKRAADRGMWGDCKDGLTTCFAPSECCSEDCEGSCTMW

SEQ ID NO: 3 - protein, 31 aa, Conus ammiralis
  PEPTIDE (1)..(31): Xaa at residues 18, 22 and 25 is Glu or gamma-carboxy-Glu; Xaa at residue 16 is Pro or hydroxy-Pro; Xaa at residues 3 and 31is Trp (D or L) or bromo-Trp (D or L)
GMXGDCKDGLTTCFAXSXCCSXDCXGSCTMX

SEQ ID NO: 4 - DNA, 295 bp, Conus ammiralis
  CDS (4)..(246)
atcatggagaaactgacaattctgcttcttgttgctgctgtactgatgtcgacccaggccctgcctcaaggtggtggagaaaaacgcccaagggagaatatcagatttttatcaaaaagaaagacaaatgctgagcgttggagggagggcagttgcacctcttggttagcgacgtgtacgcaagaccagcaatgctgtactgatgtttgttacaaaagggactactgcgccttgtgggatgaccgctgaccacaagccatctgacatcaccactctcctgttcagagtcttcaag

SEQ ID NO: 5 - protein, 81 aa, Conus ammiralis
MEKLTILLLVAAVLMSTQALPQGGGEKRPRENIRFLSKRKTNAERWREGSCTSWLATCTQDQQCCTDVCYKRDYCALWDDR

SEQ ID NO: 6 - protein, 36 aa, Conus ammiralis
  PEPTIDE (1)..(36): Xaa at residue 3 is Glu or gamma-carboxy-Glu; Xaa at residues 1, 9 and 33 is Trp (D or L) or bromo-Trp (D or L); Xaa at residues 25 and 29 is Tyr, 125I-Tyr, mono-iodo-Tyr, di-iodo-Tyr, O-sulpho-Tyr or O-phospho-Tyr
XRXGSCTSXLATCTQDQQCCTDVCXKRDXCALXDDR

SEQ ID NO: 7 - DNA, 275 bp, Conus ammiralis
  CDS (4)..(219)
atcatgcagaaactgataattctgcttcttgttgctgctgtgctgatgtcgacccaggccctgtttcaagaaaaacgcacaatgaagaagatcgattttttatcaaagggaaaggcagatgctgagaagcagaggaagcgcaattgctcggatgattggcagtattgtgaaagtcccagtgactgctgtagttgggattgtgatgtggtctgctcgggatgaactctgaccacaagtcatccgacatcaccactctcctgttcagaggcttcaag

SEQ ID NO: 8 - protein, 72 aa, Conus ammiralis
MQKLIILLLVAAVLMSTQALFQEKRTMKKIDFLSKGKADAEKQRKRNCSDDWQYCESPSDCCSWDCDVVCSG

SEQ ID NO: 9 - protein, 25 aa, Conus ammiralis
  PEPTIDE (1)..(25): Xaa at residue 10 is Glu or gamma-carboxy-Glu; Xaa at residue 12 is Pro or hydroxy-Pro; Xaa at residues 6 and 18 is Trp (D or L) or bromo-Trp (D or L); Xaa at residue 8 is Tyr, 125I-Tyr, mono- iodo-Tyr, di-iodo-Tyr, O-sulpho-Tyr or O-phospho-Tyr
NCSDDXQXCXSXSDCCSXDCDVVCS

SEQ ID NO: 10 - DNA, 280 bp, Conus ammiralis
  CDS (4)..(246)
atcatgcagaaactgataatcctgcttcttgttgctgctctactgttgtcgatccaggcggtaaatcaagaaaaacaccaacgggcaaagatcaacttgctttcaaagagaaagccacctgctgagcgttggtggcggtggggaggatgcatggcttggtttgggaaatgttcgaaggactcggaatgttgttctaatagttgtgacataacgcgctgcgagttaatgcgattcccaccagactggtgacatcgacactctcctgttcagagtcttcaag

SEQ ID NO: 11 - protein, 81 aa, Conus ammiralis
MQKLIILLLVAALLLSIQAVNQEKHQRAKINLLSKRKPPAERWWRWGGCMAWFGKCSKDSECCSNSCDITRCELMRFPPDW

SEQ ID NO: 12 - protein, 39 aa, Conus ammiralis
  PEPTIDE (1)..(39): Xaa at residues 19 and 31 is Glu or gamma-carboxy-Glu; Xaa at residues 36 and 37 is Pro or hydroxy-Pro; Xaa at residues 1, 2, 4, 10 and 39 is Trp (D or L) or bromo-Trp (D or L)
XXRXGGCMAXFGKCSKDSXCCSNSCDITRCXLMRFXXDX

SEQ ID NO: 13 - DNA, 267 bp, Conus ammiralis
  CDS (4)..(213)
atcatggagaaactgacaatcctgcttcttgttgctgctgtactgacgtcgacccaggccctgattcaaggtggtggagacgaacgccaaaaggcaaagatcaactttctttcaaggtcggaccgcgattgcaggggttacgatgcgccgtgtagctctggcgcgccatgttgtgattggtggacatgttcagcacgaaccgggcgctgtttttaggctgaccacaagccatccgacatcaccactctcctcttcagaggcttcaag

SEQ ID NO: 14 - protein, 70 aa, Conus ammiralis
MEKLTILLLVAAVLTSTQALIQGGGDERQKAKINFLSRSDRDCRGYDAPCSSGAPCCDWWTCSARTGRCF

SEQ ID NO: 15 - protein, 29 aa, Conus ammiralis
  PEPTIDE (1)..(29): Xaa at residues 8 and 14 is Pro or hydroxy-Pro; Xaa at residues 18 and 19 is Trp (D or L) or bromo-Trp (D or L); Xaa at residue 5 is Tyr, 125I-Tyr, mono-iodo-Tyr, di-iodo-Tyr, O-sulpho-Tyr or O-phospho-Tyr
DCRGXDAXCSSGAXCCDXXTCSARTGRCF

SEQ ID NO: 16 - DNA, 277 bp, Conus ammiralis
  CDS (4)..(222)
atcatgcagaaactgacaattctgcttcttgttgctgctgtgctgatgtcgacccaggccgtgcttcaagaaaaacgcccaaaggagaagatcaagtttttatcaaagaaaaagacagatgctgagaagcagcagaagcgcctttgcccggattacacggagccttgttcacatgcccatgaatgctgttcatggaattgtcataatgggcactgcacgggatgaactcggaccacaagccatcgacatcatcactctcctgttcagagtcttcaag

SEQ ID NO: 17 - protein, 73 aa, Conus ammiralis
MQKLTILLLVAAVLMSTQAVLQEKRPKEKIKFLSKKKTDAEKQQKRLCPDYTEPCSHAHECCSWNCHNGHCTG

SEQ ID NO: 18 - protein, 26 aa, Conus ammiralis
  PEPTIDE (1)..(26): Xaa at residues 7 and 14 is Glu or gamma-carboxy-Glu; Xaa at residues 3 and 8 is Pro or hydroxy-Pro; Xaa at residue 18 is Trp (D or L) or bromo-Trp (D or L); Xaa at residue 5 is Tyr, 125I-Tyr, mono-iodo-Tyr, di-iodo-Tyr, O-sulpho-Tyr or O-phospho-Tyr
LCXDXTXXCSHAHXCCSXNCHNGHCT

SEQ ID NO: 19 - DNA, 287 bp, Conus ammiralis
  CDS (4)..(228)
atcatgcagaaactgataattctgctccttgttgctgctgtactgatgtcgacccaggccatgtttcaaggtgatggagaaaaatcccggaaagcggagatcaacttttctaaaacaagaaatttggcgagaaacaagcagaaacgctgcagtagttgggcaaagtattgtgaagttgactcggaatgctgttccgaacagtgtgtaaggtcttactgcgcgatgtggtgatgacctctgaccacaagccatccgatatcaccactctcctcttcagagacttcaag

SEQ ID NO: 20 - protein, 75 aa, Conus ammiralis
MQKLIILLLVAAVLMSTQAMFQGDGEKSRKAEINFSKTRNLARNKQKRCSSWAKYCEVDSECCSEQCVRSYCAMW

SEQ ID NO: 21 - protein, 27 aa, Conus ammiralis
  PEPTIDE (1)..(27): Xaa at residues 9, 13 adn 17 is Glu or gamma-carboxy-Glu; Xaa at residue 13 is Pro or hydroxy-Pro; Xaa at residues 4 and 27 is Trp (D or L) or bromo-Trp (D or L);
CSSXAKXCXVDSXCCSXQCVRSXCAMX

SEQ ID NO: 22 - DNA, 461 bp, Conus ammiralis
  CDS (4)..(270)
gttaaaatgcatctgtcactggcacgctcagctgttttgatgttgcttctgctgtttgccttgggcaactttgttgtggtccagtcaggacagataacaagagatgtggacaatggacagctcacggacaaccgccgtaacctgcaatcgaagtggaagccagtgagtctcttcatgtcacgacggtcttgtaacaattcttgcaatgagcattccgattgcgaatcccattgtatttgcacgtttagcggatgcaaaattattttgatataaacggattgagtttgctcgtcaacaagatgtcgcactacagctcctctctacagtgtgtacatcgaccaaacgacgcatcttttatttctttgtctgttgtatttgttttcctgtgttcataacgtacagagccctttaattacctttactgctcttcacttaacctgataaccggaaggtccagtgct

SEQ ID NO: 23 - protein, 89 aa, Conus ammiralis
KMHLSLARSAVLMLLLLFALGNFVVVQSGQITRDVDNGQLTDNRRNLQSKWKPVSLFMSRRSCNNSCNEHSDCESHCICTFSGCKIILI

SEQ ID NO: 24 - protein, 28 aa, Conus ammiralis
  PEPTIDE (1)..(28): Xaa at residues 8 and 13 is Glu or gamma-carboxy-Glu
SCNNSCNXHSDCXSHCICTFSGCKIILI

SEQ ID NO: 25 - DNA, 459 bp, Conus ammiralis
  CDS (7)..(270)
gttaaaatgcatctgtcactggcacgcttagctgttttgatgttgcttctgctgtttgccttgggcaactttgttgtggtccagtcaggacagataacaagagatgtggacaatggacagctcacggacaaccgccgtaacctgcaatcgaagtggaagccagtgagtctcttcatgtcacgacggtcttgtaacaattcttgcaatgagcattccgattgcgaatcccattgtatttgcacgtttagaggatgcggagctgttaatggttgagtttgctcgtcaacatgatgtcgcactacacactacagctcctctctacagtgtgtacatcgaccaaacgacgcatcttttatttctttgtctgttgtgtttgttttcctgtgttcataacgtacagagccctttaattacttttactgctcttcacttaacctgataaccagaaggtccagtgct

SEQ ID NO: 26 - protein, 88 aa, Conus ammiralis
MHLSLARLAVLMLLLLFALGNFVVVQSGQITRDVDNGQLTDNRRNLQSKWKPVSLFMSRRSCNNSCNEHSDCESHCICTFRGCGAVNG

SEQ ID NO: 27 - protein, 27 aa, Conus ammiralis
  PEPTIDE (1)..(27): Xaa at residues 8 and 13 is Glu or gamma-carboxy-Glu
SCNNSCNXHSDCXSHCICTFRGCGAVN

SEQ ID NO: 28 - DNA, 387 bp, Conus arenatus
  CDS (19)..(240)
accaaaaccatcatcaaaatgaaactgacgtgcgtggtgatcgtcgctgtgctgttcctgacggcctgtcaactcactacagctgatgactccagaggtacgcagaagcatggtgccctgagatcgaccaccaaactctccatgttgactcggggctgcacgcctcctggtggagtttgtggttatcatggtcactgctgcgatttttgcgatacgttcggcaatttatgtgtgagtggctgacccggcatctgacctttcccccttctttgctccactatcctttttctgcctgagtcctccatacctgagagctgtcatgaaccactcaacacctactcttccggaggtttctgaggagctgcattgaaataaaagccgcattgc

SEQ ID NO: 29 - protein, 74 aa, Conus arenatus
MKLTCVVIVAVLFLTACQLTTADDSRGTQKHGALRSTTKLSMLTRGCTPPGGVCGYHGHCCDFCDTFGNLCVSG

SEQ ID NO: 30 - protein, 28 aa, Conus arenatus
  PEPTIDE (1)..(28): Xaa at residues 4 and 5 is Pro or hydroxy-Pro; Xaa at residue 11 is Tyr, 125I-Tyr, mono-iodo-Tyr, di-iodo-Tyr, O-sulpho-Tyr or O-phospho-Tyr
GCTXXGGVCGXHGHCCDFCDTFGNLCVS

SEQ ID NO: 31 - DNA, 375 bp, Conus arenatus
  CDS (24)..(260)
gacaggattgaacaaaattcaggatgtcaggattgggaatcatggtgctaacccttctacttcttgtgttcatggcaaccagtcatcaggatgcaggagagaagaaggcgatgcaaagggacgcaatcaacgtcagacggagaagatcactcactcggggagtagtaactgaggcgtgcgaagagtcctgtgaggaggaggaaaagcactgctgccacgtaaataatggagtaccctcttgtgccgttatatgctggggatagtttctcgcacactgtctcattcattattttatcagtacaagtgtaaacgagacatgtcagaaagtcgaaggttgtgcgtatttgataagtattgtttactgggatgaacgga

SEQ ID NO: 32 - protein, 79 aa, Conus arenatus
MSGLGIMVLTLLLLVFMATSHQDAGEKKAMQRDAINVRRRRSLTRGVVTEACEESCEEEEKHCCHVNNGVPSCAVICWG

SEQ ID NO: 33 - protein, 32 aa, Conus arenatus
  PEPTIDE (1)..(32): Xaa at residues 4, 7, 8, 11, 12, 13 and 14 is Glu or gamma- carboxy-Glu; Xaa at residue 25 is Pro or hydroxy-Pro; Xaa at residue 32 is Trp (D or L) or bromo-Trp (D or L)
VVTXACXXSCXXXXKHCCHVNNGVXSCAVICX

SEQ ID NO: 34 - DNA, 375 bp, Conus arenatus
  CDS (24)..(260)
gacaggattgaacaaaattcaggatgtcaggattgggaatcatggtgctaacccttctacttcttgtgttcatggcaaccagtcatcaggatgcaggagagaagcaggcgacggaaagggacgcaatcaacatcagatggagaagatcacgcactcggagaatagtaactgaggcgtgcgaagagtcctgtgaggacgaggaaaagcactgctgccacgtaaataatggagtaccctcttgtgccgttatatgctggggatagtttctcgcacactgtctcattcattattttatcagtacaagtgtaaacgagacatgtcagaaagtcgaaggttgtgcgtatttgataagtattgtttactgggatgaacgga

SEQ ID NO: 35 - protein, 79 aa, Conus arenatus
MSGLGIMVLTLLLLVFMATSHQDAGEKQATERDAINIRWRRSRTRRIVTEACEESCEDEEKHCCHVNNGVPSCAVICWG

SEQ ID NO: 36 - protein, 32 aa, Conus arenatus
  PEPTIDE (1)..(32): Xaa at residues 4, 7, 8, 11, 13 adn 14 is Glu or gamma-carboxy- Glu; Xaa at residue 25 is Pro or hydroxy-Pro; Xaa at residue 32 is Trp (D or L) or bromo-Trp (D or L)
IVTXACXXSCXDXXKHCCHVNNGVXSCAVICX

SEQ ID NO: 37 - DNA, 374 bp, Conus arenatus
  CDS (24)..(260)
gacaggattgaacaaaattcaggatgtcagaactgggaatcatggtgctaacgcttctacttcttgtgttcctggtaaccagtcatcaggatgcaggagagaagcaggcgacggaaagggacgcaatcaacatcagatggagaagatcactcactcggagaatagtaactgaggcgtgcgaagagcactgtgaggatgaggaacagttctgctgcggcttagagaatggacaacccttttgtgcccctgtttgcttcggatagtttctgtacactgtctcattaattattttatcagtacaagtgtaaacaaaacatgtcagaaagtcgaaggttgtgcgtatttgataagtattgtttgctgggacgaacgga

SEQ ID NO: 38 - protein, 79 aa, Conus arenatus
MSELGIMVLTLLLLVFLVTSHQDAGEKQATERDAINIRWRRSLTRRIVTEACEEHCEDEEQFCCGLENGQPFCAPVCFG

SEQ ID NO: 39 - protein, 32 aa, Conus arenatus
  PEPTIDE (1)..(32): Xaa at residues 4, 7, 8, 11, 13, 14 and 21 is Glu or gamma- carboxy-Glu; Xaa at residues 25 and 29 is Pro or hydroxy-Pro
IVTXACXXHCXDXXQFCCGLXNGQXFCAXVCF

SEQ ID NO: 40 - DNA, 375 bp, Conus arenatus
  CDS (24)..(260)
gacaggattgaacaaaattcaggatgtcaggattgggaatcatggtgctaacccttctacttcttgtgttcatggcaaccagtcatcaggatgcaggagagaagaaggtgatgcaaagggacgcaatcaacgtcagacggagaagatcacgcactcggagagtagtaactggggcgtgcgaagagcactgtgaggacgaggaaaagcactgctgcggcttagagaatggacaacccttttgtgcccgtctatgcttaggatagttttctgtacactgtcttattcattattttatcagtacaagtgaaaacaaagcatgtcagaaagtcgaaggttgtgcgtatttgataagtattgtttactgggatgaacgga

SEQ ID NO: 41 - protein, 79 aa, Conus arenatus
MSGLGIMVLTLLLLVFMATSHQDAGEKKVMQRDAINVRRRRSRTRRVVTGACEEHCEDEEKHCCGLENGQPFCARLCLG

SEQ ID NO: 42 - protein, 32 aa, Conus arenatus
  PEPTIDE (1)..(31): Xaa at residues 7, 8, 11, 13, 14 and 21 is Glu or gamma-carboxy- Glu; Xaa at residue 25 is Pro or hydroxy-Pro
VVTGACXXHCXDXXKHCCGLXNGQXFCARLCL

SEQ ID NO: 43 - DNA, 219 bp, Conus arenatus
  CDS (1)..(189)
atggggaaactgacaatactggttcttgttgctgctgtactgttgtcgacccaggtcatggttcaaggtgacggagatcaacctgcagctcgcaatgcagtgccaaaagacgataacccagatggagcgagtggaaagttcatgaatgttctacgtcggtctggatgtccgtggcatccttggtgtggctgatcggaatccacgattgcaatgacagcc

SEQ ID NO: 44 - protein, 63 aa, Conus arenatus
MGKLTILVLVAAVLLSTQVMVQGDGDQPAARNAVPKDDNPDGASGKFMNVLRRSGCPWHPWCG

SEQ ID NO: 45 - protein, 9 aa, Conus arenatus
  PEPTIDE (1)..(9): Xaa at residues 4 and 7 is Pro or hydroxy-Pro; Xaa at residues 5 and 8 is Trp (D or L) or bromo-Trp (D or L)
SGCXXHXXC

SEQ ID NO: 46 - DNA, 219 bp, Conus arenatus
  CDS (1)..(189)
atggggaaactgacaatactggttcttgttgctgctgtactgttgtcgacccaggtcatggttcaaggtgacggagatcaacctgcagctcgcaatgcagtgccaaaagacgataacccagatggagcgagtggaaagttcatgaatgttctacgtcggtctggatgtccgtggcgcccttggtgtggctgatcggaatccacgattgcaatgacagcc

SEQ ID NO: 47 - protein, 63 aa, Conus arenatus
MGKLTILVLVAAVLLSTQVMVQGDGDQPAARNAVPKDDNPDGASGKFMNVLRRSGCPWRPWCG

SEQ ID NO: 48 - protein, 10 aa, Conus arenatus
  PEPTIDE (1)..(10): Xaa at residues 5 and 8 is Pro or hydroxy-Pro; Xaa at residues 6 and 9 is Trp (D or L) or bromo-Trp (D or L)
ASGCXXRXXC

SEQ ID NO: 49 - DNA, 219 bp, Conus arenatus
  CDS (1)..(189)
atggggaaactgacaatactggttcttgttgctgctgtactgttgtcgacccaggtcatggttcaaggtgacggagatcaacctgcaggtcgagatgcagttccaagagacgataacccaggtggaacgagtggaaagttcatgaatgctctacgtcaatatggatgtccggtgggtctttggtgtgactgatcagaatccacgattgcaatgacagcc

SEQ ID NO: 50 - protein, 63 aa, Conus arenatus
MGKLTILVLVAAVLLSTQVMVQGDGDQPAGRDAVPRDDNPGGTSGKFMNALRQYGCPVGLWCD

SEQ ID NO: 51 - protein, 11 aa, Conus arenatus
  PEPTIDE (1)..(11): Xaa at residue 1 is Gln or pyro-Gln; Xaa at residue 5 is Pro or hydroxy-Pro; Xaa at residue 9 is Trp (D or L) or bromo-Trp D or L); Xaa at residue 2 is Tyr, 125I-Tyr, mono-iodo-Tyr, di-iodo-Tyr, O-sulpho-Tyr or O-phospho-Tyr
XXGCXVGLXCD

SEQ ID NO: 52 - DNA, 210 bp, Conus arenatus
  CDS (1)..(180)
atggggaaactgacaatactggttcttgttgctgctgtactgttgtcgacccaggtcatgtttcgagatcaacctgcacgtcgtgatgcagtgccaagagacgatagcccagatggaatgagtggagggttcatgaatgtcccacgtcggtctggatgtccgtggcaaccttggtgtggctgatcggaatccacgattgcaatgacagcc

SEQ ID NO: 53 - protein, 60 aa, Conus arenatus
MGKLTILVLVAAVLLSTQVMFRDQPARRDAVPRDDSPDGMSGGFMNVPRRSGCPWQPWCG

SEQ ID NO: 54 - protein, 9 aa, Conus arenatus
  PEPTIDE (1)..(9): Xaa at residues 4 and 7 is Pro or hydroxy-Pro; Xaa at residues 5 and 8 is Trp (D or L) or bromo-Trp (D or L)
SGCXXQXXC

SEQ ID NO: 55 - DNA, 210 bp, Conus arenatus
  CDS (1)..(180)
atggggaaactgacaatactggttcttgttgctgctgtactgttgtcgacccaggccatggttcaagatcaacctgcaggtcgagatgcagttccaagagacgataacccaggtggaacgagtggaaagttcgtgaatgctcaacgtcaatatggatgtccgccgggtctttggtgtcactgatcagaatccacgattgcaatgacagcc

SEQ ID NO: 56 - protein, 60 aa, Conus arenatus
MGKLTILVLVAAVLLSTQAMVQDQPAGRDAVPRDDNPGGTSGKFVNAQRQYGCPPGLWCH

SEQ ID NO: 57 - protein, 11 aa, Conus arenatus
  PEPTIDE (1)..(11): Xaa at residue 1 is Gln or pyro-Gln;Xaa at residues 5 and 6 is Pro or hydroxy-Pro; Xaa at residue 9 is Trp (D or L) or bromo-Trp (D or L); Xaa at residue 2 is Tyr, 125I-Tyr, mono-iodo-Tyr, di- iodo-Tyr, O-sulpho-Tyr or O-phospho-Tyr
XXGCXXGLXCH

SEQ ID NO: 58 - DNA, 213 bp, Conus aurisiacus
  CDS (1)..(189)
atgttcaccgtgtttctgttggttgtcttggcaaccactgtcgtttccatcccttcagatcgtgcatctgatggcaggaatgccgcagtcaacgagagagcgccttggctggtcccttcgacaatcacgacttgctgtggatataatccggggacaatgtgccctccttgcaggtgcgataatacctgttaaccaaaaaaaaaaaaaaaaaaa

SEQ ID NO: 59 - protein, 63 aa, Conus aurisiacus
MFTVFLLVVLATTVVSIPSDRASDGRNAAVNERAPWLVPSTITTCCGYNPGTMCPPCRCDNTC

SEQ ID NO: 60 - protein, 30 aa, Conus aurisiacus
  PEPTIDE (1)..(30): Xaa at residues 2, 6, 17, 22 and 23 is Pro or hydroxy-Pro; Xaa at residue 3 is Trp (D or L) or bromo-Trp (D or L); Xaa at residue 15 is Tyr, 125I-Tyr, mono-iodo-Tyr, di-iodo-Tyr, O-sulpho-Tyr or O-phospho-Tyr
AXXLVXSTITTCCGXNXGTMCXXCRCDNTC

SEQ ID NO: 61 - DNA, 202 bp, Conus bandanus
  CDS (1)..(183)
atgcgctgtctcccagtcttgatcattcttctgctgctgactgcatctgcacctggcgttgatgtcctaccgaagaccgaagatgatgtgcccctgtcatctgtctacgataatacaaagagtatcctacgaggacttctggacaaacgtgcttgctgtggctacaagctttgctcaccatgttaaccagcatgaaggatcc

SEQ ID NO: 62 - protein, 61 aa, Conus bandanus
MRCLPVLIILLLLTASAPGVDVLPKTEDDVPLSSVYDNTKSILRGLLDKRACCGYKLCSPC

SEQ ID NO: 63 - protein, 11 aa, Conus bandanus
  PEPTIDE (1)..(11): Xaa at residue 10 is Pro or hydroxy-Pro; Xaa at residue 5 is Tyr, 125I-Tyr, mono-iodo-Tyr, di-iodo-Tyr, O-sulpho-Tyr or O-phospho- Tyr
ACCGXKLCSXC

SEQ ID NO: 64 - DNA, 359 bp, Conus caracteristicus
  CDS (7)..(246)
ggatccatgaaactgacgtgcgtggtgatcatcgccgcgctgttcctgacggcctgtcagctcaatacagctgatgactccagagataagcaggagtaccgtgcagtgaggttgagagacggaatgcggaatttcaaaggttccaagcgcaactgcggggaacaaggtgaaggttgtgctactcgcccatgctgctctggtctgagttgcgttggcagccgtccaggaggcctgtgccagtacggctaatagtctggcatctgatatttcccctctgcactctaccttcttttgcctgatgcatgtttacttgtgtgtggtcatgaaccactcagtagctacacctccgaaggacgtgc

SEQ ID NO: 65 - protein, 80 aa, Conus caracteristicus
MKLTCVVIIAALFLTACQLNTADDSRDKQEYRAVRLRDGMRNFKGSKRNCGEQGEGCATRPCCSGLSCVGSRPGGLCQYG

SEQ ID NO: 66 - protein, 31 aa, Conus caracteristicus
  PEPTIDE (1)..(31): Xaa at residues 4 and 7 is Glu or gamma-carboxy-Glu; Xaa at residues 13 and 25 is Pro or hydroxy-Pro; Xaa at residue 31 is Tyr, Tyr, 125I-Tyr, mono-iodo-Tyr, di-iodo-Tyr, O-sulpho-Tyr or O-phospho-Tyr
NCGXQGXGCATRXCCSGLSCVGSRXGGLCQX

SEQ ID NO: 67 - DNA, 349 bp, Conus caracteristicus
  CDS (1)..(270)
atgatgtcgaaaatgggagctatgtttgtccttttgcttcttttcatcctgccatccagccagcaggaaggagatgtccaggcaagaaaaacgcacctgaagagaggcttctacggtactctggcaatgtctaccagaggatgctctggcacttgccatcgtcgtgaggacggcaagtgtcggggtacttgcgactgctccggatacagctattgtcgctgcggtgacgctcaccatttttaccgaggatgcacgtgttcgtgtcaaggttgattaattgactcttttaactcgttgaacgattgaaaaaaaaaattttagagcaatatgttcgagaaaaaccgaagac

SEQ ID NO: 68 - protein, 90 aa, Conus caracteristicus
MMSKMGAMFVLLLLFILPSSQQEGDVQARKTHLKRGFYGTLAMSTRGCSGTCHRREDGKCRGTCDCSGYSYCRCGDAHHFYRGCTCSCQG

SEQ ID NO: 69 - protein, 43 aa, Conus caracteristicus
  PEPTIDE (1)..(43): Xaa at residue 10 is Glu or gamma-carboxy-Glu;  Xaa at residues 23, 25 and 35 is Tyr, 125I-Tyr, mono-iodo-Tyr, di-iodo-Tyr, O-sulpho-Tyr or O-phospho-Tyr
GCSGTCHRRXDGKCRGTCDCSGXSXCRCGDAHHFXRGCTCSCQ

SEQ ID NO: 70 - DNA, 346 bp, Conus caracteristicus
  CDS (1)..(264)
atgatgtcgaaaatgggagctatgtttgtccttttgcttcttttcatcctgccatccagccagcaggaaggagatgtccaggcaagaaaaacgcaccggaagagcggcttctacggtactctggcaatgtctgccagaggatgctctggcacttgccatcgtcgtgaggacggcaagtgtcggggtacttgcgactgctccggatacagctattgtcgctgcggtgacgctcaccatttttaccgaggatgcacgtgtacatgttaaggttgattaattgactcttttaactcgttgaaccgattaaaaaaaaaattagacgaatatgttcgagaaaaccgaagac

SEQ ID NO: 71 - protein, 88 aa, Conus caracteristicus
MMSKMGAMFVLLLLFILPSSQQEGDVQARKTHRKSGFYGTLAMSARGCSGTCHRREDGKCRGTCDCSGYSYCRCGDAHHFYRGCTCTC

SEQ ID NO: 72 - protein, 42 aa, Conus caracteristicus
  PEPTIDE (1)..(42): Xaa at residue 10 is Glu or gamma-carboxy-Glu; Xaa at residues 23, 25 and 35 is Tyr, 125I-Tyr, mono-iodo-Tyr, di-iodo-Tyr, O-sulpho-Tyr or O-phospho-Tyr
GCSGTCHRRXDGKCRGTCDCSGXSXCRCGDAHHFXRGCTCTC

SEQ ID NO: 73 - DNA, 348 bp, Conus caracteristicus
  CDS (1)..(264)
atgatgtcgaaaatgggagctatgtttgtccttttgcttcttttcatcctgccatccagccagcaggaaggagatgtccaggcaagaaaaacgcaccggaagagcggcttctacggtactctggcaatgtctaccagaggatgctctggcacttgccgtcgtcatcgggacggcaagtgtcggggtacttgcgactgctccggatacagctattgtcgctgcggtgacgctcaccatttttaccgaggatgcacgtgtacatgttaaggttgattaattcgatcttttaactcgttgaacgattaaaaaaaaaattttagacgaatatgttcgagaaaaaccgaagac

SEQ ID NO: 74 - protein, 88 aa, Conus caracteristicus
MMSKMGAMFVLLLLFILPSSQQEGDVQARKTHRKSGFYGTLAMSTRGCSGTCRRHRDGKCRGTCDCSGYSYCRCGDAHHFYRGCTCTC

SEQ ID NO: 75 - protein, 42 aa, Conus caracteristicus
  PEPTIDE (1)..(42): Xaa at residues 23, 25 and 35 is Tyr, 125I-Tyr, mono-iodo-Tyr, di-iodo-Tyr, O-sulpho-Tyr or O-phospho-Tyr
GCSGTCRRHRDGKCRGTCDCSGXSXCRCGDAHHFXRGCTCTC

SEQ ID NO: 76 - DNA, 349 bp, Conus caracteristicus
  CDS (1)..(264)
atgatgtcgaaaatgggagctatgtttgtccttttgcttcttttcatcctgccatccagccagcaggaaggagatgtccaggcaagaaaaacgcacctgaagagaggcttctacggtactctggcaatgtctaccagaggatgctctggcacttgccgtcgtcatcgggacggcaagtgtcggggtacttgcgactgctccggatacagctattgtcgctgcggtgacgctcaccatttttaccgaggatgcacgtgtacatgttaaggttgattaattgactcttttaactcgttgaacgattaaaaaaaaaaattttagagcaatatgttcgagaaaaaccgaagac

SEQ ID NO: 77 - protein, 88 aa, Conus caracteristicus
MMSKMGAMFVLLLLFILPSSQQEGDVQARKTHLKRGFYGTLAMSTRGCSGTCRRHRDGKCRGTCDCSGYSYCRCGDAHHFYRGCTCTC

SEQ ID NO: 78 - protein, 42 aa, Conus caracteristicus; identical to SEQ ID NO: 75
  PEPTIDE (1)..(42): Xaa at residues 23, 25 and 35 is Tyr, 125I-Tyr, mono-iodo-Tyr, di-iodo-Tyr, O-sulpho-Tyr or O-phospho-Tyr

SEQ ID NO: 79 - DNA, 345 bp, Conus caracteristicus
  CDS (1)..(264)
atgatgtcgaaaatgggagctatgtttgtccttttgtttcttttcaccctgccatccagccagcaggaaggagatgtccaggcaagaaaaacgcacctgaagagaggcttctacggtactctggcaatgtcttccagaggatgctctggcacttgccatcgtcgtgaggacggcaagtgtcggggtacttgcgactgctccggatacagctattgtcgctgcggtgacgctcaccatttttaccgaggatgtacgtgtacatgttaaggttgattaattgactcttttaactcgttgaacgattaaaaaaaaatttagagcaatatgttcgagaaaaccgaagac

SEQ ID NO: 80 - protein, 88 aa, Conus caracteristicus
MMSKMGAMFVLLFLFTLPSSQQEGDVQARKTHLKRGFYGTLAMSSRGCSGTCHRREDGKCRGTCDCSGYSYCRCGDAHHFYRGCTCTC

SEQ ID NO: 81 - protein, 42 aa, Conus caracteristicus; identical to SEQ ID NO: 72
  PEPTIDE (1)..(42): Xaa at residue 10 is Glu or gamma-carboxy-Glu; Xaa at residues 23, 25 and 35 is Tyr, 125I-Tyr, mono-iodo-Tyr, di-iodo-Tyr, O-sulpho-Tyr or O-phospho-Tyr

SEQ ID NO: 82 - DNA, 344 bp, Conus caracteristicus
  CDS (1)..(264)
atgatgtcgaaaatgggagctatgtttgtccttttgcttcttttcatcctgccatccagccagcaggaaggagatgtccaggcaagaaaaacgcacctgaagagcggcttctacggtactctggcaatgtctgccagaggatgctctggcacttgccatcgtcgtcaaaacggcgagtgtcagggtacttgcgactgcgacggacacgaccattgtgactgcggtgacactctcggtacttactcaggatgcgtgtgtatatgttaaggttgattaattgactcttttaactcgttgaacgattaaaaaaatttagagcaatatgttcgagaaaaaccgaagac

SEQ ID NO: 83 - protein, 88 aa, Conus caracteristicus
MMSKMGAMFVLLLLFILPSSQQEGDVQARKTHLKSGFYGTLAMSARGCSGTCHRRQNGECQGTCDCDGHDHCDCGDTLGTYSGCVCIC

SEQ ID NO: 84 - protein, 42 aa, Conus caracteristicus
  PEPTIDE (1)..(42): Xaa at residue 13 is Glu or gamma-carboxy-Glu; Xaa at residue 35 is Tyr, 125I-Tyr, mono-iodo-Tyr, di-iodo-Tyr, O-sulpho-Tyr or O-phospho-Tyr
GCSGTCHRRQNGXCQGTCDCDGHDHCDCGDTLGTXSGCVCIC

SEQ ID NO: 85 - DNA, 422 bp, Conus caracteristicus
  CDS (7)..(258)
gttacaatgcatctgtcactggcacgctcagctgtcttgatgttgcttctgctgtttgccttggacaacttcgttggggtccagccaggacagataacaagagatgtggacaaccgccgtaaccggcaatcgcgatggaagccaaggagtctcttcaagtcacttcataaacgagcatcgtgtggagggacttgcacggaaagtgccgattgcccttccacgtgtagtacttgcttacatgctcaatgcgagtcaacatgatgtcgcactacagctcttctctacagtgtgtacatcgaccgtacgacgcatcttttatttctttggctgtttcattcgttttcttgtgttcataacatgcggagcccttccgttacctctactgctctacacttaacctgataaccagaaaatccagtact

SEQ ID NO: 86 - protein, 84 aa, Conus caracteristicus
MHLSLARSAVLMLLLLFALDNFVGVQPGQITRDVDNRRNRQSRWKPRSLFKSLHKRASCGGTCTESADCPSTCSTCLHAQCEST

SEQ ID NO: 87 - protein, 28 aa, Conus caracteristicus
  PEPTIDE (1)..(28): Xaa at residues 9 and 26 is Glu or gamma-carboxy-Glu; Xaa at residue 14 is Pro or hydroxy-Pro
ASCGGTCTXSADCXSTCSTCLHAQCXST

SEQ ID NO: 88 - DNA, 426 bp, Conus caracteristicus
  CDS (7)..(258)
gttacaatgcatctgtcactggcacgctcagctgttttgatgttgcttctgctgtttgccttggacaacttcgttggggtccaaccaggacagataactagagatgtggacaaccgccgtaacctgcaatcgcgatggaagccaaggagtctcttcaagtcacttcataaacgagcatcgtgtggagggacttgcacggaaagtgccgattgcccttccacgtgtagtacttgcttacatgctcaatgcgagtgaacatgatgtcgcactacagctcttctctacagtgtgtacatcgaccgaccgtacgacgcatcttttatttctttgtctgtttcattcgttttcttgagttcataacatgcggagcccttccgttacctctactgctctacacttaagctgataaccagaaaatccagtact

SEQ ID NO: 89 - protein, 82 aa, Conus caracteristicus
MHLSLARSAVLMLLLLFALDNFVGVQPGQITRDVDNRRNLQSRWKPRSLFKSLHKRASCGGTCTESADCPSTCSTCLHAQCE

SEQ ID NO: 90 - protein, 25 aa, Conus caracteristicus
  PEPTIDE (1)..(25): Xaa at residues 8 and 25 is Glu or gamma-carboxy-Glu; Xaa at residue 13 is Pro or hydroxy-Pro
SCGGTCTXSADCXSTCSTCLHAQCX

SEQ ID NO: 91 - DNA, 220 bp, Conus circumcisus
  CDS (28)..(189)
tgtgtgtgtgtggttctgggtccagcatttgatggcaggaatgccgcagtcaacgagagagcgccttggacggtcgttttgtccaccacgaattgctgcggttataatacgatggaattctgccctgcttgcatgtgcacttattcctgtccaaaaaagaaaaaaccaggaaaaggccgcagaaacaactgatgctccaggaccctctgaaccacgacgt

SEQ ID NO: 92 - protein, 54 aa, Conus circumcisus
FDGRNAAVNERAPWTVVLSTTNCCGYNTMEFCPACMCTYSCPKKKKPGKGRRNN

SEQ ID NO: 93 - protein, 43 aa, Conus circumcisus
  PEPTIDE (1)..(43): Xaa at residue 19 is Glu or gamma-carboxy-Glu; Xaa at residues 2, 22, 31 and 36 is Pro or hydroxy-Pro; Xaa at residue 3 is Trp (D or L) or bromo-Trp (D or L); Xaa at residues 15 and 28 is Tyr, 125 I-Tyr, mono-iodo-Tyr, di-iodo-Tyr, O-sulpho-Tyr or O-phospho-Tyr
AXXTVVLSTTNCCGXNTMXFCXACMCTXSCXKKKKXGKGRRNN

SEQ ID NO: 94 - protein, 64 aa, Conus consors
GIFVGVQPEQITRDVDKGYSTDDGHDLLSLLKQISLRACTGSCNSDSECYNFCDCIGTRCEAQK

SEQ ID NO: 95 - protein, 27 aa, Conus consors
  PEPTIDE (1)..(27): Xaa at residues 11 and 24 is Glu or gamma-carboxy-Glu; Xaa at residue 13 is Tyr, 125I-Tyr, mono-iodo-Tyr, di-iodo-Tyr, O-sulph0-Tyr or O-phospho-Tyr
ACTGSCNSDSXCXNFCDCIGTRCXAQK

SEQ ID NO: 96 - protein, 27 aa, Conus delesstii
  PEPTIDE (1)..(27): Xaa at residues 13 and 16 is Glu or gamma-carboxy-Glu; Xaa at residue 4 is Pro or hydroxy-Pro; Xaa at residue 25 is Tyr, 125I-Tyr, mono-iodo-Tyr, di-iodo-Tyr, O-sulpho-Tyr or O-phospho-Tyr
ACKXKNNLCAITXMAXCCSGFCLIXRC

SEQ ID NO: 97 - DNA, 375 bp, Conus distans
  CDS (24)..(260)
gacaggattgaacaaaattcaggatgtcaggattgggaatcatggtgctaacccttctacttcttgtgcccatggcaaccagtcaacaggatggaggagagaagcaggcgatgcaaagggacgcaatcaacgtcgcaccaggaacatcaatcactcggagaaatgtagatcaggagtgcattgacgcctgtcagctggaggacaagaattgctgtggcagaacagatggagaacccagatgtgcgaaaatctgcctcggataatttctgtacgctgtctcattcattatttcatccgtacgagtgtaaacgagacctattagaaagtcgaaggttgtgcgtaatttgataagcattgtttgctgggacgaacgga

SEQ ID NO: 98 - protein, 79 aa, Conus distans
MSGLGIMVLTLLLLVPMATSQQDGGEKQAMQRDAINVAPGTSITRRNVDQECIDACQLEDKNCCGRTDGEPRCAKICLG

SEQ ID NO: 99 - protein, 32 aa, Conus distans
  PEPTIDE (1)..(32): Xaa at residues 5, 13 and 24 is Glu or gamma-carboxy-Glu; Xaa at residue 25 is Pro or hydroxy-Pro
NVDQXCIDACQLXDKNCCGRTDGXXRCAKICL

SEQ ID NO: 100 - DNA, 376 bp, Conus distans
  CDS (24)..(260)
gacaggattgaacaaaattcaggatgtcaggattgggaatcatggtgctaacccttctacttcttgtgcccatggcaaccagtcaacaggatggaggagagaagcaggcgatgcaaagggacgcaatcaacgtcgcaccaggaacatcaatcactcggacagaaacagatcaggagtgcattgacatctgtaagcaggaggacaagaaatgctgcggcagatcaaatggagaacccacatgtgcgaaaatctgcctcggataatttctgtacgctgtctcgttcattatttcgtcagtacgagtttaaacgagacctattagaaagtcgaaggttcgtgcttaatttgataagcattgtttgctgggatgaacgga

SEQ ID NO: 101 - protein, 79 aa, Conus distans
MSGLGIMVLTLLLLVPMATSQQDGGEKQAMQRDAINVAPGTSITRTETDQECIDICKQEDKKCCGRSNGEPTCAKICLG

SEQ ID NO: 102 - protein, 32 aa, Conus distans
  PEPTIDE (1)..(32): Xaa at residues 1, 5, 13 and 24 is Glu or gamma-carboxy-Glu; Xaa at residue 25 is Pro or hydroxy-Pro
XTDQXCIDICKQXDKKCCGRSNGXXTCAKICL

SEQ ID NO: 103 - DNA, 376 bp, Conus distans
  CDS (24)..(260)
gacaggattgaacaaaattcaggatgtcaggattgggaatcatggtgctaacccttctacttcttgtgcccatggcaaccagtcaacaggatggaggagagaagcaggcgatgcaaagggacgcaatcaacgtcgcaccaggaacatcaatcactcggagagaaacagatcaggagtgcattgacacctgtgagcaggaggacaagaaatgctgcggcagaacaaatggagaacccgtatgtgcgaaaatctgcttcggataatttctgtacgctgtctcattcataatttcatcagtacgagtttaaacgagacctattagaaagtcgaaggttcgtgcttaatttgataagcattgtttgctgggatgaacgga

SEQ ID NO: 104 - protein, 79 aa, Conus distans
MSGLGIMVLTLLLLVPMATSQQDGGEKQAMQRDAINVAPGTSITRRETDQECIDTCEQEDKKCCGRTNGEPVCAKICFG

SEQ ID NO: 105 - protein, 32 aa, Conus distans
  PEPTIDE (1)..(32): Xaa at residues 1, 5, 11, 13 and 24 is Glu or gamma-carboxy-Glu; Xaa at residue 25 is Pro or hydroxy-Pro
XTDQXCIDTCXQXDKKCCGRTNGXXVCAKICF

SEQ ID NO: 106 - DNA, 250 bp, Conus ermineus
  CDS (1)..(219)
atgttcaccgtgtttctgttggttgtcttggcaaccactgtcgtttccttcacttcagatcgtgcatcggatgacaggaataccaacgacaaagcatctcgcctgctctctcacgttgtcaggggatgctgtggtaagtatcccaatgctgcctgtcatccttgcggttgtacagtgggtaggccaccgtattgtgacagacccagtggtggaggacgctgatgctccaggaccctctgaaccacgacgt

SEQ ID NO: 107 - protein, 73 aa, Conus ermineus
MFTVFLLVVLATTVVSFTSDRASDDRNTNDKASRLLSHVVRGCCGKYPNAACHPCGCTVGRPPYCDRPSGGGR

SEQ ID NO: 108 - protein, 30 aa, Conus ermineus
  PEPTIDE (1)..(30): Xaa at residue7, 13, 21, 22 and 27 is Pro or hydroxy-Pro; Xaa at residues 6 and 23 is Tyr, 125I-Tyr, mono-iodo-Tyr, di-iodo-Tyr, O-sulpho-Tyr or O-phospho-Tyr
GCCGKXXNAACHXCGCTVGRXXXCDRXSGG

SEQ ID NO: 109 - DNA, 241 bp, Conus generalis
  CDS (1)..(210)
ggatccatgatgtctaaactgggagtcttgttgaccatctgtctggttctgtttccccttactgctcttccactggatggagaacaacctgtagaccgacatgccgagcatatgcaggatgacaattcagctgcacagaacccctgggttattgccatcagacagtgttgcacgttctgcaactttggatgccagccttgttgcgtcccctgataacgtgttgatgaccaactttctcgag

SEQ ID NO: 110 - protein, 70 aa, Conus generalis
GSMMSKLGVLLTICLVLFPLTALPLDGEQPVDRHAEHMQDDNSAAQNPWVIAIRQCCTFCNFGCQPCCVP

SEQ ID NO: 111 - protein, 16 aa, Conus generalis
  PEPTIDE (1)..(16): Xaa at residue 1 is Gln or pyro-Gln; Xaa at residues 12 and 16 is Pro or hydroxy-Pro
XCCTFCNFGCQXCCVX

SEQ ID NO: 112 - DNA, 404 bp, Conus geographus
  CDS (18)..(242)
gcaagatcatcagcagaatgaacctgacgtgcgtgttgatcatcgccgtgctgtttctgacggcctgccagctcattgcagctgatgactccagagataaccagaagcaccgtgcagtgaggatgagagacgcattgaagaatttcaaagattccagggcgtgctccggtagaggttctagatgtcctccccaatgctgcatgggtttgacgtgcggtcgtgagtatccacccagatgcggttgatatacggtgaacaactgatatttcccctctgtgctctaccctcttttgcctgattcacccacacctatgtgtggtcatgaaccactcagtacctacacctctggtggcttcagaggacgtatattaaaataaaaccacattgcaatgaaaaaaaaaaaa

SEQ ID NO: 113 - protein, 75 aa, Conus geographus
MNLTCVLIIAVLFLTACQLIAADDSRDNQKHRAVRMRDALKNFKDSRACSGRGSRCPPQCCMGLTCGREYPPRCG

SEQ ID NO: 114 - protein, 27 aa, Conus geographus
  PEPTIDE (1)..(27): Xaa at residue 22 is Glu or gamma-carboxy-Glu; Xaa at residues 10, 11, 24 and 25 is Pro or hydroxy-Pro; Xaa at residue 23 is Tyr, 125I-Tyr, mono-iodo-Tyr, di-iodo-Tyr, O-sulpho-Tyr or O-phospho-Tyr
ACSGRGSRCXXQCCMGLTCGRXXXXRC

SEQ ID NO: 115 - protein, 9 aa, Conus geographus
  PEPTIDE (1)..(9): Xaa at residue 7 is Pro or hydroxy-Pro
CFIRNCXKG

SEQ ID NO: 116 - DNA, 360 bp, Conus geographus
  CDS (1)..(213)
tgctgcccgagtagcaaagaggattccctgaactgcattgagaccatggcgaccacggccacgtgcatgaagtccaacaagggggagatctactcctatgcgtgcggctactgcggcaagaagaaggagagctgtttcggcgacaaaaagccagtgactgactaccagtgccagacgcggaacattcccaacccctgcggcggcgctgctctctgaaggcaccaacagcaccaacagcacgatctcctgtgtttcgtcactgcatttatgacgtcaaaaccacgtcatgcatgatgacgacgatctcggctatggcatgtattgaagaatggaaataaacctagttttcagctgaaaaaa

SEQ ID NO: 117 - protein, 71 aa, Conus geographus
CCPSSKEDSLNCIETMATTATCMKSNKGEIYSYACGYCGKKKESCFGDKKPVTDYQCQTRNIPNPCGGAAL

SEQ ID NO: 118 - protein, 71 aa, Conus geographus
  PEPTIDE (1)..(71): Xaa at residues 7, 14, 29 and 43 is Glu or gamma-carboxy-Glu; Xaa at residues 3, 51, 63 and 65 is Pro or hydroxy-Pro; Xaa at residues 31, 33, 37 and 55 is Tyr, 125I-Tyr, mono-iodo-Tyr, di-iodo-Tyr, O-sulpho-Tyr or O-phospho-Tyr
CCXSSKXDSLNCIXTMATTATCMKSNKGXIXSXACGXCGKKKXSCFGDKKXVTDXQCQTRNIXNXCGGAAL

SEQ ID NO: 119 - DNA, 769 bp, Conus geographus
  misc_feature (1)..(769): n is unknown
cgggcgctgcattccggacgtgaaacagcatcgccagcaagtgggcatagtgcaagacactcagaacaatgacgcacatagtctganaaaataaccatgggtatgcggatgangtttagtgtgtttcngcaggttgtcntgggnaccactgtcgtttccttcacntcacgtcgtggtccaaaatctcgtcgcggggaacctattccgaccactgtaatcaactacggggagtgctgtaaggatccatcctgttgggttaaggtgaaggatttccagtgtcctggagcaagtcctcccaactgaaccacgacatgtcgccctctgcctgacctgcttcacgttccgtctctttctgccactagaactcaacaactcgatccaacagactcctactttacctccgtattctgaaactacttggatttgattgtctttaatatctactcacacttgctgttattacatcatccaaaatttaacaagaacatgaaaggtgtctgttcaaacaaaatcaggcaatgacaangggggaaagtctccantctatctgaaaactgtcacctgtcactctcttaaccaggtttanaactgantaccactanagctgttgtnccacatcangatcagnccaatttgtanngtttcctttgcaaaacttttgcctgaaattcttgaaaagaaacgctcacaatgttgggaagtgcttttnattanctgacaanntgncancatgttccntttcantaantctnaaatgnaaacctctgtt

SEQ ID NO: 120 - protein, 68 aa, Conus geographus
MGMRMMFSVFLQVVLGTTVVSFTSRRGPKSRRGEPIPTTVINYGECCKDPSCWVKVKDFQCPGASPPN

SEQ ID NO: 121 - protein, 36 aa, Conus geographus
  PEPTIDE (1)..(36): Xaa at residues 2 and 13 is Glu or gamma-carboxy-Glu; Xaa at residues 3, 5, 18, 30, 35 adn 35 is Pro or hydroxy-Pro; Xaa at residue 21 is Trp (D or L) or bromo-Trp (D or L);
GXXIXTTVINXGXCCKDXSCXVKVKDFQCXGASXXN

SEQ ID NO: 122 - DNA, 519 bp, Conus geographus
  CDS (113)..(391)
agccttgatacagagctggtatctgctgttaatacttgaaagaacaagtgctgtgagccttcatctctctctgactttagtttgggtcctggagaaaaccttgacgggcagtatgaaaatttacctgtgtcttgcttttgttctgctcctggcttctaccatagttgattcagggcttcttgataaaattgagactataagaaactggaaacgcgatgacagctattgtgatggatgcctatgcaccatattaaaaaaagagacttgcacatcgactatgagctgcaggggaacatgccgaaaagagtggccatgttgggaagaagactgctactgtactgaaatccaaggtggagcttgcgtcacaccctcagaatgcaaacctggagagtgttgaggattggagtggccagttccagcacatacagcaccatggtgccctggacaatcgtctattgaattgaatatgcctgtggcaggaatctgtcctacaaaataaaaaaatcataagttaaaaaa

SEQ ID NO: 123 - protein, 93 aa, Conus geographus
MKIYLCLAFVLLLASTIVDSGLLDKIETIRNWKRDDSYCDGCLCTILKKETCTSTMSCRGTCRKEWPCWEEDCYCTEIQGGACVTPSECKPGE

SEQ ID NO: 124 - protein, 60 aa, Conus geographus
  PEPTIDE (1)..(60): Xaa at residues 16, 36, 37, 43, 56 and 59 is Glu or gamma- carboxy-Glu; Xaa at residues 33, 52 and 57 is Pro or hydroxy-Pro; Xaa at residues 32 and 35 is Trp (D or L) or bromo-Trp (D or L);
DDSXCDGCLCTILKKXTCTSTMSCRGTCRKXXXCXXXDCXCTXIQGGACVTXSXCKXGXC

SEQ ID NO: 125 - DNA, 409 bp, Conus geographus
  CDS (17)..(313)
aacgttgacgggcagtatgaacatttacctgtgtcttgcttttcttctgttcctgccttctaccatagttgattcagggcttcttgataaaattgagacaataaggaattggagacgtgatgaaagcaagtgtgatcgatgcaattgcgccgaattaagatcatccagatgcacacaagctatcttctgccttacaccggagttatgcacaccgagcatctcatgtccgacaggtgaatgccgctgtactaagttccatcagtcaagatgcactagattcgtagaatgcgtacctaataagtgtagagacgcatagaggccagttccagcacatacagcaccatgatgccctggacaatcgtgttgttggattgaatatgcccgtggcaggaatctgtcctacaaaaaa

SEQ ID NO: 126 - protein, 99 aa, Conus geographus
MNIYLCLAFLLFLPSTIVDSGLLDKIETIRNWRRDESKCDRCNCAELRSSRCTQAIFCLTPELCTPSISCPTGECRCTKFHQSRCTRFVECVPNKCRDA

SEQ ID NO: 127 - protein, 65 aa, Conus geographus
  PEPTIDE (1)..(65): Xaa at residues 2, 12, 28, 40 and 56 is Glu or gamma-carboxy-Glu; Xaa at residues 27, 32, 37 and 59 is Pro or hydroxy-Pro
DXSKCDRCNCAXLRSSRCTQAIFCLTXXLCTXSISCXTGXCRCTKFHQSRCTRFVXCVXNKCRDA

SEQ ID NO: 128 - protein, 29 aa, Conus geographus
  PEPTIDE (1)..(29): Xaa at residues 9 and 23 is Glu or gamma-carboxy-Glu; Xaa at residue 28 is Pro or hydroxy-Pro; Xaa at residue 15 is Tyr, 125I-Tyr, mono-iodo-Tyr, di-iodo-Tyr, O-sulpho-Tyr or O-phospho-Tyr
KFLSGGFKXIVCHRXCAKGIAKXFCNCXD

SEQ ID NO: 129 - protein, 7 aa, Conus imperialis
  PEPTIDE (1)..(7): Xaa at residue 1 is Gln or pyro-Gln; Xaa at residue 6 is Trp (D or L) or bromo-Trp (D or L)
XCGQAXC

SEQ ID NO: 130 - DNA, 524 bp, Conus imperialis
  CDS (7)..(285)
gttaaaatgcatctgtcactggcaagctcagctgctttgatgttgcttctgctttttgccttgggcaacttcgttggggtccagccaggacaaataagagatctgaacaaaggacagctcaaggacaaccgccgtaacctgcaatcgcagaggaaacaaatgagtctcctcaagtcacttcatgatcgaaatgggtgtaacggcaacacgtgttccaatagcccctgccctaacaactgttattgcgatactgaggacgactgccaccctgacaggcgtgaacattagagattagagagtttccttgtcaacatgatgtcgcaccacacctctgctctgcagtgtgtacatcgaccagtcgacgcatctgttatttctttgtctgttggattgtacatcgaccagtccacgcatctgttatttctttgtctgtttgatttgttttcgtgtgttcataacacacagagcctttctattatctgtattgcaatacactttgcctgataaccagaaagtccagtgct

SEQ ID NO: 131 - protein, 93 aa, Conus imperialis
MHLSLASSAALMLLLLFALGNFVGVQPGQIRDLNKGQLKDNRRNLQSQRKQMSLLKSLHDRNGCNGNTCSNSPCPNNCYCDTEDDCHPDRREH

SEQ ID NO: 132 - protein, 32 aa, Conus imperialis
  PEPTIDE (1)..(32): Xaa at residues 22 and 31 is Glu or gamma-carboxy-Glu; Xaa at residues 13, 14 and 27 is Pro or hydroxy-Pro; Xaa at residue 18 is Tyr, 125I-Tyr, mono-iodo-Tyr, di-iodo-Tyr, O-sulpho-Tyr or O-phospho-Tyr
NGCNGNTCSNSXCXNNCXCDTXDDCHXDRRXH

SEQ ID NO: 133 - DNA, 350 bp, Conus lacterculatus
  CDS (1)..(273)
atgatgtcgaaaatgggagctatgtttgtccttttgcttcttttcaccctggcatccagccagcaggaaggagatgtccaggcaaggaaaacacacccgaagagagagttccatcgtattctgctaaggcctgacagacagtccgaaacggcttgtaggtcgctcggaagctaccaatgtatgggtaaatgccaactcggggttcattcctggtgtgaatgcatttataaccgaggtagtcagaagtctggatgcgcgtgtaggtgtcaaaagtgattaattgactcatttaactcgttgaacgatttaaaaaatccagagcaatatgttcgagaaaaaccgaagacgac

SEQ ID NO: 134 - protein, 91 aa, Conus lacterculatus
MMSKMGAMFVLLLLFTLASSQQEGDVQARKTHPKREFHRILLRPDRQSETACRSLGSYQCMGKCQLGVHSWCECIYNRGSQKSGCACRCQK

SEQ ID NO: 135 - protein, 45 aa, Conus lacterculatus
  PEPTIDE (1)..(45): Xaa at residue 1 is Gln or pyro-Gln; Xaa at residues 3 and 27 is Glu or gamma-carboxy-Glu; Xaa at residue 25 is Trp (D or L) or bromo-Trp (D or L); Xaa at residues 12 and 30 is Tyr, 125I-Tyr, mono-iodo-Tyr, di-iodo-Tyr, O-sulpho-Tyr or O-phospho-Tyr
XSXTACRSLGSXQCMGKCQLGVHSXCXCIXNRGSQKSGCACRCQK

SEQ ID NO: 136 - DNA, 318 bp, Conus lividus
  CDS (7)..(237)
ggatccatgaaactgacgtgtgtggtgatcatcgccgtgctgttcctgacggccagtcagctcattacagctgattactccagagataagcaggagtatcgtgcagagaggctgagagacgcaatggggaaattcaaaggttccaggtcgtgcggacatagtggtgcaggttgttatactcgcccttgctgccctggtctgcattgctctggcggccaagctggaggcctgtgcgtgtaatagtaataatctggcgtctgatatttccagtctgtgctctaccctcttttgcctgagtcatccatacctgtgctcgag

SEQ ID NO: 137 - protein, 77 aa, Conus lividus
MKLTCVVIIAVLFLTASQLITADYSRDKQEYRAERLRDAMGKFKGSRSCGHSGAGCYTRPCCPGLHCSGGQAGGLCV

SEQ ID NO: 138 - protein, 30 aa, Conus lividus
  PEPTIDE (1)..(30): Xaa at residues 13 and 16 is Pro or hydroxy-Pro; Xaa at residue 10 is Tyr, 125I-Tyr, mono-iodo-Tyr, di-iodo-Tyr, O-sulpho-Tyr or O-phospho-Ty
SCGHSGAGCXTRXCCXGLHCSGGQAGGLCV

SEQ ID NO: 139 - DNA, 312 bp, Conus lividus
  CDS (7)..(252)
ggatccatgaaactgacgtgtgtggtgatcatatccgtgctgttcctgacggccagtgagttccttacagctgattactccagagataagcggcagtaccgtgctgtgaggttgagagacgcaatgcggaatttcaaaggtaccagggactgcggggaatcaggtcaaggttgctatagtgtacgtccttgctgccctggtctgatttgcaaaggcaccggtggtggaggcctgtgccggccctctggcatctgatatctcccctctgtgctccaccctcttttgcctgagtcatccatacctgtgctcgag

SEQ ID NO: 140 - protein, 82 aa, Conus lividus
MKLTCVVIISVLFLTASEFLTADYSRDKRQYRAVRLRDAMRNFKGTRDCGESGQGCYSVRPCCPGLICKGTGGGGLCRPSGI

SEQ ID NO: 141 - protein, 35 aa, Conus lividus
  PEPTIDE (1)..(35): Xaa at residue 4 is Glu or gamma-carboxy-Glu; Xaa at residues 14, 17 and 32 is Pro or hydroxy-Pro; Xaa at residue 10 is Tyr, 125I- Tyr, mono-iodo-Tyr, di-iodo-Tyr, O-sulpho-Tyr or O-phospho-Tyr
DCGXSGQGCXSVRXCCXGLICKGTGGGGLCRXSGI

SEQ ID NO: 142 - protein, 6 aa, Conus magus
  PEPTIDE (1)..(6): Xaa at residue 3 is Glu or gamma-carboxy-Glu; Xaa at residue 6 is Pro or hydroxy-Pro; Xaa at residue 2 is Tyr, 125I-Tyr, mono- iodo-Tyr, di-iodo-Tyr, O-sulpho-Tyr or O-phospho-Tyr
VXXTHX

SEQ ID NO: 143 - protein, 6 aa, Conus magus
  PEPTIDE (1)..(6): Xaa at residue 3 is Glu or gamma-carboxy-Glu; Xaa at residue 6 is Pro or hydroxy-Pro; Xaa at residue 2 is Tyr, 125I-Tyr, mono- iodo-Tyr, di-iodo-Tyr, O-sulpho-Tyr or O-phospho-Tyr
RXKNSX

SEQ ID NO: 144 - protein, 7 aa, Conus magus
  PEPTIDE (1)..(7): Xaa at residue 2 is Pro or hydroxy-Pro; Xaa at residue 6 is Trp (D or L) or bromo-Trp (D or L)
ARXKNSX

SEQ ID NO: 145 - protein, 6 aa, Conus magus
  PEPTIDE (1)..(6): Xaa at residue 3 is Pro or hydroxy-Pro; Xaa at residue 7 is Trp (D or L) or bromo-Trp (D or L)
RXKNSX

SEQ ID NO: 146 - DNA, 360 bp, Conus miles
  CDS (7)..(270)
ggatccatgaaactgacgtgcgtggtgatcgtcgccgtgctgttcctgacggcctgtcaactcattactgctgcgaattacgccagagatgaacaggagtaccccgctgtgaggtcgagcgacgtgatgcaggattccgaagacttgacgttgaccaagaaatgcacggacgattctcagttctgtaacccttcgaatcatgactgctgcagtgggaagtgtatcgacgaaggagacaacggcatatgcgctatagtccctgaaaactcttaacaatgtatactgacatttccccctctgtgctccgccgtccgtggcctgactcgtccatccttgggcgtggtcatgaaccgctcggtt

SEQ ID NO: 147 - protein, 88 aa, Conus miles
MKLTCVVIVAVLFLTACQLITAANYARDEQEYPAVRSSDVMQDSEDLTLTKKCTDDSQFCNPSNHDCCSGKCIDEGDNGICAIVPENS

SEQ ID NO: 148 - protein, 36 aa, Conus miles
  PEPTIDE (1)..(36): Xaa at residues 23 and 34 is Glu or gamma-carboxy-Glu; Xaa at residues 10 and 33 is Pro or hydroxy-Pro
CTDDSQFCNXSNHDCCSGKCIDXGDNGICAIVXXNS

SEQ ID NO: 149 - DNA, 357 bp, Conus miles
  CDS (7)..(270)
ggatccatgaaactgacgtgtgtggtgatcgtcgccgtgctgttcctgacggcctgtcaactcattactgctgcgaattacgccagagatgaacaggagtaccctgctgtgaggtcgagcgacgtgatgcaggattccgaagacctgacgttgaccaagaaatgcacggaggattctcagttctgtaacccttcgaatcatgactgctgcagtgggaagtgtatcgacgaaggagacaacggcatatgcgctatagtccctgaaaactcttaacaatgtatactgacatttccccctctgtgctccgccgtccgtggcctgactcgtccatccttgggcgtggtcatgaaccgctcg

SEQ ID NO: 150 - protein, 88 aa, Conus miles
MKLTCVVIVAVLFLTACQLITAANYARDEQEYPAVRSSDVMQDSEDLTLTKKCTEDSQFCNPSNHDCCSGKCIDEGDNGICAIVPENS

SEQ ID NO: 151 - protein, 36 aa, Conus miles
  PEPTIDE (1)..(36): Xaa at residues 3, 23 and 34 is Glu or gamma-carboxy-Glu; Xaa at residues 10 and 33 is Pro or hydroxy-Pro
CTXDSQFCNXSNHDCCSGKCIDXGDNGICAIVXXNS

SEQ ID NO: 152 - DNA, 327 bp, Conus miliaris
  CDS (12)..(239)
ggatccatgaactgacgtgtgtggtgatcatcgccgtgctgttcctgacggcctgtcaactcactacagctgtgacttcctccagaggtcaacagaagcatcgtgctctgaggtcaactgacaaaaactccaggatgaccaagcgttgcacgcctccaggtggactctgttaccatgcttatccctgctgcagcaagacttgcaatctcgataccagccaatgtgagcctaggtggtcatgaaccactcaataccctctcctctggaggcttcagaggaactacattgaaataaaaccgcattgcaacgaaaaaaaaaaaaaaaaaa

SEQ ID NO: 153 - protein, 76 aa, Conus miliaris
LTCVVIIAVLFLTACQLTTAVTSSRGQQKHRALRSTDKNSRMTKRCTPPGGLCYHAYPCCSKTCNLDTSQCEPRWS

SEQ ID NO: 154 - protein, 31 aa, Conus miliaris
  PEPTIDE (1)..(31): Xaa at residue 27 is Glu or gamma-carboxy-Glu; Xaa at residues 3, 4, 13 and 28 is Pro or hydroxy-Pro; Xaa at residue 30 is Trp (D or L) or bromo-Trp (D or L); Xaa at residues 9 and 12 is Tyr, 125 I-Tyr, mono-iodo-Tyr, di-iodo-Tyr, O-sulpho-Tyr or O-phospho-Tyr
CTXXGGLCXHAXXCCSKTCNLDTSQCXXRXS

SEQ ID NO: 155 - DNA, 193 bp, Conus monachus
  CDS (28)..(162)
tgtgtgtgtgtggttctgggtccagcatctgatgtcaggaatgccgcagtccacgaaagacagaaggatctggtcgttacggccaccacgacttgctgtggttataatccgatgacaatgtgccctccttgcatgtgcactaatacctgcaaaaaaagtggctgatgctccaggaccctctgaaccacgacgt

SEQ ID NO: 156 - protein, 45 aa, Conus monachus
SDVRNAAVHERQKDLVVTATTTCCGYNPMTMCPPCMCTNTCKKSG

SEQ ID NO: 157 - protein, 33 aa, Conus monachus
  PEPTIDE (1)..(33): Xaa at residue 1 is Gln or pyro-Gln; Xaa at residues 17, 22 and 23 is Pro or hydroxy-Pro; Xaa at residue 15 is Tyr, 125I-Tyr, mono-iodo-Tyr, di-iodo-Tyr, O-sulpho-Tyr or O-phospho-Ty
XKDLVVTATTTCCGXNXMTMCXXCMCTNTCKKS

SEQ ID NO: 158 - DNA, 350 bp, Conus monachus
  CDS (1)..(282)
atgatgtcgaaaatgggagctatgtttgtccttttgcttcttttcaccctggcatccagccagcaggaaggagatgtccaggcaaggaaaacaagcctgaagagcgacttctatcgtgctctgagagggtatgacagacagtgcactcttgtcaacaattgtgaccggaacggtgagcgtgcgtgtaacggtgattgctcttgcgagggccagatttgtaaatgcggttatagagtcagtcctgggaagtcaggatgcgcgtgtacttgtagaaatgccaaatgaatcatttaactcgttgaaagattttttaaaaatccagagctatatgttcgagaaaaaccgaagac

SEQ ID NO: 159 - protein, 94 aa, Conus monachus
MMSKMGAMFVLLLLFTLASSQQEGDVQARKTSLKSDFYRALRGYDRQCTLVNNCDRNGERACNGDCSCEGQICKCGYRVSPGKSGCACTCRNAK

SEQ ID NO: 160 - protein, 48 aa, Conus monachus
  PEPTIDE (1)..(48): Xaa at residue 1 is Gln or pyro-Gln; Xaa at residues 13 and 23 is Glu or gamma-carboxy-Glu; Xaa at residue 35 is Pro or hydroxy- Pro; Xaa at residue 31 is Tyr, 125I-Tyr, mono-iodo-Tyr, di- iodo-Tyr, O-sulpho-Tyr or O-phospho-Tyr
XCTLVNNCDRNGXRACNGDCSCXGQICKCGXRVSXGKSGCACTCRNAK

SEQ ID NO: 161 - DNA, 211 bp, Conus pennaceus
  CDS (1)..(192)
atgcgctgtctcccagtcttcgtcattcttctgctgctgactgcatctgcacctagcgttgatgccaaagttcatctgaagaccaaaggtgatgggcccctgtcatctttccgagataatgcaaagagtaccctacaaagacttcaggacaaaagcacttgctgtggctttaagatgtgtattccttgtcgttaaccagcatgaaggatcc

SEQ ID NO: 162 - protein, 64 aa, Conus pennaceus
MRCLPVFVILLLLTASAPSVDAKVHLKTKGDGPLSSFRDNAKSTLQRLQDKSTCCGFKMCIPCR

SEQ ID NO: 163 - protein, 13 aa, Conus pennaceus
  PEPTIDE (1)..(13): Xaa at residue 11 is Pro or hydroxy-Pro
STCCGFKMCIXCR

SEQ ID NO: 164 - DNA, 450 bp, Conus pennaceus
  CDS (1)..(234)
atgttgcttctgctgtttgccttgggcagcttcgttgtggtccagtcaggacagataacaagagatgtggacaatgggcagctcgcggacaaccgccgtaccctgcgatcgcagtggaagcaagtgagtttcttcaagtcacttgataaacgactgacttgtaacgatccttgccagatgcattccgattgcggcatatgtgaatgcgtggaaaataaatgcatatttttcatgtaaacggattgagtttgcttgtcaacacaatgtcgcactgcagctcttctctaccggtgggtacatcgaccaaacgacgcatcttttatttctttgtctgtttcgtttgttctcctgtgttcataacgtacagagccctttaactacccttactgctcttcacttaacctgataacctgaaggtccggtgcagctggcgtagccttcacagtttcg

SEQ ID NO: 165 - protein, 78 aa, Conus pennaceus
MLLLLFALGSFVVVQSGQITRDVDNGQLADNRRTLRSQWKQVSFFKSLDKRLTCNDPCQMHSDCGICECVENKCIFFM

SEQ ID NO: 166 - protein, 27 aa, Conus pennaceus
  PEPTIDE (1)..(27): Xaa at residues 17 and 20 is Glu or gamma-carboxy-Glu; Xaa at residue 6 is Pro or hydroxy-Pro
LTCNDXCQMHSDCGICXCVXNKCIFFM

SEQ ID NO: 167 - DNA, 413 bp, Conus pulicarius
  CDS (1)..(243)
atgaaactgacgtgtgtggtgatcgtcgccgtgctgttcctgacggcctgtcaactcagtacagctgatgactccagagatgagcagcaggatcctttggtgaggtcgcatcgtgaggagcagaaagccgaggaccccaagacggccgagagatgttcagatttcggatccgactgtgttcctgctactcataactgctgcagtggtgaatgttttggcttcgaggacttcggcttatgcacgtaaaactggtctgacgtctgatattcccccctctgtccttcatcctcttttgcctgattcatccatacctatatgtgctcctgaaccgctgtgtacctttaccctggtggcttcagaggacgttatatcaaaataaaaccgcgttgcaatgacaaaaaaaaaaaaaaaaa

SEQ ID NO: 168 - protein, 81 aa, Conus pulicarius
MKLTCVVIVAVLFLTACQLSTADDSRDEQQDPLVRSHREEQKAEDPKTAERCSDFGSDCVPATHNCCSGECFGFEDFGLCT

SEQ ID NO: 169 - protein, 30 aa, Conus pulicarius
  PEPTIDE (1)..(30): Xaa at residues 19 and 24 is Glu or gamma-carboxy-Glu; Xaa at residue 10 is Pro or hydroxy-Pro
CSDFGSDCVXATHNCCSGXCFGFXDFGLCT

SEQ ID NO: 170 - DNA, 375 bp, Conus purpurascens
  CDS (24)..(260)
gacaggattgaacaaaattcaggatgtcaagatttggaatcatggtgctaacctttctacttcttgtgtccatggcaaccagccatcgttatgcaagagggaagcaggcgacgcgaaggaacgcaatcaacatcagacggagaagcacaccaaaaactgaggcgtgcgaagaggtctgtgagctggaagaaaagcactgctgctgcataagaagtgacggacccaaatgttcccgtaagtgcctgttgtcaatcttctgttagtttctgtacactgtctcattcattatcttatcagtacaagtgtaaacgagacatgtcagaaagtcgaaggttgtgcgtaatttgataagtattgtttgctgggatgaacgga

SEQ ID NO: 171 - protein, 79 aa, Conus purpurascens
MSRFGIMVLTFLLLVSMATSHRYARGKQATRRNAINIRRRSTPKTEACEEVCELEEKHCCCIRSDGPKCSRKCLLSIFC

SEQ ID NO: 172 - protein, 37 aa, Conus purpurascens
  PEPTIDE (1)..(37): Xaa at residues 4, 7, 8, 11, 13 and 14 is Glu or gamma-carboxy- Glu; Xaa at residues 3 and 25 is Pro or hydroxy-Pro
XKTXACXXVCXLXXKHCCCIRSDGXKCSRKCLLSIFC

SEQ ID NO: 173 - DNA, 373 bp, Conus purpurascens
  CDS (24)..(260)
gacaggattgaacaaaattcaggatgtcaggattgggaatcatggtgctaacccttctacttcttgtgtccatggcaaccaaccatcaggatagaggagagaagcaggtgacgcaaagggacgcaatcaacgtcagacggagaagatcaatcacccagcaagtcgtatctgaggagtgcaaaaagtactgtaagaaacagaacaagaattgctgcagcagtaaacatgaagaacccagatgtgccaagatatgcttcggatagtttctgtacacggtctcattcattattttatcagtacaagttaaacgagacctatcagaagtcgaaggttgtgcataatttgataaacattgtttgctgggatgaacgga

SEQ ID NO: 174 - protein, 79 aa, Conus purpurascens
MSGLGIMVLTLLLLVSMATNHQDRGEKQVTQRDAINVRRRRSITQQVVSEECKKYCKKQNKNCCSSKHEEPRCAKICFG

SEQ ID NO: 175 - protein, 32 aa, Conus purpurascens
  PEPTIDE (1)..(32): Xaa at residues 4, 5, 23 and 24 is Glu or gamma-carboxy-Glu; Xaa at residue 25 is Pro or hydroxy-Pro; Xaa at residue 9 is Tyr, 125I-Tyr, mono-iodo-Tyr, di-iodo-Tyr, O-sulpho-Tyr or O-phospho-Tyr
VVSXXCKKXCKKQNKNCCSSKHXXXRCAKICF

SEQ ID NO: 176 - protein, 24 aa, Conus purpurascens
  PEPTIDE (1)..(24): Xaa at residue 8 is Glu or gamma-carboxy-Glu; Xaa at residue 12 is Pro or hydroxy-Pro; Xaa at residue 22 is Tyr, 125I-Tyr, mono- iodo-Tyr, di-iodo-Tyr, O-sulpho-Tyr or O-phospho-Tyr
DCCGVKLXMCHXCLCDNSCKNXGK

SEQ ID NO: 177 - DNA, 235 bp, Conus purpurascens
  CDS (1)..(204)
atgttcaccgtgtttctgttggttgtcttggcaaccactgtcgtttccttcacttcagatcgtgcatcggatgacaggaataccaacgacaaagcatctcgcctgctctctcacgttgtcaggggatgctgtggtagctatcccaatgctgcctgtcatccttgcggttgtaaagataggccatcgtattgtggtcaaggacgctgatgctccaggaccctctgaaccacgacgt

SEQ ID NO: 178 - protein, 68 aa, Conus purpurascens
MFTVFLLVVLATTVVSFTSDRASDDRNTNDKASRLLSHVVRGCCGSYPNAACHPCGCKDRPSYCGQGR

SEQ ID NO: 179 - protein, 25 aa, Conus purpurascens
  PEPTIDE (1)..(25): Xaa at residues 7, 13 and 20 is Pro or hydroxy-Pro; Xaa at residues 6 and 22 is Tyr, 125I-Tyr, mono-iodo-Tyr, di-iodo- Tyr, O-sulpho-Tyr or O-phospho-Tyr
GCCGSXXNAACHXCGCKDRXSXCGQ

SEQ ID NO: 180 - DNA, 229 bp, Conus purpurascens
  CDS (1)..(198)
atgttcaccgtgtttctgttggttgtcttggcaaccactgtcgtttccttcaccgtagatcgtgcaactgatggcaggagtgctgcagccatagcgtttgccctgatcgctccgaccgtccgggaaggatgctgttctaatcctgcctgtcatccttgcggttgtaaagataggccatcgtattgtggtcaaggacgctgatgctccaggaccctctgaaccacgacgt

SEQ ID NO: 181 - protein, 66 aa, Conus purpurascens
MFTVFLLVVLATTVVSFTVDRATDGRSAAAIAFALIAPTVREGCCSNPACHPCGCKDRPSYCGQGR

SEQ ID NO: 182 - protein, 23 aa, Conus purpurascens
  PEPTIDE (1)..(23): Xaa at residue 1 is Glu or gamma-carboxy-Glu; Xaa at residues 7, 11 and 18 is Pro or hydroxy-Pro; Xaa at residue 20 is Tyr, 125I- Tyr, mono-iodo-Tyr, di-iodo-Tyr, O-sulpho-Tyr or O- phospho-Tyr
XGCCSNXACHXCGCKDRXSXCGQ

SEQ ID NO: 183 - DNA, 334 bp, Conus purpurascens
  CDS (1)..(261)
atgatgtcgaaaatgggagctatgtttgtccttttgcttcttttcaccctggcatccagccagcaggaaggagatgtccaggcaaggaaaacacgcctgacgagggacttctatcgtactctgccagtgtctactagaggatgcagcggctccccttgttttaaaaacaaaacgtgtcgggatgaatgcatatgcggcggcttatccaattgttggtgtggctacggcggtagtcgaggatgcaagtgtacatgtagagagtgattaatcgactctttaactcgttgaattatttaaaaaatccagagcaatatgttcgagaaaaaccgaagac

SEQ ID NO: 184 - protein, 87 aa, Conus purpurascens
MMSKMGAMFVLLLLFTLASSQQEGDVQARKTRLTRDFYRTLPVSTRGCSGSPCFKNKTCRDECICGGLSNCWCGYGGSRGCKCTCRE

SEQ ID NO: 185 - protein, 41 aa, Conus purpurascens
  PEPTIDE (1)..(41): Xaa at residue 16 is Glu or gamma-carboxy-Glu; Xaa at residue 6 is Pro or hydroxy-Pro; Xaa at residue 26 is Trp (D or L) or bromo-Trp (D or L); Xaa at residue 29 is Tyr, 125I-Tyr, mono- iodo-Tyr, di-iodo-Tyr, O-sulpho-Tyr or O-phospho-Tyr
GCSGSXCFKNKTCRDXCICGGLSNCXCGXGGSRGCKCTCRX

SEQ ID NO: 186 - DNA, 327 bp, Conus purpurascens
  CDS (29)..(256)
cgacctcaagagggatcgatagcagttcatgatgtctaaactgggagccttgttgaccatctgtctgcttctgtttcccattactgctcttctgatggatggagatcaacctgcagaccgacctgcagaacgtatggattacgacatttcatctgaggtgcatcgtttgcttgaaaggagacacccgccctgttgcatgtacggcagatgccgtcgatatcccggatgctctagtgcctcttgttgccagggaggataacgtgttgatgaccaactttgttacacggctacgtcaagtgtctactgaataagtaaaacgattgcagt

SEQ ID NO: 187 - protein, 76 aa, Conus purpurascens
MMSKLGALLTICLLLFPITALLMDGDQPADRPAERMDYDISSEVHRLLERRHPPCCMYGRCRRYPGCSSASCCQGG

SEQ ID NO: 188 - protein, 24 aa, Conus purpurascens
  PEPTIDE (1)..(24): Xaa at residues 2, 3 and 14 is Pro or hydroxy-Pro; Xaa at residue residues 7 and 13 is Tyr, 125I-Tyr, mono-iodo-Tyr, di-iodo-Tyr, O-sulpho-Tyr or O-phospho-Tyr
HXXCCMXGRCRRXXGCSSASCCQG

SEQ ID NO: 189 - protein, 24 aa, Conus purpurascens
  PEPTIDE (1)..(24): Xaa at residues 2, 3, 12 and 14 is Pro or hydroxy-Pro; Xaa at residues 7 and 17 is Tyr, 125I-Tyr, mono-iodo-Tyr, di-iodo-Tyr, O-sulpho-Tyr or O-phospho-Tyr
GXXCCLXGSCRXFXGCXNALCCRK

SEQ ID NO: 190 - DNA, 400 bp, Conus quercinus
  CDS (7)..(237)
ggatccatgaaactgacgtgcgtggtgatcatcgccgtgctgtttctgacagccagtcagctcgttacagctgattacaccagagataaatggcaataccctgcagcgagtttgagaggcggaatgtggaatttgagagataccagggcgtgctcgcaagtaggtgaagcttgttttcctcagaaaccttgctgccctggattcctttgcaatcacatcggaggcatgtgccaccactagtaacagtctggcatctgatatttcccctctgcgctccaccctcttttggctgattcatccttacctgtgtgtggtcatgaaccactcagtagctacacctctggtggcttcagaggacgtatatcaaaataaaaccacattgcaaaaaaaaaaaaaaaaa

SEQ ID NO: 191 - protein, 77 aa, Conus quercinus
MKLTCVVIIAVLFLTASQLVTADYTRDKWQYPAASLRGGMWNLRDTRACSQVGEACFPQKPCCPGFLCNHIGGMCHH

SEQ ID NO: 192 - protein, 30 aa, Conus quercinus
  PEPTIDE (1)..(30): Xaa at residue 7 is Glu or gamma-carboxy-Glu; Xaa at residues 11, 14 and 17 is Pro or hydroxy-Pro
ACSQVGXACFXQKXCCXGFLCNHIGGMCHH

SEQ ID NO: 193 - protein, 11 aa, Conus quercinus
  PEPTIDE (1)..(11): Xaa at residue 4 is Pro or hydroxy-Pro
DCQXCGHNVCC

SEQ ID NO: 194 - DNA, 336 bp, Conus radiatus
  CDS (2)..(241)
catgaactgtctcgtactggctttggttaccatcggtcttctggctgcaacaaccgcagcccctctggacaccaccacggtcctcctcagcacaactacacgcgatgtcaagggctgtgtgtacgagggcatagagtacagtgtcggagagacctaccaggcagactgcaacacgtgtcgctgtgatggctttgacctggctacatgcaccgtcgcgggctgcacaggctttggacccgagtgattggtactattccacacctagcaatgttcacactggaaccggaacttgatactaccttctaaatataatcaatttgtttcaaaaggcccaaa

SEQ ID NO: 195 - protein, 80 aa, Conus radiatus
MNCLVLALVTIGLLAATTAAPLDTTTVLLSTTTRDVKGCVYEGIEYSVGETYQADCNTCRCDGFDLATCTVAGCTGFGPE

SEQ ID NO: 196 - protein, 43 aa, Conus radiatus
  PEPTIDE (1)..(43): Xaa at residues 5, 8, 13 and 43 is Glu or gamma-carboxy-Glu; Xaa at residue 42 is Pro or hydroxy-Pro; Xaa at residues 4,9 and 15 is Tyr, 125I-Tyr, mono-iodo-Tyr, di-iodo-Tyr, O-sulpho-Tyr or O-phospho-Tyr
GCVXXGIXXSVGXTXQADCNTCRCDGFDLATCTVAGCTGFGXX

SEQ ID NO: 197 - DNA, 536 bp, Conus radiatus
  CDS (87)..(296)
gtgagagtccaacagcccaaacctttcaactcactatgtggcagttgcagttttcaacgtctggacaggattcaacaaaattcaggatgtcaggattgggaatcatggtgctaacccttctacttcttgtgtccatggcaaccagtcgtcaggatagaggagtgggacagctgatgccacgcgtctcgttcaaagcctgcaaatcaaattatgattgcccccagcgtttcaaatgctgcagttacacctggaatggatccagtggatactgtaaacgtgtttgctatctttatcgttagtgtaatacacaaagtgactctgttcattcctctccatcatctctttagaaacaacacggtgtcgagatcgtttctttgtgatgaagagtagtatcacgggcagagttcactagagatctcaaatgaaaaacaagattatttagtaagttggggaaaatctggatctcgaaaagattccttgaaaactccgtatttaacacgcttgagagatgataataaagaattctgaaagacaaa

SEQ ID NO: 198 - protein, 70 aa, Conus radiatus
MSGLGIMVLTLLLLVSMATSRQDRGVGQLMPRVSFKACKSNYDCPQRFKCCSYTWNGSSGYCKRVCYLYR

SEQ ID NO: 199 - protein, 34 aa, Conus radiatus
  PEPTIDE (1)..(34): Xaa at residue 9 is Pro or hydroxy-Pro; Xaa at residue 19 is Trp (D or L) or bromo-Trp (D or L); Xaa at residues 6, 17 and 33 is Tyr, 125I-Tyr, mono-iodo-Tyr, di-iodo-Tyr, O-sulpho-Tyr or O-phospho-Tyr
ACKSNXDCXQRFKCCSXTXNGSSGXCKRVCXLXR

SEQ ID NO: 200 - DNA, 356 bp, Conus radiatus
  CDS (1)..(279)
atgatgtcgaaaatgggagctatgtttgtccttttgcttcttttcaccctggcatccagccagcaggaaggagatgtccaggcaaggaaaacacacccgaagagagagttccaacgtattctgctaaggtctggcagaaagtgcaatttcgacaaatgtaaaggtaccggagtctacaattgtggggaatcctgctcatgcgaaggtttgcacagttgtcgctgcacttataacatcggttctatgaagtctggatgcgcgtgtatttgtacatactattaatgattaattgactcgtttaactcgttgaacgatttaaaaaatccagagcaatatgttcgagaaaaaccgaagac

SEQ ID NO: 201 - protein, 93 aa, Conus radiatus
MMSKMGAMFVLLLLFTLASSQQEGDVQARKTHPKREFQRILLRSGRKCNFDKCKGTGVYNCGESCSCEGLHSCRCTYNIGSMKSGCACICTYY

SEQ ID NO: 202 - protein, 47 aa, Conus radiatus
  PEPTIDE (1)..(47): Xaa at residues 17 and 22 is Glu or gamma-carboxy-Glu; Xaa at residues 13, 31, 46 and 47 is Tyr, 125I-Tyr, mono-iodo-Tyr, di- iodo-Tyr, O-sulpho-Tyr or O-phospho-Tyr
KCNFDKCKGTGVXNCGXSCSCXGLHSCRCTXNIGSMKSGCACICTXX

SEQ ID NO: 203 - DNA, 338 bp, Conus radiatus
  CDS (1)..(264)
atgatgtcgaaaatgggagctatgtttgtccttttgcttcttttcaccctggcatccaggcagcaggaaggagatgtccaggcaaggaaaacacgcctgacgagcgacttctatagtgttctgcaaaggtatggactaggatgcgctggcacttgtggttcaagcagcaattgtgttagagattattgtgactgcccaaaacccaattgttactgcactggcaaaggctttcgtcaaccaggatgcgggtgttcatgtttggggtgattaattggctcttttaactcgttgaacgatttaaaaaatccagagcaatatgttcgagaaaaaccgaagac

SEQ ID NO: 204 - protein, 88 aa, Conus radiatus
MMSKMGAMFVLLLLFTLASRQQEGDVQARKTRLTSDFYSVLQRYGLGCAGTCGSSSNCVRDYCDCPKPNCYCTGKGFRQPGCGCSCLG

SEQ ID NO: 205 - protein, 44 aa, Conus radiatus
  PEPTIDE (1)..(44): Xaa at residues 23, 25 and 37 is Pro or hydroxy-Pro; Xaa at residues 1, 19 and 28 is Tyr, 125I-Tyr, mono-iodo-Tyr, di- iodo-Tyr, O-sulpho-Tyr or O-phospho-Tyr
XGLGCAGTCGSSSNCVRDXCDCXKXNCXCTGKGFRQXGCGCSCL

SEQ ID NO: 206 - DNA, 375 bp, Conus sponsalis
  CDS (24)..(260)
gacaggattgaacaaaattcaggatgtcaggattgggaatcatggtgctgacccttttgcttcttgtgtccatggcaaccagccataaggatggaggagagaagcaggcgatgcaaagggacgcaatcaacgtcagactgagaagatcactcactcggagagcagtaactgaggcgtgcacggaggactgtaagactcaggacaagaagtgctgcggcgaaatgaatggacaacacacatgtgccaagatatgcctcggatagtctctgtacgctgtctcattcattatctcatcagtacaagtgtaaacgagacaggtcagaaagtcgaaggttgttcgaaatttgataagcattgtttactgggacgaacgga

SEQ ID NO: 207 - protein, 79 aa, Conus sponsalis
MSGLGIMVLTLLLLVSMATSHKDGGEKQAMQRDAINVRLRRSLTRRAVTEACTEDCKTQDKKCCGEMNGQHTCAKICLG

SEQ ID NO: 208 - protein, 32 aa, Conus sponsalis
  PEPTIDE (1)..(32): Xaa at residues 4, 8 and 20 is Glu or gamma-carboxy-Glu
AVTXACTXDCKTQDKKCCGXMNGQHTCAKICL

SEQ ID NO: 209 - protein, 8 aa, Conus stercusmuscarum
  PEPTIDE (1)..(8): Xaa at residues 3 and 6 is Pro or hydroxy-Pro; Xaa at residue 4 is D-Trp
GCXXQXVC

SEQ ID NO: 210 - protein, 9 aa, Conus striatus
  PEPTIDE (1)..(9): Xaa at residue 7 is Pro or hydroxy-Pro
CIIRNCXRG

SEQ ID NO: 211 - DNA, 238 bp, Conus striatus
  CDS (4)..(96)
aggtcgactcgctgcttgcctgacggaacgtcttgcctttttagtaggatcagatgctgcggtacttgcagttcaatcttaaagtcatgtgtgagctgatccagcggttgatcttcctccctctgtgctccatccttttctgcctgagttctccttacctgagagtggtcatgaaccactcatcacctactcttctggaggcttcagaggagctacagtgaaataaaagccgcattgc

SEQ ID NO: 212 - protein, 31 aa, Conus striatus
STRCLPDGTSCLFSRIRCCGTCSSILKSCVS

SEQ ID NO: 213 - protein, 28 aa, Conus striatus
  PEPTIDE (1)..(28): Xaa at residue 3 is Pro or hydroxy-Pro
CLXDGTSCLFSRIRCCGTCSSILKSCVS

SEQ ID NO: 214 - DNA, 707 bp, Conus striatus
  CDS (171)..(539)
cggcttctaatacgactcactatagggcaagcagtggtaacaacgcagagtacgcggggggacggcagaccagctggggaccagacagacgtcaaacagcatcgcagtcaggtgtggagatcccaagacacccagaagaaggagacagaagagttatcgttcgtaacacaatggccatgaacatgtcgatgacactctgcatgtttgtaatggtcgtcgtggcagccactgtcattgattccactcagttacaagaaccagatctcagtcgcatgcgacgcagcgggcctgctgactgttgcaggatgaaagagtgttgcaccgacagagtgaacgagtgtctacagcgctattctggccgggaagataaattcgtttcgttttgttatcaggaggccacagtcacatgtggatcttttaacgaaatcgtgggctgttgctatggatatcaaatgtgcatgatacgagttgtgaaaccgaacagtctaagtggggcccatgaggcgtgcaaaaccgtttcttgtggtaacccttgcgcttgaggtgtcctcgcgccacgtcacctgtgtacagcgccgtcaccagagccctgatctttatgcccttatctgtctttttgctctttcactctctgaagtcttgaggtttgttccattcttgtcaatcatctcacgcgcatccaagtaaataaaggtgacgtgacaaac

SEQ ID NO: 215 - protein, 123 aa, Conus striatus
MAMNMSMTLCMFVMVVVAATVIDSTQLQEPDLSRMRRSGPADCCRMKECCTDRVNECLQRYSGREDKFVSFCYQEATVTCGSFNEIVGCCYGYQMCMIRVVKPNSLSGAHEACKTVSCGNPCA

SEQ ID NO: 216 - protein, 86 aa, Conus striatus
  PEPTIDE (1)..(86): Xaa at residues 11, 19, 28, 38, 48 and 74 is Glu or gamma- carboxy-Glu; Xaa at residues 3, 66 and 84 is Pro or hydroxy-Pro; Xaa at residues 24, 26, 54 and 56 is Tyr, 125I-Tyr, mono-iodo- Tyr, di-iodo-Tyr, O-sulpho-Tyr or O-phospho-Tyr
SGXADCCRMKXCCTDRVNXCLQRXSGRXDKFVSFCXQXATVTCGSFNXIVGCCXGXQMCMIRVVKXNSLSGAHXACKTVSCGNXCA

SEQ ID NO: 217 - protein, 14 aa, Conus striatus
  PEPTIDE (1)..(14): Xaa at residues 1 and 3 is Trp (D or L) or bromo-Trp (D or L)
XSXRMGNGDRRSDQ

SEQ ID NO: 218 - DNA, 343 bp, Conus striolatus
  CDS (1)..(276)
atgatgtcgaaaatgggagctatgtttgtccttttgcttcttttgaccctggcatccagccagcaggagggagatgtccaggcaaggaaaacaagcctgaagagcgacttctatcgtgctctgagaccgtatgacagacagtgcacttttgtcaacaattgtcaacagaacggtgcgtgtaacggtgattgctcttgcggggaccagatttgtaaatgcggttatagaatcagtcctgggaggtcaggatgcgcgtgtacttgtagaaatgccaaatgaatcacttaactcgttgaaagatttttaaaaatccagagctatatgttcgagaaaaaccgaagac

SEQ ID NO: 219 - protein, 92 aa, Conus striolatus
MMSKMGAMFVLLLLLTLASSQQEGDVQARKTSLKSDFYRALRPYDRQCTFVNNCQQNGACNGDCSCGDQICKCGYRISPGRSGCACTCRNAK

SEQ ID NO: 220 - protein, 46 aa, Conus striolatus
  PEPTIDE (1)..(46): Xaa at residue 1 is Gln or pyro-Gln; Xaa at residue 33 is Pro or hydroxy-Pro; Xaa at residue 29 is Tyr, 125I-Tyr, mono-iodo-Tyr, di-iodo-Tyr, O-sulpho-Tyr or O-phospho-Tyr
XCTFVNNCQQNGACNGDCSCGDQICKCGXRISXGRSGCACTCRNAK

SEQ ID NO: 221 - DNA, 398 bp, Conus tessulatus
  CDS (7)..(240)
ggatccatgaaactgacgtgtgtggtgatcatcgccgtgctgttcctgacggcctgtcaattcattatagctgatttctccagagataagcgggtacatcgtgcagagaggttgagagacataatgcagaatttcagaggtaccaggtcgtgcgcggaatttggtgaagtttgtagttctaccgcttgctgccctgatttggattgcgttgaggcctattcacccatctgtctctgggaatagtctggcatctgatatttcccgtctgtgctctacctacttctgccggattcatccatacctatgtgtggccatgaaccactcagtacctacacctctggtggcttcctagggacgtatatcaaaataaaaccacattgcaaaaaaaaaaaaaaaaa

SEQ ID NO: 222 - protein, 78 aa, Conus tessulatus
MKLTCVVIIAVLFLTACQFIIADFSRDKRVHRAERLRDIMQNFRGTRSCAEFGEVCSSTACCPDLDCVEAYSPICLWE

SEQ ID NO: 223 - protein, 31 aa, Conus tessulatus
  PEPTIDE (1)..(31): Xaa at residues 4, 7 and 22 is Glu or gamma-carboxy-Glu; Xaa at residues 16 and 26 is Pro or hydroxy-Pro; Xaa at residue 30 is Trp (D or L) or bromo-Trp (D or L); Xaa at residue 24 is Tyr, 125I- Tyr, mono-iodo-Tyr, di-iodo-Tyr, O-sulpho-Tyr or O-phospho-Tyr
SCAXFGXVCSSTACCXDLDCVXAXSXICLXX

SEQ ID NO: 224 - DNA, 273 bp, Conus textile
cgattgcaggggttacgatgcgccgtgtagctctggcgcgccatgttgtgattggtggacatgttcagcacgaaccaaccgctgtttttaggctgaccacaagccatccgacatcaccactctcctcttcagaggcttcaaggctttttgttctccttttgaagaatctttacgagtgaacaaacaagtagaatagcacgtttttccccctttgaaaaatcaataatggaggttaaacaaaactgtcttcttcaataaagattttatcataat

SEQ ID NO: 225 - protein, 50 aa, Conus textile
IQGGGDERQKAKINFLSRSDRDCRGYDAPCSSGAPCCDWWTCSARTNRCF

SEQ ID NO: 226 - protein, 29 aa, Conus textile
  PEPTIDE (1)..(29): Xaa at residues 8 and 14 is Pro or hydroxy-Pro; Xaa at residues 18 and 19 is Trp (D or L) or bromo-Trp (D or L); Xaa at residue 5 is Tyr, 125I-Tyr, mono-iodo-Tyr, di-iodo-Tyr, O-sulpho-Tyr or O-phospho-Tyr
DCRGXDAXCSSGAXCCDXXTCSARTNRCF

SEQ ID NO: 227 - protein, 23 aa, Conus textile
  PEPTIDE (1)..(23): Xaa at residue 16 is Glu or gamma-carboxy-Glu; Xaa at residues 3, 11, 12, 22 and 23 is Pro or hydroxy-Pro; Xaa at residues 4, 5 and 14 is Tyr, 125I-Tyr, mono-iodo-Tyr, di-iodo-Tyr, O-sulpho- Tyr or O-phospho-Tyr
NCXXCVVXCCXXAXCXASGCRXX

SEQ ID NO: 228 - DNA, 205 bp, Conus textile
  CDS (1)..(186)
atgcactgtctcccaatcttcgtcattcttctgctgctgactgcatctggacctagcgttgatgcccaactgaagaccaaagatgatgtgcccctgtcatctttccgagatcatgcaaagagtaccctacgaagacttcaggacaaacagacttgctgtggctataggatgtgtgttccttgtggttaaccagcatgaaggatcc

SEQ ID NO: 229 - protein, 62 aa, Conus textile
MHCLPIFVILLLLTASGPSVDAQLKTKDDVPLSSFRDHAKSTLRRLQDKQTCCGYRMCVPCG

SEQ ID NO: 230 - protein, 12 aa, Conus textile
  PEPTIDE (1)..(12): Xaa at residue 1 is Gln or pyro-Gln; Xaa at residue 11 is Pro or hydroxy-Pro; Xaa at residue 6 is Tyr, 125I-Tyr, mono-iodo-Tyr, di-iodo-Tyr, O-sulpho-Tyr or O-phospho-Tyr
XTCCGXRMCVXC

SEQ ID NO: 231 - DNA, 115 bp, Conus textile
  CDS (2)..(94)
gttatggagcgattgctatagttggttaggatcatgtattgcgccctcgcagtgttgttctgaggtttgtgattattactgccgcctatggcgatgaactcggaccacaagccat

SEQ ID NO: 232 - protein, 31 aa, Conus textile
LWSDCYSWLGSCIAPSQCCSEVCDYYCRLWR

SEQ ID NO: 233 - protein, 28 aa, Conus textile
  PEPTIDE (1)..(28): Xaa at residue 18 is Glu or gamma-carboxy-Glu; Xaa at residue 13 is Pro or hydroxy-Pro; Xaa at residues 5 and 27 is Trp (D or L) or bromo-Trp (D or L); Xaa at residue 3, 22 and 23 is Tyr, 125I-Tyr , mono-iodo-Tyr, di-iodo-Tyr, O-sulpho-Tyr or O-phospho-Tyr
DCXSXLGSCIAXSQCCSXVCDXXCRLXR

SEQ ID NO: 234 - DNA, 279 bp, Conus textile
  CDS (7)..(126)
agctgacgaatgaaaaattccgagaatgtcaagctcagcaagagaaaatgtgtggaacaatggaaatactgcacccgagagtccttatgttgcgcgggtttgtgtttgtttagtttctgcattctataacgctaatccagagtcgtatattccgtctaagctccacctggcactgtctggtatgttcctgccagtgactggtctcataccgcttagactctggtccgtcttctctgcaaccacaggagaacgtgcattattacaataaacgcatactgc

SEQ ID NO: 235 - protein, 40 aa, Conus textile
RMKNSENVKLSKRKCVEQWKYCTRESLCCAGLCLFSFCIL

SEQ ID NO: 236 - protein, 27 aa, Conus textile
  PEPTIDE (1)..(27): Xaa at residues 4 and 12 is Glu or gamma-carboxy-Glu; Xaa at residue 6 is Trp (D or L) or bromo-Trp (D or L); Xaa at residue 8 is Tyr, 125I-Tyr, mono-iodo-Tyr, di-iodo-Tyr, O-sulpho-Tyr or O-phospho-Tyr
KCVXQXKXCTRXSLCCAGLCLFSFCIL

SEQ ID NO: 237 - DNA, 510 bp, Conus textile
  CDS (223)..(471)
cagagccgctctggtgtgcagacctgtctccagccctccgtctccctgatcggtggttctgcctgcatagctgtcttctccacgaagctttccacaggtataaataacgcttcagtctcccgtcctgtattgggccgccgttacaagccagaccgatacagccaggtccagtctactttgcgagtgagttaaaagctccagcattctaccagcatcaccagaatgaaggtgagcagcgtgctgatcgtggctacgctgacactgaccgcaggccagctggttagtgcttcttcccattactcaaaagatgtccagattcttccttctgtgagatcagctgacgaagtggaaaattccgagaatgtcaggctcagcaagagaagatgtgtggaacaatgggaagtctgcggcataatcttgttctcctcatcatgttgcgggcagttgtgtttgtttggtttctgcgttctataacgctaatccagagtcgtatattccgtctaagctcca

SEQ ID NO: 238 - protein, 83 aa, Conus textile
MKVSSVLIVATLTLTAGQLVSASSHYSKDVQILPSVRSADEVENSENVRLSKRRCVEQWEVCGIILFSSSCCGQLCLFGFCVL

SEQ ID NO: 239 - protein, 29 aa, Conus textile
  PEPTIDE (1)..(29): Xaa at residues 3 and 6 is Glu or gamma-carboxy-Glu; Xaa at residue 5 is Trp (D or L) or bromo-Trp (D or L)
CVXQXXVCGIILFSSSCCGQLCLFGFCVL

SEQ ID NO: 240 - protein, 27 aa, Conus textile
  PEPTIDE (1)..(27): Xaa at residues 9 and 13 is Glu or gamma-carboxy-Glu; Xaa at resi residues 4, 7 and 23 is Tyr, 125I-Tyr, mono-iodo-Tyr, di-iodo- Tyr, O-sulpho-Tyr or O-phospho-Tyr
CGGXSTXCXVDSXCCSDNCVRSXCTLF

SEQ ID NO: 241 - protein, 27 aa, Conus textile
  PEPTIDE (1)..(27): Xaa at residues 8 and 13 is Glu or gamma-carboxy-Glu
GCNNSCQXHSDCXSHCICTSRGCGAVN

SEQ ID NO: 242 - DNA, 373 bp, Conus tulipa
  CDS (22)..(258)
caggattgaacaaaattcaggatgtcaggattgggaatcatggtgctaacccttctacttcttgtgtccatggcaaccagtcatcgttatgcaagagaaaagcaggcgacgcgaagggacgcagtcaacgtcagacggagaagcagaccaaaaacaaaggagtgcgaaaggtactgtgagctggaggaaaagcactgctgctgcataagaagtaacggacccaaatgttccagaatatgcatattcaaattttggtgttagttttctgtacactgtccattcattatcttatcagtacaagtgtaaacgagacatgtcagaaagtcgaaggttgtgcgtaatttgataagcattgtttactgggacgaacgga

SEQ ID NO: 243 - protein, 79 aa, Conus tulipa
MSGLGIMVLTLLLLVSMATSHRYAREKQATRRDAVNVRRRSRPKTKECERYCELEEKHCCCIRSNGPKCSRICIFKFWC

SEQ ID NO: 244 - protein, 37 aa, Conus tulipa
  PEPTIDE (1)..(37): Xaa at residues 5, 7, 11, 13 and 14 is Glu or gamma-carboxy-Glu; Xaa at residues 1 and 25 is Pro or hydroxy-Pro; Xaa at residue 36 is Trp (D or L) or bromo-Trp (D or L);
XKTKXCXRXCXLXXKHCCCIRSNGXKCSRICIFKFXC

SEQ ID NO: 245 - DNA, 381 bp, Conus tulipa
  CDS (22)..(267)
caggattgaacaaaattcaggatgtcaggattgggaatcatggtgctaacccttctccttcttgtgctaatgacaaccagtcatcaggatgcaggagagaagcaggcgatgcaaagggacgcaaagaacttcagtcggagaagattagtcattcggagaccaaaaacaagggagtgcgaaatgcagtgtgagcaggaggagaaacactgctgccgcgtaagagatggtacgggccaatgtgcccctaagtgcttgggaattaactggtagtttctgtacactgtctcattcattatcttatcagtacacgtgtaacgagacatgtcagaaagtcgaaggtagtgcgtaatttgataagcattgtttactgggacgaacgga

SEQ ID NO: 246 - protein, 82 aa, Conus tulipa
MSGLGIMVLTLLLLVLMTTSHQDAGEKQAMQRDAKNFSRRRLVIRRPKTRECEMQCEQEEKHCCRVRDGTGQCAPKCLGINW

SEQ ID NO: 247 - protein, 36 aa, Conus tulipa
  PEPTIDE (1)..(36): Xaa at residues 5, 7, 11, 13 and 14 is Glu or gamma-carboxy-Glu; Xaa at residues 1 and 29 is Pro or hydroxy-Pro; Xaa at residue 36 is Trp (D or L) or bromo-Trp (D or L)
XKTRXCXMQCXQXXKHCCRVRDGTGQCAXKCLGINX

SEQ ID NO: 248 - DNA, 363 bp, Conus tulipa
  CDS (1)..(264)
atgatgtcgaaaatgggagctatgtttgtccttttgcttcttttcaccctggcatccagccagcaggaaggagatgtccaggcaaggaaaacacgcctgaagagcgacttctatcgtgctctgccaaggtttggcccaatatgcacttgttttaaaagccagaactgtcggggttcttgtgaatgcatgtcacctcccggttgttactgcagtaacaatggcattcgtgaacgaggatgctcgtgtacatgtccagggactggttgaatgatttgaaaaattcagagcaatatgttgcagaaaaaccgaagaccgagacttctcacaataaatccataaagacattaaaaaaaaaaaaaaaaa

SEQ ID NO: 249 - protein, 88 aa, Conus tulipa
MMSKMGAMFVLLLLFTLASSQQEGDVQARKTRLKSDFYRALPRFGPICTCFKSQNCRGSCECMSPPGCYCSNNGIRERGCSCTCPGTG

SEQ ID NO: 250 - protein, 44 aa, Conus tulipa
  PEPTIDE (1)..(44): Xaa at residues 18 and 34 is Glu or gamma-carboxy-Glu; Xaa at residues 3, 22, 23 and 42 is Pro or hydroxy-Pro; Xaa at residue 26 is Tyr, 125I-Tyr, mono-iodo-Tyr, di-iodo-Tyr, O-sulpho-Tyr or O-phospho-Tyr
FGXICTCFKSQNCRGSCXCMSXXGCXCSNNGIRXRGCSCTCXGT

SEQ ID NO: 251 - DNA, 383 bp, Conus tulipa
  CDS (1)..(276)
atgatgtcgaaaatgggagctatgtttgtccttttgcttcttttcaccctggcatccagccagcaggaaggagatgtccaggcaaggaaaacacgcctgaagagcgacttctatcgtactctggcaatatctgacagaggatgcactggcaactgtgattggacgtgtagcggtgattgcagctgccagggcacatctgactcgtgtcactgcattccaccaaaatcaataggcaacagatgccggtgtcagtgtaaaagaaaaatcgaaattgactgattcttttaactcgttgaacgatttaaaaatcagaccaatatgtaggcagaaaaccgaagactctgagactctcgtaataatcgtaagcaaaaaaaaaaaaaaaa

SEQ ID NO: 252 - protein, 92 aa, Conus tulipa
MMSKMGAMFVLLLLFTLASSQQEGDVQARKTRLKSDFYRTLAISDRGCTGNCDWTCSGDCSCQGTSDSCHCIPPKSIGNRCRCQCKRKIEID

SEQ ID NO: 253 - protein, 46 aa, Conus tulipa
  PEPTIDE (1)..(46): Xaa at residue 44 is Glu or gamma-carboxy-Glu; Xaa at residues 27 and 28 is Pro or hydroxy-Pro; Xaa at residue 8 is Trp (D or L) or bromo-Trp (D or L)
GCTGNCDXTCSGDCSCQGTSDSCHCIXXKSIGNRCRCQCKRKIXID

SEQ ID NO: 254 - DNA, 404 bp, Conus virgo
  CDS (7)..(243)
ggatccatgaaactgacgtgtgtggtgatcatcactgtgctgttcctgacggccagtcagctcattacagctgattactccagagatcagcggcagtaccgtgcagtgaggttgggagatgaaatgcggaatttcaaaggtgccagggactgcgggggacaaggtgaaggttgttatactcaaccttgctgccctggtctgcggtgccgtggcggcggtactggaggaggcgtatgccagctgtagtaatagtttggcatctgatatttcccctctgtgctccaccctcttttgcctgattcatccttacctatgtgtggtcatgaaccactcagtagctacacctctggtggattcagagaacgtatatcaaaataaaaccacattgcaataaaaaaaaaaaa

SEQ ID NO: 255 - protein, 79 aa, Conus virgo
MKLTCVVIITVLFLTASQLITADYSRDQRQYRAVRLGDEMRNFKGARDCGGQGEGCYTQPCCPGLRCRGGGTGGGVCQL

SEQ ID NO: 256 - protein, 32 aa, Conus virgo
  PEPTIDE (1)..(32): Xaa at residue 7 is Glu or gamma-carboxy-Glu; Xaa at residues 13 and 16 is Pro or hydroxy-Pro; Xaa at residue 10 is Tyr, 125I-Tyr, mono-iodo-Tyr, di-iodo-Tyr, O-sulpho-Tyr or O-phospho-Tyr
DCGGQGXGCXTQXCCXGLRCRGGGTGGGVCQL

SEQ ID NO: 257 - DNA, 285 bp, Conus radiatus
  CDS (4)..(225)
atcatgcagaaactgacaatcctgcttcttgttgctgctatactgatgtcgacccaggtcctgattcaaggtggtggagaaaaacgccaaaaagtcaacattttttcaaaaagaaagacagatgctgagacctggtgggagggcgaatgctctaattggttaggaagttgttcgacgccctcaaattgctgtctcaagagttgtaatgggcactgcacattgtggtgatgaactctgaccacaaagccatccaacatcaccgctctcctcttcagagtcttcaag

SEQ ID NO: 258 - protein, 74 aa, Conus radiatus
MQKLTILLLVAAILMSTQVLIQGGGEKRQKVNIFSKRKTDAETWWEGECSNWLGSCSTPSNCCLKSCNGHCTLW

SEQ ID NO: 259 - protein, 31 aa, Conus radiatus
  PEPTIDE (1)..(31): Xaa at residues 3 and is Glu or gamma-carboxy-Glu; Xaa at residue 16 is Pro or hydroxy-Pro; Xaa at residues 1, 2, 9 and 31 is Trp (D or L) or bromo-Trp (D or L)
XXXGXCSNXLGSCSTXSNCCLKSCNGHCTLX

SEQ ID NO: 260 - DNA, 296 bp, Conus radiatus
  CDS (1)..(237)
atcatgcagaaactgataatcctgcttcttgttgctgctgtactgatgtccacccaggccctgattcaaggtggtggaggaaaacgccaacaggcaaagagcaagtatttttccgaaagaaaggcacctgctaagcgttggtttggacacgaagaatgcacttattggttggggccttgtgaggtggacgacacgtgttgttctgccagttgtgagtccaagttctgcgggttgtggtgatggacactgaccacaagtcatcctacatcgccactctcctgttcagagtcttcaag

SEQ ID NO: 261 - protein, 79 aa, Conus radiatus
IMQKLIILLLVAAVLMSTQALIQGGGGKRQQAKSKYFSERKAPAKRWFGHEECTYWLGPCEVDDTCCSASCESKFCGLW

SEQ ID NO: 262 - protein, 33 aa, Conus radiatus
  PEPTIDE (1)..(33): Xaa at residues 5, 6, 15 adn 26 is Glu or gamma-carboxy-Glu; Xaa at residue 13 is Pro or hydroxy-Pro; Xaa at residues 1, 10 and 33 is Trp (D or L) or bromo-Trp (D or L);
XFGHXXCTXXLGXCXVDDTCCSASCXSKFCGLX

SEQ ID NO: 263 - DNA, 306 bp, Conus wittigi
  CDS (7)..(231)
ggatccatgaaactgacgtgtgtggtgatcatcgccttgctgttcctgacggcctgtcagctcattacggctgattactccagagatgagcagtctggcagtacagtgcggtttctagacagaccacggcgttttggttcgttcataccgtgcgcccgtttaggtgaaccatgtaccatatgctgccgtcctttgaggtgccgtgaaagcggaacacccacatgtcaagtgtgattgtctggcatctgatatttcccctctgtgccctaccctcttttgcctgagtcatccatacctgtgctcgag

SEQ ID NO: 264 - protein, 75 aa, Conus wittigi
MKLTCVVIIALLFLTACQLITADYSRDEQSGSTVRFLDRPRRFGSFIPCARLGEPCTICCRPLRCRESGTPTCQV

SEQ ID NO: 265 - protein, 33 aa, Conus wittigi
  PEPTIDE (1)..(33): Xaa at residues 12 and 25 is Glu or gamma-carboxy-Glu; Xaa at residues 6, 13, 20 and 29 is Pro or hydroxy-Pro
FGSFIXCARLGXXCTICCRXLRCRXSGTXTCQV

SEQ ID NO: 266 - DNA, 381 bp, Conus regius
  CDS (7)..(240)
ggatccatgaaactgacgtgcgtggtgatcatggcctcgctgttcctggcggcctgtcaattccttacagctggaggtgactcaagaagtaagcagcggtatcctgattggaggctgggctaccgaaagtccaagttgatggctaagaagacgtgcctggaacataacaaactatgttggtatgatagagactgctgcaccatatattgtaatgaaaacaaatgcggcgtgaaacctcaatgaatgtttcacacacacacacacacacacacacacacacacacacacacacacacacacacacacacacatctggcgtctgaccattccccctctgtgctctatcctcttgttcctgagtcatccatacctgtgctcgag

SEQ ID NO: 267 - protein, 78 aa, Conus regius
MKLTCVVIMASLFLAACQFLTAGGDSRSKQRYPDWRLGYRKSKLMAKKTCLEHNKLCWYDRDCCTIYCNENKCGVKPQ

SEQ ID NO: 268 - protein, 30 aa, Conus regius
  PEPTIDE (1)..(30): Xaa at residues 4 and 22 is Glu or gamma-carboxy-Glu; Xaa at residue 29 is Pro or hydroxy-Pro; Xaa at residue 10 is Trp (D or L) or bromo-Trp (D or L); Xaa at residues 11 and 19 is Tyr, 125I-Tyr, mono-iodo-Tyr, di-iodo-Tyr, O-sulpho-Tyr or O-phospho-Tyr
TCLXHNKLCXXDRDCCTIXCNXNKCGVKXQ

SEQ ID NO: 269 - DNA, 285 bp, Conus radiatus; identical to SEQ ID NO: 257
  CDS (1)..(225)

SEQ ID NO: 270 - protein, 75 aa, Conus radiatus
IMQKLTILLLVAAILMSTQVLIQGGGEKRQKVNIFSKRKTDAETWWEGECSNWLGSCSTPSNCCLKSCNGHCTLW

SEQ ID NO: 271 - protein, 31 aa, Conus radiatus; identical to SEQ ID NO: 259
  PEPTIDE (1)..(31): Xaa at residues 3 and 5 is Glu or gamma-carboxy-Glu; Xaa at residue 16 is Pro or hydroxy-Pro; Xaa at residues 1,2, 9 and 31 is Trp (D or L) or bromo-Trp (D or L)

SEQ ID NO: 272 - DNA, 296 bp, Conus radiatus; identical to SEQ ID NO: 260
  CDS (4)..(237)

SEQ ID NO: 273 - protein, 78 aa, Conus radiatus
MQKLIILLLVAAVLMSTQALIQGGGGKRQQAKSKYFSERKAPAKRWFGHEECTYWLGPCEVDDTCCSASCESKFCGLW

SEQ ID NO: 274 - protein, 33 aa, Conus radiatus; identical to SEQ ID NO: 262
  PEPTIDE (1)..(33): Xaa at residues 5, 6, 15 and 26 is Glu or gamma-carboxy-Glu; Xaa at residue 13 is Pro or hydroxy-Pro; Xaa at residues 1, 10 and 33 is Trp (D or L) or bromo-Trp (D or L);

SEQ ID NO: 275 - DNA, 387 bp, Conus spurius
  CDS (21)..(212)
ggaagctgactacaagcagaatgcgctgtctcccagtcttcgtcattcttctgctgctgattccatctgcacctagcactgatgcccgaccgaagaccaaagatgatgtgcgcctggcatctttccacggtaaggcaaagcgaaccctacaaatacctagggggaatatccactgttgcacaaaatatcagccgtgctgttcttcaccatcataaagggaaatgactttgatgagacccctgcgaactgtccctggatgtgaaatttggaaacgagactgttcctttcgcgcgtgttcgtggaatttcgaatggtcgttaataacacgctgcctcttgcaaactacaatctctctgtcctttatctgtggactggatgtcaacactg

SEQ ID NO: 276 - protein, 64 aa, Conus spurius
MRCLPVFVILLLLIPSAPSTDARPKTKDDVRLASFHGKAKRTLQIPRGNIHCCTKYQPCCSSPS

SEQ ID NO: 277 - protein, 17 aa, Conus spurius
  PEPTIDE (1)..(17): Xaa at residues 11 and 16 is Pro or hydroxy-Pro; Xaa at residue 9 is Tyr, 125I-Tyr, mono-iodo-Tyr, di-iodo-Tyr, O-sulpho-Tyr or O-phospho-Tyr
GNIHCCTKXQXCCSSXS

SEQ ID NO: 278 - DNA, 206 bp, Conus nobilis
  CDS (1)..(183)
atgcgctgtctcccagtcttcgtcattcttctgctgctgactgcatctgcaccaagcgttgatgcccgaccgaagaccaaagatgatgtgctccgggcatctttccgcgataatgcaaagagtaccctacaaagactttggaacaaacgcatctgctgccccataattctttggtgctgtggttaaccagcatgaagttcccagga

SEQ ID NO: 279 - protein, 61 aa, Conus nobilis
MRCLPVFVILLLLTASAPSVDARPKTKDDVLRASFRDNAKSTLQRLWNKRICCPIILWCCG

SEQ ID NO: 280 - protein, 10 aa, Conus nobilis
  PEPTIDE (1)..(10): Xaa at residue 4 is Pro or hydroxy-Pro; Xaa at residue 8 is Trp (D or L) or bromo-Trp (D or L)
ICCXIILXCC

SEQ ID NO: 281 - DNA, 205 bp, Conus betulinus
  CDS (1)..(183)
  misc_feature (1)..(205): n is unknown
atgcgctgtctcccagtcttcatcattcttctggtgctgattgcatctgcacctaccgttgatgcccgaccaaagatcgaagatgatgagtccctggcatctttccatgntcatnaaccaccatnanngntncancttttgaacaaacgcaattgctgcccagactctcctccgtgctgtcattaaccagcatgaaggttcagga

SEQ ID NO: 282 - protein, 61 aa, Conus betulinus
  misc_feature (1)..(61): Xaa is unknown
MRCLPVFIILLVLIASAPTVDARPKIEDDESLASFHXHXPPXXXTLLNKRNCCPDSPPCCH

SEQ ID NO: 283 - protein, 11 aa, Conus betulinus
  PEPTIDE (1)..(11): Xaa at residues 4, 7 and 8 is Pro or hydroxy-Pro
NCCXDSXXCCH

SEQ ID NO: 284 - DNA, 569 bp, Conus purpurascens
ggaattccaaatgatgtaattactgactacatggtcatagtgtatacccattgaaaaatttctatgacatttcagttgttagatcatccagttccacagatggaaagacagagagatagtagcttgcaagtggcagcgtgttgttaacgaccattcgacattccatgaacacgtgtgaaaggagcagtctgctttccaaatctgacatccagggacagtttgcaggggtctcatccaaagtcatcttcctttatcccaaagtacagcaccgcatctgttttggacagcaaccgcgtttcttccaaaatctttgtagggttccttttgcattatcgtggaaagatgccaggggcatatcatctttggtcttcggatgagcatcaacgcaaggtgcagatggaatcagcagcagaagaatgacgaagactggcagacagcgcattctgcttgtagtcagcttccgaattccaagccgaattctgcagatatccatcacactggcggccgctcgagcatgcatctagagggcccaattcgccctatagtgagtcgtatgacaattcactggc

SEQ ID NO: 285 - protein, 63 aa, Conus purpurascens
MRCLPVFVILLLLIPSAPCVDAHPKTKDDMPLASFHDNAKGTLQRFWKKRGCCPKQMRCCTLG

SEQ ID NO: 286 - protein, 12 aa, Conus purpurascens
  PEPTIDE (1)..(12): Xaa at residue 4 is Pro or hydroxy-Pro
GCCXKQMRCCTL

SEQ ID NO: 287 - DNA, 221 bp, Conus ammiralis
  CDS (21)..(206)
ggaagctgactacaagcagaatgcactgtctcccagtcgtcgtcattcttctgctgctgactgcatctggtggacctagcgttgatgcccgactgaagaccaaagatgatgtgcccctgtcatctttccgcgataatacaaagagtatcctacaaagactttggaagcgaggcaactgctgtgaattttgggagttttgctgtgattaaccagcatgaagg

SEQ ID NO: 288 - protein, 62 aa, Conus ammiralis
MHCLPVVVILLLLTASGGPSVDARLKTKDDVPLSSFRDNTKSILQRLWKRGNCCEFWEFCCD

SEQ ID NO: 289 - protein, 12 aa, Conus ammiralis
  PEPTIDE (1)..(12): Xaa at residues 5 and 8 is Glu or gamma-carboxy-Glu; Xaa at residue 7 is Trp (D or L) or bromo-Trp (D or L)
GNCCXFXXFCCD

SEQ ID NO: 290 - DNA, 209 bp, Conus dalli
  CDS (21)..(194)
ggaagctgactacaagcagaatgcactgtctcccagtcttcgtcattcttctgctgctgactgcatctggacctagcgttgatgcccaaccgaagaccgaagttgatgtgcccctgtcatctttccgcgataatgcaaagcgtgccctacaaagacttccgcgttgctgtgaatattggaagttgtgctgtggttaaccagcatgaagg

SEQ ID NO: 291 - protein, 58 aa, Conus dalli
MHCLPVFVILLLLTASGPSVDAQPKTEVDVPLSSFRDNAKRALQRLPRCCEYWKLCCG

SEQ ID NO: 292 - protein, 9 aa, Conus dalli
  PEPTIDE (1)..(9): Xaa at residue 1 is Glu or gamma-carboxy-Glu; Xaa at residue 5 is Trp (D or L) or bromo-Trp (D or L); Xaa at residue 4 is Tyr, 125I-Tyr, mono-iodo-Tyr, di-iodo-Tyr, O-sulpho-Tyr or O-phospho-Tyr
CCXXXKLCC

SEQ ID NO: 293 - DNA, 218 bp, Conus omaria
  CDS (21)..(203)
ggaagctgactacaagcagaatgcgctgtctcccagtcttcgtcattcttctgctgctaactgcatctgcacctagcgttgatgcccgaccgaaggccaaagatgatgtgcccctggcatctttccgtgataatgcaaagagtaccctacaaagacttcaggacaaacgcgtttgctgtggctataagtttttttgctgtcgttaaccagcatgaagg

SEQ ID NO: 294 - protein, 61 aa, Conus omaria
MRCLPVFVILLLLTASAPSVDARPKAKDDVPLASFRDNAKSTLQRLQDKRVCCGYKFFCCR

SEQ ID NO: 295 - protein, 11 aa, Conus omaria
  PEPTIDE (1)..(11): Xaa at residue 5 is Tyr, 125I-Tyr, mono-iodo-Tyr, di-iodo-Tyr, O-sulpho-Tyr or O-phospho-Tyr
VCCGXKFFCCR

SEQ ID NO: 296 - DNA, 212 bp, Conus aulicus
  CDS (21)..(197)
ggaagctgactacaagcagaatgcgctgtctcccagtcttcgtcattcttctgctgctgactgcatctgcacctaacgttgatgcccaaccgaagaccaaagatgatgtgcccctggcatctttgcacgatgatgcaaagagtgcactacaacattggaaccaacgctgctgccccatgatctattggtgctgtagttaaccagcatgaagg

SEQ ID NO: 297 - protein, 59 aa, Conus aulicus
MRCLPVFVILLLLTASAPNVDAQPKTKDDVPLASLHDDAKSALQHWNQRCCPMIYWCCS

SEQ ID NO: 298 - protein, 10 aa, Conus aulicus
  PEPTIDE (1)..(10): Xaa at residue 3 is Pro or hydroxy-Pro; Xaa at residue 7 is Trp (D or L) or bromo-Trp (D or L); Xaa at residue 6 is Tyr, 125I-Tyr, mono-iodo-Tyr, di-iodo-Tyr, O-sulpho-Tyr or O-phospho-Tyr
CCXMIXXCCS

SEQ ID NO: 299 - DNA, 212 bp, Conus aulicus
  CDS (21)..(197)
ggaagctgactacaagcagaatgcactgtctcccagtcttcgtcattcttctgctgctgactgcatctgcacctaacgttgatgcccaaccgaagaccaaagatgatgtgcccctggcatctttgcacgatgatgcaaagagtgcactacaacattggaaccaacgctgctgccccgagatctattggtgctgtagttaaccagcatgaagg

SEQ ID NO: 300 - protein, 59 aa, Conus aulicus
MHCLPVFVILLLLTASAPNVDAQPKTKDDVPLASLHDDAKSALQHWNQRCCPEIYWCCS

SEQ ID NO: 301 - protein, 10 aa, Conus aulicus
  PEPTIDE (1)..(10): Xaa at residue 4 is Glu or gamma-carboxy-Glu; Xaa at residue 3 is Pro or hydroxy-Pro; Xaa at residue 7 is Trp (D or L) or bromo- Trp (D or L); Xaa at residue 6 is Tyr, 125I-Tyr, mono-iodo-Tyr, di-iodo-Tyr, O-sulpho-Tyr or O-phospho-Tyr
CCXXIXXCCS

SEQ ID NO: 302 - DNA, 215 bp, Conus ammiralis
  CDS (21)..(200)
ggaagctgactacaagcagaatgcgctgtctcccagtcttcgtcattcttctgctgctgattgcatctgcacctagcgttgatgcccaaccgaagaccaaagatgatgtgtccctggcatctttgcacgataatataaagagtactctacaaacactttggaacaaacgctgctgcccccctgtgatttggtgctgtggttaaccagcataaagg

SEQ ID NO: 303 - protein, 60 aa, Conus ammiralis
MRCLPVFVILLLLIASAPSVDAQPKTKDDVSLASLHDNIKSTLQTLWNKRCCPPVIWCCG

SEQ ID NO: 304 - protein, 9 aa, Conus ammiralis
  PEPTIDE (1)..(9): Xaa at residues 3 and 4 is Pro or hydroxy-Pro; Xaa at residue 7 is Trp (D or L) or bromo-Trp (D or L)
CCXXVIXCC

SEQ ID NO: 305 - DNA, 215 bp, Conus aulicus
  CDS (21)..(200)
ggaagctgactacaagcagaatgcgctgtctcccagtcttcgtcattcttctgctgctgactgcatctggacctagcgttgatgcccgaccgaagaccaaagatgatgtgcctctgtcatctttccgcgataacgcaaagagtatcctacaaagacgttggaacaactattgctgcacgaatgagctttggtgctgtggttaaccagcatgaagg

SEQ ID NO: 306 - protein, 60 aa, Conus aulicus
MRCLPVFVILLLLTASGPSVDARPKTKDDVPLSSFRDNAKSILQRRWNNYCCTNELWCCG

SEQ ID NO: 307 - protein, 13 aa, Conus aulicus
  PEPTIDE (1)..(13): Xaa at residue 9 is Glu or gamma-carboxy-Glu; Xaa at residues 1 and 11 is Trp (D or L) or bromo-Trp (D or L); Xaa at residue 4 is Tyr, 125I-Tyr, mono-iodo-Tyr, di-iodo-Tyr, O-sulpho-Tyr or O-phospho-Tyr
XNNXCCTNXLXCC

SEQ ID NO: 308 - DNA, 218 bp, Conus dalli
  CDS (21)..(203)
ggaagctgactacaagcagaatgcactgtctcccagtcttcgtcattcttctgctgctgactgcatctggacctagcgttgatgcccgaccgaagaccgaagatgatgtgcccctgtcatctttccgcgataatacaaagagtaccctacaaagacttttgaagccagtcaactgctgtcctattgatcaatcttgctgttcttaaccagcatgaagg

SEQ ID NO: 309 - protein, 61 aa, Conus dalli
MHCLPVFVILLLLTASGPSVDARPKTEDDVPLSSFRDNTKSTLQRLLKPVNCCPIDQSCCS

SEQ ID NO: 310 - protein, 13 aa, Conus dalli
  PEPTIDE (1)..(13): Xaa at residues 1 and 6 is Pro or hydroxy-Pro
XVNCCXIDQSCCS

SEQ ID NO: 311 - DNA, 239 bp, Conus consors
  CDS (7)..(228)
ggatccatgttcaccgtgtttctgttggttgtcttggcaaccactgtcgtttccatcccttcagatcgtgcatctgaaggcaggaatgccgtagtccacgagagagcgcctgagctggtcgttacggccaccacgacttgctgtggttatgatccgatgacaatatgccctccttgcatgtgcactcattcctgtccaccaaaaagaaaaccaggccgcagaaacgactgatgctcgag

SEQ ID NO: 312 - protein, 74 aa, Conus consors
MFTVFLLVVLATTVVSIPSDRASEGRNAVVHERAPELVVTATTTCCGYDPMTICPPCMCTHSCPPKRKPGRRND

SEQ ID NO: 313 - protein, 36 aa, Conus consors
  PEPTIDE (1)..(36): Xaa at residue 3 is Glu or gamma-carboxy-Glu; Xaa at residues 2, 17, 22, 23, 31, 32 and 36 is Pro or hydroxy-Pro;  Xaa at residue 15 is Tyr, 125I-Tyr, mono-iodo-Tyr, di-iodo-Tyr, O-sulpho-Tyr or O-phospho-Tyr
AXXLVVTATTTCCGXDXMTICXXCMCTHSCXXKRKX

SEQ ID NO: 314 - DNA, 272 bp, Conus aurisiacus
  CDS (7)..(237)
ggatccatgttcaccgtgtttctgttggttgtcttggcaaccactgtcgtttccatcccttcagatcgtgcatctgatggcaggaatgccgcagtcaacgagagacaatcttggctggtcccttcgacaatcacgacttgctgtggatatgatccggggacaatgtgccctccttgcaggtgcaataatacctgtaaaccaaaaaaaccaaaaccaggaaaaggccgcagaaacgactgatgctccaggaccctctgaaccacgacctcgag

SEQ ID NO: 315 - protein, 77 aa, Conus aurisiacus
MFTVFLLVVLATTVVSIPSDRASDGRNAAVNERQSWLVPSTITTCCGYDPGTMCPPCRCNNTCKPKKPKPGKGRRND

SEQ ID NO: 316 - protein, 39 aa, Conus aurisiacus
  PEPTIDE (1)..(39): Xaa at residue 1 is Gln or pyro-Gln; Xaa at residues 6, 17, 22, 23, 32, 35 and 37 is Pro or hydroxy-Pro; Xaa at residue 3 is Trp (D or L) or bromo-Trp (D or L); Xaa at residue 15 is Tyr, 125I-Tyr , mono-iodo-Tyr, di-iodo-Tyr, O-sulpho-Tyr or O-phospho-Tyr
XSXLVXSTITTCCGXDXGTMCXXCRCNNTCKXKKXKXGK

SEQ ID NO: 317 - DNA, 266 bp, Conus consors
  CDS (7)..(231)
ggatccatgttcaccgtgtttctgttggttgtcttggcaaccactgtcgtttccatcccttcagatcgtgcatctgatggcaggaatgccgtagtccacgagagagcgcctgagctggtcgttacggccaccacgacttgctgtggttatgatccgatgacatggtgcccttcttgcatgtgcacttattcctgtccccaccaaaggaaaaaaccaggccgcagaaacgactgatgctccaggaccctctgaaccacgacctcgag

SEQ ID NO: 318 - protein, 75 aa, Conus consors
MFTVFLLVVLATTVVSIPSDRASDGRNAVVHERAPELVVTATTTCCGYDPMTWCPSCMCTYSCPHQRKKPGRRND

SEQ ID NO: 319 - protein, 37 aa, Conus consors
  PEPTIDE (1)..(37): Xaa at residue 3 is Glu or gamma-carboxy-Glu; Xaa at residues 2, 17, 22, 31 and 37 is Pro or hydroxy-Pro; Xaa at residue 20 is Trp (D or L) or bromo-Trp (D or L);
AXXLVVTATTTCCGXDXMTXCXSCMCTXSCXHQRKKX

SEQ ID NO: 320 - DNA, 260 bp, Conus magus
  CDS (7)..(231)
  misc_feature (1)..(260): n is unknown
ggatccatgttcaccgtgtttctgttggttgtcttggcaaccagtgtcgtttccatcccttcagatcgtgcatctgatggcgggaatgccgtagtccacgagagagcgcctgagctggtcgttacggccaccacgacttgctgtggttatgatccgatgacaatatgccctccctgcatgtgcactcattcctgtccaccaaaaggaaaaccaggccgcaggaacgactgatgtccaggacctctgaaccacgacncgag

SEQ ID NO: 321 - protein, 74 aa, Conus magus
MFTVFLLVVLATSVVSIPSDRASDGGNAVVHERAPELVVTATTTCCGYDPMTICPPCMCTHSCPPKGKPGRRND

SEQ ID NO: 322 - protein, 36 aa, Conus magus
  PEPTIDE (1)..(36): Xaa at residue 3 is Glu or gamma-carboxy-Glu; Xaa at residues 2, 17, 22, 23, 31, 32 and 36 is Pro or hydroxy-Pro; Xaa at residue 15 is Tyr, 125I-Tyr, mono-iodo-Tyr, di-iodo-Tyr, O-sulpho-Tyr or O-phospho-Tyr
AXXLVVTATTTCCGXDXMTICXXCMCTHSCXXKGKX

SEQ ID NO: 323 - DNA, 251 bp, Conus aurisiacus
  CDS (23)..(202)
gaattcgcccttgaggatccgtgtggttctgggtccagaacctgatggcaggaatgccgcagtcaacgagagacagaaatggctggtccattcgaaaatcacgtattgctgtggttataataagatggacatgtgccctccttgcatgtgcacttattcctgtccccccctaaaaaaaaaaagaccaggccgcagaaacgactgatgctccaggaccctctgaaccacgacctcgagcgaagggcgaattc

SEQ ID NO: 324 - protein, 60 aa, Conus aurisiacus
VVLGPEPDGRNAAVNERQKWLVHSKITYCCGYNKMDMCPPCMCTYSCPPLKKKRPGRRND

SEQ ID NO: 325 - protein, 38 aa, Conus aurisiacus
  PEPTIDE (1)..(38): Xaa at residue 1 is Gln or pyro-Gln; Xaa at residues 22, 23, 31, 32 and 38 is Pro or hydroxy-Pro; Xaa at residue 3 is Trp (D or L) or bromo-Trp (D or L); Xaa at residues 11, 15 and 28  is Tyr, 125I -Tyr, mono-iodo-Tyr, di-iodo-Tyr, O-sulpho-Tyr or O-phospho-Tyr
XKXLVHSKITXCCGXNKMDMCXXCMCTXSCXXLKKKRX

SEQ ID NO: 326 - DNA, 212 bp, Conus aurisiacus
  CDS (23)..(163)
gaattcgcccttgaggatccgtgtggttctgggtccagcatttgatggcaggaatgccgcagtcaacgagagagcgccttggacggtcgttacggccaccacgaattgctgcggtattaccgggccaggctgccttccttgccgttgtactcaaacatgtggctgatgctccaggaccctctgaaccacgacctcgagcgaagggcgaattc

SEQ ID NO: 327 - protein, 47 aa, Conus aurisiacus
VVLGPAFDGRNAAVNERAPWTVVTATTNCCGITGPGCLPCRCTQTCG

SEQ ID NO: 328 - protein, 29 aa, Conus aurisiacus
  PEPTIDE (1)..(29): Xaa at residues 2, 18 and 22 is Pro or hydroxy-Pro; Xaa at residue 3 is Trp (D or L) or bromo-Trp (D or L)
AXXTVVTATTNCCGITGXGCLXCRCTQTC

SEQ ID NO: 329 - DNA, 218 bp, Conus marmoreus
  CDS (21)..(203)
ggaagctgactacaagcagaatgcgctgtctcccagtcttgatcattcttctgctgctgactgcatctgcacctggcgttgttgtcctaccgaagaccgaagatgatgtgcccatgtcatctgtctacggtaatggaaagagtatcctacgagggattctgaggaacggtgtttgctgtggctataagttgtgccttccatgttaaccagcatgaagg

SEQ ID NO: 330 - protein, 61 aa, Conus marmoreus
MRCLPVLIILLLLTASAPGVVVLPKTEDDVPMSSVYGNGKSILRGILRNGVCCGYKLCLPC

SEQ ID NO: 331 - protein, 13 aa, Conus marmoreus
  PEPTIDE (1)..(13): Xaa at residue 12 is Pro or hydroxy-Pro; Xaa at residue 7 is Tyr, 125I-Tyr, mono-iodo-Tyr, di-iodo-Tyr, O-sulpho-Tyr or O-phospho-Tyr
NGVCCGXKLCLXC

SEQ ID NO: 332 - DNA, 238 bp, Conus pennaceus
  CDS (28)..(219)
ggaattcggaagctgactacaagcagaatgcgctgtctcccagtcttcgtcattcttctgctgctgactgcatctgcacctagcgttgatgccaaagttcatctgaagaccaaaggtgatgggcccctgtcatctttccgagataatgcaaagagtaccctacaaagacttcaggacaaaagcacttgctgtggctttaagatgtgtatcccttgtagttaaccagcatgaaggatcc

SEQ ID NO: 333 - protein, 64 aa, Conus pennaceus
MRCLPVFVILLLLTASAPSVDAKVHLKTKGDGPLSSFRDNAKSTLQRLQDKSTCCGFKMCIPCS

SEQ ID NO: 334 - protein, 13 aa, Conus pennaceus
  PEPTIDE (1)..(13): Xaa at residue 11 is Pro or hydroxy-Pro
STCCGFKMCIXCS

SEQ ID NO: 335 - DNA, 231 bp, Conus pennaceus
  CDS (27)..(212)
gaattcggaagctgactacaagcagaatgcgttgtctcccagtcttcgtcattcttctgctgctgactgcatctggacctagcgttgatgcccgactgaagaccaaagatgatgtgcccctgtcatctttccgagataatgcaaagagtaccctacaaagacttcaggacaaacgcctttgctgtggcttttggatgtgtattccttgtaattaaccagcatgaaggatcc

SEQ ID NO: 336 - protein, 62 aa, Conus pennaceus
MRCLPVFVILLLLTASGPSVDARLKTKDDVPLSSFRDNAKSTLQRLQDKRLCCGFWMCIPCN

SEQ ID NO: 337 - protein, 12 aa, Conus pennaceus
  PEPTIDE (1)..(12): Xaa at residue 10 is Pro or hydroxy-Pro; Xaa at residue 6 is Trp (D or L) or bromo-Trp (D or L)
LCCGFXMCIXCN

SEQ ID NO: 338 - DNA, 244 bp, Conus pennaceus
  CDS (40)..(225)
  misc_feature (1)..(244): n is unknown
gaattctcccttggaattctgaagctgactacaancagaatgcgttgtctcccactcttcgtcattcttctgctgctgactgcatctggacctactgttgatgcccgactgaagaccaaagatgatgtgcccctgtcatctttccgagataatgcaaagagtaccctacaaagacttcaggacaaaagcacttgctgtggctttaagatgtgtattccttgtggttaaccagcatgaaggatcc

SEQ ID NO: 339 - protein, 62 aa, Conus pennaceus
MRCLPLFVILLLLTASGPTVDARLKTKDDVPLSSFRDNAKSTLQRLQDKSTCCGFKMCIPCG

SEQ ID NO: 340 - protein, 12 aa, Conus pennaceus
  PEPTIDE (1)..(12): Xaa at residue 11 is Pro or hydroxy-Pro
STCCGFKMCIXC

SEQ ID NO: 341 - DNA, 250 bp, Conus episcopatus
  misc_feature (1)..(250): n is unknown
gaattcgcccttggaattcggaagctgactacaagcagaatgcgctgtctcccagtcttcgtcattcttctgctgctgactgcatctggacctantgttgatgccaaagttcatctgaagaccaaaggtgatgggcccctgtcatctttccgagataatgcaaagagtaccctacaaagacttcaggacaaaagcacttgctgtggctataggatgtgtgttccttgtggttaaccagcatgaaggatcc

SEQ ID NO: 342 - protein, 64 aa, Conus episcopatus
MRCLPVFVILLLLTASGPSVDAKVHLKTKGDGPLSSFRDNAKSTLQRLQDKSTCCGYRMCVPCG

SEQ ID NO: 343 - protein, 12 aa, Conus episcopatus
  PEPTIDE (1)..(12): Xaa at residue 11 is Pro or hydroxy-Pro;  Xaa at residue 6 is Tyr , 125I-Tyr, mono-iodo-Tyr, di-iodo-Tyr, O-sulpho-Tyr or O-phospho-Tyr
STCCGXRMCVXC

SEQ ID NO: 344 - DNA, 827 bp, Conus marmoreus
  CDS (82)..(264)
ggcgaatacacctggcaggtactcaacgaacttcaggacacattcttttcacctggacactggaaactgacaacaggcagaatgcgctgtctcccagtcttgatcattcttctgctgctgactgcatctgcacctggcgttgttgtcctaccgaagaccgaagatgatgtgcccatgtcatctgtctacggtaatggaaagagtatcctacgaggaattctgaggaacggtgtttgctgtggctataagttgtgccatccatgttaaccagcatgaagggaaatgactttggatgagacccctgcgaactgtccctggatgtgaaatttggaaagcagactgttcctttcgcacgtattcgtggaatttcgaatggtcgtaaacaacacgctgccacttgcaggctactatctctctgtcctttcatctgtggaaatggatgatctaacaactgaaatatcagaaatttttcaatggctatacactatgaccatgtagtcagtaattatatcatttggaccttttgaaatatttttcaatatgtaaagtttttgcaccctggaaaggtcttttggagttaaatattttagtatgttatgttttgcatacaagttatagaatgctgtctttctttttgttcccacatcaatggtgggggcagaaattatttgttttggtcaatgtaattatgacctgcatttagtgctatagtgattgcattttcagcgtggaatgtttaatctgcaaacagaaagtggttgatcgactaataaagatttgcatggcacaaaaaaaaaaaaaaaaagtactctgcgttgttactcgag

SEQ ID NO: 345 - protein, 61 aa, Conus marmoreus
MRCLPVLIILLLLTASAPGVVVLPKTEDDVPMSSVYGNGKSILRGILRNGVCCGYKLCHPC

SEQ ID NO: 346 - protein, 13 aa, Conus marmoreus
  PEPTIDE (1)..(13): Xaa at residue 12 is Pro or hydroxy-Pro; Xaa at residue 7 is Tyr, 125I-Tyr, mono-iodo-Tyr, di-iodo-Tyr, O-sulpho-Tyr or O-phospho-Tyr
NGVCCGXKLCHXC

SEQ ID NO: 347 - protein, 12 aa, Conus marmoreus
  PEPTIDE (1)..(12): Xaa at residue 11 is Pro or hydroxy-Pro; Xaa at residue 6 is Tyr, 125I-Tyr, mono-iodo-Tyr, di-iodo-Tyr, O-sulpho-Tyr or O-phospho-Tyr
GVCCGXKLCHXC

SEQ ID NO: 348 - DNA, 202 bp, Conus bandanus; identical to SEQ ID NO: 61
  CDS (1)..(183)

SEQ ID NO: 349 - protein, 61 aa, Conus bandanus; identical to SEQ ID NO: 62

SEQ ID NO: 350 - protein, 11 aa, Conus bandanus
  PEPTIDE (1)..(11): Xaa at residue 10 is Pro or hydroxy-Pro; Xaa at residue 5 is Tyr, 125I-Tyr, mono-iodo-Tyr, di-iodo-Tyr, O-sulpho-Tyr or O-phospho-Tyr
ACCGXKLCSXC

SEQ ID NO: 351 - DNA, 221 bp, Conus aulicus
  CDS (21)..(206)
ggaagctgactacaagcagaatgcgctgtctcccagtcttcgtcattcttctgctgctgactgcatctggacctagcgttgatgcccgactgaagaccaaagatgatgtgcccctgtcatctttccgagataatgcaaagagtaccctacaaagacatcaggacaaaagcgtttgctgtggctataagctgtgttttccttgtggttaaccagcatgaagg

SEQ ID NO: 352 - protein, 62 aa, Conus aulicus
MRCLPVFVILLLLTASGPSVDARLKTKDDVPLSSFRDNAKSTLQRHQDKSVCCGYKLCFPCG

SEQ ID NO: 353 - protein, 12 aa, Conus aulicus
  PEPTIDE (1)..(12): Xaa at residue 11 is Pro or hydroxy-Pro; Xaa at residue 6 is Tyr, 125I-Tyr, mono-iodo-Tyr, di-iodo-Tyr, O-sulpho-Tyr or O-phospho-Tyr
SVCCGXKLCFXC

SEQ ID NO: 354 - DNA, 312 bp, Conus textile
  CDS (3)..(50)
  misc_feature (1)..(312): n is unknown
caggatccaatggggtttgttgtggctataggatgtgtgttccttgtggttaaccagcatgaagggaaatgactttggatgagacccctgcgaactgtccctggatgtgagatttggaaagcagactgttcattttgcacgtgttcgtggaatttcgaatggtcgttaacaacacgctgccacttgcaagctactatctctctgtccttttatctgtggaactgtatgatctaacaactgaaatatcatananatttttcaatgggtatncactatgcatatgatcatgtagggttcaaggggtcaagatnc

SEQ ID NO: 355 - protein, 16 aa, Conus textile
GSNGVCCGYRMCVPCG

SEQ ID NO: 356 - protein, 13 aa, Conus textile
  PEPTIDE (1)..(13): Xaa at residue 12 is Pro or hydroxy-Pro; Xaa at residue 7 is Tyr, 125I-Tyr, mono-iodo-Tyr, di-iodo-Tyr, O-sulpho-Tyr or O-phospho-Tyr
NGVCCGXRMCVXC

SEQ ID NO: 357 - DNA, 205 bp, Conus textile; identical to SEQ ID NO: 228
  CDS (1)..(186)

SEQ ID NO: 358 - protein, 62 aa, Conus textile; identical to SEQ ID NO: 229

SEQ ID NO: 359 - protein, 12 aa, Conus textile
  PEPTIDE (1)..(12): Xaa at residue 1 is Gln or pyro-Gln; Xaa at residue 11 is Pro or hydroxy-Pro; Xaa at residue 6 is Tyr, 125I-Tyr, mono-iodo-Tyr, di--iodo-Tyr, O-sulpho-Tyr or O-phospho-Tyr
XTCCGXRMCVXC

SEQ ID NO: 360 - DNA, 221 bp, Conus ammiralis
  CDS (21)..(206)
agaagctgactacaagcagaatgcactacctcccagtcttcgtcattcttctgctgctgactgcatctggacctagcgttgatgcccaactgaagaccaaagatgatgtgcccctgtcatctttccgagataatgcaaagagtaccctacgaagactccagtacaaacaggcttgctgtggctttaagatgtgtgttccttgtggttaaccagcatgaagg

SEQ ID NO: 361 - protein, 62 aa, Conus ammiralis
MHYLPVFVILLLLTASGPSVDAQLKTKDDVPLSSFRDNAKSTLRRLQYKQACCGFKMCVPCG

SEQ ID NO: 362 - protein, 12 aa, Conus ammiralis
  PEPTIDE (1)..(12): Xaa at residue 1 is Gln or pyro-Gln; Xaa at residue 11 is Pro or hydroxy-Pro
XACCGFKMCVXC

SEQ ID NO: 363 - DNA, 211 bp, Conus pennaceus; identical to SEQ ID NO: 161
  CDS (1)..(192)

SEQ ID NO: 364 - protein, 64 aa, Conus pennaceus; identical to SEQ ID NO: 162

SEQ ID NO: 365 - protein, 13 aa, Conus pennaceus
  PEPTIDE (1)..(13): Xaa at residue 13 is Pro or hydroxy-Pro
STCCGFKMCIXCR

SEQ ID NO: 366 - DNA, 304 bp, Conus pennaceus
  CDS (3)..(50)
  misc_feature (1)..(304): n is unknown
caggatccaatggggtttgttgtggcttttggatgtgtattccttgtaattaaccagcatgaagggaaatgactttggataagacccctgcgaactgtccttggatgtgagatttggaaagcagactgttccttttgcacgtgttcgtggaatttcgaatggtcgttaacaacacgctgccacttgcaagctactatctctctgtcctttcatctgtggaactgtatgatctaacaactgaaatatcatagaaatttttcaatgggtatacactatgcatatgaccatgtangggtcaacagnc

SEQ ID NO: 367 - protein, 16 aa, Conus pennaceus
GSNGVCCGFWMCIPCN

SEQ ID NO: 368 - protein, 14 aa, Conus pennaceus
  PEPTIDE (1)..(14): Xaa at residue 12 is Pro or hydroxy-Pro; Xaa at residue 8 is Trp (D or L) or bromo-Trp (D or L)
NGVCCGFXMCIXCN

SEQ ID NO: 369 - DNA, 218 bp, Conus omaria
  CDS (21)..(203)
ggaagctgactacaagcagaatgcgctgtctcccagtcttcgtcattcttctgctgctgactgcatctgcacctagcgttgatgcccgaccgaaggccaaagatgatgtgcccctgtcatctttccgtgataatgcaaagagtaccctacaaagacttcaggacaaagacgtttgctgttacgttagaatgtgtccttgtcgttaaccagcatgaagg

SEQ ID NO: 370 - protein, 61 aa, Conus omaria
MRCLPVFVILLLLTASAPSVDARPKAKDDVPLSSFRDNAKSTLQRLQDKDVCCYVRMCPCR

SEQ ID NO: 371 - protein, 12 aa, Conus omaria
  PEPTIDE (1)..(12): Xaa at residue 10 is Pro or hydroxy-Pro; Xaa at residue 5 is Tyr, 125I-Tyr, mono-iodo-Tyr, di-iodo-Tyr, O-sulpho-Tyr or O-phospho-Tyr
DVCCXVRMCXCR

SEQ ID NO: 372 - protein, 84 aa, Conus radiatus
  PEPTIDE (1)..(84): Xaa at residues 27, 32, 38, 39 and 41 is Glu or gamma-carboxy-Glu ; Xaa at residues 2, 5, 17, 23, 43, 45 and 58 is Pro or hydroxy- Pro;Xaa at residue 8 is Tyr, 125I-Tyr, mono-iodo-Tyr, di-iodo- Tyr, O-sulpho-Tyr or O-phospho-Tyr
HXTKXCMXCSFGQCVGXHICCGXTGCXMGTAXANMCSXXDXDXIXCQVFGSDCALNNXDNIHGHCVADGICCVDDTCTTHLGCL

SEQ ID NO: 373 - DNA, 218 bp, Conus tessulatus
  CDS (7)..(174)
ggatccatgttcaccgtgtttctgttggttgtcttggcaaccactgttgtttccttcagtgcagatcgtgccaacgtcaaagcgtctgacctgatcgcccaggccaccagagacggctgtccaccacatcccgttcctggcatgcataagtgcatgtgtactaatacatgtggttgaagacgctgatgctccaggaccctctgaaccacgacctcgag

SEQ ID NO: 374 - protein, 56 aa, Conus tessulatus
MFTVFLLVVLATTVVSFSADRANVKASDLIAQATRDGCPPHPVPGMHKCMCTNTCG

SEQ ID NO: 375 - protein, 20 aa, Conus tessulatus
  PEPTIDE (1)..(20): Xaa at residues 4, 5, 7 and 9 is Pro or hydroxy-Pro
DGCXXHXVXGMHKCMCTNTC

SEQ ID NO: 376 - DNA, 536 bp, Conus geographus
  CDS (400)..(510)
  misc_feature (1)..(536): n is unknown
anntagantntgtcgtantannggatcntaantantgnntcganatgatnangagtgataaatgannggtgcactnntanttangntnntangatnnnnatattatnntannnnntaananatatnggtnnggannaagaagantaaaagtanngnttngtgaaanaangannnnatgttnnanntcataacnnnaatgtaaataatanacgnnccagtgtgaaannntntcnnnnataaaaattctntntntnaangtnnntgtntgngtgtgtgtgtgtgtgtgtgtgtgtgngtgtgtgngtgtgtgtgtgtgtgtgtgtgtgtgtgngtgtgtgtntgtgngtgtgtgtgtgtgtgtgtgtgtgtgtgtgtgtgtntgtggttctgggtccagcatctgatgncagggatgacacagccaaagacgaagggtctnacatggacaaattggtcgagaaaaaagaatgttgccatcctgcctgtggcaaacactacagttgtggacgctgatgctccagggtntgaaggancaa

SEQ ID NO: 377 - protein, 37 aa, Conus geographus
  misc_feature (1)..(37): Xaa is unknown
SDXRDDTAKDEGSXMDKLVEKKECCHPACGKHYSCGR

SEQ ID NO: 378 - protein, 13 aa, Conus geographus
  PEPTIDE (1)..(13): Xaa at residue 1 is Glu or gamma-carboxy-Glu; Xaa at residue 5 is Pro or hydroxy-Pro; Xaa at residue 11 is Tyr, 125I-Tyr, mono- iodo-Tyr, di-iodo-Tyr, O-sulpho-Tyr or O-phospho-Tyr
XCCHXACGKHXSC

SEQ ID NO: 379 - DNA, 217 bp, Conus geographus
  CDS (7)..(183)
ggatccatgttcaccgtgtttctgttggtggtcttggcaaccactgtcgtttccttcccttcagaacgtgcatctgatggcagggatgacacagccaaagacgaagggtctgacatggagaaattggtcgagaaaaaagaatgttgcaatcctgcctgtggcagacacttcagttgtggacgctgatgctccaggaccctctgaaccacgactcgag

SEQ ID NO: 380 - protein, 59 aa, Conus geographus
MFTVFLLVVLATTVVSFPSERASDGRDDTAKDEGSDMEKLVEKKECCNPACGRHFSCGR

SEQ ID NO: 381 - protein, 13 aa, Conus geographus
  PEPTIDE (1)..(13): Xaa at residue 1 is Glu or gamma-carboxy-Glu; Xaa at residue 5 is Pro or hydroxy-Pro
XCCNXACGRHFSC

SEQ ID NO: 382 - DNA, 224 bp, Conus striatus
  CDS (7)..(207)
ggatccatgttcaccgtgtttctgttggttgtcttggcaaccactgtcgtttccttcacttcagatcgtgcatctgatggcagggatgacgaagccaaagacgaaaggtctgacatgcacgaatcggaccggaaaggacgcgcatactgttgccatcctgcctgtggcccaaactatagttgtggcacctcatgctccaggaccctctgaaccacgacctcgag

SEQ ID NO: 383 - protein, 67 aa, Conus striatus
MFTVFLLVVLATTVVSFTSDRASDGRDDEAKDERSDMHESDRKGRAYCCHPACGPNYSCGTSCSRTL

SEQ ID NO: 384 - protein, 22 aa, Conus striatus
  PEPTIDE (1)..(22): Xaa at residues 6 and 10 is Pro or hydroxy-Pro; Xaa at residues 2 and 12 is Tyr, 125I-Tyr, mono-iodo-Tyr, di-iodo-Tyr, O-sulpho- Tyr or O-phospho-Tyr
AXCCHXACGXNXSCGTSCSRTL

SEQ ID NO: 385 - DNA, 224 bp, Conus striatus
  CDS (7)..(189)
ggatccatgttcaccgtgtttctgttggttgtcttggcaaccactgtcgtttccttcacttcagatcgtgcatctgatggcagggatgacgaagccaaagacgaaaggtctgacatgcacgaatcggaccggaaaggacgcgcatactgttgccatcctgtctgtggcaaaaactttgattgtggacgctgatgctccaggaccctctgaaccacgacctcgag

SEQ ID NO: 386 - protein, 61 aa, Conus striatus
MFTVFLLVVLATTVVSFTSDRASDGRDDEAKDERSDMHESDRKGRAYCCHPVCGKNFDCGR

SEQ ID NO: 387 - protein, 14 aa, Conus striatus
  PEPTIDE (1)..(14): Xaa at residue 6 is Pro or hydroxy-Pro; Xaa at residue 2 is Tyr, 125I-Tyr, mono-iodo-Tyr, di-iodo-Tyr, O-sulpho-Tyr or O-phospho-Tyr
AXCCHXVCGKNFDC

SEQ ID NO: 388 - DNA, 224 bp, Conus rattus
  CDS (7)..(207)
ggatccatgttcaccgtgtttctgttggttgtcttggcaaccactgtcgtttccttcccttcagatcgtgcatctgatggcagggatgacgaagccaaagacgaaaggtctgacatgcacgaatcggaccggaatggacgcggatgctgttgcaatcctgcctgtggcccaaactatggttgtggcacctcatgctccaggaccctctgaaccacgacctcgag

SEQ ID NO: 389 - protein, 67 aa, Conus rattus
MFTVFLLVVLATTVVSFPSDRASDGRDDEAKDERSDMHESDRNGRGCCCNPACGPNYGCGTSCSRTL

SEQ ID NO: 390 - protein, 22 aa, Conus rattus
  PEPTIDE (1)..(22): Xaa at residues 6 and 10 is Pro or hydroxy-Pro; Xaa at residue 12 is Tyr, 125I-Tyr, mono-iodo-Tyr, di-iodo-Tyr, O-sulpho-Tyr or O-phospho-Tyr
GCCCNXACGXNXGCGTSCSRTL

SEQ ID NO: 391 - DNA, 230 bp, Conus arenatus
  CDS (7)..(195)
ggatccatgttcaccgtgtttctgttggttgtcttggcaaccactgtggattccttcactccagttcgtacttctgttggcaggagtgctgcagccaacgcgtttgaccggatcgctctgaccgccaggcaagattattgctgtaccattcccagctgttgggatcgctataaagagagatgtagacacatacgctgatgctccaggaccctctgaaccacgaccttgag

SEQ ID NO: 392 - protein, 63 aa, Conus arenatus
MFTVFLLVVLATTVDSFTPVRTSVGRSAAANAFDRIALTARQDYCCTIPSCWDRYKERCRHIR

SEQ ID NO: 393 - protein, 22 aa, Conus arenatus
  PEPTIDE (1)..(22): Xaa at residue 1 is Gln or pyro-Gln; Xaa at residue 16 is Glu or gamma-carboxy-Glu; Xaa at residue 8 is Pro or hydroxy-Pro; Xaa at residue 11 is Trp (D or L) or bromo-Trp (D or L);
XDXCCTIXSCXDRXKXRCRHIR

SEQ ID NO: 394 - DNA, 230 bp, Conus eburneus
  CDS (7)..(195)
ggatccatgttcaccgtgtttctgttggttgtcttggcaaccactgtggattccttcacttcagttcgtacttccgttggcaggagtgctgcagccaacgcgtttgaccggatcgctctgaccgccaggcaagattattgctgtaccattcccagctgttgggatcgctataaagagagatgtagacacatacgctgatgctccaggaccctctgaaccacgacctcgag

SEQ ID NO: 395 - protein, 63 aa, Conus eburneus
MFTVFLLVVLATTVDSFTSVRTSVGRSAAANAFDRIALTARQDYCCTIPSCWDRYKERCRHIR

SEQ ID NO: 396 - protein, 22 aa, Conus eburneus
  PEPTIDE (1)..(22): Xaa at residue 1 is Gln or pyro-Gln; Xaa at residue 16 is Glu or gamma-carboxy-Glu; Xaa at residue 8 is Pro or hydroxy-Pro; Xaa at residue 11 is Trp (D or L) or bromo-Trp (D or L);
XDXCCTIXSCXDRXKXRCRHIR

SEQ ID NO: 397 - DNA, 221 bp, Conus miles
  CDS (7)..(177)
ggatccatgttcaccgtgtttctgttggttgtcttggcaactgctgttcttccagtcactttagatcgtgcatctgatggaaggaatgcagcagccaacgccaaaacgcctcgcctgatcgcgccattcatcagggattattgctgtcatagaggtccctgtatggtatggtgtggttgaagccgctgctgctccaggaccctctgaaccacgacctcgag

SEQ ID NO: 398 - protein, 57 aa, Conus miles
MFTVFLLVVLATAVLPVTLDRASDGRNAAANAKTPRLIAPFIRDYCCHRGPCMVWCG

SEQ ID NO: 399 - protein, 13 aa, Conus miles
  PEPTIDE (1)..(13): Xaa at residue 8 is Pro or hydroxy-Pro; Xaa at residue 12 is Trp (D or L) or bromo-Trp (D or L); Xaa at residue 2 is Tyr, 125I-Tyr , mono-iodo-Tyr, di-iodo-Tyr, O-sulpho-Tyr or O-phospho-Tyr
DXCCHRGXCMVXC

SEQ ID NO: 400 - DNA, 218 bp, Conus jDedius
  CDS (7)..(174)
ggatccatgttcaccgtgtttctgttggttgtcttggcaaccactgtcgtttccaactcttcagatcgtggtccagcatctaataaaaggaagaatgccgcaatgcttgacatgatcgctcaacacgccataaggggttgctgttccgatcctcgctgtagatatagatgtcgttgaagacgctgctgctccaggaccctctgaaccacgacctcgag

SEQ ID NO: 401 - protein, 56 aa, Conus jDedius
MFTVFLLVVLATTVVSNSSDRGPASNKRKNAAMLDMIAQHAIRGCCSDPRCRYRCR

SEQ ID NO: 402 - protein, 13 aa, Conus jDedius
  PEPTIDE (1)..(13): Xaa at residue 6 is Pro or hydroxy-Pro;Xaa at residue 10 is Tyr, 125I-Tyr, mono-iodo-Tyr, di-iodo-Tyr, O-sulpho-Tyr or O-phospho-Tyr
GCCSDXRCRXRCR

SEQ ID NO: 403 - protein, 17 aa, Conus omaria
  PEPTIDE (1)..(17): Xaa at residues 6 and 13 is Pro or hydroxy-Pro
GCCSHXACNVNNXHICG

SEQ ID NO: 404 - protein, 17 aa, Conus omaria
  PEPTIDE (1)..(17): Xaa at residues 6 and 13 is Pro or hydroxy-Pro; C terminus contai ns free hydroxyl
GCCSHXACNVNNXHICG

SEQ ID NO: 405 - DNA, 224 bp, Conus quercinus
  CDS (7)..(189)
ggatccatgttcaccgtgtttctgttggttgtcttggcaaccactgtcacttcagatcgtgtatctaatggcaggaaagctgcagccaaattcaaagcgcctgccctgatggagctgtccgtcaggcaaggatgctgttcagatcctgcctgtgccgtgagcaatccagacatctgtggcggaggacgctgatgctccaggaccctctgaaccacgacctcgag

SEQ ID NO: 406 - protein, 61 aa, Conus quercinus
MFTVFLLVVLATTVTSDRVSNGRKAAAKFKAPALMELSVRQGCCSDPACAVSNPDICGGGR

SEQ ID NO: 407 - protein, 19 aa, Conus quercinus
  PEPTIDE (1)..(19): Xaa at residue 1 is Gln or pyro-Gln; Xaa at residues 7 and 14 is Pro or hydroxy-Pro
XGCCSDXACAVSNXDICGG

SEQ ID NO: 408 - DNA, 230 bp, Conus bandanus
  CDS (7)..(186)
ggatccatgttcaccgtgtttctgttggttgtcttggcaaccactgttgtttccttcacttcaaatcgtgcatttcgtcgtaggaatgccgtagccaaagcgtctgacctgatcgctctgaacgccaggagaccagaatgctgtactcatcctgcctgtcacgtgagtcatccagaactctgtggttgaagacgctgacgctccaggaccctctgaaccacgacctcgag

SEQ ID NO: 409 - protein, 60 aa, Conus bandanus
MFTVFLLVVLATTVVSFTSNRAFRRRNAVAKASDLIALNARRPECCTHPACHVSHPELCG

SEQ ID NO: 410 - protein, 17 aa, Conus bandanus
  PEPTIDE (1)..(17): Xaa at residues 2 and 15 is Glu or gamma-carboxy-Glu; Xaa at residues 1, 7 and 14 is Pro or hydroxy-Pro
XXCCTHXACHVSHXXLC

SEQ ID NO: 411 - DNA, 242 bp, Conus marmoreus
  CDS (7)..(198)
ggatccatgttcaccgtgtttctgttggttgtcttggcaaccactgttgtttccttcacttcaaatcgtgttctggatccagcatttcgtcgtaggaatgccgcagccaaagcgtctgacctgatcgctctgaacgccaggagaccagaatgctgtactcatcctgcctgtcacgtgagtaatccagaactctgtggctgaagacgctgatgctccaggaccctctgaaccacgacctcgag

SEQ ID NO: 412 - protein, 64 aa, Conus marmoreus
MFTVFLLVVLATTVVSFTSNRVLDPAFRRRNAAAKASDLIALNARRPECCTHPACHVSNPELCG

SEQ ID NO: 413 - protein, 17 aa, Conus marmoreus
  PEPTIDE (1)..(17): Xaa at residues 2 and 15 is Glu or gamma-carboxy-Glu; Xaa at residues 1, 7 and 14 is Pro or hydroxy-Pro
XXCCTHXACHVSNXXLC

SEQ ID NO: 414 - DNA, 218 bp, Conus miles
  CDS (7)..(174)
ggatccatgttcaccgtgtttctgttggttgtcttggcaaccactgtcgtttccgtcacttcatatcgtgcatctcatggcaggaaggacgcagccgacctgagcgctctgaacgacaacaataattgctgtaaccatcctgcctgtgccgggaaaaattcagatctttgtggttgaagacgctgctgctccaggaccctctgaaccacgacctcgag

SEQ ID NO: 415 - protein, 56 aa, Conus miles
MFTVFLLVVLATTVVSVTSYRASHGRKDAADLSALNDNNNCCNHPACAGKNSDLCG

SEQ ID NO: 416 - protein, 15 aa, Conus miles
  PEPTIDE (1)..(15): Xaa at residue 5 is Pro or hydroxy-Pro
CCNHXACAGKNSDLC

SEQ ID NO: 417 - protein, 16 aa, Conus magus
  PEPTIDE (1)..(16): Xaa at residue 11 is Glu or gamma-carboxy-Glu; Xaa at residue 6 is Pro or hydroxy-Pro; Xaa at residue 4 is Tyr, 125I-Tyr, mono- iodo-Tyr, di-iodo-Tyr, O-sulpho-Tyr or O-phospho-Tyr
GCCXHXTCHLXHSNLC

SEQ ID NO: 418 - DNA, 224 bp, Conus nobilis
  CDS (7)..(189)
  misc_feature (1)..(224): n is unknown
ggatccatgttcaccgtgtttctgttggttgtcttggcaaccactgttgtttccttcacttcagatcgtgcatctgatggcaggaatgccgcagccaaagcttctgacctgattgctttgaccgtcaggggatgctgtgagcgacctccctgtcgctggcaaaatccagatctttgtggtggaaggcgctganattccaggaccctctgaaccacgacctcgag

SEQ ID NO: 419 - protein, 61 aa, Conus nobilis
MFTVFLLVVLATTVVSFTSDRASDGRNAAAKASDLIALTVRGCCERPPCRWQNPDLCGGRR

SEQ ID NO: 420 - protein, 17 aa, Conus nobilis
  PEPTIDE (1)..(17): Xaa at residue 4 is Glu or gamma-carboxy-Glu; Xaa at residues 6, 7 and 13 is Pro or hydroxy-Pro; Xaa at residue 10 is Trp (D or L) or bromo-Trp (D or L)
GCCXRXXCRXQNXDLCG

SEQ ID NO: 421 - DNA, 233 bp, Conus atlanticus
  CDS (7)..(198)
ggatccatgttcaccgtgtttctgttggttgtcttggcaaccacagtcgtttccttcacttcagatagtgcatttgatagcaggaatgtcgcagccaacgacaaagtgtctgacatgatcgctctgaccgccaggagaacatgctgttcccgtcctacctgtagaatggaatatccagaactttgtggtggaagacgctgatactccaggaccctctgaaccacgacctcgag

SEQ ID NO: 422 - protein, 64 aa, Conus atlanticus
MFTVFLLVVLATTVVSFTSDSAFDSRNVAANDKVSDMIALTARRTCCSRPTCRMEYPELCGGRR

SEQ ID NO: 423 - protein, 17 aa, Conus atlanticus
  PEPTIDE (1)..(17): Xaa at residues 11 and 14 is Glu or gamma-carboxy-Glu; Xaa at residues 6 and 13 is Pro or hydroxy-Pro; Xaa at residue 12 is Tyr, 125I-Tyr, mono-iodo-Tyr, di-iodo-Tyr, O-sulpho-Tyr or O-phospho-Tyr
TCCSRXTCRMXXXXLCG

SEQ ID NO: 424 - DNA, 227 bp, Conus quercinus
  CDS (7)..(192)
  misc_feature (1)..(227): n is unknown
ggatccatgttcaccgtgtttctgttggttgtcttggcaatcacggtggtttccttcacctcagatcatgcatctgatggcaggaataccgcagccaacgacaaagcgtctaaactgatggctcttacgaacgaatgctgtgacaatcctccgtgcaagtcgagtaatccagatttgtgtgactggagaagctgatgctccaggaccctntgaaccacgacctcgag

SEQ ID NO: 425 - protein, 62 aa, Conus quercinus
MFTVFLLVVLAITVVSFTSDHASDGRNTAANDKASKLMALTNECCDNPPCKSSNPDLCDWRS

SEQ ID NO: 426 - protein, 21 aa, Conus quercinus
  PEPTIDE (1)..(21): Xaa at residue 2 is Glu or gamma-carboxy-Glu; Xaa at residues 7, 8 and 14 is Pro or hydroxy-Pro; Xaa at residue 19 is Trp (D or L) or bromo-Trp (D or L)
NXCCDNXXCKSSNXDLCDXRS

SEQ ID NO: 427 - DNA, 221 bp, Conus leopardus
  CDS (7)..(177)
ggatccatgttcaccgtgtttctgttggttgtcttggcaaccacggtcgtttccctcactttagatcgtgcatctggtggcaggagatctggagccgacaacatgattgctcttctgatcatcagaaaatgctgttccaatcccgcctgtaacaggtataatccagcaatttgtgattgaagacgctaatgctccaggaccctctgaaccacgacctcgag

SEQ ID NO: 428 - protein, 57 aa, Conus leopardus
MFTVFLLVVLATTVVSLTLDRASGGRRSGADNMIALLIIRKCCSNPACNRYNPAICD

SEQ ID NO: 429 - protein, 16 aa, Conus leopardus
  PEPTIDE (1)..(16): Xaa at residues 5 and 12 is Pro or hydroxy-Pro; Xaa at residue 10 is Tyr, 125I-Tyr, mono-iodo-Tyr, di-iodo-Tyr, O-sulpho-Tyr or O-phospho-Tyr
CCSNXACNRXNXAICD

SEQ ID NO: 430 - DNA, 224 bp, Conus emaciatus
  CDS (7)..(180)
ggatccatgttcaccgtgtttctgttggttctcttggcaaccactgtcactttacatcgtgcatctaatggcaggaatgccgcagccagcaggaaagcgtctgccctgatcgctcagatcgccggtagagactgctgtaactttcctgcttgtgccgcgagtaatccaggcctttgtacttgaagacgctgctgctccaggaccctctgaaccacgacctcgag

SEQ ID NO: 431 - protein, 58 aa, Conus emaciatus
MFTVFLLVLLATTVTLHRASNGRNAAASRKASALIAQIAGRDCCNFPACAASNPGLCT

SEQ ID NO: 432 - protein, 17 aa, Conus emaciatus
  PEPTIDE (1)..(17): Xaa at residues 6 and 13 is Pro or hydroxy-Pro
DCCNFXACAASNXGLCT

SEQ ID NO: 433 - DNA, 215 bp, Conus victor
  CDS (7)..(180)
ggatccatgttcaccgtgtttctgttggttgtcttggcaaccaccatcgtttcctccactttagatcgtgcatctgatggcatgaatgctgcagcgtctgacctgatcgctctgagcatcaggagatgctgttcttctcctccctgtttcgcgagtaatccagcttgtggtagacgacgctgatgctccaggaccctctgaaccacgacctcgag

SEQ ID NO: 434 - protein, 58 aa, Conus victor
MFTVFLLVVLATTIVSSTLDRASDGMNAAASDLIALSIRRCCSSPPCFASNPACGRRR

SEQ ID NO: 435 - protein, 14 aa, Conus victor
  PEPTIDE (1)..(14): Xaa at residues 5, 6 and 13 is Pro or hydroxy-Pro
CCSSXXCFASNXAC

SEQ ID NO: 436 - DNA, 230 bp, Conus cinereus gubba
  CDS (7)..(195)
ggatccatgttcaccgtgtttctgttggttgtcctggcaaccactatcgtttcctccacttcaggtcatgcatttgatggcaggaatgctgcagccgactacaaagggtctgaattgcttgctatgaccgtcaggggaggatgctgttcctttcctccctgtatcgcaaataatcctttttgtgctggaagacgctgatgctccaggaccctctgaaccacgacctcgag

SEQ ID NO: 437 - protein, 63 aa, Conus cinereus gubba
MFTVFLLVVLATTIVSSTSGHAFDGRNAAADYKGSELLAMTVRGGCCSFPPCIANNPFCAGRR

SEQ ID NO: 438 - protein, 17 aa, Conus cinereus gubba
  PEPTIDE (1)..(17): Xaa at residues 7, 8 and 14 is Pro or hydroxy-Pro
GGCCSFXXCIANNXFCA

SEQ ID NO: 439 - DNA, 221 bp, Conus flavidus
  CDS (7)..(177)
ggatccatgttcaccgtgtttctgttggttgtcttcgcatcctctgtcactttagatcgtgcatctcatggcaggtatatcccagtcgtcgacagagcgtctgccctgatggctcaggccgaccttagaggttgctgttccaatcctccttgttcctatcttaatccagcctgtggttaaagacgctgccgctccaggaccctctgaaccacgacctcgag

SEQ ID NO: 440 - protein, 57 aa, Conus flavidus
MFTVFLLVVFASSVTLDRASHGRYIPVVDRASALMAQADLRGCCSNPPCSYLNPACG

SEQ ID NO: 441 - protein, 15 aa, Conus flavidus
  PEPTIDE (1)..(15): Xaa at residues 6, 7 and 13 is Pro or hydroxy-Pro; Xaa at residue 10 is Tyr, 125I-Tyr, mono-iodo-Tyr, di-iodo-Tyr, O-sulpho-Tyr or O-phospho-Tyr
GCCSNXXCSXLNXAC

SEQ ID NO: 442 - DNA, 221 bp, Conus emaciatus
  CDS (7)..(177)
ggatccatgttcaccgtgtttctgttggttgtcttcgcatcctctgtcactttagatcgtgcatctcatggcaggtatgccgcagtcgtcaacagagcgtctgccctgatggctcatgccgcccttcgagattgctgttccgatcctccttgtgctcataataatccagactgtcgttaaagacgctgctgctccaggaccctctgaaccacgacctcgag

SEQ ID NO: 443 - protein, 57 aa, Conus emaciatus
MFTVFLLVVFASSVTLDRASHGRYAAVVNRASALMAHAALRDCCSDPPCAHNNPDCR

SEQ ID NO: 444 - protein, 16 aa, Conus emaciatus
  PEPTIDE (1)..(16): Xaa at residues 6, 7 and 13 is Pro or hydroxy-Pro
DCCSDXXCAHNNXDCR

SEQ ID NO: 445 - DNA, 230 bp, Conus generalis
  CDS (7)..(195)
ggatccatgttcaccgtgtttctgttggttgtcttggcaactactgtcgtttccttcacttcagatcgtgggtctgatggcaggaatgccgcagccaaggacaaagcgtctgacctggtcgctctgaccgtcaagggatgctgttctaatcctccctgttacgcgaataatcaagcctattgtaatggaagacgctgatgctccaggaccctctgaaccacgacctcgag

SEQ ID NO: 446 - protein, 63 aa, Conus generalis
MFTVFLLVVLATTVVSFTSDRGSDGRNAAAKDKASDLVALTVKGCCSNPPCYANNQAYCNGRR

SEQ ID NO: 447 - protein, 17 aa, Conus generalis
  PEPTIDE (1)..(17): Xaa at residues 6 and 7 is Pro or hydroxy-Pro; Xaa at residues 9 and 15 is Tyr, 125I-Tyr, mono-iodo-Tyr, di-iodo-Tyr, O-sulpho-Tyr or O-phospho-Tyr
GCCSNXXCXANNQAXCN

SEQ ID NO: 448 - DNA, 212 bp, Conus wittigi
  CDS (7)..(195)
ggatccatgttcaccgtgtttctgttggttgtcctggcaaccactgtcgtttcccccactagagatcgtgcatctggtgtcaggaatgttgttgcaacaagctttcagactctgacccacgatgaatgctgtgcacacccttcctgttggaaggccgaagacctgatttgtactaatcaacgtcgcaggaccctctgaaccacgacctcgag

SEQ ID NO: 449 - protein, 63 aa, Conus wittigi
MFTVFLLVVLATTVVSPTRDRASGVRNVVATSFQTLTHDECCAHPSCWKAEDLICTNQRRRTL

SEQ ID NO: 450 - protein, 25 aa, Conus wittigi
  PEPTIDE (1)..(25): Xaa at residues 2 and 13 is Glu or gamma-carboxy-Glu; Xaa at residue 7 is Pro or hydroxy-Pro; Xaa at residue 10 is Trp (D or L) or bromo-Trp (D or L)
DXCCAHXSCXKAXDLICTNQRRRTL

SEQ ID NO: 451 - DNA, 230 bp, Conus caracteristicus
  CDS (7)..(195)
ggatccatgttcaccgtgtttctgttggttgtcttggcaaccactgtcgtttccttcacttcagatcgtgcgtctgaaggcaggaatgctgcagccaaggacaaagcgtctgacctggtggctctgagagtcaggggatgctgtgccattcgtgaatgtcgcttgcagaatgcagcgtattgtggtggaatatcctgatgctccaggaccctctgaaccacgacctcgag

SEQ ID NO: 452 - protein, 63 aa, Conus caracteristicus
MFTVFLLVVLATTVVSFTSDRASEGRNAAAKDKASDLVALRVRGCCAIRECRLQNAAYCGGIS

SEQ ID NO: 453 - protein, 20 aa, Conus caracteristicus
  PEPTIDE (1)..(20): Xaa at residue 7 is Glu or gamma-carboxy-Glu; Xaa at residue 15 is Tyr, 125I-Tyr, mono-iodo-Tyr, di-iodo-Tyr, O-sulpho-Tyr or O-phospho-Tyr
GCCAIRXCRLQNAAXCGGIS

SEQ ID NO: 454 - DNA, 568 bp, Conus betulinus
  CDS (395)..(508)
  misc_feature (1)..(568): n is unknown
agtaattnatatannagaaagnaananaaaannatanagaatttaagtaatntaagaanngaganagtgaatagnagntaagtagannaaganaggtaganagnanangnggangntagntaatagatannntatngaganattantagcngtatananaagaaaagagggnaanngaaatgnngnaannataantantanngatngannngnaagtgnnaagngtanaaggaanaacaaantngttgtntaatntgnntgngtgtgtntgtgtgngtgtgtgtgtgtgngtgngtgtgtntgtgngnntgtgtgngngngngngtgtgtgtgtgngtgtgtgtgtgtgtgtgtgtgtgtgtgtgtgtgtgngtgtgtggttctggatccagcatctggtggcaggaaggctgcagccaaagcgtctaaccggatcgctctgaccgtcaggagtgcaacatgctgttattatcctccctgttacgaggcttatccagaaagttgtctgtaacgtgaatcatccagacctttgtggctgaagaccctgatgctccaggggcaagttcaa

SEQ ID NO: 455 - protein, 38 aa, Conus betulinus
SGGRKAAAKASNRIALTVRSATCCYYPPCYEAYPESCL

SEQ ID NO: 456 - protein, 19 aa, Conus betulinus
  PEPTIDE (1)..(19): Xaa at residues 12 and 16 is Glu or gamma-carboxy-Glu; Xaa at residues 8, 9 and 13 is Pro or hydroxy-Pro; Xaa at residues 6, 7, 11 and 14 is Tyr, 125I-Tyr, mono-iodo-Tyr, di-iodo-Tyr, O-sulpho-Tyr or O-phospho-Tyr
SATCCXXXXCXXAXXXSCL

SEQ ID NO: 457 - protein, 29 aa, Conus textile
DCRGYDAPCSSGAPCCDWWTCSARTNRCF

SEQ ID NO: 458 - protein, 31 aa, Conus ammiralis
GMWGDCKDGLTTCFAPSECCSEDCEGSCTMW

SEQ ID NO: 459 - protein, 36 aa, Conus ammiralis
WREGSCTSWLATCTQDQQCCTDVCYKRDYCALWDDR

SEQ ID NO: 460 - protein, 25 aa, Conus ammiralis
NCSDDWQYCESPSDCCSWDCDVVCS

SEQ ID NO: 461 - protein, 39 aa, Conus ammiralis
WWRWGGCMAWFGKCSKDSECCSNSCDITRCELMRFPPDW

SEQ ID NO: 462 - protein, 29 aa, Conus ammiralis
DCRGYDAPCSSGAPCCDWWTCSARTGRCF

SEQ ID NO: 463 - protein, 26 aa, Conus ammiralis
LCPDYTEPCSHAHECCSWNCHNGHCT

SEQ ID NO: 464 - protein, 27 aa, Conus ammiralis
CSSWAKYCEVDSECCSEQCVRSYCAMW

SEQ ID NO: 465 - protein, 32 aa, Conus pennaceus
  PEPTIDE (1)..(32): Xaa at residues 14 and 26 is gamma-carboxy-Glu; Xaa at residue 31 is hydroxy-Pro
DCTSWFGRCTVNSXCCSNSCDQTYCXLYAFXS

SEQ ID NO: 466 - protein, 32 aa, Conus gloriamaris
ECRAWYAPCSPGAQCCSLLMCSKATSRCILAL

SEQ ID NO: 467 - protein, 27 aa, Conus textile
  PEPTIDE (1)..(27): Xaa is gamma-carboxy-Glu
CKTYSKYCXADSXCCTXQCVRSYCTLF

SEQ ID NO: 468 - protein, 29 aa, Conus marmoreus
  PEPTIDE (1)..(29): Xaa is gamma-carboxy-Glu
NGQCEDVWMPCTSNWXCCSLDCEMYCTQI

SEQ ID NO: 469 - protein, 27 aa, Conus marmoreus
  PEPTIDE (1)..(27): Xaa is gamma-carboxy-Glu
CGGWSTYCEVDEXCCSESCVRSYCTLF

SEQ ID NO: 470 - protein, 26 aa, Conus marmoreus
  PEPTIDE (1)..(26): Xaa is gamma-carboxy-Glu
NGGCKATWMSCSSGWXCCSMSCDMYC

SEQ ID NO: 471 - protein, 33 aa, Conus radiatus
  PEPTIDE (1)..(33): Xaa at residues 1, 10 and 33 is bromo-Trp; Xaa at residues 5, 6, 15 and 26 is gamma-carboxy-Glu
XFGHXXCTYXLGPCXVDDTCCSASCXSKFCGLX

SEQ ID NO: 472 - protein, 31 aa, Conus radiatus
WWEGECSNWLGSCSTPSNCCLKSCNGHCTLW

SEQ ID NO: 473 - protein, 26 aa, Conus textile
  PEPTIDE (1)..(26): Xaa at residues 3 and 8 is hydroxy-Pro;  Xaa at residues 7 and 14 is gamma-carboxy-Glu
LCXDYTXXCSHAHXCCSWNCYNGHCT

SEQ ID NO: 474 - protein, 28 aa, Conus textile
DCYSWLGSCIAPSQCCSEVCDYYCRLWR

SEQ ID NO: 475 - protein, 34 aa, Conus textile
  PEPTIDE (1)..(34): Xaa  is gamma-carboxy-Glu
WLECSVWFSHCTKDSXCCSNSCDQTYCTLMPPDW

SEQ ID NO: 476 - protein, 31 aa, Conus textile
  PEPTIDE (1)..(31): Xaa is gamma-carboxy-Glu
GMWGECKDGLTTCLAPSXCCSEDCEGSCTMW

SEQ ID NO: 477 - protein, 34 aa, Conus textile
  PEPTIDE (1)..(34): Xaa is gamma-carboxy-Glu
DWWDDGCSVWGPCTVNAXCCSGDCHETCIFGWEV

SEQ ID NO: 478 - protein, 39 aa, Conus textile
  PEPTIDE (1)..(39): Xaa is gamma-carboxy-Glu
WWRWGGCMAWFGLCSRDSXCCSNSCDVTRCELMPFPPDW

SEQ ID NO: 479 - protein, 27 aa, Conus textile
  PEPTIDE (1)..(27): Xaa is gamma-carboxy-Glu
CGGYSTYCXVDSXCCSDNCVRSYCTLF

SEQ ID NO: 480 - protein, 43 aa, Conus caracteristicus
GCSGTCHRREDGKCRGTCDCSGYSYCRCGDAHHFYRGCTCSCQ

SEQ ID NO: 481 - protein, 42 aa, Conus caracteristicus
GCSGTCHRREDGKCRGTCDCSGYSYCRCGDAHHFYRGCTCTC

SEQ ID NO: 482 - protein, 42 aa, Conus caracteristicus
GCSGTCRRHRDGKCRGTCDCSGYSYCRCGDAHHFYRGCTCTC

SEQ ID NO: 483 - protein, 42 aa, Conus caracteristicus; identical to SEQ ID NO: 482

SEQ ID NO: 484 - protein, 42 aa, Conus caracteristicus; identical to SEQ ID NO: 481

SEQ ID NO: 485 - protein, 42 aa, Conus caracteristicus
GCSGTCHRRQNGECQGTCDCDGHDHCDCGDTLGTYSGCVCIC

SEQ ID NO: 486 - protein, 45 aa, Conus lacterculatus
QSETACRSLGSYQCMGKCQLGVHSWCECIYNRGSQKSGCACRCQK

SEQ ID NO: 487 - protein, 48 aa, Conus monachus
QCTLVNNCDRNGERACNGDCSCEGQICKCGYRVSPGKSGCACTCRNAK

SEQ ID NO: 488 - protein, 41 aa, Conus purpurascens
GCSGSPCFKNKTCRDECICGGLSNCWCGYGGSRGCKCTCRE

SEQ ID NO: 489 - protein, 47 aa, Conus radiatus
KCNFDKCKGTGVYNCGESCSCEGLHSCRCTYNIGSMKSGCACICTYY

SEQ ID NO: 490 - protein, 44 aa, Conus radiatus
YGLGCAGTCGSSSNCVRDYCDCPKPNCYCTGKGFRQPGCGCSCL

SEQ ID NO: 491 - protein, 46 aa, Conus striolatus
QCTFVNNCQQNGCANGDCSCGDQICKCGYRISPGRSGCACTCRNAK

SEQ ID NO: 492 - protein, 44 aa, Conus tulipa
FGPICTCFKSQNCRGSCECMSPPGCYCSNNGIRERGCSCTCPGT

SEQ ID NO: 493 - protein, 46 aa, Conus tulipa
GCTGNCDWTCSGDCSCQGTSDSCHCIPPKSIGNRCRCQCKRKIEID

SEQ ID NO: 494 - protein, 15 aa, Conus textile
ECCEDGWCCTAAPLT

SEQ ID NO: 495 - protein, 15 aa, Conus textile
GCCEDGWCCTAAPLT

SEQ ID NO: 496 - protein, 20 aa, Conus marmoreus
NGCCRAGDCCSRFEIKENDF

SEQ ID NO: 497 - protein, 11 aa, Conus marmoreus
NGCCRAGDCCS

SEQ ID NO: 498 - protein, 10 aa, Conus marmoreus
NACCIVRQCC

SEQ ID NO: 499 - protein, 10 aa, Conus quercinus
GCCARLTCCV

SEQ ID NO: 500 - protein, 10 aa, Conus quercinus
GCCAMLTCCV

SEQ ID NO: 501 - protein, 12 aa, Conus purpurascens
GCCPKQMRCCTL

SEQ ID NO: 502 - protein, 11 aa, Conus caracteristicus
CCPRRLACCII

SEQ ID NO: 503 - protein, 10 aa, Conus caracteristicus
CCPNKPCCFI

SEQ ID NO: 504 - protein, 13 aa, Conus geographus
ZGWCCKENIACCI

SEQ ID NO: 505 - protein, 13 aa, Conus geographus
ZGWCCKENIACCV

SEQ ID NO: 506 - protein, 15 aa, Conus imperialis
DWNSCCGKNPGCCPW

SEQ ID NO: 507 - protein, 11 aa, Conus betulinus
NCCPDSPPCCH

SEQ ID NO: 508 - protein, 12 aa, Conus ammiralis
GNCCEFWEFCCD

SEQ ID NO: 509 - protein, 9 aa, Conus dalli
CCEYWKLCC

SEQ ID NO: 510 - protein, 11 aa, Conus omaria
VCCGYKFFCCR

SEQ ID NO: 511 - protein, 11 aa, Conus aulicus
FCCPVIRYCCW

SEQ ID NO: 512 - protein, 11 aa, Conus aulicus
FCCPFIRYCCW

SEQ ID NO: 513 - protein, 10 aa, Conus aulicus
CCPMIYWCCS

SEQ ID NO: 514 - protein, 10 aa, Conus aulicus
CCPEIYWCCS

SEQ ID NO: 515 - protein, 10 aa, Conus nobilis
ICCPIILWCC

SEQ ID NO: 516 - protein, 9 aa, Conus ammiralis
CCPPVIWCC

SEQ ID NO: 517 - protein, 11 aa, Conus textile
CCQTFYWCCVQ

SEQ ID NO: 518 - protein, 13 aa, Conus aulicus
WNNYCCTNELWCC

SEQ ID NO: 519 - protein, 13 aa, Conus gloriamaris
LCCVTEDWCCEWW

SEQ ID NO: 520 - protein, 11 aa, Conus gloriamaris
VCCRPVQDCCS

SEQ ID NO: 521 - protein, 13 aa, Conus dalli
PVNCCPIDQSCCS

SEQ ID NO: 522 - protein, 17 aa, Conus spurius
GNIHCCTKYQPCCSSPS

SEQ ID NO: 523 - protein, 12 aa, Conus textile
ZTCCGYRMCVPC

SEQ ID NO: 524 - protein, 11 aa, Conus bandanus
ACCGYKLCSPC

SEQ ID NO: 525 - protein, 13 aa, Conus pennaceus
STCCGFKMCIPCR

SEQ ID NO: 526 - protein, 13 aa, Conus pennaceus
STCCGFKMCIPCS

SEQ ID NO: 527 - protein, 12 aa, Conus pennaceus
STCCGFKMCIPC

SEQ ID NO: 528 - protein, 12 aa, Conus episcopatus
STCCGYRMCVPC

SEQ ID NO: 529 - protein, 13 aa, Conus marmoreus
NGVCCGYKLCLPC

SEQ ID NO: 530 - protein, 12 aa, Conus pennaceus
LCCGFWMCIPCN

SEQ ID NO: 531 - protein, 13 aa, Conus marmoreus
  PEPTIDE (1)..(13): Xaa is hydroxy-Pro
NGVCCGYKLCHXC

SEQ ID NO: 532 - protein, 12 aa, Conus marmoreus
  PEPTIDE (1)..(12): Xaa is hydroxy-Pro
GVCCGYKLCHXC

SEQ ID NO: 533 - protein, 11 aa, Conus bandanus
ACCGYKLCSPC

SEQ ID NO: 534 - protein, 12 aa, Conus aulicus
SVCCGYKLCFPC

SEQ ID NO: 535 - protein, 13 aa, Conus textile
NGVCCGYRMCVPC

SEQ ID NO: 536 - protein, 12 aa, Conus textile
ZTCCGYRMCVPC

SEQ ID NO: 537 - protein, 12 aa, Conus ammiralis
ZACCGFKMCVPC

SEQ ID NO: 538 - protein, 13 aa, Conus pennaceus
STCCGFKMCIPCR

SEQ ID NO: 539 - protein, 14 aa, Conus pennaceus
NGVCCGFWMCIPCN

SEQ ID NO: 540 - protein, 12 aa, Conus omaria
DVCCYVRMCPCR

SEQ ID NO: 541 - protein, 7 aa, Conus imperialis
ZCGQAWC

SEQ ID NO: 542 - protein, 8 aa, Conus stercusmuscarum
  PEPTIDE (1)..(8): Xaa is hydroxy-Pro
GCXWQPVC

SEQ ID NO: 543 - protein, 11 aa, Conus arenatus
  PEPTIDE (1)..(11): Xaa is hydroxy-Pro
ZYGCXXGLWCH

SEQ ID NO: 544 - protein, 10 aa, Conus arenatus
ASGCPWRPWC

SEQ ID NO: 545 - protein, 11 aa, Conus arenatus
ZYGCPVGLWCD

SEQ ID NO: 546 - protein, 9 aa, Conus arenatus
SGCPWQPWC

SEQ ID NO: 547 - protein, 9 aa, Conus arenatus
SGCPWHPWC

SEQ ID NO: 548 - protein, 30 aa, Conus ermineus
  PEPTIDE (1)..(30): Xaa is hydroxy-Pro
GCCGKYXNAACHXCGCTVGRXXYCDRXSGG

SEQ ID NO: 549 - protein, 25 aa, Conus purpurascens
GCCGSYPNAACHPCGCKDRPSYCGQ

SEQ ID NO: 550 - protein, 23 aa, Conus purpurascens
EGCCSNPACHPCGCKDRPSYCGQ

SEQ ID NO: 551 - protein, 32 aa, Conus arenatus
VVTEACEESCEEEEKHCCHVNNGVPSCAVICW

SEQ ID NO: 552 - protein, 32 aa, Conus arenatus
IVTEACEESCEDEEKHCCHVNNGVPSCAVICW

SEQ ID NO: 553 - protein, 32 aa, Conus arenatus
IVTEACEEHCEDEEQFCCGLENGQPFCAPVCF

SEQ ID NO: 554 - protein, 32 aa, Conus arenatus
VVTGACEEHCEDEEKHCCGLENGQPFCARLCL

SEQ ID NO: 555 - protein, 32 aa, Conus distans
NVDQECIDACQLEDKNCCGRTDGEPRCAKICL

SEQ ID NO: 556 - protein, 32 aa, Conus distans
ETDQECIDICKQEDKKCCGRSNGEPTCAKICL

SEQ ID NO: 557 - protein, 32 aa, Conus distans
ETDQECIDTCEQEDKKCCGRTNGEPVCAKICF

SEQ ID NO: 558 - protein, 37 aa, Conus purpurascens
PKTEACEEVCELEEKHCCCIRSDGPKCSRKCLLSIFC

SEQ ID NO: 559 - protein, 32 aa, Conus purpurascens
VVSEECKKYCKKQNKNCCSSKHEEPRCAKICF

SEQ ID NO: 560 - protein, 32 aa, Conus sponsalis
AVTEACTEDCKTQDKKCCGEMNGQHTCAKICL

SEQ ID NO: 561 - protein, 37 aa, Conus tulipa
PKTKECERYCELEEKHCCCIRSNGPKCSRICIFKFWC

SEQ ID NO: 562 - protein, 36 aa, Conus tulipa
PKTRECEMQCEQEEKHCCRVRDGTGQCAPKCLGINW

SEQ ID NO: 563 - protein, 9 aa, Conus geographus
CFIRNCPKG

SEQ ID NO: 564 - protein, 9 aa, Conus striatus
CIIRNCPRG

SEQ ID NO: 565 - protein, 28 aa, Conus arenatus
GCTPPGGVCGYHGHCCDFCDTFGNLCVS

SEQ ID NO: 566 - protein, 27 aa, Conus geographus
ACSGRGSRCPPQCCMGLTCGREYPPRC

SEQ ID NO: 567 - protein, 31 aa, Conus caracteristicus
NCGEQGEGCATRPCCSGLSCVGSRPGGLCQY

SEQ ID NO: 568 - protein, 23 aa, Conus textile
NCPYCVVYCCPPAYCEASGCRPP

SEQ ID NO: 569 - protein, 27 aa, Conus delesstii
  PEPTIDE (1)..(27): Xaa at residue 4 is hydroxy-Pro; Xaa at residues 13 and 16 is gamm-carboxy-Glu
ACKXKNNLCAITXMAXCCSGFCLIYRC

SEQ ID NO: 570 - protein, 30 aa, Conus lividus
SCGHSGAGCYTRPCCPGLHCSGGQAGGLCV

SEQ ID NO: 571 - protein, 35 aa, Conus lividus
DCGESGQGCYSVRPCCPGLICKGTGGGGLCRPSGI

SEQ ID NO: 572 - protein, 31 aa, Conus miliaris
CTPPGGLCYHAYPCCSKTCNLDTSQCEPRWS

SEQ ID NO: 573 - protein, 36 aa, Conus miles
CTDDSQFCNPSNHDCCSGKCIDEGDNGICAIVPENS

SEQ ID NO: 574 - protein, 36 aa, Conus miles
CTEDSQFCNPSNHDCCSGKCIDEGDNGICAIVPENS

SEQ ID NO: 575 - protein, 30 aa, Conus pulicarius
CSDFGSDCVPATHNCCSGECFGFEDFGLCT

SEQ ID NO: 576 - protein, 30 aa, Conus quercinus
ACSQVGEACFPQKPCCPGFLCNHIGGMCHH

SEQ ID NO: 577 - protein, 28 aa, Conus striatus
CLPDGTSCLFSRIRCCGTCSSILKSCVS

SEQ ID NO: 578 - protein, 31 aa, Conus tessulatus
SCAEFGEVCSSTACCPDLDCVEAYSPICLWE

SEQ ID NO: 579 - protein, 27 aa, Conus textile
KCVEQWKYCTRESLCCAGLCLFSFCIL

SEQ ID NO: 580 - protein, 29 aa, Conus textile
CVEQWEVCGIILFSSSCCGQLCLFGFCVL

SEQ ID NO: 581 - protein, 32 aa, Conus virgo
DCGGQGEGCYTQPCCPGLRCRGGGTGGGVCQL

SEQ ID NO: 582 - protein, 33 aa, Conus wittigi
FGSFIPCARLGEPCTICCRPLRCRESGTPTCQV

SEQ ID NO: 583 - protein, 30 aa, Conus regius
TCLEHNKLCWYDRDCCTIYCNENKCGVKPQ

SEQ ID NO: 584 - protein, 34 aa, Conus radiatus
ACKSNYDCPQRFKCCSYTWNGSSGYCKRVCYLYR

SEQ ID NO: 585 - protein, 24 aa, Conus purpurascens
  PEPTIDE (1)..(24): Xaa is hydroxy-Pro
GXXCCLYGSCRXFXGCYNALCCRK

SEQ ID NO: 586 - protein, 24 aa, Conus purpurascens
HPPCCMYGRCRRYPGCSSASCCQG

SEQ ID NO: 587 - protein, 36 aa, Conus consors
APELVVTATTTCCGYDPMTICPPCMCTHSCPPKRKP

SEQ ID NO: 588 - protein, 39 aa, Conus aurisiacus
ZSWLVPSTITTCCGYDPGTMCPPCRCNNTCKPKKPKPGK

SEQ ID NO: 589 - protein, 37 aa, Conus consors
APELVVTATTTCCGYDPMTWCPSCMCTYSCPHQRKKP

SEQ ID NO: 590 - protein, 36 aa, Conus magus
APELVVTATTTCCGYDPMTICPPCMCTHSCPPKGKP

SEQ ID NO: 591 - protein, 38 aa, Conus aurisiacus
ZKWLVHSKITYCCGYNKMDMCPPCMCTYSCPPLKKKRP

SEQ ID NO: 592 - protein, 29 aa, Conus aurisiacus
APWTVVTATTNCCGITGPGCLPCRCTQTC

SEQ ID NO: 593 - protein, 13 aa, Conus geographus
ECCHPACGKHYSC

SEQ ID NO: 594 - protein, 13 aa, Conus geographus
ECCNPACGRHFSC

SEQ ID NO: 595 - protein, 22 aa, Conus striatus
AYCCHPACGPNYSCGTSCSRTL

SEQ ID NO: 596 - protein, 14 aa, Conus striatus
AYCCHPVCGKNFDC

SEQ ID NO: 597 - protein, 22 aa, Conus rattus
GCCCNPACGPNYGCGTSCSRTL

SEQ ID NO: 598 - protein, 22 aa, Conus arenatus
ZDYCCTIPSCWDRYKERCRHIR

SEQ ID NO: 599 - protein, 22 aa, Conus eburneus
ZDYCCTIPSCWDRYKERCRHIR

SEQ ID NO: 600 - protein, 13 aa, Conus miles
DYCCHRGPCMVWC

SEQ ID NO: 601 - protein, 13 aa, Conus jDedius
GCCSDPRCRYRCR

SEQ ID NO: 602 - protein, 17 aa, Conus omaria
GCCSHPACNVNNPHICG

SEQ ID NO: 603 - protein, 17 aa, Conus omaria
GCCSHPACNVNNPHICG

SEQ ID NO: 604 - protein, 19 aa, Conus quercinus
ZGCCSDPACAVSNPDICGG

SEQ ID NO: 605 - protein, 17 aa, Conus bandanus
PECCTHPACHVSHPELC

SEQ ID NO: 606 - protein, 17 aa, Conus marmoreus
PECCTHPACHVSNPELC

SEQ ID NO: 607 - protein, 15 aa, Conus miles
CCNHPACAGKNSDLC

SEQ ID NO: 608 - protein, 16 aa, Conus magus
GCCYHPTCHLEHSNLC

SEQ ID NO: 609 - protein, 17 aa, Conus nobilis
GCCERPPCRWQNPDLCG

SEQ ID NO: 610 - protein, 17 aa, Conus atlanticus
TCCSRPTCRMEYPELCG

SEQ ID NO: 611 - protein, 21 aa, Conus quercinus
NECCDNPPCKSSNPDLCDWRS

SEQ ID NO: 612 - protein, 16 aa, Conus leopardus
CCSNPACNRYNPAICD

SEQ ID NO: 613 - protein, 17 aa, Conus emaciatus
DCCNFPACAASNPGLCT

SEQ ID NO: 614 - protein, 14 aa, Conus victor
CCSSPPCFASNPAC

SEQ ID NO: 615 - protein, 17 aa, Conus cinereus gubba
GGCCSFPPCIANNPFCA

SEQ ID NO: 616 - protein, 15 aa, Conus flavidus
GCCSNPPCSYLNPAC

SEQ ID NO: 617 - protein, 16 aa, Conus emaciatus
DCCSDPPCAHNNPDCR

SEQ ID NO: 618 - protein, 17 aa, Conus generalis
GCCSNPPCYANNQAYCN

SEQ ID NO: 619 - protein, 25 aa, Conus wittigi
DECCAHPSCWKAEDLICTNQRRRTL

SEQ ID NO: 620 - protein, 20 aa, Conus caracteristicus
GCCAIRECRLQNAAYCGGIS

SEQ ID NO: 621 - protein, 19 aa, Conus betulinus
SATCCYYPPCYEAYPESCL

SEQ ID NO: 622 - protein, 6 aa, Conus magus
  PEPTIDE (1)..(6): Xaa is gamma-carboxy-Glu
VYXTHP

SEQ ID NO: 623 - protein, 14 aa, Conus striatus
WSWRMGNGDRRSDQ

SEQ ID NO: 624 - protein, 11 aa, Conus quercinus
DCQPCGHNVCC

SEQ ID NO: 625 - protein, 29 aa, Conus geographus
  PEPTIDE (1)..(29): Xaa is gamma-carboxy-Glu
KFLSGGFKXIVCHRYCAKGIAKEFCNCPD

SEQ ID NO: 626 - protein, 6 aa, Conus magus
RPKNSW

SEQ ID NO: 627 - protein, 7 aa, Conus magus
  PEPTIDE (1)..(7): Xaa is hydroxy-Pro
ARXKNSW

SEQ ID NO: 628 - protein, 6 aa, Conus magus
  PEPTIDE (1)..(6): Xaa is hydroxy-Pro
RXKNSW

SEQ ID NO: 629 - protein, 71 aa, Conus geographus; identical to SEQ ID NO: 117

SEQ ID NO: 630 - protein, 65 aa, Conus geographus
DESKCDRCNCAELRSSRCTQAIFCLTPELCTPSISCPTGECRCTKFHQSRCTRFVECVPNKCRDA

SEQ ID NO: 631 - protein, 60 aa, Conus geographus
DDSYCDGCLCTILKKETCTSTMSCRGTCRKEWPCWEEDCYCTEIQGGACVTPSECKPGEC

SEQ ID NO: 632 - protein, 43 aa, Conus radiatus
GCVYEGIEYSVGETYQADCNTCRCDGFDLATCTVAGCTGFGPE

SEQ ID NO: 633 - protein, 86 aa, Conus striatus
SGPADCCRMKECCTDRVNECLQRYSGREDKFVSFCYQEATVTCGSFNEIVGCCYGYQMCMIRVVKPNSLSGAHEACKTVSCGNPCA

SEQ ID NO: 634 - protein, 24 aa, Conus purpurascens
DCCGVKLEMCHPCLCDNSCKNYGK

SEQ ID NO: 635 - protein, 36 aa, Conus geographus
GEPIPTTVINYGECCKDPSCWVKVKDFQCPGASPPN

SEQ ID NO: 636 - protein, 16 aa, Conus generalis
QCCTFCNFGCQPCCVP

SEQ ID NO: 637 - protein, 20 aa, Conus tessulatus
DGCPPHPVPGMHKCMCTNTC

SEQ ID NO: 638 - protein, 84 aa, Conus radiatus
HPTKPCMYCSFGQCVGPHICCGPTGCEMGTAEANMCSEEDEDPIPCQVFGSDCALNNPDNIHGHCVADGICCVDDTCTTHLGCL